(19) United States
(12) Patent Application Publication
(10) Pub. No.: US 2003/0013660 A1
(43) Pub. Date: Jan. 16, 2003

(54) Dioxolane analogs for improved inter-cellular delivery

(75) Inventors: Giorgio Attardo, Laval (CA); Boulos Zacharie, Laval (CA); Rabindra Rej, Montreal (CA); Jean-Francois Lavallee, Mille-Isles (CA); Louis Vaillancourt, Mascouche (CA); Real Denis, Montreal (CA); Sophie Levesque, Mirabel (CA)

Correspondence Address: MILLEN, WHITE, ZELANO & BRANIGAN, P.C., 2200 CLARENDON BLVD., SUITE 1400, ARLINGTON, VA, 22201, US

(73) Assignee: Shire BioChem Inc., Laval (CA)

(21) Appl. No.: 09/976,249

(22) Filed: Oct. 15, 2001

Related U.S. Application Data
(60) Provisional application No. 60/288,424, filed on May 4, 2001.
(60) Provisional application No. 60/239,885, filed on Oct. 13, 2000.

(51) Int. Cl. A61K 31/675; A61K 31/665; A61K 31/522; A61K 38/05; A61K 38/06; A61K 31/513; A61K 31/404; A61K 31/4025; A61K 31/357

(52) U.S. Cl. 514/18; 514/19; 514/81; 514/86; 514/101; 514/263.23; 514/269; 514/467; 514/414; 514/422

(57) ABSTRACT

Dioxolane analogs of the following formula:

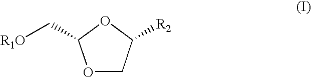

wherein R1 and R2 are defined herein, are useful in the treatment of cancer. For example, the compounds can be used to treat patients with cancer in which the cancer cells are deficient in nucleoside or nucleoside base transporters.

### FIELD OF THE INVENTION

The present invention is related to nucleoside analogs for treating cancer, in particular dioxolane nucleoside analogs.

### BACKGROUND OF THE INVENTION

Neoplastic diseases, characterized by the proliferation of cells not subject to the normal control of cell growth, are a major cause of death in humans. In the United States only, a total of over about 1 million new cancer cases occurred for the year of 1995 (CA, Cancer J. Clin., 1995:45:8:30) cancer deaths in the United States for 1995 was more than about 500,000.

The usefulness of known cytotoxic agents is compromised by dose limiting toxicities such as myelosuppression as well as the resistance of treated tumors. In view of the proven effectiveness of chemotherapy in the treatment of responsive tumors, efforts have been undertaken to develop novel compounds with either an improved therapeutic index or with reduced cross-resistance.

Antimetabolites, such as nucleoside analogs, have been used in anticancer treatment regimens. Some of the more commonly used analogs include gemcitabine (dFdC), 5-fluorouracil (5-FU), cytosine arabinoside (Ara-C, cytarabine), 6-thioguanine (TG) and 6-mercaptopurine (MP). This class of compounds is generally toxic to adult tissues that retain a high rate of cell proliferation: bone marrow, intestinal mucosa, hair follicles and gonads.

5-FU is used most commonly in breast and gastrointestinal cancer patients. Major side effects associated with 5-FU administration include bone marrow and mucous membrane toxicities; and minor side effects include skin rashes, conjunctivitis and ataxia. Ara-C, used in the treatment of acute myelocytic leukemia, may cause myelosuppression and gastrointestinal toxicity. TG and MP, used primarily in leukemia patients and rarely in solid tumors, are associated with toxicities similar to that of Ara-C.

β-D-ddC has been investigated by Scanlon et al. in circumvention of human tumor drug resistance (WO 91/07180). Human leukemia cells resistant to cisplatin have shown enhanced sensitivity to β-D-ddC. However, β-D-ddC has been linked to the development of peripheral neuropathy (Yarchoan, et al, Lancet, i:76, 1988) and therefore exhibits in vivo toxicity.

More recently, β-L-Dioxolane cytidine (troxacitabine) was reported to demonstrate anticancer activity (Grove et al. Cancer Research 55, 3008-3011, Jul. 15, 1995). There is therefore a need for anticancer agents that are easy to synthesize and display an improved therapeutic index and efficacy against refractory tumors.

### SUMMARY OF THE INVENTION

It is known that gemcitabine and cytarabine enter cancer cells by nucleoside or nucleobase transporter proteins. Mackey et al., supra; White et al. (1987). J. Clin. Investig. 79, 380-387; Wiley et al. (1982); J. Clin. Investig. 69, 479-489; and Gati et al. (1997), Blood 90, 346-353. Further, it has been reported that troxacitabine also enters cancer cells by way of nucleoside or nucleobase transporter proteins (NTs). [Grove et al., Cancer Research (56), p. 4187-91 (1996)] However, recent studies show that troxacitabine actually enters cancer cells predominately by the mechanism of passive diffusion, rather than by nucleoside transporters. Cytarabine may also enter cells by passive diffusion, but only during a high-dose therapy regimen.

Also, resistance of cancer cells to treatment by anticancer agents has been linked to a deficiency of nucleoside or nucleobase transporter proteins in the cancer cells. (Mackey et al. (1998), supra; Mackey et al. (1998b). Drug Resistance Updates 1, 310-324; Ullman et al. (1988), J. Biol. Chem. 263, 12391-12396; and references cited above.

Thus, in accordance with the invention, cancer treatments are provided in which the anticancer agents utilized enter cells by mechanisms other than through the use of nucleoside or nucleobase transporter proteins, particularly by passive diffusion. Transport through the cell membrane is facilitated by the presence of lipophilic structures. Thus, in accordance with the invention, entry of anticancer agents into cancer cells by passive diffusion is enhanced by providing the agents with lipophilic structures.

Further, in accordance with the invention, patients with cancers resistant to agents that are transported by nucleoside or nucleobase transporter proteins can be treated with anticancer agents that enter the cells predominately by passive diffusion.

Further, in accordance with the invention, patients with cancers resistant to agents that are transported by nucleoside or nucleobase transporter proteins can be treated with dosages of anticancer agents that increase the entry into the cells by passive diffusion.

In accordance with one aspect of the invention, there is provided a method of treating a patient having a cancer which is resistant to gemcitabine, cytarabine, or both, by administering an anticancer agent that enters the cell predominately by a mechanism other than via nucleoside or nucleobase transporter proteins, particularly by passive diffusion. In the context of the invention, predominately means that the agent enters the cell by the specified mechanism to a greater degree than any one of the other individual transport mechanisms does.

In accordance with another aspect of the invention, there is provided a method of treating a patient having a cancer in which the cancer cells are deficient in nucleoside or nucleobase transporter proteins by administering an anticancer agent that enters the cell predominately by a mechanism other than via nucleoside or nucleobase transporter proteins, particularly that enter the cells predominately by passive diffusion.

In accordance with another aspect of the invention, there is provided a method of treating a patient having a cancer which is resistant to gemcitabine, cytarabine, and/or troxacitabine, by administering to the patient an anticancer agent, for example, a gemcitabine, cytarabine or troxacitabine derivative, that possesses a lipophilic structure to facilitate entry thereof into the cancer cells, particularly by passive diffusion. In accordance with another aspect of the invention, there is provided a method of treating a patient having a cancer, which is resistant to troxacitabine because of poor uptake, by administering an anticancer agent, for example, a troxacitabine derivative, which has a greater lipophilicity than troxacitabine.

According to a further aspect of the invention, there is provided a method for treating a patient having a cancer that is resistant to gemcitabine and/or cytarabine comprising administering to said patient a dioxolane nucleoside compound of the following formula (I):

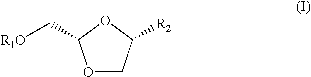

wherein:

R₁ is H; C₁₋₂₄ alkyl; C₂₋₂₄ alkenyl; C₆₋₂₄ aryl; trityl; C₆₋₂₄-aryl-C₁₋₂₄-alkyl; C₆₋₂₄-aryl-C₂₋₂₄-alkenyl; C₅₋₂₀ heteroaromatic ring; C₃-₂₀ non-aromatic ring optionally containing 1-3 heteroatoms selected from the group comprising O, N, or S; —C(O)R₆; —C(O)OR₆; —C(O)NHR₆; or an amino acid radical or a dipeptide or tripeptide chain or mimetic thereof, wherein the amino acid radicals are selected from the group comprising Glu, Gly, Ala, Val, Leu, Ile, Pro, Phe, Tyr, Trp, Ser, Thr, Cys, Met, Asn and Gln (the amino acid chain preferably contains at least one amino acid other than Gly), and which in each case is optionally terminated by —R₇;

R₁ can also be a P(O) (OR′)₂ group wherein R′ is in each case independently H, C₁₋₂₄ alkyl, C₂₋₂₄ alkenyl, C₆₋₂₄ aryl, C₇₋₁₈ arylmethyl, C₂₋₁₈ acyloxymethyl, C₃₋₈ alkoxycarbonyloxymethyl, or C₃₋₈ S-acyl-2-thioethyl, saleginyl, t-butyl, phosphate or diphosphate;

R₁ can also be monophosphate, diphosphate, triphosphate or mimetics thereof;

R₂ is

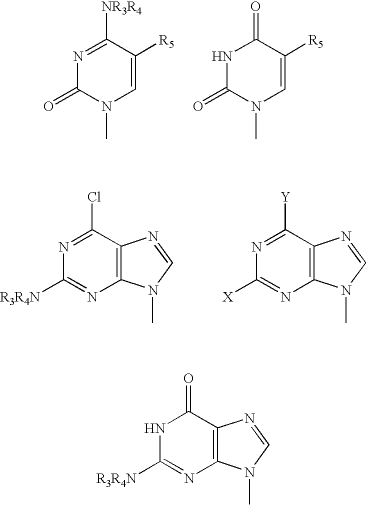

R₃ and R₄ are in each case independently H; C₁₋₂₄ alkyl; C₂₋₂₄ alkenyl; C₆₋₂₄ aryl; C₆₋₂₄-aryl-C₁₋₂₄-alkyl; C₆₋₂₄-aryl-C₂₋₂₄-alkenyl; C₅₋₁₈ heteroaromatic ring; C₃₋₂₀ non-aromatic ring optionally containing 1-3 heteroatoms selected from the group comprising O, N, or S; —C(O)R₆; —C(O)OR₆; —C(O)NHR₆ or an amino acid radical or a dipeptide or tripeptide chain or mimetics thereof, wherein the amino acids radicals are selected from the group comprising Glu, Gly, Ala, Val, Leu, Ile, Pro, Phe, Tyr, Trp, Ser, Thr, Cys, Met, Asn and Gln (the amino acid chain preferably contains at least one amino acid other than Gly), and which in each case is optionally terminated by —R₇;

R₃ and R₄ together can also be ═CH—N(C₁₋₄-alkyl)₂;

R₆ is, in each case, H, C₁₋₂₄ alkyl, C₂₋₂₄ alkenyl,

C₀₋₂₄ alkyl-C₆₋₂₄ aryl, C₆₋₂₄-aryl-C₁₋₂₄-alkyl; C₆₋₂₄-aryl-C₂₋₂₄-alkenyl; C₀₋₂₄ alkyl-C₅₋₂₀ heteroaromatic ring, C₃₋₂₀ non-aromatic ring optionally containing 1-3 heteroatoms selected from the group comprising O, N or S;

R₇ is, in each case, C₁₋₂₄ alkyl, C₂₋₂₄ alkenyl, C₆₋₂₄ aryl, C₆₋₂₄-aryl-C₁₋₂₄-alkyl; C₆₋₂₄-aryl-C₂₋₂₄-alkenyl; C₅₋₂₀ heteroaromatic ring, C₃₋₂₀ non-aromatic ring optionally containing 1-3 heteroatoms selected from the group comprising O, N or S, —C(O)R₆ or —C(O)OR₆; and

X and Y are each independently Br, Cl, I, F, OH, OR₃ or NR₃R₄ and at least one of X and Y is NR₃R₄; or

a pharmaceutically acceptable salt thereof.

According to a further aspect of the invention, there is provided a method for treating a patient having a cancer that is resistant to gemcitabine, cytarabine and/or troxacitabine comprising administering to the patient a compound according to formula (I) wherein at least one of R₁, R₃ and R₄ is other than H, and if R₃ and R₄ are both H and R₁ is —C(O)R₆ or —C(O)OR₆, then R₆ is other than H.

According to a further aspect of the invention, there is provided a method of treating a patient with cancer, wherein the cancer cells are deficient in one or more nucleoside or nucleobase transporter proteins, comprising administering to the patient a compound according to formula (I). According to a further aspect of the invention, there is provided a method for treating a patient with cancer, wherein the cancer cells are deficient in nucleoside or nucleobase transporter proteins, comprising administering to the patient a compound according to formula (I), wherein at least one of R₁, R₃ and R₄ is other than H, and if R₃ and R₄ are both H and R₁ is —C(O)R₆ or —C(O)OR₆, then R₆ is other than H.

In accordance with another aspect of the invention, there is provided a method for treating a patient with cancer, comprising determining that a compound enters cancer cells predominately by passive diffusion, and administering the compound to the patient, wherein the compound is a compound according to the formula (I). In accordance with another aspect of the invention, there is provided a method for treating a patient with cancer, comprising administering to the patient a compound which has been determined to enter cancer cells predominately by passive diffusion, wherein the compound is in accordance with formula (I). In accordance with a further aspect of the invention, there is provided a method of treating a patient with cancer, comprising determining that a compound does not enter cancer cells predominately by nucleoside or nucleobase transporter proteins, and administering the compound to the patient, wherein the compound is a compound according to the formula (I).

In accordance with an additional aspect of the invention there are provided anticancer compounds having lipophilic structures, wherein the compounds are of the following formula (I′):

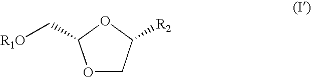

wherein:

R₁ is H; C₁₋₂₄ alkyl; C₂₋₂₄ alkenyl; C₆₋₂₄ aryl; trityl; C₆₋₂₄-aryl-C₁₋₂₄-alkyl; C6-24-aryl-C₂₋₂₄-alkenyl; C₅₋₂₀ heteroaromatic ring; C₃₋₂₀ non-aromatic ring optionally containing 1-3 heteroatoms selected from the group comprising O, N, or S; —C(O)R₆; —C(O)OR₆; —C(O)NHR₆; or an amino acid radical or a dipeptide or tripeptide chain or mimetic thereof, wherein the amino acid radicals are selected from the group comprising Glu, Gly, Ala, Val, Leu, Ile, Pro, Phe, Tyr, Trp, Ser. Thr, Cys, Met, Asn and Gln (the amino acid chain preferably contains at least one amino acid other than Gly), and which in each case is optionally terminated by —R₇;

R₁ can also be a P(O) (OR′)₂ group wherein R′ is in each case independently H, C₁l₂₄ alkyl, C₂₂₄ alkenyl, C6-24 aryl, C₇₋₁₈ arylmethyl, C₂₋₁₈ acyloxymethyl, C₃₋₈ alkoxycarbonyloxymethyl, or C₃₋₈ S-acyl-2-thioethyl, saleginyl, t-butyl, phosphate or diphosphate;

R₁ can also be monophosphate, diphosphate, triphosphate or mimetics thereof;

R₂ is

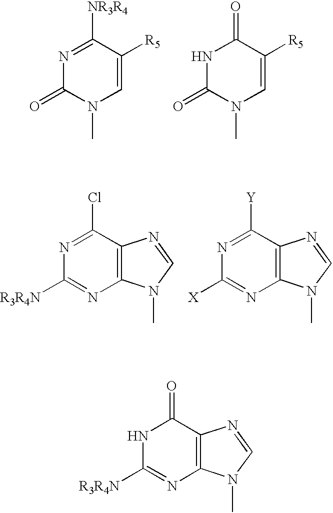

R₃ and R₄ are in each case independently H; C₁₋₂₄ alkyl; C₂₋₂₄ alkenyl; C₆₋₂₄ aryl; C₆₋₂₄-aryl-C₁₋₂₄-alkyl; C₆₋₂₄-aryl-C₂₋₂₄-alkenyl; C₅₋₁₈ heteroaromatic ring; C₃₋₂₀ non-aromatic ring optionally containing 1-3 heteroatoms selected from the group comprising O, N, or S; —C(O)R₆; —C(O)OR₆; —C(O)NHR₆ or an amino acid radical or a dipeptide or tripeptide chain or mimetics thereof, wherein the amino acids radicals are selected from the group comprising Glu, Gly, Ala, Val, Leu, Ile, Pro, Phe, Tyr, Trp, Ser. Thr, Cys, Met, Asn and Gin (the amino acid chain preferably contains at least one amino acid other than Gly), and which in each case is optionally terminated by —R₇;

R₃ and R₄ together can also be ═CH—N(C₁₋₄-alkyl)₂;

R₆ is, in each case, H, C₁₋₂₄ alkyl, C₂₋₂₄ alkenyl, C₀₋₂₄ alkyl-C₆₋₂₄ aryl, C₆₋₂₄-aryl-C₁₋₂₄-alkyl; C₆₋₂₄-aryl-C₂₋₂₄-alkenyl; C₀₋₂₄ alkyl-C₅₋₂₀ heteroaromatic ring, C₃₋₂₀ non-aromatic ring optionally containing 1-3 heteroatoms selected from the group comprising O, N or S;

R₇ is, in each case, C₁₋₂₄ alkyl, C₂₋₂₄ alkenyl, C₆₋₂₄ aryl, C₆₋₂₄-aryl-C₁₋₂₄-alkyl; C₆₋₂₄-aryl-C₂₋₂₄-alkenyl; C₅₋₂₀ heteroaromatic ring, C₃₋₂₀ non-aromatic ring optionally containing 1-3 heteroatoms selected from the group comprising O, N or S, —C(O)R₆ or —C(O)OR₆; and

X and Y are each independently Br, Cl, I, F, OH, OR₃ or NR₃R₄ and at least one of X and Y is NR₃R₄; or a pharmaceutically acceptable salt thereof.

X and Y are each independently Br, Cl, I, F, OH, OR₃ or NR₃R₄ and at least one of X and Y is NR₃R₄; or a pharmaceutically acceptable salt thereof;

with the proviso that at least one of R₁, R₃ and R₄ is C7-24 alkyl;

C₇-₂₄ alkenyl;

C₆₋₂₄ aryl;

C₅₋₂₀ heteroaromatic ring;

C₄₋₂₀ non-aromatic ring optionally containing 1-3 heteroatoms selected from the group comprising O, N, or S;

—C(O)R₆ in which R₆ is, C₇₋₂₄ alkyl, C₇₋₂₄ alkenyl, C₀₋₂₄ alkyl-C₆₋₂₄ aryl, C₆₋₂₄-aryl-C₁₋₂₄-alkyl; C₆₋₂₄-aryl-C₂₋₂₄-alkenyl; C₀₋₂₄ alkyl-C₅₋₂₀ heteroaromatic ring, C₃₋₂₀ non-aromatic ring optionally containing 1-3 heteroatoms selected from the group comprising O, N or S;

—C(O)OR₆ in which R₆ is C₇₋₂₄ alkyl, C₇₋₂₄ alkenyl, C₀₋₂₄ alkyl-C₆₋₂₄ aryl, C₆₋₂₄-aryl-C₁₋₂₄-alkyl; C₆₋₂₄-aryl-C₂₋₂₄-alkenyl; C₀₋₂₄ alkyl-C₅₋₂₀ heteroaromatic ring, C₃₋₂₀ non-aromatic ring optionally containing 1-3 heteroatoms selected from the group comprising O, N or S; or

a dipeptide or tripeptide or mimetic thereof where the amino acid radicals are selected from the group comprising Glu, Gly, Ala, Val, Leu, Ile, Pro, Phe, Tyr, Trp, Ser, Thr, Cys, Met, Asn and Gln (and the amino acid chain preferably contains at least one amino acid other than Gly), and which is optionally terminated by —R₇.

In an embodiment of the present invention, the R₆ group is connected to the rest of the molecule at a tertiary or quaternary carbon. A tertiary carbon is defined as a carbon atom which has only one hydrogen atom directly attached to it. A quaternary carbon is defined as a carbon atom with no hydrogen atoms attached to it.

In an alternate embodiment of the present invention, the R₆ group is selected as to provide steric hindrance in the vicinity of the carbonyl group.

Upon further study of the specification and claims, further aspects and advantages of the invention will become apparent to those skilled in the art.

As mentioned above, recent studies have shown that troxacitabine, a L-nucleoside analog, enters cancer cells predominately by passive diffusion, rather than by nucleoside or nucleobase transporter proteins. While this invention is not intended to be limited by any theoretical explanation, it is believed that this property of troxacitabine is at least in part attributed to the dioxolane structure. Further, due to its L-configuration, troxacitabine is a poor substrate for deoxycytidine deaminase. (Grove et al. (1995), Cancer Res. 55, 3008-3011) Formula (I) encompasses compounds which are nucleoside analogs having a dioxolane structure and which exhibit the L-configuration. In addition, formula (I) encompasses compounds which exhibit a lipophilic structure. In the case of compounds encompassed by formula (I), the lipophilic structures are provided through modification of the hydroxymethyl structure of the dioxolane sugar moiety and/or modification of amino groups of the base moiety.

In the compounds of formula (I), preferably at least one of R¹, R³ and R⁴ provides a lipophilic structure. Thus, preferably at least one of R¹, R³ and R⁴ is other than H and, if R³ and R⁴ are each H and R¹ is C(O)R⁶, C(O)OR⁶ or C(O)NHR⁶ then R⁶ is other than H.

R² is preferably a cytosine base structure, as in the case of troxacitabine. In particular, R² is preferably

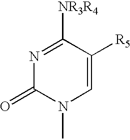

The following are examples of compounds in accordance with the invention:

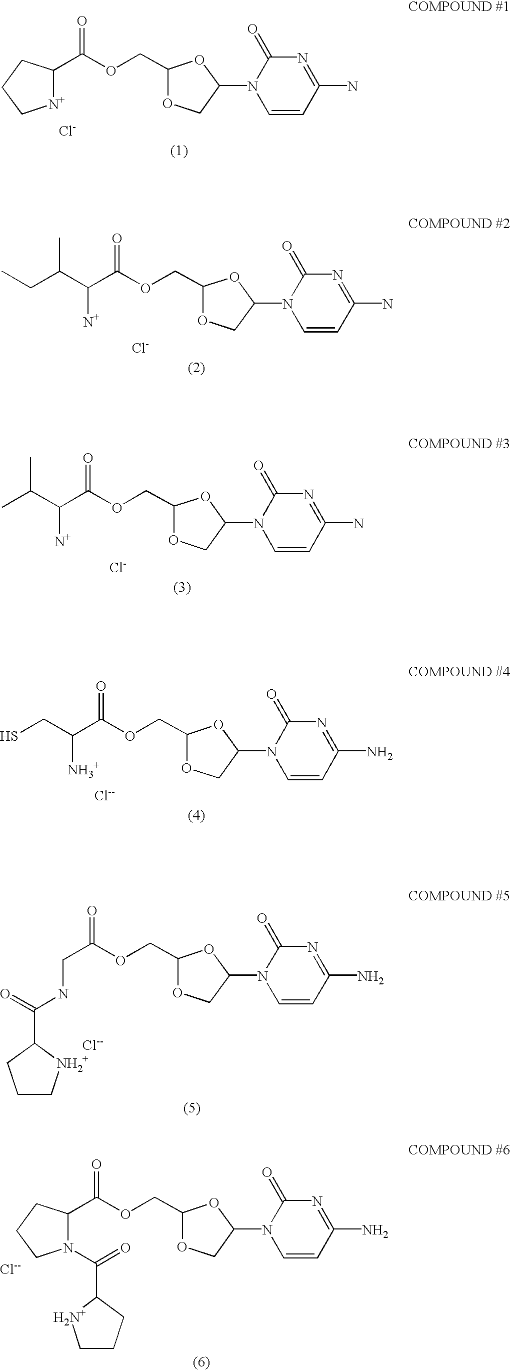

The following compounds 38 to 281 are also compounds in accordance with the invention:

| No. | Name | Structure |
|---|---|---|
| 38 | 4-AMINO-1-(2- DIMETHOXYMETHOXYMETHYL- [1,3]DIOXOLAN-4-YL)-1H- PYRIMIDIN-2-ONE | 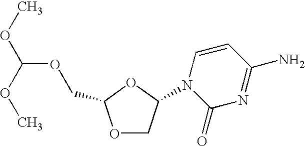 |
| 39 | 4-AMINO-1-(2- DIETHOXYMETHOXYMETHYL- [1,3]DIOXOLAN-4-YL)-1H- PYRIMIDIN-2-ONE | 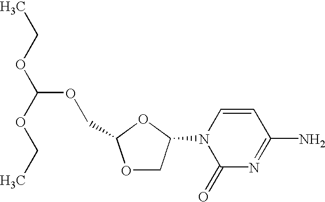 |
| 40 | 4-AMINO-1-[2- ([1,3]DIOXOLAN-2- YLOXYMETHYL)- [1,3]DIOXOLAN-4-YL]-1H- PYRIMIDIN-2-ONE | 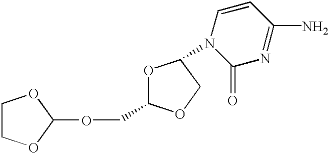 |
| 41 | 4-AMINO-1-[2- (TETRAHYDRO-PYRAN-2- YLOXYMETHYL)- [1,3]DIOXOLAN-4-YL]-1H- PYRIMIDIN-2-ONE | 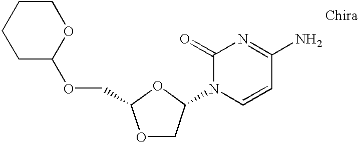 |
| 42 | CARBONIC ACID 4-(4- AMINO-2-OXO-2H- PYRIMIDIN-1-YL)- [1,3]DIOXOLAN-2- YLMETHYL ESTER PHENYL ESTER | 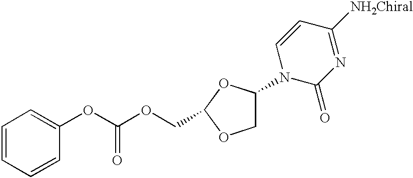 |
| 43 | CARBONIC ACID 4-(2-OXO- 4-PHENOXYCARBONYLAMINO- 2H-PYRIMIDIN-1-YL)- [1,3]DIOXOLAN-2- YLMETHYL ESTER PHENYL ESTER | 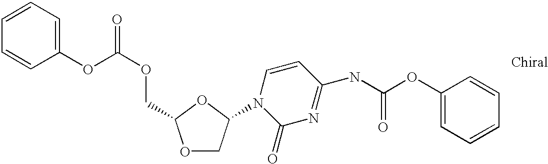 |
| 44 | [1-(2-HYDROXYMETHYL- [1,3]DIOXOLAN-4-YL)-2- OXO-1,2-DIHYDRO- PYRIMIDIN-4-YL]- CARBAMIC ACID PHENYL ESTER | 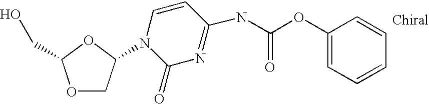 |
| 45 | [1-(2-HYDROXYMETHYL- [1,3]DIOXOLAN-4-YL)-2- OXO-1,2-DIHYDRO- PYRIMIDIN-4-YL]- CARBAMIC ACID ETHYL ESTER | 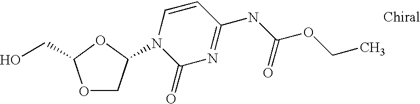 |
| 46 | CARBONIC ACID 4-(4- AMINO-2-OXO-2H- PYRIMIDIN-1-YL)- [1,3]DIOXOLAN-2- YLMETHYL ESTER ETHYL ESTER | 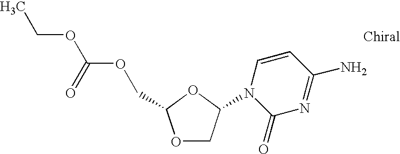 |
| 47 | CARBONIC ACID 4-(4- ETHOXYCARBONYLAMINO-2- OXO-2H-PYRIMIDIN-1-YL)- [1,3]DIOXOLAN-2- YLMETHYL ESTER ETHYL ESTER | 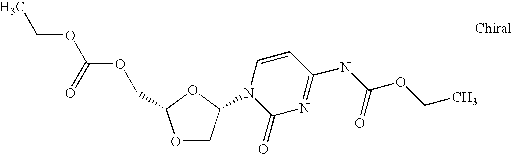 |
| 48 | BUTYL-CARBAMIC ACID 4- (4-AMINO-2-OXO-2H- PYRIMIDIN-1-YL)- [1,3]DIOXOLAN-2- YLMETHYL ESTER | 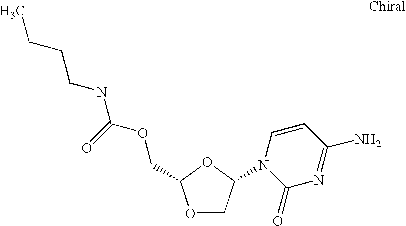 |
| 49 | N-[1-(2-HYDROXYMETHYL- [1,3]DIOXOLAN-4-YL)- CYTOSYL]-2,2-DIMETHYL- PROPIONAMIDE | 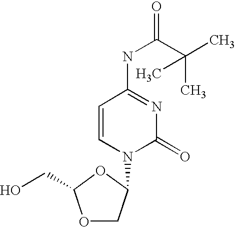 |
| 50 | [1-(2-HYDROXYMETHYL- [1,3]DIOXOLAN-4-YL)- CYTOSYL]-CARBAMIC ACID BENZYL ESTER | 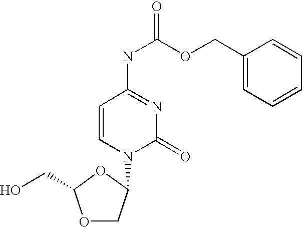 |
| 51 | 4-(4- BENZYLOXYCARBONYLAMINOC YTOSYL)-[1,3]DIOXOLAN- 2-YLMETHYL BENZYL CARBONATE | 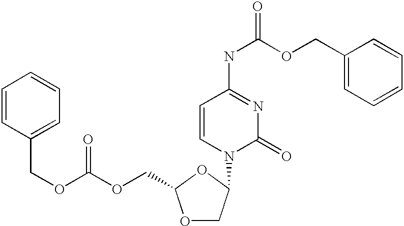 |
| 52 | (2S,4S)-2- PHENYLACETOXYMETHYL-4- CYTOSIN-1′-YL-1,3- DIOXOLANE | 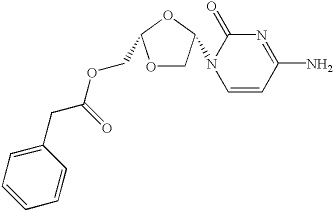 |
| 53 | 4-AMINO-1-(2- TRITYLOXYMETHYL- [1,3]DIOXOLAN-4-YL)-1H- PYRIMIDIN-2-ONE | 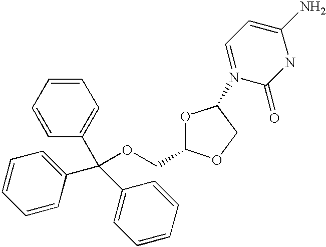 |
| 54 | 4-AMINO-1-[2-(1- METHOXY-1-METHYL- ETHOXYMETHYL)- [1,3]DIOXOLAN-4-YL]-1H- PYRIMIDIN-2-ONE | 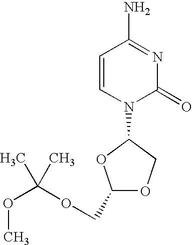 |
| 55 | OCTANOIC ACID [1-(2- HYDROXYMETHYL- [1,3]DIOXOLAN-4-YL)-2- OXO-1, 2-DIHYDRO- PYRIMIDIN-4-YL]-AMIDE | 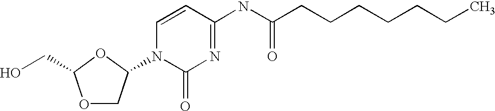 |
| 56 | 4-AMINO-1-(2- BENZYLOXYMETHOXYMETHYL- [1,3]DIOXOLAN-4-YL)-1H- PYRIMIDIN-2-ONE | 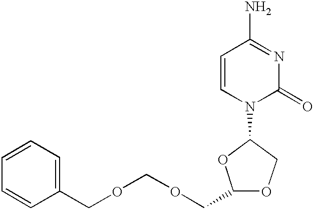 |
| 57 | CARBONIC ACID 4-(4- AMINO-2-OXO-2H- PYRIMIDIN-1-YL)- [1,3]DIOXOLAN-2- YLMETHYL ESTER BENZYL ESTER | 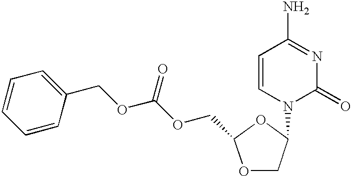 |
| 58 | 2,2-DIMETHYL-PROPIONIC ACID 4-(4-AMINO-2-OXO- 2H-PYRIMIDIN-1-YL)- [1,3]DIOXOLAN-2- YLMETHOXYMETHYL ESTER | 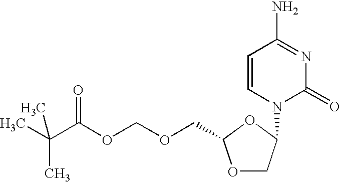 |
| 59 | [1-(2-HYDROXYMETHYL- [1,3]DIOXOLAN-4-YL)-2- OXO-1,2-DIHYDRO- PYRIMIDIN-4-YL]- CARBAMIC ACID BUTYL ESTER | 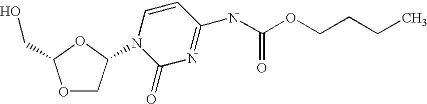 |
| 60 | (2S,4S)--2- HYDROXYMETHYL-4-N-[2″- (2″′-NITROPHENYL)-2″- METHYLPROPIONYL]- CYTOSINE-1′-YL-1,3- DIOXOLANE | 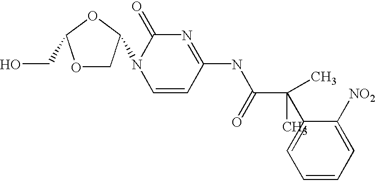 |
| 61 | [1-(2-HYDROXYMETHYL- [1,3]DIOXOLAN-4-YL)-2- OXO-1,2-DIHYDRO- PYRIMIDIN-4-YL]- CARBAMIC ACID HEXYL ESTER | 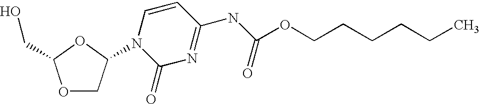 |
| 62 | 4-AMINO-1-[2-(2- METHOXY- ETHOXYMETHOXYMETHYL)- [1,3]DIOXOLAN-4-YL]-1H- PYRIMIDIN-2-ONE | 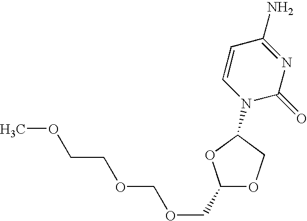 |
| 63 | CARBONIC ACID 4-[4-(4- METHOXY- PHENOXYCARBONYLAMINO)- 2-OXO-2H-PYRIMIDIN-1- YL]-[1,3]DIOXOLAN-2- YLMETHYL ESTER 4- METHOXY-PHENYL ESTER | 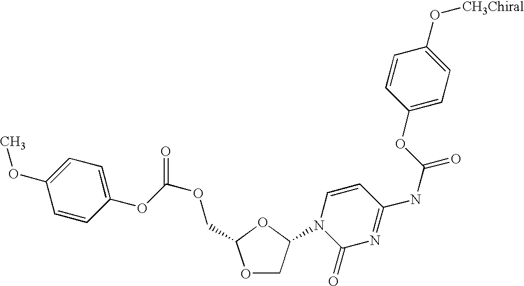 |
| 64 | (2S,4S)-2-(2″-METHYL- HEXANOICOXYMETHYL)-4- (4′-NN- DIMETHYLAMINOMETHYLENE- CYTOSIN-1′-YL)-1,3- DIOXOLANE | 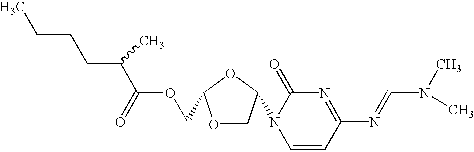 |
| 65 | (2S,4S)-2-(2″-ETHYL- HEXANOICOXYMETHYL)-4- (4′-N,N- DIMETHYLAMINOMETHYLENE- CYTOSIN-1′-YL)-1,3- DIOXOLANE | 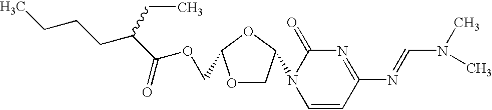 |
| 66 | 6-(Benzyl-tert- butoxycarbonyl-amino)- hexanoic acid 4-(4- amino-2-oxo-2H- pyrimidin-1-yl)- [1,3]dioxolan-2- ylmethyl ester | 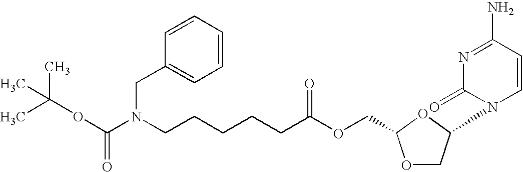 |
| 67 | CARBONIC ACID 4-(4- AMINO-2-OXO-2H- PYRIMIDIN-1-YL)- [1,3]DIOXOLAN-2- YLMETHYL ESTER ISOPROPYL ESTER TRIFLUOROACETATE SALT | 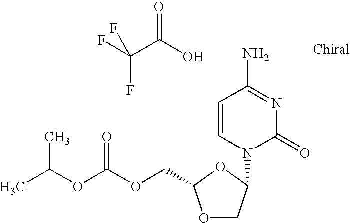 |
| 68 | CARBONIC ACID 4-(4- AMINO-2-OXO-2H- PYRIMIDIN-1-YL)- [1,3]DIOXOLAN-2- YLMETHOXYMETHYL ESTER ISOPROPYL ESTER TRIFLUOROACETIC ACID SALT | 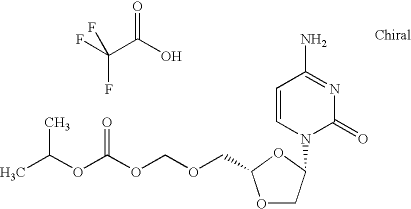 |
| 69 | (2S,4S)-2-(2″- METHYLPHENYLACETOXY)MET HYL-4-CYTOSTN-1′-YL- 1,3-DIOXOLANE | 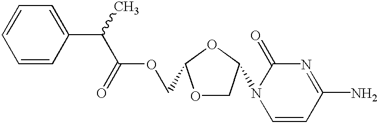 |
| 70 | (2S,4S)-2-(2″- METHYLPHENYLACETOXY)MET HYL-4-(4′-N,N- DIMETHYLAMINOMETHYLENE- CYTOSIN-1′-YL)-1,3- DIOXOLANE | 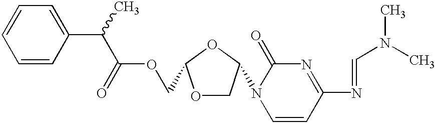 |
| 71 | [1-(2-HYDROXYMETHYL- [1,3]DIOXOLAN-4-YL)-2- OXO-1,2-DIHYDRO- PYRIMIDIN-4-YL]- CARBAMIC ACID PENTYL ESTER | 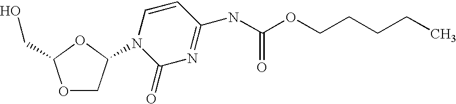 |
| 72 | (2S,4S)-2-(2″- DIMETHYLHEXANOICOXYMETH YL)-4-(4′-N,N- DIMETHYLAMINOMETHYLENE- CYTOSIN-1′-YL)-1,3- DIOXOLANE | 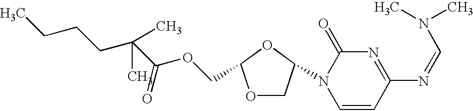 |
| 73 | [1-(2-HYDROXYMETHYL- [1,3]DIOXOLAN-4-YL)-2- OXO-1,2-DIHYDRO- PYRIMIDIN-4-YL]- CARBAMIC ACID 4- METHOXY-PHENYL ESTER | 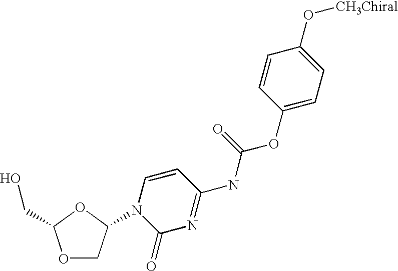 |
| 74 | 1-(2-ALLYLOXYMETHYL- [1,3]DIOXOLAN-4-YL)-4- AMINO-1H-PYRIMIDIN-2- ONE | 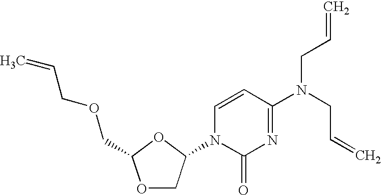 |
| 75 | 4-AMINO-1-(2(S)- ETHOXYMETHYL- [1,3]DIOXOLAN-4(S)-YL)- 1H-PYRIMIDIN-2-ONE | 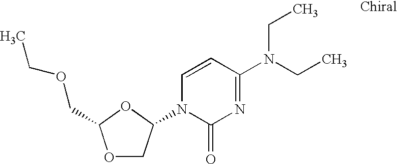 |
| 76 | N-[1-(2(S)-D- RIBOSYLOXYMETHYL- [1,3]DIOXOLAN-4-YL)-2- OXO-1,2-DIHYDRO- PYRIMIDTN-4-YL]- ACETAMIDE | 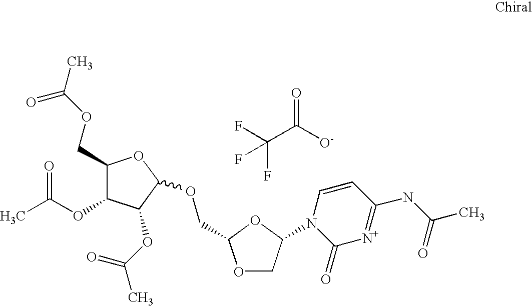 |
| 77 | Benzyl-{5-[1-(2- hydroxymethyl- [1,3]dioxolan-4-yl)-2- oxo-1,2-dihydro- pyrimidin-4- ylcarbamoyl]-pentyl}- carbamic acid tert- butyl ester | 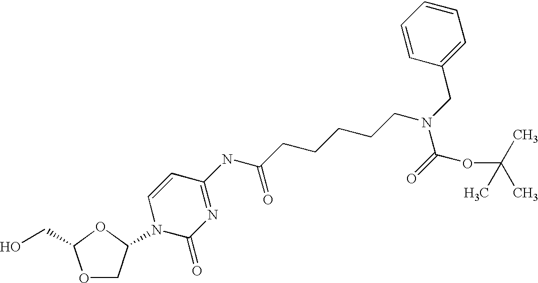 |
| 78 | 6-(Benzyl-tert- butoxycarbonyl-amino)- hexanoic acid 4-{4-[6- (benzyl-tert- butoxycarbonyl-amino)- hexanoylamino]-2-oxo- 2H-pyrimidin-1-yl}- [1,3]dioxolan-2- ylmethyl ester | 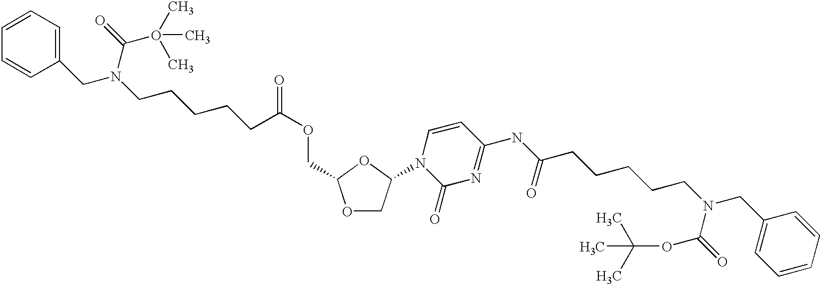 |
| 79 | 2,2,2-TRICHLORO- ACETIMIDIC ACID 4-(4- AMINO-2-OXO-2H- PYRIMIDIN-1-YL)- [1,3]DIOXOLAN-2- YLMETHYL ESTER | 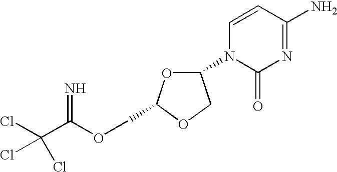 |
| 80 | PENTANEDIOIC ACID 4-[4- (4-METHOXYCARBONYL- BUTYRYLAMINO)-2-OXO- 2#H!-PYRIMIDIN-1-YL]- [1,3]DIOXOLAN-2- YLMETHYL ESTER METHYL ESTER | 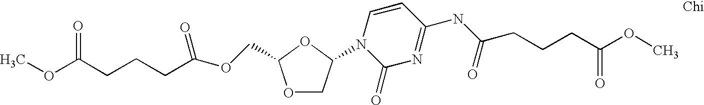 |
| 81 | 4-[1-(2-HYDROXYMETHYL- [1,3]DIOXOLAN-4-YL)-2- OXO-1,2-DIHYDRO- PYRIMIDIN-4- YLCARBAMOYL]-BUTYRIC ACID METHYL ESTER | 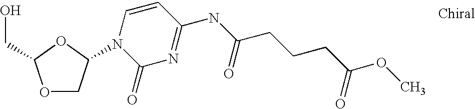 |
| 82 | PENTANEDIOIC ACID 4-(4- AMINO-2-OXO-2#H!- PYRIMIDIN-1-YL)- [1,3]DIOXOLAN-2- YLMETHYL ESTER METHYL ESTER | 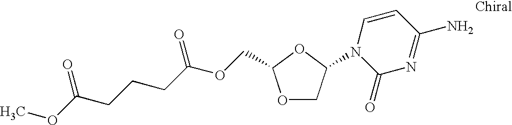 |
| 83 | 6-Benzylamino-hexanoic acid 4-(4-amino-2-oxo- 2H-pyrimidin-1-yl)- [1,3]dioxolan-2- ylmethyl ester bis trifluoroacetate salt | 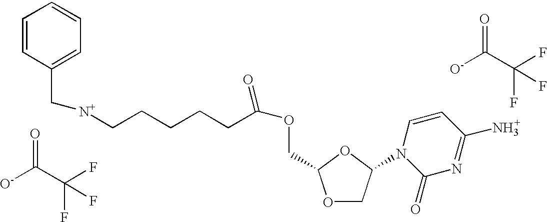 |
| 84 | 6-Benzylamino-hexanoic acid 4-(4-amino-2-oxo- 2H-pyrimidin-1-yl)- [1,3]dioxolan-2- ylmethyl ester | 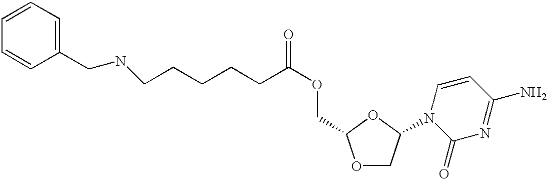 |
| 85 | 4-AMINO-1-[2-(3,4- DIHYDROXY-5- HYDROXYMETHYL- TETRAHYDROFURAN-2- YLOXYMETHYL)- [1,3]DIOXOLAN-4-YL]- 1HPYIMIDIN-2-ONE, TRIFLUOROACETIC ACID SALT | 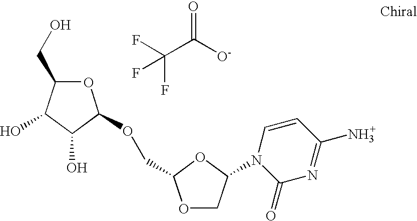 |
| 86 | (2S,4S)-2-(2″-METHYL- HEXANOICOXYMETHYL)-4- CYTOSIN-1′-YL-1,3- DIOXOLANE HYDROCHLORIDE | 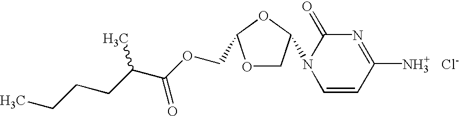 |
| 87 | (2S,4S)-2-(2″,6″- DIMETHYLBENZOYLOXYMETHY L)-4-(4′-N,N- DIMETHYLAMINOMETHLYENE- CYTOSIN-1′-YL)-1,3- DIOXOLANE | 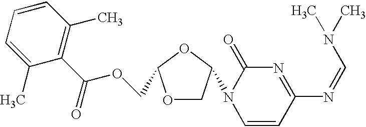 |
| 88 | 1-[2-(4-NITRO- PHENOXYCARBONYLOXYMETHY L)-[1,3]DIOXOLAN-4-YL]- 2-OXO-1,2-DIHYDRO- PYRIMIDIN-4-YL- AMMONIUM; CHLORIDE | 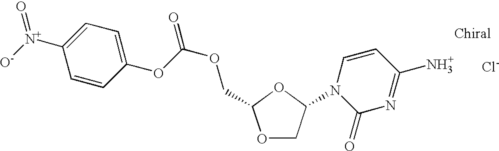 |
| 89 | 1-(2-HYDROXYMETHYL- [1,3]DIOXOLAN-4-YL)-4- (3-CINNAMYL)-1H- PYRIMIDIN-2-ONE TRIFLUORO-ACETATE SALT | 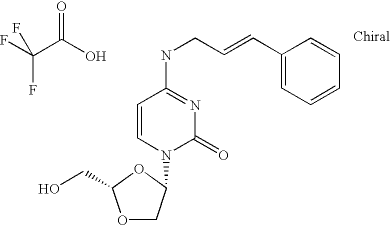 |
| 90 | 4-AMINO-1-[2-(3- CINNAMYLOXYMETHYL)- [1,3]DIOXOLAN-4-YL]-1H- PYRIMIDIN-2-ONE TRIFLUOROACETATE SALT | 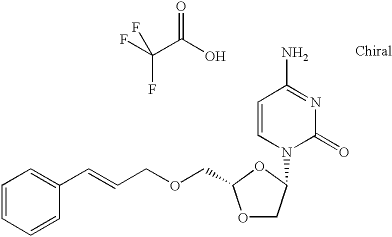 |
| 91 | 4-AMINO-1-[2-(1-ETHOXY- ETHOXYMETHYL)- [1,3]DIOXOLAN-4-YL]-1H- PYRIMIDIN-2-ONE | 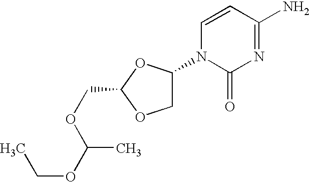 |
| 92 | 4-AMINO-1-[2-(1- CYCLOHEXYLOXY- ETHOXYMETHYL)- [1,3]DIOXOLAN-4-YL]-1H- PYRIMIDIN-2-ONE | 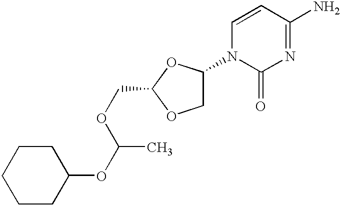 |
| 93 | 1-(2′(S)-ETHOXYMETHYL- [1,3]DIOXOLAN-4′(S)- YL)-4-ETHYLAMINO-1H- PYRIMIDIN-2-ONE | 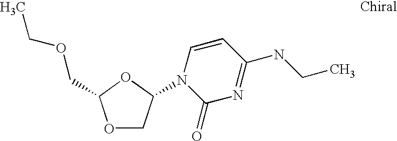 |
| 94 | [1-(2-Hydroxymethyl- [1,3]dioxolan-4-yl)-2- oxo-1,2-dihydro- pyrimidin-4-yl]- carbamic acid 2- isopropyl-5-methyl- cyclohexyl ester | 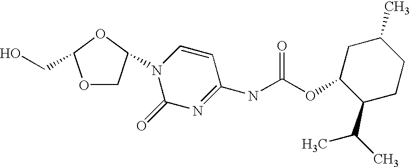 |
| 95 | Carbonic acid 4-(4- amino-2-oxo-2#H!- pyrimidin-1-yl)- [1,3]dioxolan-2- ylmethyl ester 2- isopropyl-5-methyl- cyclohexyl ester | 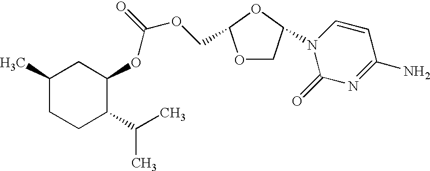 |
| 96 | 2-METHYL-HEXANOIC ACID [1-(2-HYDROXYMETHYL- [1,3]DIOXOLAN-4-YL)-2- OXO-1,2-DIHYDRO- PYRIMIDIN-4-YL]-AMIDE | 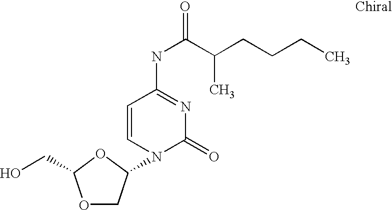 |
| 97 | 4-AMINO-1-[2-(1-BUTOXY- ETHOXYMETHYL)- [1,3]DIOXOLAN-4-YL]-1H- PYRIMIDIN-2-ONE | 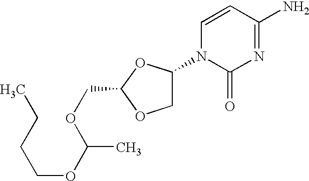 |
| 98 | (2S,4S) 4-AMINO-1-(2- BENZYLOXYMETHYL- [1,3]DIOXOLAN-4-YL)-1H- PYRIMIDIN-2-ONE | 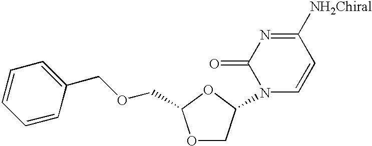 |
| 99 | 2-ETHYL-HEXANOIC ACID [1-(2-HYDROXYMETHYL- [1,3]DIOXOLAN-4-YL)-2- OXO-1,2-DIHYDRO- PYRIMIDIN-4-YL]-AMIDE | 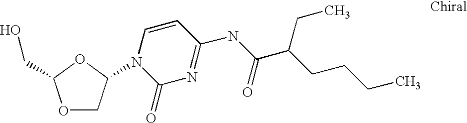 |
| 100 | 2,4,6-Triisopropyl- benzoic acid 4-(4- amino-2-oxo-2H- pyrimidin-1-yl)- [1,3]dioxolan-2- ylmethyl ester | 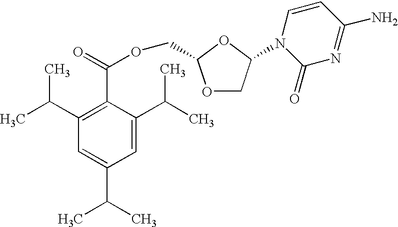 |
| 101 | ADAMANTANE-1-CARBOXYLIC ACID 4-(4- BENZYLOXYCARBONYLAMINO- 2-OXO-2H-PYRIMIDIN-1- YL)-[1,3]DIOXOLAN-2- YLMETHYL ESTER | 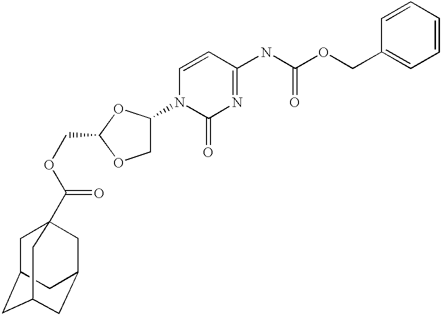 |
| 102 | ADAMANTANE-1-CARBOXYLIC ACID 4-{4-[(ADAMANTANE- 1-CARBONYL)-AMINO]-2- OXO-2H-PYRIMIDIN-1-YL}- [1,3]DIOXOLAN-2- YLMETHYL ESTER | 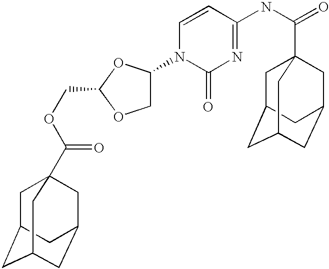 |
| 103 | CARBONIC ACID 4-[4-(4- CHLORO- PHENOXYCARBONYLAMINO)- 2-OXO-2H-PYRIMIDIN-1- YL]-[1,3]DIOXOLAN-2- YLMETHYL ESTER 4- CHLORO-PHENYL ESTER | 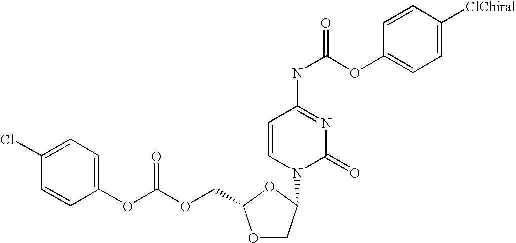 |
| 104 | [1-(2-HYDROXYMETHYL- [1,3]DIOXOLAN-4-YL)-2- OXO-1,2-DIHYDRO- PYRIMIDIN-4-YL]- CARBAMIC ACID 4-CHLORO- PHENYL ESTER TRIFLUOROACETATE SALT | 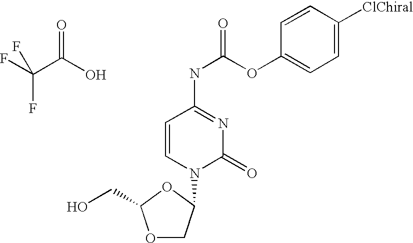 |
| 105 | CARBONIC ACID 4-(4- AMINO-2-OXO-2H- PYRIMIDIN-1-YL)- [1,3]DIOXOLAN-2- YLMETHYL ESTER 4- CHLORO-PHENYL ESTER TRIFLUOROACETATE SALT | 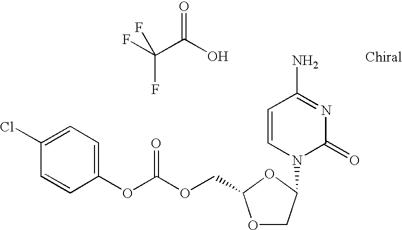 |
| 106 | (2S,4S)-2-(2″- METHYLPHENYLACETOXY)MET HYL-4-(CYTOSIN-1′-YL)- 1,3-DIOXOLANE HYDROCHLORIDE | 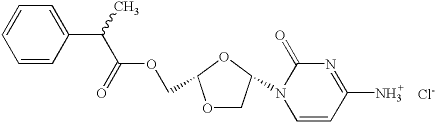 |
| 107 | 2,2-DIMETHYLHEXANOIC ACID 4-(4-AMINO-2-OXO- 2H-PYRIMIDIN-1-YL)-1,3- DIOXOLAN-2-YLMETHYL ESTER HYDROCHLORIDE | 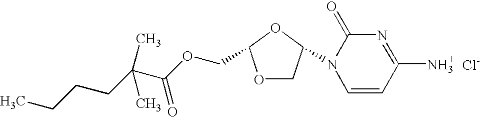 |
| 108 | 1-BENZYL-3-[1-(2- HYDROXYMETHYL- [1,3]DIOXOLAN-4-YL)-2- OXO-1,2-DIHYDRO- PYRIMIDIN-4-YL]-UREA | 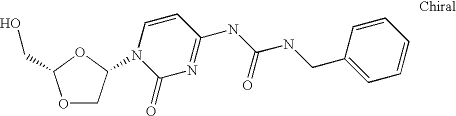 |
| 109 | BENZYL-CARBAMIC ACID 4- [4-(3-BENZYL-UREIDO)-2- OXO-2#H!-PYRIMIDIN-1- YL]-[1,3]DIOXOLAN-2- YLMETHYL ESTER | 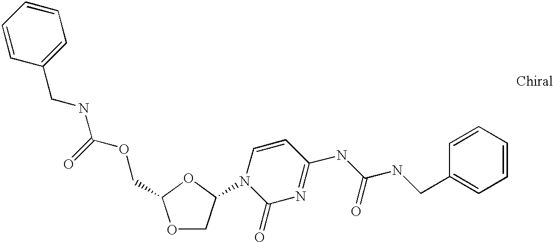 |
| 110 | ADAMANTANE-1-CARBOXYLIC ACID 4-(4-AMINO-2-OXO- 2H-PYRIMIDIN-1-YL)- [1,3]DIOXOLAN-2- YLMETHYL ESTER | 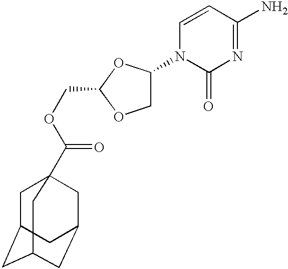 |
| 111 | 5-(BENZYL-TERT- BUTOXYCARBONYL-AMINO)- PENTANOIC ACID 4-(4- AMINO-2-OXO-2H- PYRIMIDIN-1-YL)- [1,3]DIOXOLAN-2- YLMETHYL ESTER | 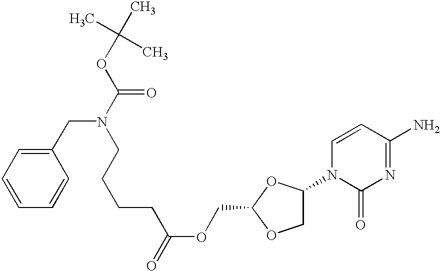 |
| 112 | CARBONIC ACID 4(S)-(4′- AMINO-2′-OXO-2H- PYRIMIDIN-1′-YL)- [1,3]DIOXOLAN-2(S)- YLMETHYL ESTER 4- (5″, 6″-DIMETHOXY-1″- OXO-INDAN-2″- YLIDENEMETHYL)-2,6- DIMETHYL-PHENYL ESTER | 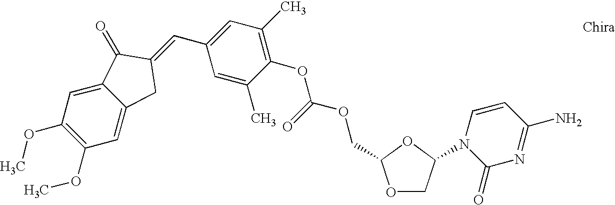 |
| 113 | 4-AMINO-1-[2-(1- METHOXY- CYCLOHEXYLOXYMETHYL)- [1,3]DIOXOLAN-4-YL]-1H- PYRIMIDIN-2-ONE | 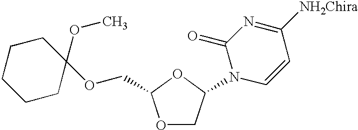 |
| 114 | 5-(BENZYL-TERT- BUTOXYCARBONYL-AMINO)- PENTANOIC ACID 4-{4-[5- (BENZYL-TERT- BUTOXYCARBONYL-AMINO)- PENTANOYLAMINO]-2-OXO- 2H!PYRIMIDIN-1-YL}- [1,3]DIOXOLAN-2- YLMETHYL ESTER | 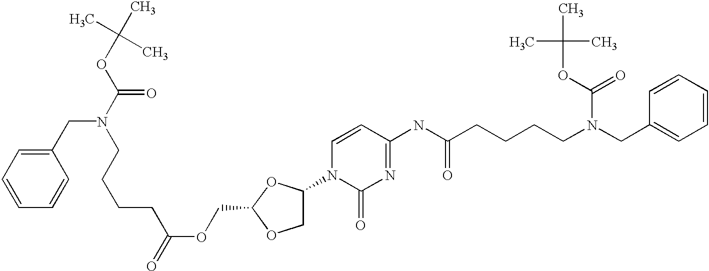 |
| 115 | BENZYL-{4-[1-(2- HYDROXYMETHYL- [1,3]DIOXOLAN-4-YL)-2- OXO-1,2-DIHYDRO- PYRIMIDIN-4- YLCARBAMOYL]-BUTYL}- CARBAMIC ACID TERT!- BUTYL ESTER | 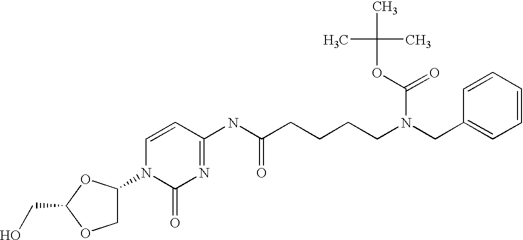 |
| 116 | CARBONIC ACID 4-(4- BENZYLOXYCARBONYLAMINO- 2-OXO-2H-PYRIMIDIN-1- YL)-[1,3]DIOXOLAN-2- YLMETHYL ESTER 4- METHOXY-PHENYL ESTER | 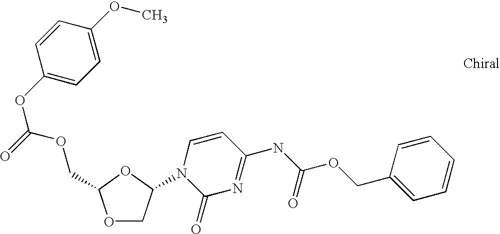 |
| 117 | 4-AMINO-1-{2-[1-(1,1- DIMETHYL-PROPOXY)- ETHOXYMETHYL]- [1,3]DIOXOLAN-4-YL}-1H- PYRIMIDIN-2-ONE | 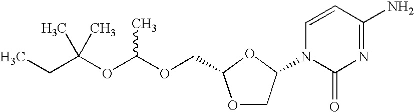 |
| 118 | CARBONIC ACID 4-(4- AMINO-2-OXO-2H- PYRIMIDIN-1-YL)- [1,3]DIOXOLAN-2- YLMETHYL ESTER 4- METHOXY-PHENYL ESTER | 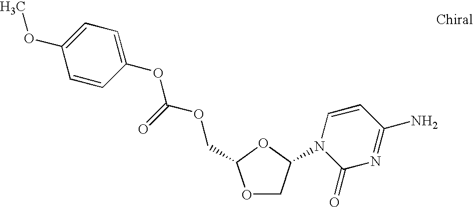 |
| 119 | HEXYL-CARBANIC ACID 4- [4-(3-HEXYL-UREIDO)-2- OXO-2#H!-PYRIMIDIN-1- YL]-[1,3]DIOXOLAN-2- YLMETHYL ESTER | 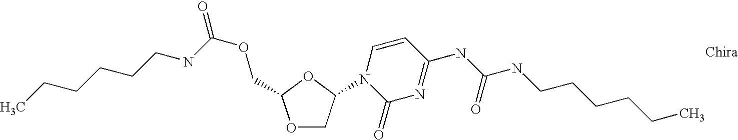 |
| 120 | 1-HEXYL-3-[1-(2- HYDROXYMETHYL- [1,3]DIOXOLAN-4-YL)-2- OXO-1,2-DIHYDRO- PYRIMIDIN-4-YL]-UREA | 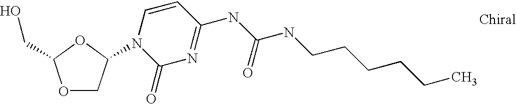 |
| 121 | HEXYL-CARBAMIC ACID 4- (4-AMINO-2-OXO-2H- PYRIMIDIN-1-YL)- [1,3]DIOXOLAN-2- YLMETHYL ESTER | 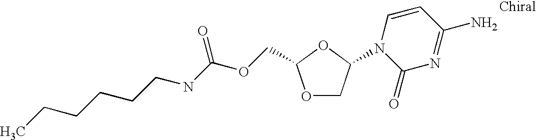 |
| 122 | CARBONIC ACID 4-(4- BENZYLOXYCARBONYLAMINO- 2-OXO-2H-PYRIMIDIN-1- YL)-[1,3]DIOXOLAN-2- YLMETHYL ESTER HEXYL ESTER | 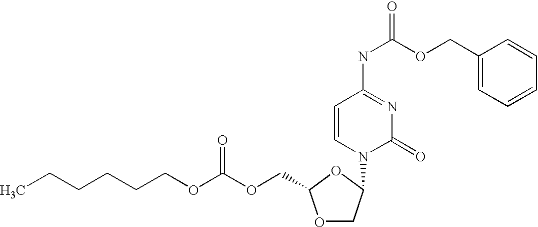 |
| 123 | 4-AMINO-1-{2-[BIS-(4- METHOXY-PHENYL)-PHENYL- METHOXYMETHYL]- [1,3]DIOXOLAN-4-YL}-1H- PYRIMIDIN-2-ONE | 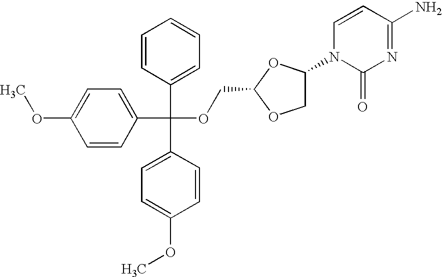 |
| 124 | {1-[2-(4-ISOPROPYL- PHENYLCARBAMOYLOXYMETHY L)-[1,3]DIOXOLAN-4-YL]- 2-OXO-1,2-DIHYDRO- PYRIMIDIN-4-YL}- CARBAMIC ACID BENZYL ESTER | 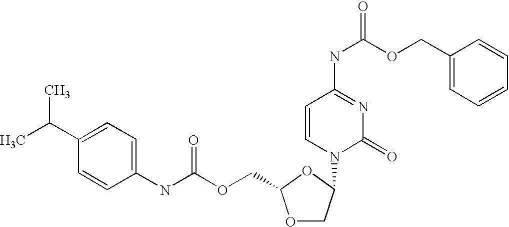 |
| 125 | Benzyl-{5-[1-(2- hydroxymethyl- [1,3]dioxolan-4-yl)-2- oxo-1,2-dihydro- pyrimidin-4- ylcarbamoyl]-5-methyl- hexyl}-carbamic acid tert-butyl ester | 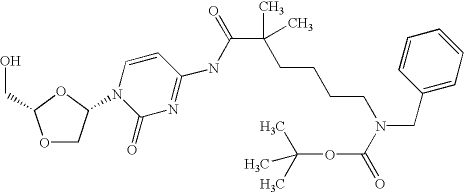 |
| 126 | CARBONIC ACID 4-(4- AMINO-2-OXO-2H- PYRIMIDIN-1-YL)- [1,3]DIOXOLAN-2- YLMETHYL ESTER HEXYL ESTER | 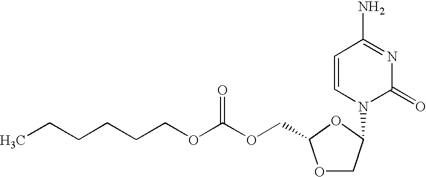 |
| 127 | (4-ISOPROPYL-PHENYL)- CARBAMIC ACID 4-(4- AMINO-2-OXO-2H- PYRIMIDIN-1-YL)- [1,3]DIOXOLAN-2- YLMETHYL ESTER | 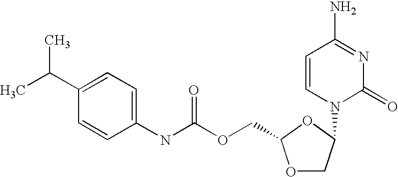 |
| 128 | 4-AMINO-1-[5-(2-METHYL- 4-OXO-4#H!- BENZO[1,3]DIOXIN-2- YLOXYMETHYL)- TETRAHYDRO-FURAN-2-YL]- 1#H!-PYRIMIDIN-2-ONE; COMPOUND WITH TRIFLUORO-ACETIC ACID | 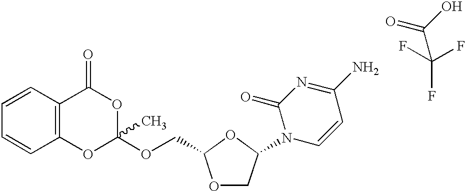 |
| 129 | (2S,4S)-2-(1″- ADMANTANEACETOXY)METHYL -4-(4′-N,N- DIMETHYLAMINOMETHYLENE- CYTOSIN-1′-YL)-1,3- DIOXOLANE | 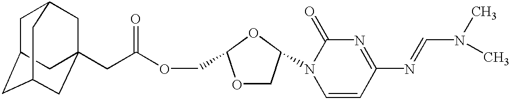 |
| 130 | (2S,4S)-2-(2″- DIPHENYLACETOXYMETHYL)- 4-(4′-N,N- DIMETHYLAMINOMETHYLENE- CYTOSIN-1′-YL)-1,3- DIOXOLANE | 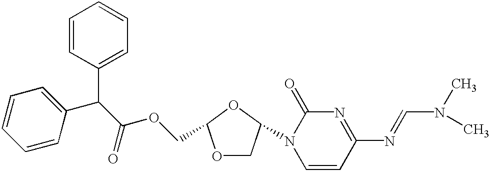 |
| 131 | (2S,4S)-2- (BENZYLOXYCARBONYL-L- VALINOXYMETHYL)-4-(4′- N,N- DIMETHYLAMINOMETHYLENE- CYTOSIN-1′-YL)-1,3- DIOXOLANE | 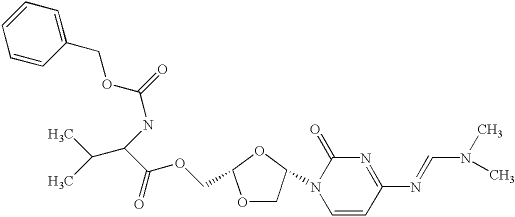 |
| 132 | 6-(Benzyl-tert- butoxycarbonyl-amino)- 2,2-dimethyl-hexanoic acid 4-[4- (dimethylamino- methyleneamino)-2-oxo- 2H-pyrimidin-1-yl]- [1,3]dioxolan-2- ylmethyl ester | 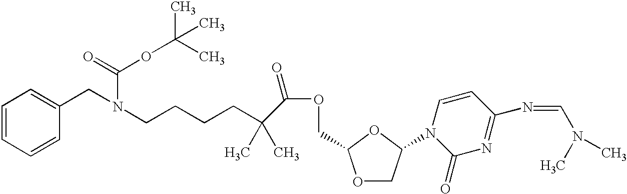 |
| 133 | 2,2-Dimethyl-propionic acid 4-[4- (dimethylamino- methyleneamino)-2-oxo- 2H-pyrimidin-1-yl]- [1,3]dioxolan-2- ylmethyl ester | 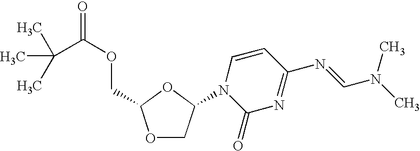 |
| 134 | 4-AMINO-1-{2-[(4- METHOXY-PHENYL)- DIPHENYL- METHOXYMETHYL]- [1,3]DIOXOLAN-4-YL}-1H- PYRIMIDIN-2-ONE | 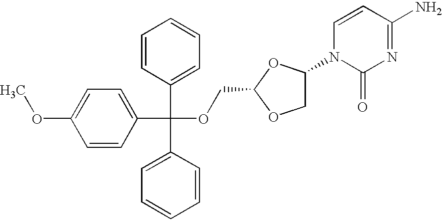 |
| 135 | DIHEXYLCARBAMIC ACID 4(S)-(4′-AMINO-2′-OXO- 2H-PYRIMIDIN-1′-YL)- [1,3]DIOXOLAN-2(S)- YLMETHYL ESTER | 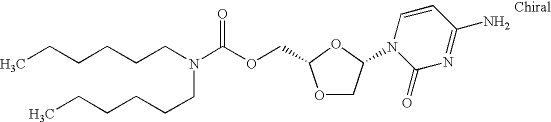 |
| 136 | 4-(BENZO[1,3]DITHIOL-2- YLAMINO)-1-(2- HYDROXYMETHYL- [1,3]DIOXOLAN-4-YL)- 1H!PYRIMIDIN-2-ONE | 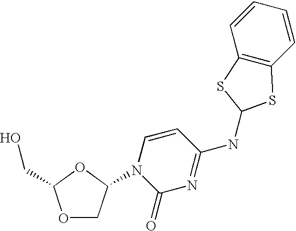 |
| 137 | DECYL-CARBAMIC ACID 4- (4-AMINO-2-OXO-2H- PYRIMIDIN-1-YL)- [1,3]DIOXOLAN-2- YLMETHYL ESTER | 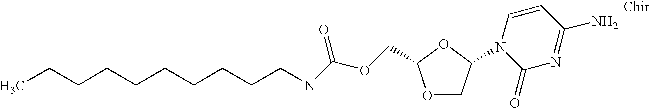 |
| 138 | 4-AMINO-1-[2- (BENZO[1,3]DITHIOL-2- YLOXYMETHYL)- [1,3]DIOXOLAN-4-YL]-1H- PYRIMIDIN-2-ONE | 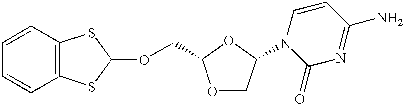 |
| 139 | 4-AMINO-1-[2- (DIMETHOXY-PHENYL- METHOXYMETHYL)- [1,3]DIOXOLAN-4-YL]-1H- PYRIMIDIN-2-ONE | 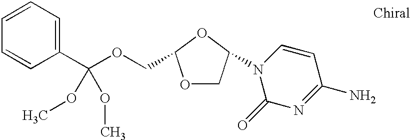 |
| 140 | BENZYL-METHYL-CARBAMIC ACID 4-(4-AMINO-2-OXO- 2H-PYRIMIDIN-1-YL)- [1,3]DIOXOLAN-2- YLMETHYL ESTER | 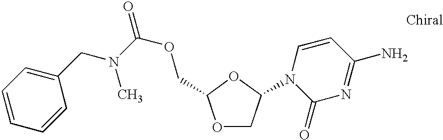 |
| 141 | 4-AMINO-1-[2-(1,1- DIMETHOXY- PENTYLOXYMETHYL)- [1,3]DIOXOLAN-4-YL]-1H- PYRIMIDIN-2-ONE | 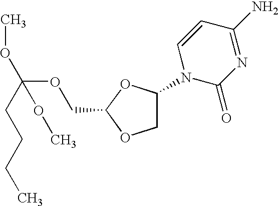 |
| 142 | (2S,4S)-2-(2″- DIMETHYLPHENYLACETOXY)M ETHYL-4-(4′-N,N- DIMETHYLAMINOMETHYLENE- CYTOSTN-1,-YL)-1,3- DIOXOLANE | 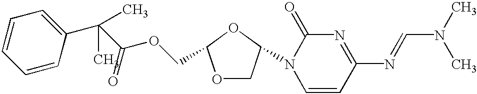 |
| 143 | (2S,4S)-2-(4″-N,N- DIMETHYLAMINOPHENYLACET OXY)METHYL-4-(4′-N,N- DIMETHYLAMINOMETHYLENE- CYTOSIN-1′-YL)-1,3- DIOXOLANE | 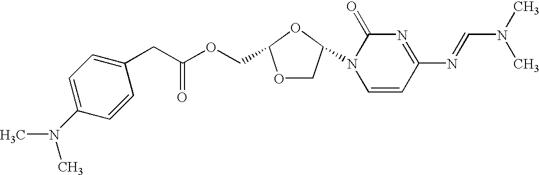 |
| 144 | 4-(9-PHENYL-9#H!- XANTHEN-9-YLAMINO)-1- [2-(9-PHENYL-9#H!- XANTHEN-9-YLOXYMETHYL)- [1,3]DIOXOLAN-4-YL]- 1#H!-PYRIMIDIN-2-ONE | 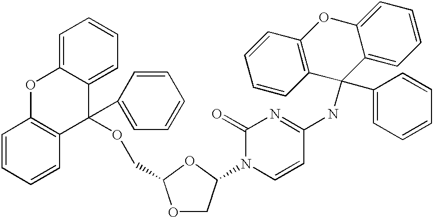 |
| 145 | 1-(2-HYDROXYMETHYL- [1,3]DIOXOLAN-4-YL)-4- (9-PHENYL-9#H!-XANTHEN- 9-YLAMINO)-1#H!- PYRIMIDIN-2-ONE | 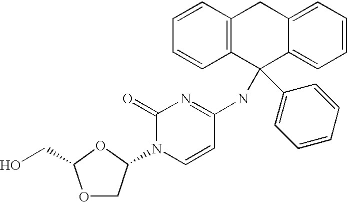 |
| 146 | 4-AMINO-1-[2-(9-PHENYL- 9#H!-XANTHEN-9- YLOXYMETHYL)- [1,3]DIOXOLAN-4-YL]- 1#H!-PYRIMIDIN-2-ONE | 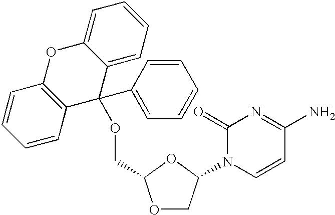 |
| 147 | THIOCARBONIC ACID O- [4(S)-(4′-AMINO-2′-OXO- 2H-PYRIMIDIN-1′-YL)- [1,3]DIOXOLAN-2(S)- YLMETHYL] ESTER O- PHENYL ESTER | 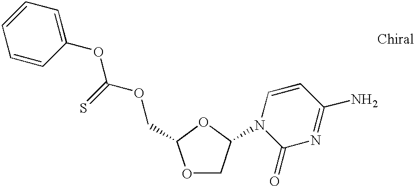 |
| 148 | Acetic acid 6-acetoxy- 5-acetoxymethyl-2-[4- (4- benzyloxycarbonylamino- 2-oxo-2H-pyrimidin-1- yl)-[1,3]dioxolan-2- ylmethoxy]-2-methyl- tetrahydro- [1,3]dioxolo[4,5- b]pyran-7-yl ester | 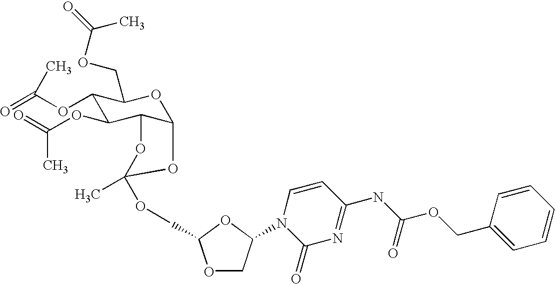 |
| 149 | 6-(Benzyl-tert- butoxycarbonyl-amino)- methyl-hexanoic acid 4-[4-(dimethylamino- methyleneamino)-2-oxo- 2H-pyrimidin-1-yl]- [1,3]dioxolan-2- ylmethyl ester | 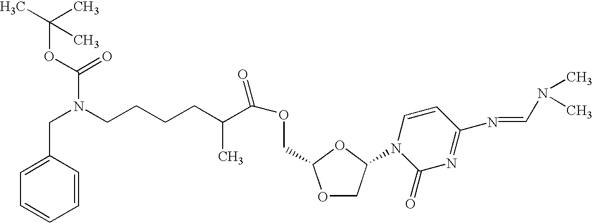 |
| 150 | CARBONIC ACID HEXYL ESTER 4-(4- HEXYLOXYCARBONYLAMINO- 2-OXO-2H-PYRIMIDIN-1- YL)-[1,3]DIOXOLAN-2- YLMETHYL ESTER | 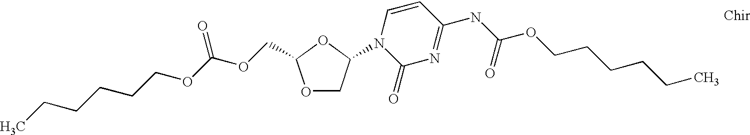 |
| 151 | Acetic acid 6-acetoxy- 5-acetoxymethyl-2-[4- (4-amino-2-oxo-2H- pyrimidin-1-yl)- [1,3]dioxolan-2- ylmethoxy]-2-methyl- tetrahydro- [1,3]dioxolo [4,5- b]pyran-7-yl ester | 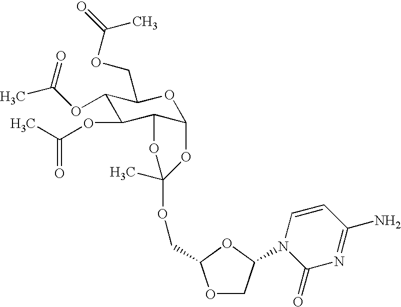 |
| 152 | 4-[(BENZOTRIAZOL-1- YLMETHYL)-AMINO]-1-(2- HYDROXYMETHYL- [1,3]DIOXOLAN-4-YL)-1H- PYRIMIDIN-2-ONE | 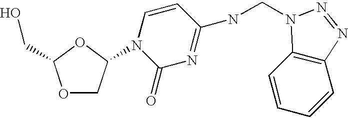 |
| 153 | BENZOIC ACID 4-(4- BENZYLOXYCARBONYLAMINO- 2-OXO-2H-PYRIMIDIN-1- YL)-[1,3]DIOXOLAN-2- YLMETHYL ESTER | 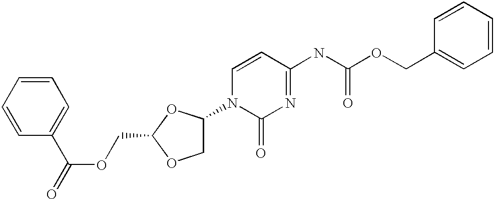 |
| 154 | 4-AMINO-1-[2-(1- BENZYLOXY-1-METHYL- ETHOXYMETHYL)- [1,3]DIOXOLAN-4-YL]-1H- PYRIMIDIN-2-ONE | 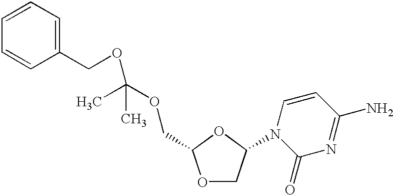 |
| 155 | (2S,4S)-2-[2″-(2″′- NITROPHENYL)-2″- METHYLPROPIONYLOXYMETHY L]-4-CYTOSIN-1′-YL-1,3- DIOXOLANE | 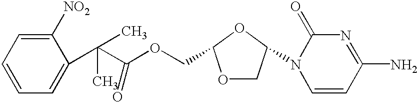 |
| 156 | (2S,4S)-2-(N,N- DIMETHYL-L- VALINYLOXYMETHYL)-4- CYTOSIN-1′-YL-1,3- DIOXOLANE | 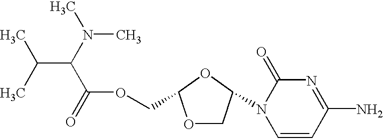 |
| 157 | (2S,4S)-(3″-DIPHENYL- 2″- METHYLPROPIOXYMETHYL)- 4-CYTOSIN-1′-YL-1,3- DIOXOLANE | 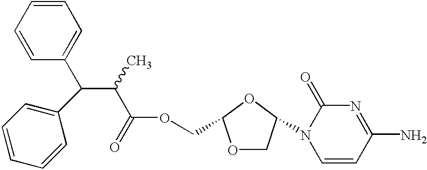 |
| 158 | Benzyl-{5-[1-(2- hydroxymethyl- [1,3]dioxolan-4-yl)-2- oxo-1,2-dihydro- pyrimidin-4- ylcarbamoyl]-hexyl}- carbamic acid tert- butyl ester | 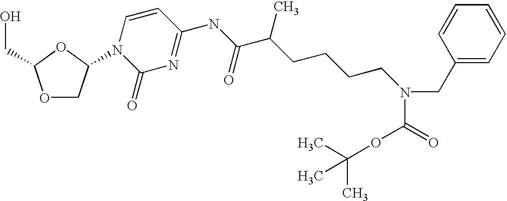 |
| 159 | CARBONIC ACID 4-[4-(4- CHLORO- BUTOXYCARBONYLAMINO)-2- OXO-2H-PYRIMIDIN-1-YL]- [1,3]DIOXOLAN-2- YLMETHYL ESTER 4- CHLORO-BUTYL ESTER | 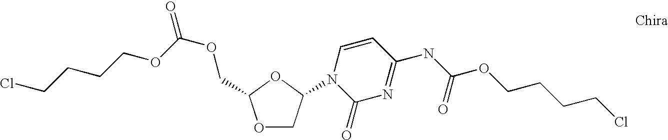 |
| 160 | [1-(2-HYDROXYMETHYL- [1,3]DIOXOLAN-4-YL)-2- OXO-1,2-DIHYDRO- PYRIMIDIN-4-YL]- CARBAMIC ACID 4-CHLORO- BUTYL ESTER | 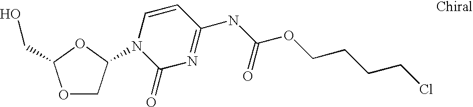 |
| 161 | 2,6-Dimethyl-benzoic acid 4-(4-amino-2-oxo- 2H-pyrimidin-1-yl)- [1,3]dioxolan-2- ylmethyl ester | 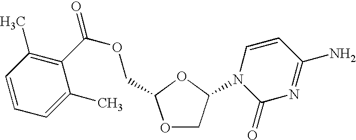 |
| 162 | 1-[2-(2,6-DIMETHYL- BENZOYLOXYMETHYL)- [1,3]DIOXOLAN-4-YL]-2- OXO-1,2-DIHYDRO PYRIMIDIN-4-YL- AMMONIUM; CHLORIDE | 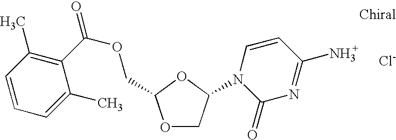 |
| 163 | BENZOIC ACID 4-(4- AMINO-2-OXO-2H- PYRIMIDIN-1-YL)- [1,3]DIOXOLAN-2- YLMETHYL ESTER | 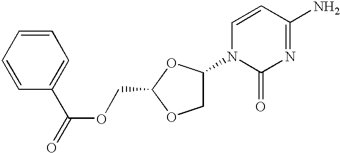 |
| 164 | CARBONIC ACID 4-(4- AMINO-2-OXO-2H- PYRIMIDIN-1-YL)- [1,3]DIOXOLAN-2- YLMETHYL ESTER 3- DIMETHYLAMINO-PROPYL ESTER TRIFLUORO-ACETIC ACID SALT | 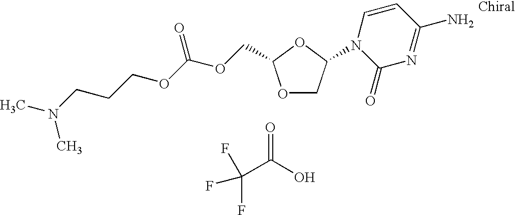 |
| 165 | N-{[1-(2-HYDROXYMETHYL- [1,3]DIOXOLAN-4-YL)-2- OXO-1,2-DIHYDRO- PYRIMIDIN-4-YLAMINO]- METHYL}-BENZAMIDE | 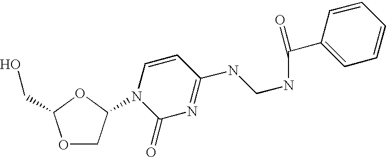 |
| 166 | 5-(Benzyl-tert- butoxycarbonyl-amino)- 2,2-dimethyl-5-oxo- pentanoic acid 4-[4- (dimethylamino- methyleneamino)-2-oxo- 2H-pyrimidin-1-yl]- [1,3]dioxolan-2- ylmethyl ester | 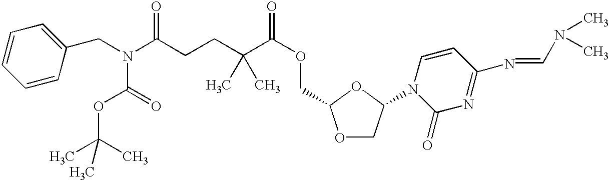 |
| 167 | [1-(2-HYDROXYMETHYL- [1,3]DIOXOLAN-4-YL)-2- OXO-1,2-DIHYDRO- PYRIMIDIN-4-YL]- CARBAMIC ACID 2- BENZENESULFONYL-ETHYL ESTER | 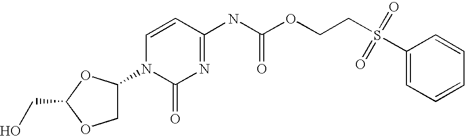 |
| 168 | N-[1-(2-HYDROXYMETHYL- [1,3]DIOXOLAN-4-YL)-2- OXO-1,2-DIHYDRO- PYRIMIDIN-4-YL]-4- NITRO- BENZENESULFONAMIDE | 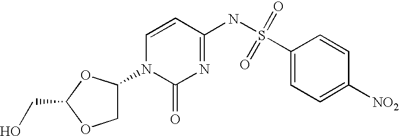 |
| 169 | [1-(2-HYDROXYMETHYL- [1,3]DIOXOLAN-4-YL)-2- OXO-1,2-DIHYDRO- PYRIMIDIN-4-YL]- CARBAMIC ACID 4- DIMETHYLAMINO-BUTYL ESTER TRIFLUOROACETIC ACID SALT | 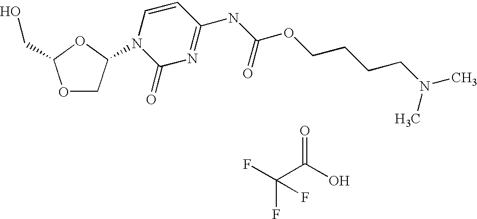 |
| 170 | 4-AMINO-1-[2-(DIETHOXY- PHENYL-METHOXYMETHYL)- [1,3]DIOXOLAN-4-YL]-1H- PYRIMIDIN-2-ONE | 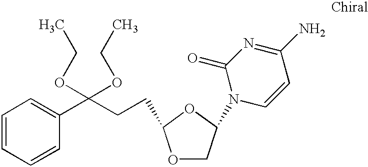 |
| 171 | (S,S) 4-(DI-PROP-2′- YNYL-AMINO)-1-(2″- HYDROXYMETHYL- [1,3]DIOXOLAN-4″-YL)- 1H-PYRIMIDIN-2-ONE | 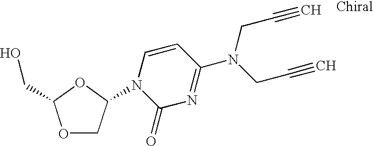 |
| 172 | 1-(2-HYDROXYMETHYL- [1,3]DIOXOLAN-4-YL)-4- (PHENYLAMINOMETHYL- AMINO)-1H-PYRIMIDIN-2- ONE | 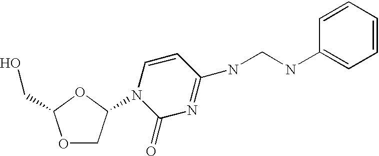 |
| 173 | (S,S)-4-AMINO-1-(2′- PROP-2′-YNYLOXYMETHYL- [1,3]DIOXOLAN-4′-YL)- 1H-PYRIMIDIN-2-ONE | 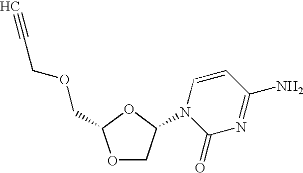 |
| 174 | 4-METHOXY-BENZOIC ACID 4-[4-(4-METHOXY- BENZOYLAMINO)-2-OXO-2H- PYRIMIDIN-1-YL]- [1,3]DIOXOLAN-2- YLMETHYL ESTER | 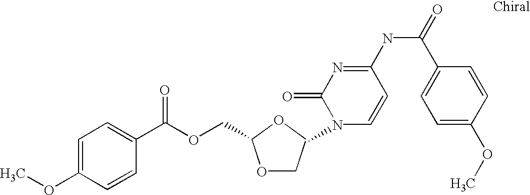 |
| 175 | N-[1-(2-HYDROXYMETHYL- [1,3]DIOXOLAN-4-YL)-2- OXO-1,2-DIHYDRO- PYRIMIDIN-4-YL]-4- METHOXY-BENZAMIDE | 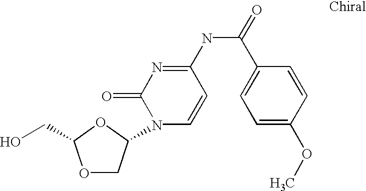 |
| 176 | 4-METHOXY-BENZOIC ACID 4-(4-AMINO-2-OXO-2H- PYRIMIDIN-1-YL)- [1,3]DIOXOLAN-2- YLMETHYL ESTER | 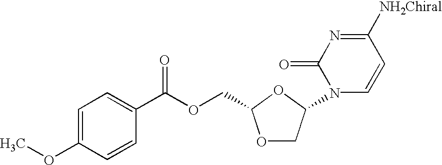 |
| 177 | 4-AMINO-1-(2- TRIMETHOXYMETHOXYMETHYL -[1,3]DIOXOLAN-4-YL)- 1H-PYRIMIDIN-2-ONE | 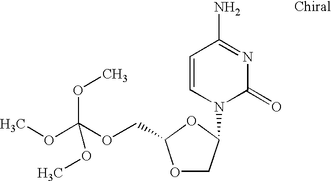 |
| 178 | (S,S)-4-AMINO-1-(2′- ETHOXYMETHYL- [1,3]DIOXOLAN-4′-YL)- 1H-PYRIMIDIN-2-ONE | 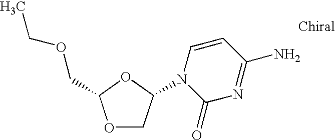 |
| 179 | (S,S)-1-(2′- ALLYLOXYMETHYL- [1,3]DIOXOLAN-4′-YL)-4- AMINO-1H-PYRIMIDIN-2- ONE | 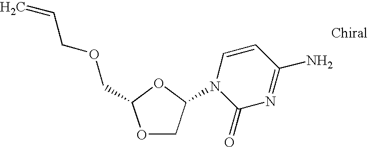 |
| 180 | (S,S)-1-(2′- ETHOXYMETHYL- [1,3]DIOXOLAN-4′-YL)-4- ETHYLAMINO-1H- PYRIMIDIN-2-ONE | 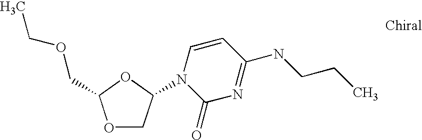 |
| 181 | CARBONIC ACID 4-NITRO- BENZYL ESTER 4-[4-(4- NITRO- BENZYLOXYCARBONYLAMINO) -2-OXO-2H-PYRIMIDIN-1- YL]-[1,3]DIOXOLAN-2- YLMETHYL ESTER | 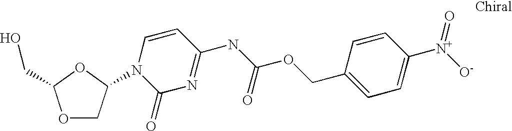 |
| 182 | [1-(2-HYDROXYMETHYL- [1,3]DIOXOLAN-4-YL)-2- OXO-1,2-DIHYDRO- PYRIMIDIN-4-YL]- CARBAMIC ACID 4-NITRO- BENZYL ESTER | 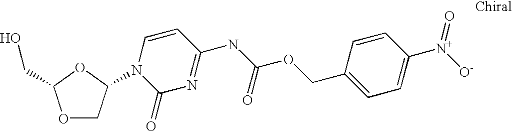 |
| 183 | CARBONIC ACID 4-(4- AMINO-2-OXO-2H- PYRIMIDIN-1-YL)- [1,3]DIOXOLAN-2- YLMETHYL ESTER 4-NITRO- BENZYL ESTER HYDROCHLORIDE SALT | 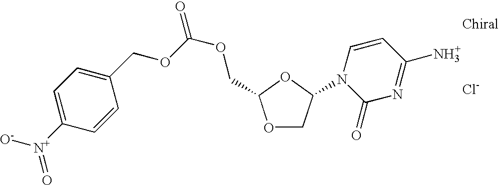 |
| 184 | 3,4,6-TRI-O-BENZOYL- 1,2-O-(1-(4-AMINO-2- OXO-2H-PYRIMIDIN-1-YL)- [1,3]DIOXOLAN-2- YLMETHYLOXY)-BENZYL)- □^{˜}D-GLUCOPYRANOSe | 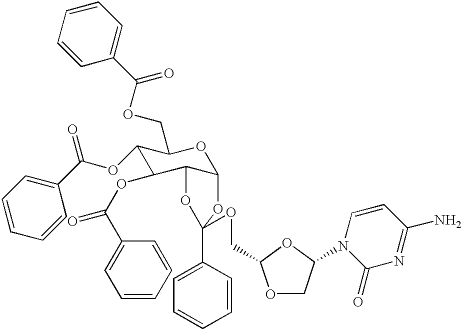 |
| 185 | 4-AMINO-1-{2-[TRIS-(4- METHOXY-PHENYL)- METHOXYMETHYL]- [1,3]DIOXOLAN-4-YL}-1H- PYRIMIDIN-2-ONE | 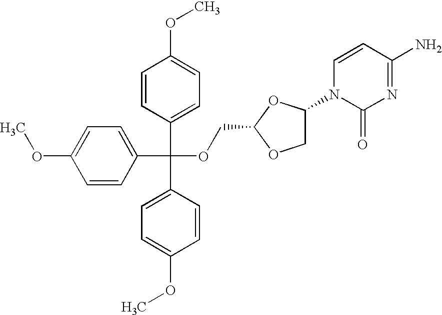 |
| 186 | 3,5-DI-TERT-BUTYL- BENZOIC ACID 4-(4- AMINO-2-OXO-2H- PYRIMIDIN-1-YL)- [1,3]DIOXOLAN-2- YLMETHYL ESTER | 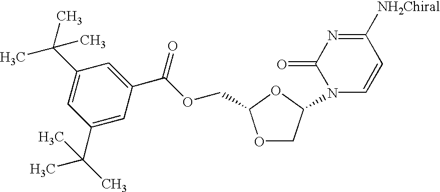 |
| 187 | 3,4-DICHLORO-BENZOIC ACID 4-(4-AMINO-2-OXO- 2H-PYRIMIDIN-1-YL)- [1,3]DIOXOLAN-2-YL METHYL ESTER | 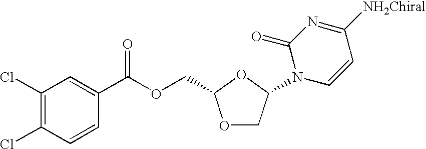 |
| 188 | N-[1-(2-HYDROXYMETHYL- [1,3]DIOXOLAN-4-YL)-2- OXO-1,2-DIHYDRO- PYRIMIDIN-4-YL]-2,4- DINITRO- BENZENESULFONAMIDE | 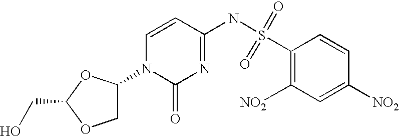 |
| 189 | 4-TRIFLUOROMETHYL- BENZOIC ACID 4-(4- AMINO-2-OXO-2H- PYRIMIDIN-1-YL)- [1,3]DIOXOLAN-2-YL METHYL ESTER | 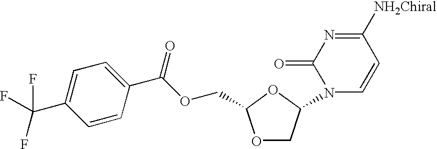 |
| 190 | 2-FLUORO-BENZOIC ACID 4-(4-AMINO-2-OXO-2H- PYRIMIDIN-1-YL)- [1,3]DIOXOLAN-2-YL METHYL ESTER | 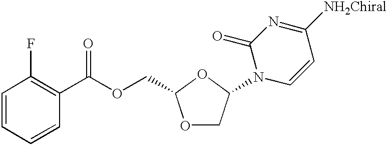 |
| 191 | 4-HEXYL-BENZOIC ACID 4- (4-AMINO-2-OXO-2H- PYRIMIDIN-1-YL)- [1,3]DIOXOLAN-2-YL METHYL ESTER | 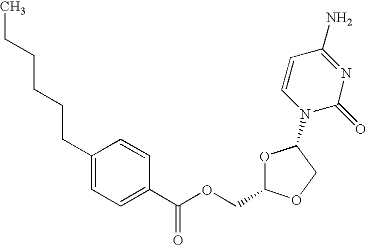 |
| 192 | 6-TERT!- BUTOXYCARBONYLAMINO- HEXANOIC ACID 4-[4-(6- TERT- BUTOXYCARBONYLAMINO- HEXANOYLAMINO)-2-OXO- 2H-PYRIMIDIN-1-YL]- [1,3]DIOXOLAN-2-YL METHYL ESTER | 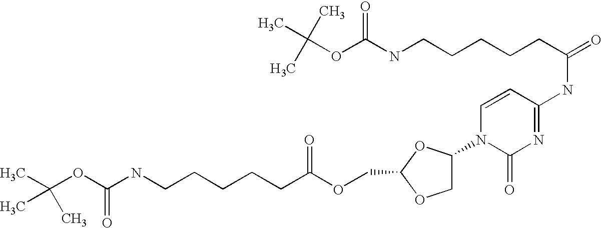 |
| 193 | {5-[1-(2-HYDROXYMETHYL- [1,3]DIOXOLAN-4-YL)-2- OXO-1,2-DIHYDRO- PYRIMIDIN-4- YLCARBAMOYL]-PENTYL}- CARBAMIC ACID TERT- BUTYL ESTER | 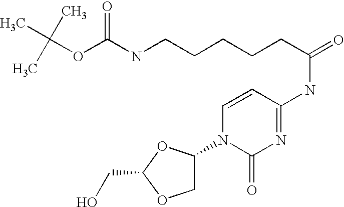 |
| 194 | 6-TERT!- BUTOXYCARBONYLAMINO- HEXANOIC ACID 4-(4- AMINO-2-OXO-2H- PYRIMIDIN-1-YL)- [1,3]DIOXOLAN-2- YLMETHYL ESTER | 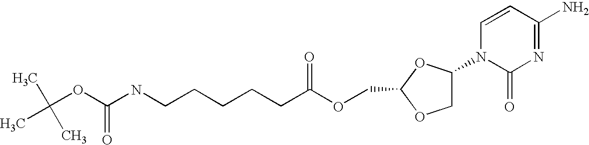 |
| 195 | 4-AMINO-1-{2- [DIMETHOXY-(4-METHOXY- PHENYL)-METHOXYMETHYL]- [1,3]DIOXOLAN-4-YL}- 1#H!-PYRIMIDIN-2-ONE | 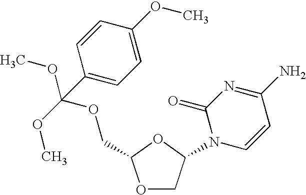 |
| 196 | 8-PHENYL-OCTANOIC ACID 4-[2-OXO-4-(8-PHENYL- OCTANOYLAMINO)-2H- PYRIMIDIN-1-YL]- [1,3]DIOXOLAN-2-YL METHYL ESTER | 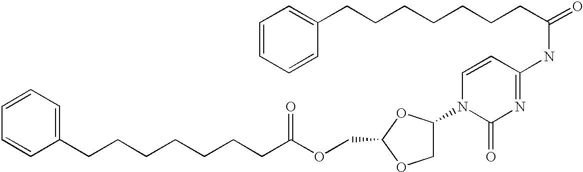 |
| 197 | 8-PHENYL-OCTANOIC ACID [1-(2-HYDROXYMETHYL- [1,3]DIOXOLAN-4-YL)-2- OXO-1,2-DIHYDRO- PYRIMIDIN-4-YL]-AMIDE | 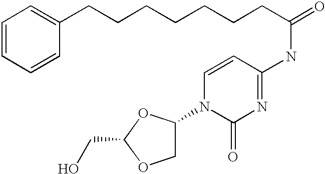 |
| 198 | 8-PHENYL-OCTANOIC ACID 4-(4-AMINO-2-OXO-2H- PYRIMIDIN-1-YL)- [1,3]DIOXOLAN-2-YL METHYL ESTER | 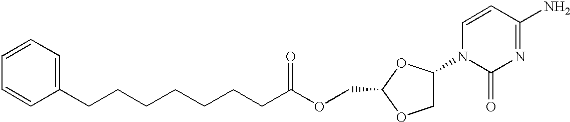 |
| 199 | 4-Amino-1-(2- triethoxymethoxymethyl- [1,3]dioxolan-4-yl)-1H- pyrimidin-2-one | 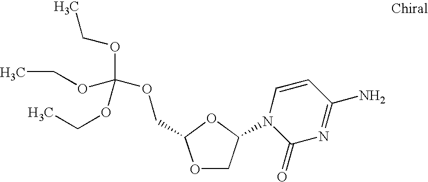 |
| 200 | 4-AMINO-1-[2- (DIMETHOXY-#P!-TOLYL- METHOXYMETHYL)- [1,3]DIOXOLAN-4-YL]- 1#H!-PYRIMIDIN-2-ONE | 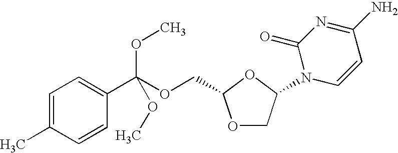 |
| 201 | 3-[4-(4-AMINO-2-OXO-2H- PYRIMIDIN-1-YL)- [1,3]DIOXOLAN-2-YL METHOXY]-ACRYLIC ACID ETHYL ESTER | 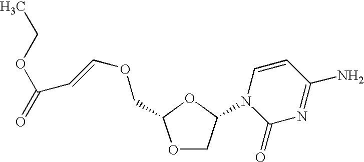 |
| 202 | ACETIC ACID 4-{1-[2-(4- ACETOXY- BENZYLOXYCARBONYLOXYMET HYL)-[1,3]DIOXOLAN-4- YL]-2-OXO-1,2-DIHYDRO- PYRIMIDIN-4-YL CARBAMOYLOXYMETHYL}- PHENYL ESTER | 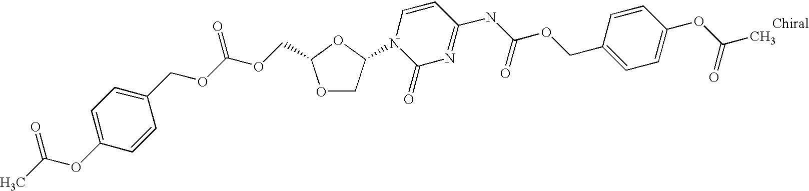 |
| 203 | ACETIC ACID 4-[1-(2- HYDROXYMETHYL- [1,3]DIOXOLAN-4-YL)-2- OXO-1,2-DIHYDRO- PYRIMIDIN-4- YLCARBAMOYLOXYMETHYL]- PHENYL ESTER | 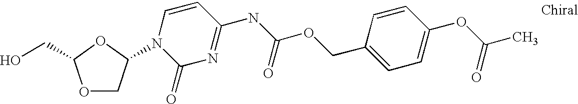 |
| 204 | 4-NITRO-BENZOIC ACID 4- (4-AMINO-2-OXO-2H- PYRIMIDIN-1-YL)- [1,3]DIOXOLAN-2-YL METHYL ESTER | 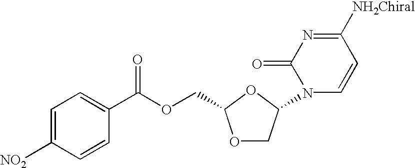 |
| 205 | DITHIOCARBONIC ACID O- [4-(4-AMINO-2-OXO-2H- PYRIMIDIN-1-YL)- [1,3]DIOXOLAN-2-YL METHYL] ESTER S-PHENYL ESTER | 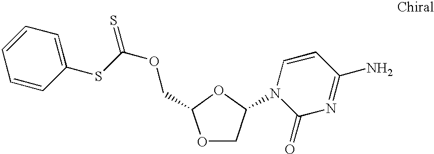 |
| 206 | 2-CHLORO-BENZOIC ACID 4-(4-AMINO-2-OXO-2#H!- PYRIMIDIN-1-YL)- [1,3]DIOXOLAN-2-YL METHYL ESTER | 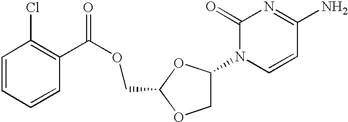 |
| 207 | 7-ISOPROPYL-2,4A- DIMETHYL- 1,2,3,4,4A,4B,5,6,10,10 A-DECAHYDRO- PHENANTHRENE-2- CARBOXYLIC ACID [1-(2- HYDROXYMETHYL- [1,3]DIOXOLAN-4-YL)-2- OXO-1,2-DIHYDRO- PYRIMIDIN-4-YL]-AMIDE | 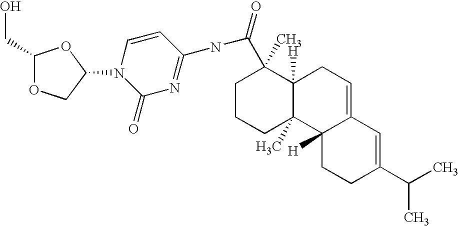 |
| 208 | DODECANOIC ACID [1-(2- HYDROXYMETHYL- [1,3]DIOXOLAN-4-YL)-2- OXO-1,2-DIHYDRO- PYRIMIDIN-4-YL]-AMIDE | 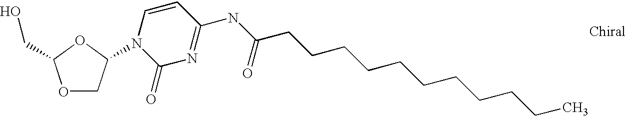 |
| 209 | BIPHENYL-2-CARBOXYLIC ACID 4-(4-AMINO-2-OXO- 2#H!-PYRIMIDIN-1-YL)- [1,3]DIOXOLAN-2-YL METHYL ESTER | 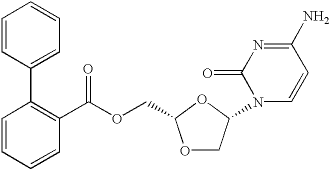 |
| 210 | 4-PENTYL- BICYCLO[2.2.2]OCTANE-1- CARBOXYLIC ACID [1-(2- HYDROXYMETHYL- [1,3]DIOXOLAN-4-YL)-2- OXO-1,2-DIHYDRO- PYRIMIDIN-4-YL]-AMIDE | 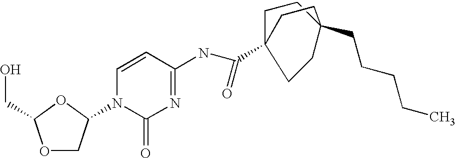 |
| 211 | 4-PENTYL- BICYCLO[2.2.2]OCTANE-1- CARBOXYLIC ACID 4-(4- AMINO-2-OXO-2H- PYRIMIDIN-1-YL)- [1,3]DIOXOLAN-2-YL METHYL ESTER | 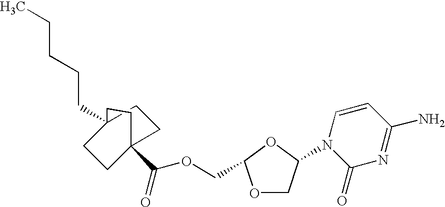 |
| 212 | 2,2-DIMETHYL-PROPIONIC ACID 4-(1-{2-[4-(2,2- DIMETHYL-PROPIONYLOXY)- BENZYLOXYCARBONYLOXYMET HYL]-[1,3]DIOXOLAN-4- YL}-2-OXO-1,2-DIHYDRO- PYRIMIDIN-4- YLCARBAMOYLOXYMETHYL)- PHENYL ESTER | 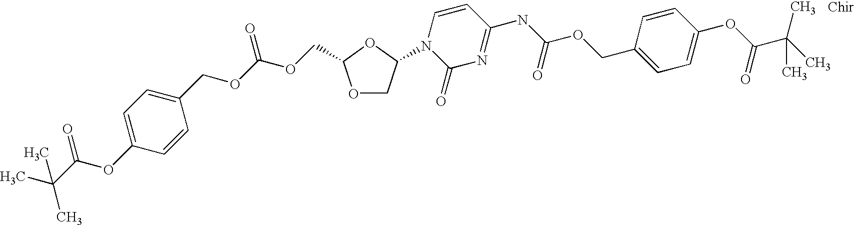 |
| 213 | 2,2-DIMETHYL-PROPIONIC ACID 4-[1-(2- HYDROXYMETHYL- [1,3]DIOXOLAN-4-YL)-2- OXO-1,2-DIHYDRO- PYRIMIDIN-4- YLCARBAMOYLOXYMETHYL]- PHENYL ESTER | 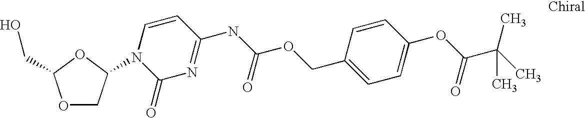 |
| 214 | {6-[4-(4-AMINO-2-OXO- 2H-PYRIMIDIN-1-YL)- [1,3]DIOXOLAN-2- YLMETHOXYCARBONYLAMINO]-HEXYL}-BENZYL-CARBAMIC ACID TERT-BUTYL ESTER | 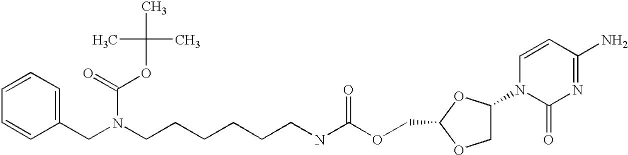 |
| 215 | (3-PHENYL-PROPYL)- CARBAMIC ACID 4-(4- AMINO-2-OXO-2H- PYRIMIDIN-1-YL)- [1,3]DIOXOLAN-2-YL METHYL ESTER | 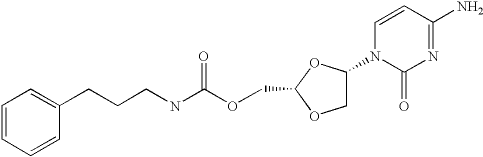 |
| 216 | Octadec-9-enoic acid [1-(2-hydroxymethyl- [1,3]dioxolan-4-yl)-2- oxo-1,2-dihydro- pyrimidin-4-yl]-amide | 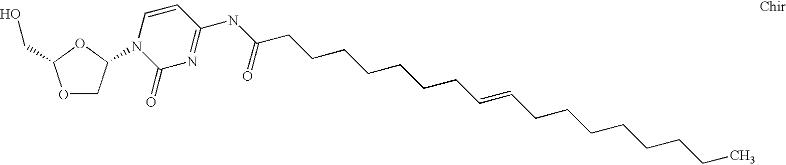 |
| 217 | OCTADECA-9,12-DIENOIC ACID [1-(2- HYDROXYMETHYL- [1,3]DIOXOLAN-4-YL)-2- OXO-1,2-DIHYDRO- PYRIMIDIN-4-YL]-AMIDE | 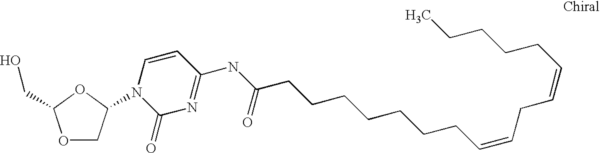 |
| 218 | 2,2-DIETHYL-HEXANOIC ACID 4-(4-AMINO-2-OXO- 2H-PYRIMIDIN-1-YL)- [1,3]DIOXOLAN-2-YL METHYL ESTER | 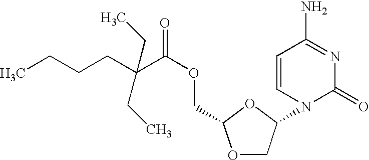 |
| 219 | OCTADEC-9-ENOIC ACID [1-(2-HYDROXYMETHYL- [1,3]DIOXOLAN-4-YL)-2- OXO-1,2-DIHYDRO- PYRIMIDIN-4-YL]-AMIDE | 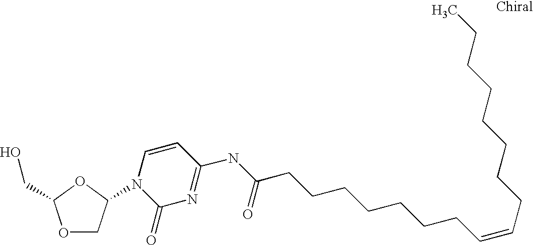 |
| 220 | BIPHENYL-2-CARBOXYLIC ACID 4-(4-AMINO-2-OXO- 2H-PYRIMIDIN-1-YL)- [1,3]DIOXOLAN-2-YL METHYL ESTER | 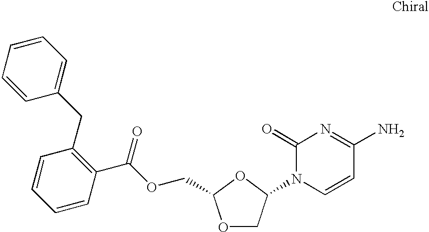 |
| 221 | N,N-Dibutyl-N′-[1-(2- hydroxymethyl [1,3]dioxolan-4-yl)-2- oxo-1,2-dihydro- pyrimidin-4-yl]- formamidine | 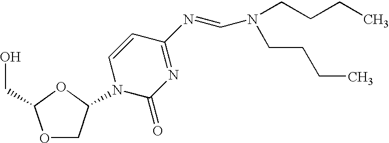 |
| 222 | N′-[1-(2-HYDROXYMETHYL- [1,3]DIOXOLAN-4-YL)-2- OXO-1,2-DIHYDRO- PYRIMIDIN-4-YL]-N,N- DIMETHYL-FORMAMIDINE | 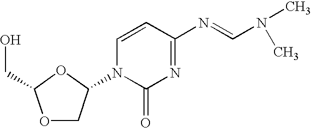 |
| 223 | 1-PHENYL- CYCLOPROPANECARBOXYLIC ACID 4-(4-AMINO-2-OXO- 2H-PYRIMIDIN-1-YL)- [1,3]DIOXOLAN-2-YL METHYL ESTER | 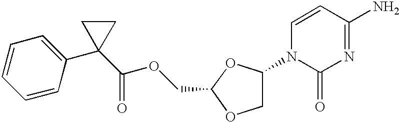 |
| 224 | 2-METHYL-2-(2-NITRO- PHENYL)PROPIONIC ACID 4-(4-AMINO-2-OXO-2H- PYRIMIDIN-1-YL)- [1,3]DIOXOLAN-2- YLMETHYL ESTER HYDROCHLORIDE SALT | 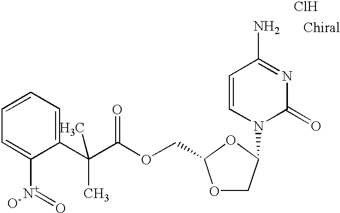 |
| 225 | 1-PHENYL- CYCLOHEXANECARBOXYLIC ACID [1-(2- HYDROXYMETHYL- [1,3]DIOXOLAN-4-YL)-2- OXO-1,2-DIHYDRO- PYRIMIDIN-4-YL]-AMIDE | 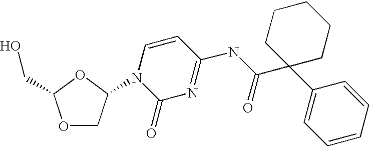 |
| 226 | 1-PHENYL- CYCLOHEXANECARBOXYLIC ACID 4-(4-AMINO-2-OXO- 2H-PYRIMIDIN-1-YL)- [1,3]DIOXOLAN-2-YL METHYL ESTER | 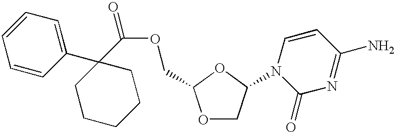 |
| 227 | 2,2-DIMETHYL-8-PHENYL- OCTANOIC ACID [1-(2- HYDROXYMETHYL- [1,3]DIOXOLAN-4-YL)-2- OXO-1,2-DIHYDRO- PYRIMIDIN-4-YL]-AMIDE | 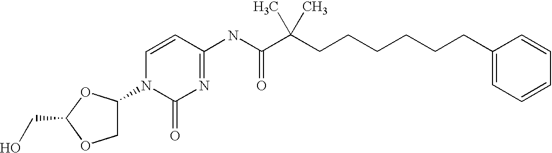 |
| 228 | N′-[1-(2-HYDROXYMETHYL- [1,3]DIOXOLAN-4-YL)-2- OXO-1,2-DIHYDRO- PYRIMIDIN-4-YL]-N,N- DIMETHYL-ACETAMIDINE | 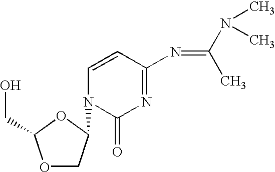 |
| 229 | 1-PHENYL- CYCLOPENTANECARBOXYLIC ACID [1-(2- HYDROXYMETHYL- [1,3]DIOXOLAN-4-YL)-2- OXO-1,2-DIHYDRO- PYRIMIDIN-4-YL]-AMIDE | 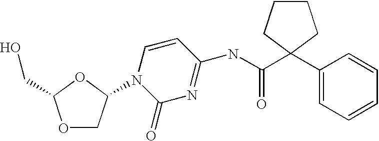 |
| 230 | N′-[1-(2-HYDROXYMETHYL- [1,3]DIOXOLAN-4-YL)-2- OXO-1,2-DIHYDRO- PYRIMIDIN-4-YL]-N,N- DIISOPROPYL-FORMAMIDINE | 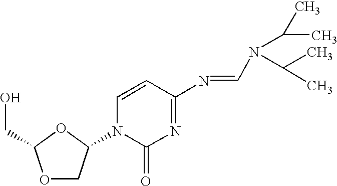 |
| 231 | HEXAHYDRO-2,5-METHANO- PENTALENE-3A-CARBOXYLIC ACID [1-(2- HYDROXYMETHYL- [1,3]DIOXOLAN-4-YL)-2- OXO-1,2-DIHYDRO- PYRIMIDIN-4-YL]-AMIDE | 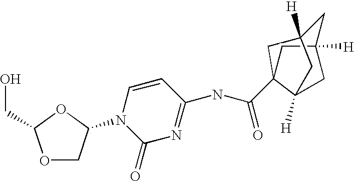 |
| 232 | HEXAHYDRO-2,5-METHANO- PENTALENE-3A-CARBOXYLIC ACID 4-(4-AMINO-2-OXO- 2H-PYRIMIDIN-1-YL)- [1,3]DIOXOLAN-2-YL METHYL ESTER | 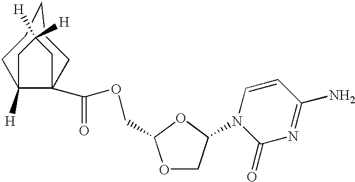 |
| 233 | 2,2-DIETHYL-8-PHENYL- OCTANOIC ACID 4-(4- AMINO-2-OXO-2H- PYRIMIDIN-1-YL)- [1,3]DIOXOLAN-2-YL METHYL ESTER | 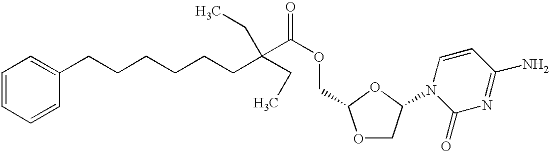 |
| 234 | 5-(2,5-DIMETHYL- PHENOXY)-2,2-DIMETHYL- PENTANOIC ACID [1-(2- HYDROXYMETHYL- [1,3]DIOXOLAN-4-YL)-2- OXO-1,2-DIHYDRO- PYRIMIDIN-4-YL]-AMIDE | 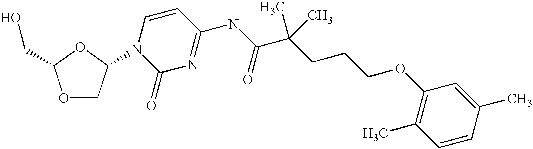 |
| 235 | 1,2,2,3-TETRAMETHYL- CYCLOPENTANECARBOXYLIC ACID [1-(2- HYDROXYMETHYL- [1,3]DIOXOLAN-4-YL)-2- OXO-1,2-DIHYDRO- PYRIMIDIN-4-YL]-AMIDE | 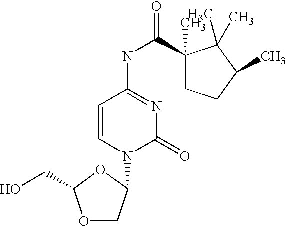 |
| 236 | 4-(1-BENZYL-PYRROLIDIN- 2-YLIDENEAMINO)-1-(2- HYDROXYMETHYL- [1,3]DIOXOLAN-4-YL)-1H- PYRIMIDIN-2-ONE | 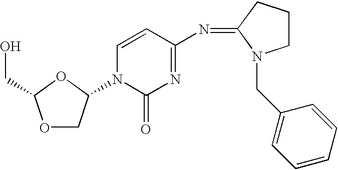 |
| 237 | 4-AMINO-1-{2-[4-(2,5- DIMETHYL-PHENOXY)-1,1- DIMETHYL-BUTOXYMETHYL]- [1,3]DIOXOLAN-4-YL}-1H- PYRIMIDIN-2-ONE | 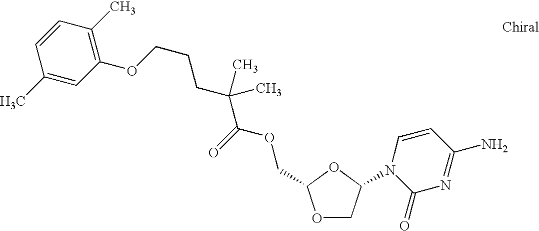 |
| 238 | 2,2-DIMETHYL-8-PHENYL- OCTANOIC ACID 4-(4- AMINO-2-OXO-2H- PYRIMIDIN-1-YL)- [1,3]DIOXOLAN-2-YL METHYL ESTER | 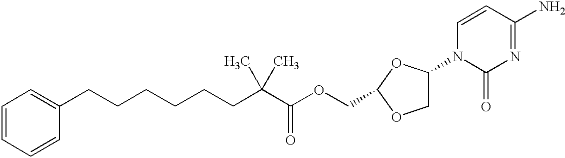 |
| 239 | 4-PENTYL- CYCLOHEXANECARBOXYLIC ACID [1-(2- HYDROXYMETHYL- [1,3]DIOXOLAN-4-YL)-2- OXO-1,2-DIHYDRO- PYRIMIDIN-4-YL]-AMIDE | 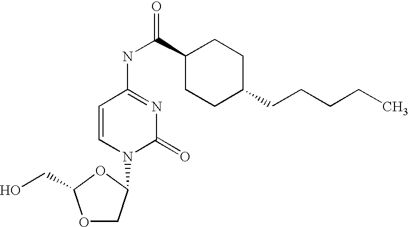 |
| 240 | 4-PENTYL- CYCLOHEXANECARBOXYLIC ACID 4-(4-AMINO-2-OXO- 2H-PYRIMIDIN-1-YL)- [1,3]DIOXOLAN-2-YL METHYL ESTER | 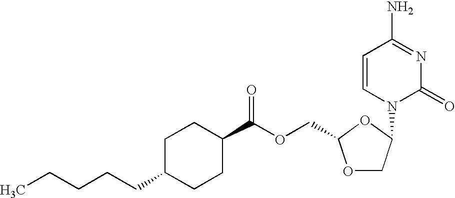 |
| 241 | N-[1-(2-HYDROXYMETHYL- [1,3]DIOXOLAN-4-YL)-2- OXO-1,2-DIHYDRO- PYRIMIDIN-4-YL]-2,2- DIPHENYL-ACETAMIDE | 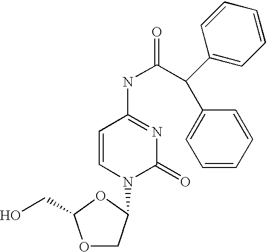 |
| 242 | N-[1-(2-HYDROXYMETHYL- [1,3]DIOXOLAN-4-YL)-2- OXO-1,2-DIHYDRO- PYRIMIDIN-4-YL]-2-(4- ISOBUTYL-PHENYL)- PROPIONAMIDE | 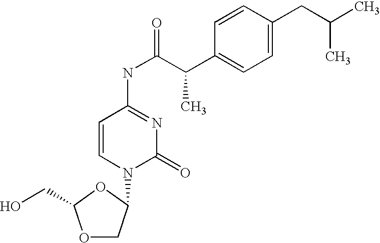 |
| 243 | 2-(4-ISOBUTYL-PHENYL)- PROPIONIC ACID 4-(4- AMINO-2-OXO-2H- PYRIMIDIN-1-YL)- [1,3]DIOXOLAN-2-YL METHYL ESTER | 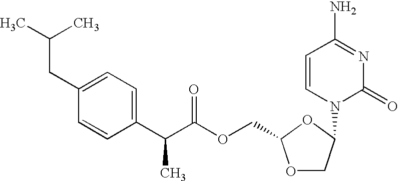 |
| 244 | DIPHENYL-CARBAMIC ACID 4-[4-(DIMETHYLAMINO- METHYLENEAMINO)-2-OXO- 2H-PYRIMIDIN-1-YL]- [1,3]DIOXOLAN-2-YL METHYL ESTER | 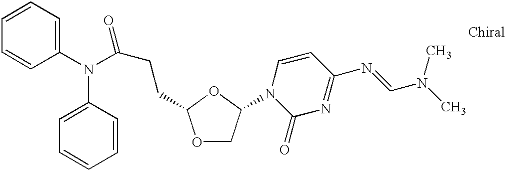 |
| 245 | 2-METHYL-8-PHENYL- OCTANOIC ACID 4-(4- AMINO-2-OXO-2H- PYRIMIDIN-1-YL)- [1,3]DIOXOLAN-2-YL METHYL ESTER | 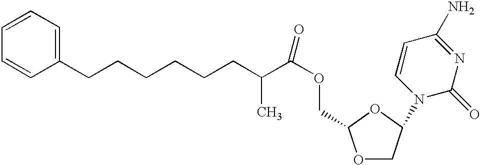 |
| 246 | DIPHENYL-CARBAMIC ACID 4-(4-AMINO-2-OXO-2H- PYRIMIDIN-1-YL)- [1,3]DIOXOLAN-2-YL METHYL ESTER | 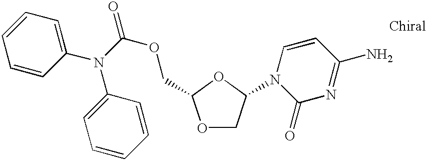 |
| 247 | 2-Methyl-8-phenyl- octanoic acid [1-(2- hydroxymethyl- [1,3]dioxolan-4-yl)-2- oxo-1,2-dihydro- pyrimidin-4-yl]-amide | 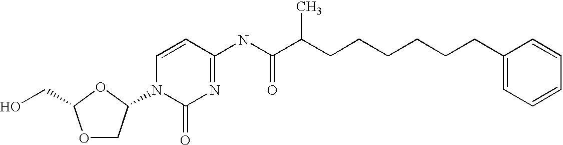 |
| 248 | 4-PENTYL- BICYCLO[2.2.2]OCTANE-1- CARBOXYLIC ACID 4-(4- AMINO-2-OXO-2H- PYRIMIDIN-1-YL)- [1,3]DIOXOLAN-2- YLMETHYL ESTER; HYDROCHLORIDE SALT | 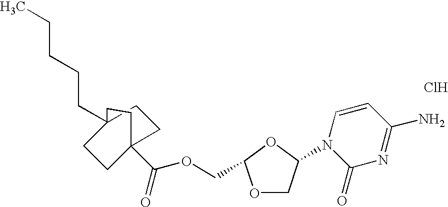 |
| 249 | #N!-[1-(2- HYDROXYMETHYL- [1,3]DIOXOLAN-4-YL)-2- OXO-1,2-DIHYDRO- PYRIMIDIN-4-YL]-3- METHYL-2-PHENYL- BUTYRAMIDE | 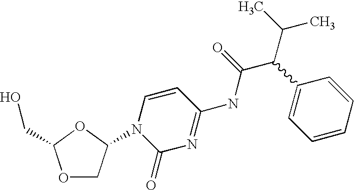 |
| 250 | [1-(2-HYDROXYMETHYL- [1,3]DIOXOLAN-4-YL)-2- OXO-1,2-DIHYDRO- PYRIMIDIN-4-YL]- CARBAMIC ACID 4-PENTYL- PHENYL ESTER | 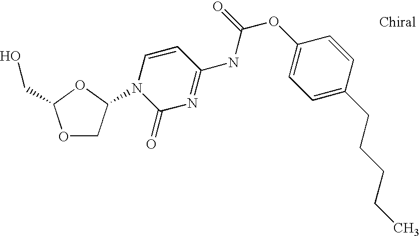 |
| 251 | Adamantane-1-carboxylic acid 4-(4-amino-2-oxo- 2H-pyrimidin-1-yl)- [1,3]dioxolan-2-yl methyl ester | 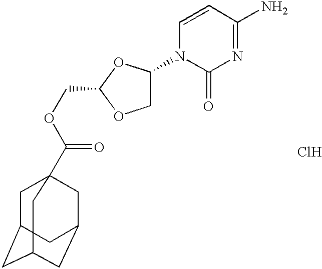 |
| 252 | 4-HEXYL-BENZOIC ACID 4- (4-AMINO-2-OXO-2H- PYRIMIDIN-1-YL)- [1,3]DIOXOLAN-2-YL METHYL ESTER; HYDROCHLORIDE SALT | 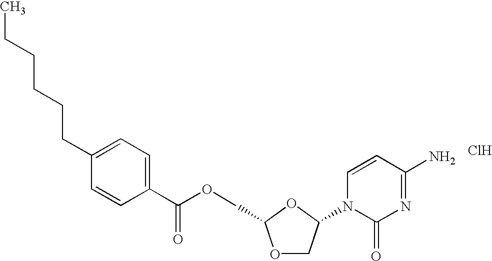 |
| 253 | 2-OXO-1-[2-(1-PHENYL- CYCLOHEXANECARBONYLOXYM ETHYL)-[1,3]DIOXOLAN-4- YL]-1,2-DIHYDRO- PYRIMIDIN-4-YL- AMMONIUM; CHLORIDE | 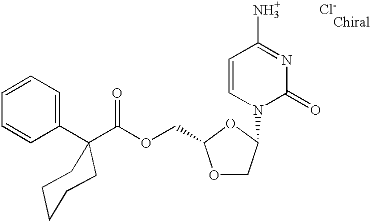 |
| 254 | {1-[1-(2-HYDROXYMETHYL- [1,3]DIOXOLAN-4-YL)-2- OXO-1,2-DIHYDRO- PYRIMIDIN-4-YL CARBAMOYL]-3-METHYL- BUTYL}-CARBAMIC ACID BENZYL ESTER | 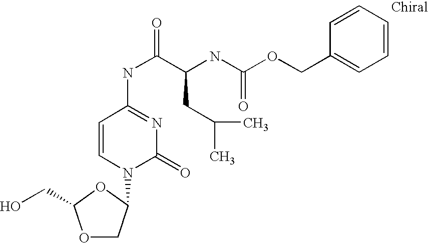 |
| 255 | [4-(4-AMINO-2-OXO-2H- PYRIMIDIN-1-YL)- [1,3]DIOXOLAN-2-YL METHOXY]- PHOSPHONO-ACETATE BIS- AMMONIUM SALT | 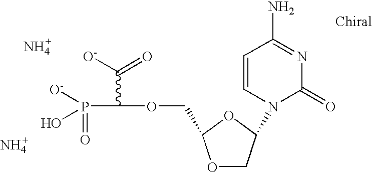 |
| 256 | 2-tert-Butyl-8-phenyl- octanoic acid 4-(4- amino-2-oxo-2H- pyrimidin-1-yl)- [1,3]dioxolan-2-yl methyl ester | 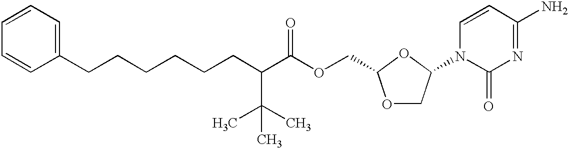 |
| 257 | 2-AMINO-4-METHYL- PENTANOIC ACID [1-(2- HYDROXYMETHYL- [1,3]DIOXOLAN-4-YL)-2- OXO-1,2-DIHYDRO- PYRIMIDIN-4-YL]-AMIDE | 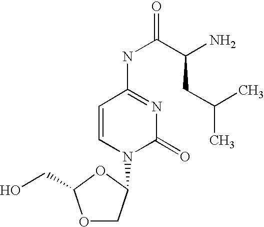 |
| 258 | BENZOIC ACID 4-(4- ACETYLAMINO-2-OXO-2H- PYRIMIDIN-1-YL)- [1,3]DIOXOLAN-2-YL METHYL ESTER | 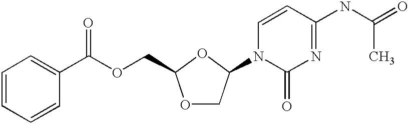 |
| 259 | BENZOIC ACID 4-(4- ACETYLAMINO-2-OXO-2H- PYRIMIDIN-1-YL)- [1,3]DIOXOLAN-2-YL METHYL ESTER | 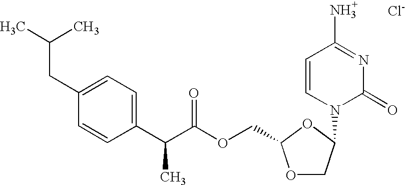 |
| 260 | 1-{2-[2-(4-ISOBUTYL- PHENYL)- PROPIONYLOXYMETHYL]- [1,3]DIOXOLAN-4-YL]-2- OXO-1,2-DIHYDRO- PYRIMIDIN-4-YL- AMMONIUM; CHLORIDE | 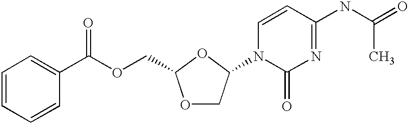 |
| 261 | 8-Phenyl-octanoic acid 4-(4-amino-2-oxo-2H- pyrimidin-1-yl)- [1,3]dioxolan-2-yl methyl ester hydrochloride | 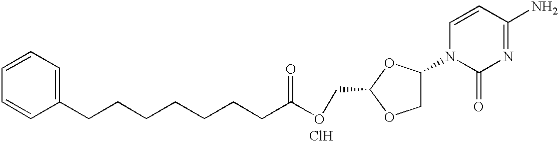 |
| 262 | 3-METHYL-2-PHENYL- BUTYRIC ACID 4-(4- AMINO-2-OXO-2H- PYRIMIDIN-1-YL)- [1,3]DIOXOLAN-2- YLMETHYL ESTER | 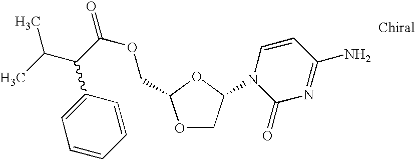 |
| 263 | (1-{1-[1-(2- HYDROXYMETHYL- [1,3]DIOXOLAN-4-YL)-2- OXO-1,2-DIHYDRO- PYRIMIDIN-4- YLCARBANOYL]-3-METHYL- BUTYLCARBAMOYL}-ETHYL)- CARBAMIC ACID TERT- BUTYL ESTER | 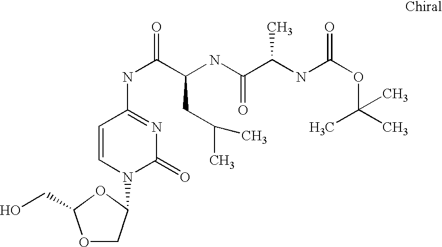 |
| 264 | 2-OXO-1-[2-(4-PENTYL- CYCLOHEXANECARBONYLOXYM ETHYL)-[1,3]DIOXOLAN-4- YL]-1,2-DIHYDRO- PYRIMIDIN-4-YL-AMMONIUM CHLORIDE | 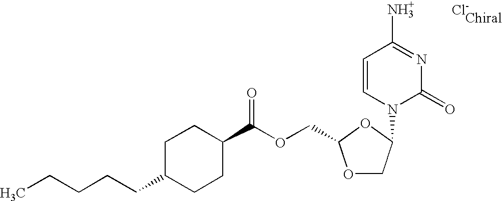 |
| 265 | 2-(2-AMINO- PROPIONYLAMINO)-4- METHYL-PENTANOIC ACID [1-(2-HYDROXYMETHYL- [1,3]DIOXOLAN-4-YL)-2- OXO-1,2-DIHYDRO- PYRIMIDIN-4-YL]-AMIDE, BIS TRIFLUOROACETIC ACID SALT | 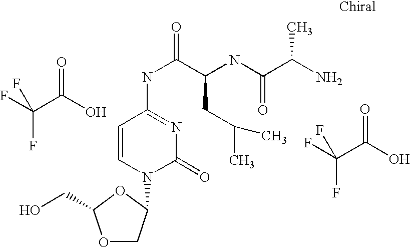 |
| 266 | 2-ETHYL-8-PHENYL- OCTANOIC ACID 4-(4- AMINO-2-OXO-2H- PYRIMIDIN-1-YL)- [1,3]DIOXOLAN-2- YLMETHYL ESTER | 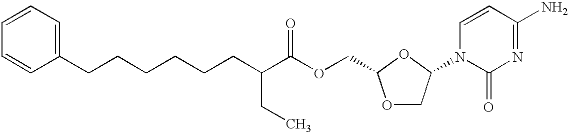 |
| 267 | [1-(1-{1-[1-(2- HYDROXYMETHYL- [1,3]DIOXOLAN-4-YL)-2- OXO-1,2-DIHYDRO- PYRIMIDIN-4- YLCARBAMOYL]-3-METHYL- BUTYLCARBAMOYL}- ETHYLCARBAMOYL)-3- METHYL-BUTYL]-CARBAMIC ACID BENZYL ESTER | 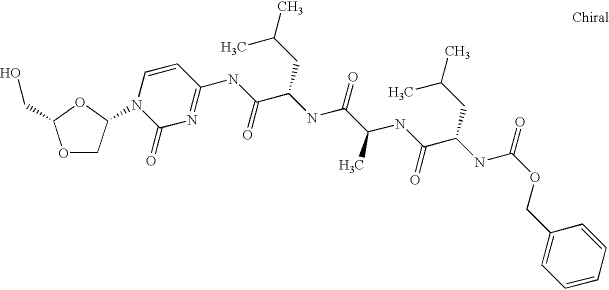 |
| 268 | 2-METHYL-8-PHENYL- OCTANOIC ACID 4-(4- AMINO-2-OXO-2H- PYRIMIDIN-1-YL)- [1,3]DIOXOLAN-2- YLMETHYL ESTER HYDROCHLORIDE | 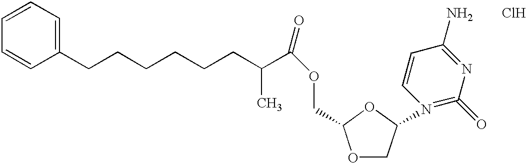 |
| 269 | 2,2-DIMETHYL-8-PHENYL- OCTANOIC ACID 4-(4- AMINO-2-OXO-2H- PYRIMIDIN-1-YL)- [1,3]DIOXOLAN-2- YLMETHYL ESTER HYDROCHLORIDE | 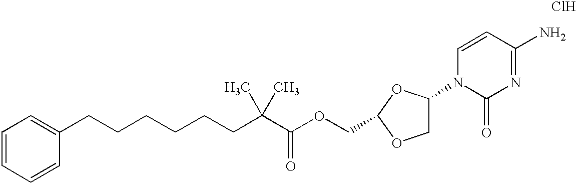 |
| 270 | BIS-(4-OCTYL-PHENYL)- CARBAMIC ACID 4-(4- AMINO-2-OXO-2H- PYRIMIDIN-1-YL)- [1,3]DIOXOLAN-2- YLMETHYL ESTER | 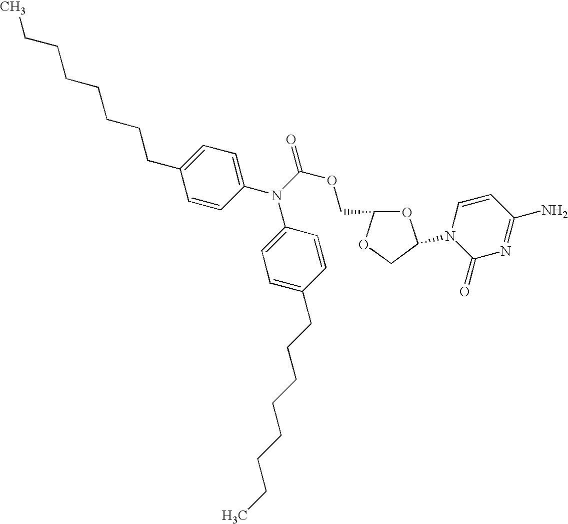 |
| 272 | 2-AMINO-4-METHYL- PENTANOIC ACID (1-{1- [1-(2-HYDROXYMETHYL- [1,3]DIOXOLAN-4-YL)-2- OXO-1,2-DIHYDRO- PYRIMIDIN-4-YL CARBAMOYL]-3-METHYL- BUTYLCARBAMOYL}-ETHYL)- AMIDE | 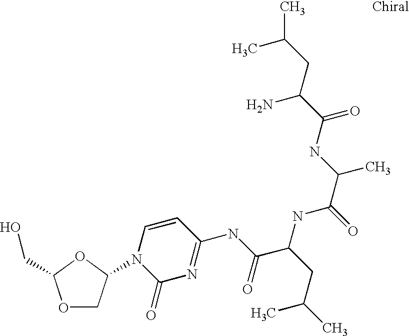 |
| 275 | ISOBUTYRIC ACID 4-(4- AMINO-2-OXO-2H- PYRIMIDIN-1-YL)- [1,3]DIOXOLAN-2-YL METHYL ESTER | 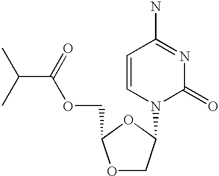 |
| 276 | 6-METHYL-HEPTANOIC ACID 4-[4-(6-METHYL- HEPTANOYLAMINO)-2-OXO- 2H-PYRIMIDIN-1-YL]- [1,3]DIOXOLAN-2-YL METHYL ESTER | 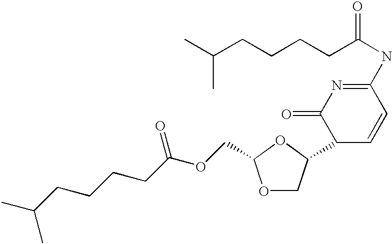 |
| 277 | 6-METHYL-HEPTANOIC ACID [1-(2-HYDROXYMETHYL- [1,3]DIOXOLAN-4-YL)-2- OXO-1,2-DIHYDRO- PYRIMIDIN-4-YL]-AMIDE | 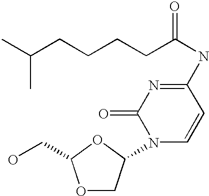 |
| 278 | 3-METHYL-BUTYRIC ACID 4-(4-AMINO-2-OXO-2H- PYRIMIDIN-1-YL)- [1,3]DIOXOLAN-2-YL METHYL ESTER | 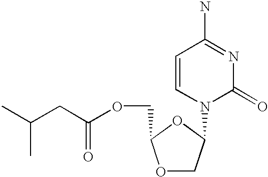 |
| 279 | 2,2-DIMETHYL-PROPIONIC ACID 4-(4-AMINO-2-OXO- 2H-PYRIMIDIN-1-YL)- [1,3]DIOXOLAN-2-YL METHYL ESTER | 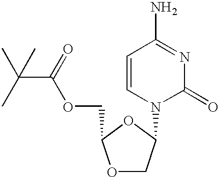 |
| 280 | 2-Amino-N-[1-(2- hydoxymethyl- [1,3]dioxolan-4-yl)-2- oxo-1,2-dihydro- pyrimidin-4-yl]-3- methyl-butyramide; trifluoroacetic acid salt | 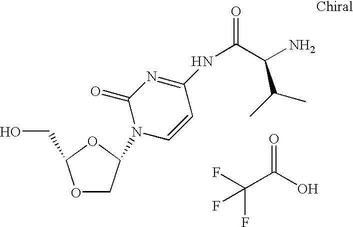 |
| 281 | 7-ISOPROPYL-2,4A- DIMETHYL- 1,2,3,4,4A,4B,5,6,10,10 A-DECAHYDRO- PHENANTHRENE-2- CARBOXYLIC ACID [1-(2- HYDROXYMETHYL- [1,3]DIOXOLAN-4-YL)-2- OXO-1,2-DIHYDRO- PYRIMIDIN-4-YL]-ESTER | 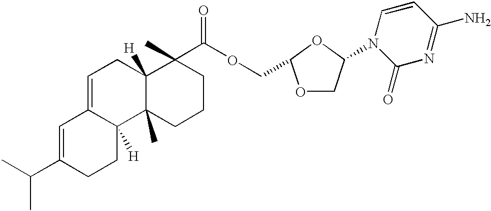 |

The following are examples of additional compounds in accordance with the invention:

[1-(2-Hydroxymethyl-[1,3]dioxolan-4-yl)-2-oxo-1,2-dihydro-pyrimidin-4-yl]-carbamic acid butyl ester

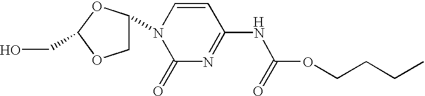

[1-(2-Hydroxymethyl-[1,3]dioxolan-4-yl)-2-oxo-1,2-dihydro-pyrimidin-4-yl]-carbamic acid pentyl ester

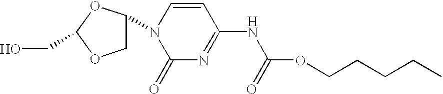

[1-(2-Hydroxymethyl-[1,3]dioxolan-4-yl)-2-oxo-1,2-dihydro-pyrimidin-4-yl]-carbamic acid hexyl ester

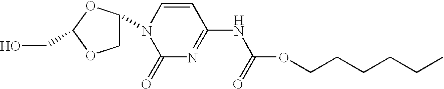

Hexanoic acid [1-(2-hydroxymethyl-[1,3]dioxolan-4-yl)-2-oxo-1,2-dihydro-pyrimidin-4-yl]-amide

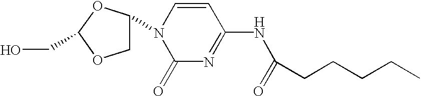

Heptanoic acid [1-(2-hydroxymethyl-[1,3]dioxolan-4-yl)-2-oxo-1,2-dihydro-pyrimidin-4-yl] -amide

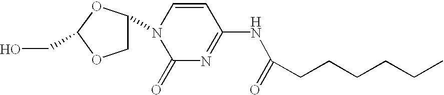

Octanoic acid [1-(2-hydroxymethyl-[1,3]dioxolan-4-yl)-2-oxo-1,2-dihydro-pyrimidin-4-yl]-amide

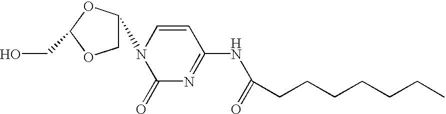

[1-(2-Hydroxymethyl-[1,3]dioxolan-4-yl)-2-oxo-1,2-dihydro-pyrimidin-4-yl]-carbamic acid 3-dimethylamino-propyl ester

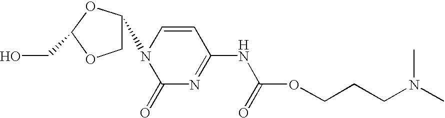

[1-(2-Hydroxymethyl-[1,3]dioxolan-4-yl)-2-oxo-1,2-dihydro-pyrimidin-4-yl]-carbamic acid 4-dimnethylamino-butyl ester

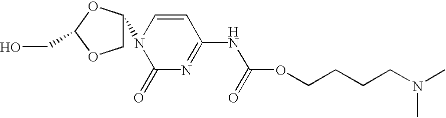

[1-(2-Hydroxymethyl-[1,3]dioxolan-4-yl)-2-oxo-1,2-dihydro-pyrimidin-4-yl]-carbamic acid 5-dimethylamino-pentyl ester

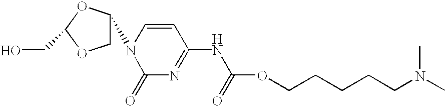

5-Dimethylamino-pentanoic acid [1-(2-hydroxymethyl-[1,3]dioxolan-4-yl)-2-oxo-1,2-dihydro-pyrimidin-4-yl]-amide

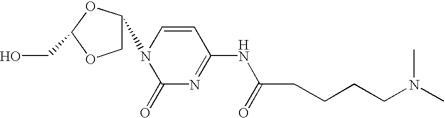

6-Dimethylamino-hexanoic acid [1-(2-hydroxymethyl-[1,3]dioxolan-4-yl)-2-oxo-1,2-dihydro-pyrimidin-4-yl]-amide

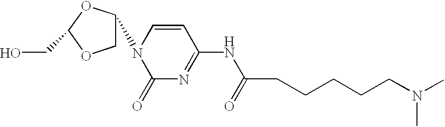

7-Dimethylamino-heptanoic acid [1-(2-hydroxymethyl-[1,3]dioxolan-4-yl)-2-oxo-1,2-dihydro-pyrimidin-4-yl]-amide

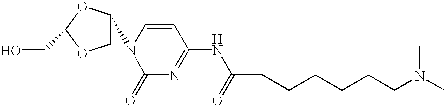

Acetic acid 4-(4-amino-2-oxo-2H-pyrimidin-1-yl)-[1,3]dioxolan-2-ylmethoxymethyl ester

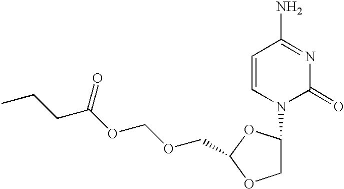

Butyric acid 4-(4-amino-2-oxo-2H-pyrimidin-1-yl)-[1,3]dioxolan-2-ylmethoxymethyl ester

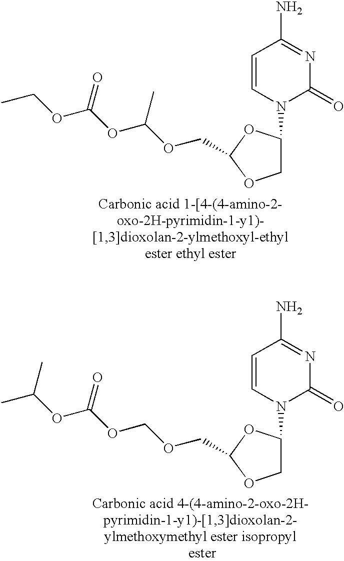

(2S, 4S) N-[1-(2-Hydroxymethyl-[1,3]dioxolan-4-yl)-2-oxo-1,2-dihydro-pyrimidin-4-yl]-2-piperidin-4-yl-acetamide trifluoroacetate salt

(2S, 4S) Piperidin-4-yl-acetic acid 4-(4-amino-2-oxo-2H-pyrimidin-1-yl)-[1,3]dioxolan-2-ylmethyl ester trifluoroacetate salt

(2S, 4S) 2-Amino-3-methyl-butyric acid 4-(4-amino-2-oxo-2H-pyrimidin-1-yl)-[1,3]dioxolan-2-ylmethyl ester trifluoroacetate salt

(2S, 4S) 2-Amino-N-[1-(2-hydroxymethyl-[1,3]dioxolan-4-yl)-2-oxo-1,2-dihydro-pyrimidin-4-yl]-3-methyl-butyramide trifluoroacetate salt

(2S, 4S) 4-Amino-1-[2-(tetrahydro-pyran-2-yloxymethyl)-[1,3]dioxolan-4-yl]-1H-pyrimidin-2-one

Additional exemplary compounds are illustrated below:

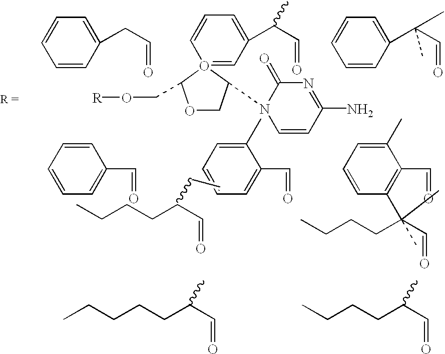

Further examples are:

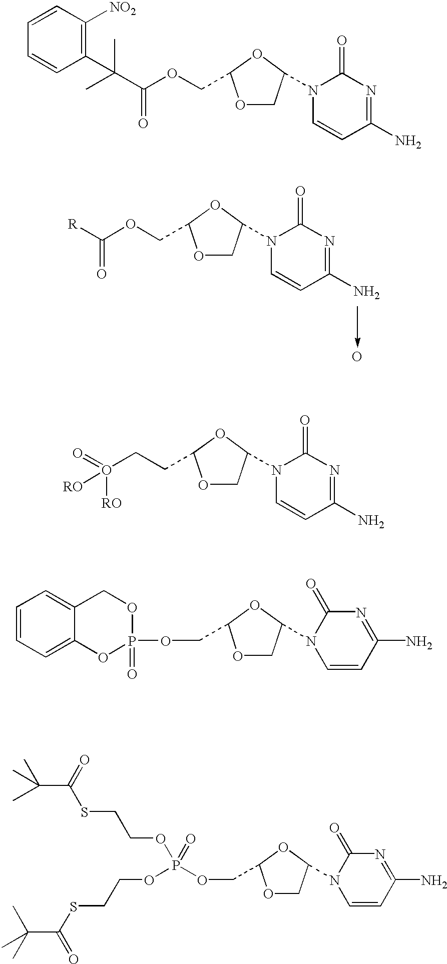

The compounds of formula (I) have a cis geometrical configuration. Moreover, the compounds of formula (I) exhibit the ‘‘unnatural’’ nucleoside configuration, that is they are L-enantiomers. Preferably, the compounds of formula (I) are provided substantially free of the corresponding D-enantiomers, that is to say no more than about 5% w/w of the corresponding D-nucleoside, preferably no more than about 2% w/w, in particular less than about 1% w/w is present.

The compounds formula (I) include compounds in which the hydrogen of the 2-hydroxymethyl group and/or one or both of the hydrogens of a base amino group(s) is replaced by alkyl, alkenyl, aryl, a heteroaromatic group or a nonaromatic ring group, or are replaced by —C(O)R⁶ or —C(O)OR⁶ groups in which R⁶ is alkyl, alkenyl, aryl optionally substituted by alkyl, a heteroaromatic group optionally substituted by alkyl, or a nonaromatic ring group.

With regard to the compounds of formula (I), unless otherwise specified, any alkyl or alkenyl moiety present advantageously contains up to 24 carbon atoms, particularly 4 to 18 carbon atoms. Any aryl moiety present preferably contains 6 to 24 carbon atoms, for example, phenyl, napthyl, and biphenyl groups.

In the compounds of formula (I), R¹, R³ and/or R⁴ can also exhibit an amino acid radical or an amino acid chain. Unless specified otherwise, the term “amino acid” used herein includes naturally-occurring amino acids as well as non natural analogs as those commonly used by those skilled in the art of chemical synthesis and peptide chemistry. A list of non natural amino acids may be found in “The Peptides”, vol. 5, 1983, Academic Press, Chapter 6 by D. C. Roberts and F. Vellaccio. Example of naturally occurring amino acid includes alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), ornithine (Orn), proline (Pro), serine (Ser), threonine (Thr), tryptophan (Trp), tyrosine (Tyr), and valine (Val). Preferably, the amino acid radical or amino acid chain exhibits at least one amino acid radical selected from Ala, Glu, Val, Leu, Ile, Pro, Phe, Tyr or Typ.

By the term “amino acid residue” and “amino acid chain residue” is meant an amino acid or amino acid chain preferably lacking the carboxy terminal hydroxyl group. For example, the amino acid residue of serine is preferably:

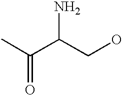

Pharmaceutically acceptable salts of the compounds of formula (I)include those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toleune-p-sulphonic, tartaric, acetic, citric, methanesulphonic, formic, benzoic, malonic, naphthalene-2-sulphonic and benzenesulphonic acids. Other acids such as oxalic, while not in themselves pharmaceutically acceptable, may be useful as intermediates in obtaining the compounds of the invention and their pharmaceutically acceptable acid addition salts.

Salts derived from appropriate bases include alkali metal (e.g. sodium), alkaline earth metal (e.g. magnesium), ammonium and NR₄+ (where R is C₁₋₄ alkyl) salts.

The compounds of the invention either themselves possess anticancer activity and/or are metabolizable to such compounds.

By the term “amino acid chain” is meant two or more, prererably 2 to 6, amino acid residues covalently bound via a peptide or thiopeptide bond.

The alkyl groups, including alkylene structures, can be straight chain or branched. In addition, within the alkyl or alkylene groups, one or more CH₂ can be replaced, in each case independently, by —O—, —CO—, —S—, —SO₂—, —NH—, N (C₁₋₄—alkyl)—, —N (C₆₋₁₀—aryl)—, —CS—, —C═NH—, or —N (CO—O— C₁₋₄-alkyl)-, in manner in which O atoms are not directly bonded to one another. In addition, one or more —CH₂ CH₂— can be replaced, in each case independently, by —CH═CH— or —C [symbol] C—. Further, alkyl and alkenyl groups can be optionally substituted by halogen, e.g., Cl and F.

Aryl can be unsubstituted or optionally substituted by one or more of NO₂, C₁₋₈-alkyl, C₁₋₈-alkoxy, —COOH, —CO—O—C₁₋₈-alkyl and halo (e.g. Cl and F) groups.

The non-aromatic C₃₋₂₀ groups, which optionally contain 1-3 heteroatoms, are unsubstituted or optionally substituted by one or more of C₁₋₈-alkyl, C₁₋₈-alkoxy, OH, C₁₋₈-hydroxyalkyl, and —CO—O—C₁₋₈-alkyl groups. By the term “heteroaromatic” is meant an unsaturated ring structure containing 5 to 10 ring atoms wherein 1 to 3 ring atoms are each selected from N, O and S. Examples of heteroaromatic groups include but are not limited to: furyl, thiophenyl, pyrrolyl, imidazolyl, pyrazoyl, oxazolyl, isoxazolyl, thiazolyl, isothiazolyl, pyridyl, pyrimidinyl, triazolyl, tetrazolyl, oxadrazolyl, thiadiazolyl, thiopyranyl, pyrazinyl, benzofuryl, benzothiophenyl, indolyl, benzimidazolyl, benzopyrazolyl, benzoxazolyl, benzisoxazolyl, benzothiozolyl, benzisothiazolyl, benzoxadiazolyl, quinolinyl, isoquinolinyl, carbazolyl, acridinyl, cinnolinyl and quinazolinyl.

Nonaromatic ring groups preferably contain 3-20 ring atoms in which 1-3 ring atoms are in each case selected from N, O and S. Preferred nonaromatic ring groups include cyclopropyl, cyclobutyl, cyclopentyl, cyclohexyl, piperazinyl, piperidinyl, morpholinyl, thiomorpholinyl, pyrrolidinyl, adamantyl or quinuclidinyl.

The compounds of formula (I) include ester compounds. Such esters can be obtained by, for example, esterification of the 2-hydroxymethyl groups with a fatty acid. Typically fatty acids contain 4-22 carbon atoms. Examples of ester compounds of formula (I) include compounds in which at least one of R₁, R₃ or R₄ is acetyl, propionyl, butyryl, valeryl, caprioic, caprylic, capric, lauric, myristic, palmitic, stearic, oleic, linoleic, or linolenic.

There is thus provided as a further aspect of the invention, methods for treating solid tumors. A further aspect of the invention, is a method of treating liver cancer or metastasis thereof, lung cancer, renal cancer, colon cancer, pancreatic cancer, uterine cancer, ovarian cancer, breast cancer, bladder cancer, melanoma and lymphoma.

Compounds of the invention can be tested for use against cancers using any of a variety of art-recognized in vitro models [e.g., inhibition of proliferation of cell lines such as tumor cell lines, as described herein and, for example, in Bowlin et al. (1998). Proc. Am. Assn. for Cancer Res. 39, #4147] or animal models [e.g., leukemic (Gourdeau et al. (2000). Cancer Chemotherapy and Pharmacology) or solid tumor (Grove et al. (1997). Cancer Res. 57: 3008-3011; Kadhim et al. (1997). Cancer Res. 57: 4803-4810; Rabbani et al. (1998). Cancer Res.58: 3461; Weitman et al. (2000). Clinical Cancer Res. 6: 1574-1578)] xenograft animal models. See, also, U.S. Pat. No. 5,817,667. Clinical tests of safety (absence of toxicity) and efficacy are carried out and evaluated using conventional testing methods.

Nucleosides can enter cells by any of a variety of mechanisms. As used herein, the term “nucleoside” means a nucleoside, nucleoside analog, modified nucleoside, or the like, for example any of the nucleoside “prodrugs” described above. Mechanisms of nucleoside uptake include, e.g., uptake by nucleoside or nucleobase transporter proteins (NT), including sodium-independent, bidirectional equilibrative transporters such as, e.g., the es or ei transporters; by sodium-dependent, inwardly directed concentrative transporters such as, e.g., cit, cib, cif, csg, and cs; by nucleobase transporters; or by passive diffusion. For a discussion of the properties of some NTs, see, e.g., Mackey et al. (1981). Cancer Research 58, 4349-4357 and Mackey et al. (1998). Drug Resistance Updates 1, 310-324, which are incorporated in their entirety by reference herein.

Methods (tests) for determining the mechanism(s) by which a nucleoside enters a cell are conventional in the art. Some such methods are described, e.g., in Gourdeau et al. (2000). “Troxacitabine has an Unusual Pattern of Cellular Uptake and Metabolism that Results in Differential Chemosensitivity to Cytosine-Containing Nucleosides in Solid-Tumor and Leukemic Cell Lines” (submitted for publication and attached hereto as an appendix) and Paterson et al. (1991) “Plasma membrane transport of nucleosides, nucleobases and nucleotides: an overview,” in Imai & Nakazawa, eds., Role of adenosine and adenosine nucleotides in the biological system, Elsevier Science Publishers, which are incorporated in their entirety by reference herein. Typical methods include, for example:

1) NT inhibitor studies: measuring the ability of a nucleoside of interest to inhibit proliferation of cells, e.g., cancer (malignant) cells, or measuring the uptake of a labeled nucleoside of interest into a cell, wherein the nucleoside is administered to the cell in the presence or absence of one or more inhibitors of nucleoside transporters. Such inhibitors include, e.g., NBMPR (nitrobenzylmercaptopurine), which is specific for the es transporter; dipyridamole, which is specific for the es and the ei NTs; and dilazep, which is specific for the NTs encoded by the genes hCNT1 and hCNT2, respectively. Reduction of activity or of uptake of a nucleoside of interest by an inhibitor of a particular NT implicates that NT in the mechanism of entry of the nucleoside into the cell; whereas the absence of such a reduction suggests that the NT is not involved. Methods to perform such assays are conventional and are disclosed, e.g., in Mackey et al., supra and in Examples 1-4.

2) Competition studies: measuring the kinetics of uptake of a labeled nucleoside which is known to be transported by a particular NT in the presence or absence of a large molar excess (e.g., about a 100 to 1000-fold excess) of an unlabeled nucleoside of interest. If the nucleoside of interest competes with the labeled nucleoside for the NT, thereby reducing or abolishing the amount of uptake of the labeled nucleoside, this implicates that NT in the mechanism of uptake of the nucleoside of interest. By contrast, the lack of such competition suggests that the NT is not involved in the uptake of the nucleoside of interest. See, e.g., Example 31 (hCNT3 experiment). Cell proliferation studies such as those described above can also be studied by comparable competition assays.

3) Competition with uridine: measuring the kinetics of uptake of a labeled nucleoside of interest in the presence of a large molar excess (e.g., about 100 to 1000-fold) of unlabeled uridine. Uridine is generally regarded as a “universal permeant,” which can be taken up by cells by all of the reported human NTs. If a large excess of uridine does not inhibit the uptake of a nucleoside of interest, this indicates that the nucleoside is not transported by at least any of the currently known nuceoside transporters and, therefore, this is consistent with entry into the cell by passive diffusion.

4) Competition with the nucleoside of interest, itself: measuring the kinetics of uptake of a labeled nucleoside of interest in the presence or absence of a large molar excess (e.g., about 100 to 1000-fold) of that nucleoside, itself, in unlabeled form. Reduction of the amount of labeled nucleoside taken up by a cell when excess unlabeled nucleoside is present suggests that a molecule with affinity for the nucleoside (e.g., a nucleoside transporter) participates in the uptake mechanism. By contrast, unchanged or increased transport of the labeled nucleoside indicates that the mechanism of uptake is by passive diffusion. See, e.g., Example 30 (HeLa cells; DU 145 cells), which demonstrates that uptake of ³H-troxacitabine is not inhibited by a large excess of unlabeled troxacitabine, indicating that the mechanism of uptake of troxacitabine in these cells is passive diffusion.

Any of the preceding tests can be carried out with any of a variety of cells which express a defined number of well-characterized nucleoside or nucleobase transporters. In addition to cell lines which naturally express defined numbers of NTs, mutant cell lines have been isolated which are deficient in one or more NTs, and/or one or more NTs can be introduced into a cell by conventional genetic recombinant methods. Genes encoding many NTs have been cloned (see, e.g., Griffiths et al. (1997) Nat. Med. 3: 89-93; Crawford et al. (1998) J. Biol. Chem. 273: 5288-5293; Griffiths et al. (1997) Biochem. J. 328: 739-743; Ritzel et al. (1997) Am. J. Physiol. 272: C707-C_{714;} Wang et al. (1997) Am. J. Physiol 273: F1058-F1065) or can be cloned by conventional methods; and methods of subcloning these genes into appropriate expression vectors are conventional. See, e.g., Sambrook, J. et al. (1989). Molecular Cloning, a Laboratory Manual. Cold Spring Harbor Laboratory Press, Cold Spring Harbor, N.Y. for methods of cloning, subcloning, and expressing genes. A typical example of a panel of cell lines expressing different combinations of NTs is disclosed, e.g., in Mackey et al., supra.

5) Studies with artificial membranes, e.g., reconstituted proteoliposomes comprising known NTs: measuring the kinetics of uptake of a labeled nuceoside of interest, e.g., in the presence or absence of inhibitors. See, e.g., Mackey et al., supra.

It will be further appreciated that the amount of a compound of the invention required for use in treatment will vary not only with the particular compound selected but also with the route of administration, the nature of the condition being treated and the age and condition of the patient and will be ultimately at the discretion of the attendant physician or veterinarian.

In a preferred dosage regimen (regime, schedule), the compound a nucleoside analog of the invention) is administered to a patient at least daily for a period of about 2 to 10 consecutive days, preferably for about 3 to 7, more preferably for about 4 to 6, most preferably for about 5 days. This treatment is repeated, for example, every 2 to 5 weeks, preferably ever 3 to 4 weeks, particularly about every 4 weeks.

The amount of nucleoside analog to be administered using the above dosage regimen can be determined by conventional, routine procedures, e.g., administering increasing amounts of the compound in order to determine the maximum tolerated dose.

For troxacitabine administration to a patient having a solid tumor, a preferred dosage range is about 1.2 to about 1.8 mg/m²/day, more preferably about 1.5 mg/m²/day. Sufficient time is allowed for the patient to recover from this treatment (e.g., for the patient to recover an adequate white blood count to withstand another round of therapy). Generally the time for recovery is about 2-5 weeks. After the recovery period, another round of daily doses is administered as above. A compound of the invention is preferably administered daily as described above about every 2 to 5 weeks, more preferably about every 3 to 4 or every 3 to 5 weeks. This dosage regimen can be repeated as necessary.

For troxacitabine administration to a patient having leukemia, higher amounts of the drug can be tolerated. The preferred dosage range for troxacitabine for this indication is about 3 to about 8 mg/m²/day, preferably about 5 to about 8 mg/m²/day, and most preferably about 8 mg/m²/day. For treatment of leukemia, only one cycle of administration is generally required, although additional cycles can be administered, provided that the drug does not reach toxic levels.

Optimal dosages for any of the nucleoside analogs of the invention can be determined without undue experimentation. Using the daily dosage regimen (schedule) described above, one of skill in the art can routinely determine, using conventional methods, the maximum tolerable dosage for any of the nucleosides described herein. Optimal dosages will vary, of course, with parameters such as age, weight and physical condition of the patient, nature and stage of the disease, stability and formulation of the compound, route of administration, or the like. In general, because nucleosides modified with lipophilic substituents undergo more efficient passive diffusion through cell membranes than does troxicitabine, the dosages used for these nucleoside analogs can be lower than those for troxacitabine, for example, 10 to 100 fold lower.

Compounds of the invention can be administered, using the dosage regimens and dosage amounts discussed above, to any patient having cancer who would benefit from the treatment. For example, the patient to be treated can exhibit cancer cells that are resistant to one or more of other, commonly administered, anticancer drugs, e.g., gemcitabine or ara-C (cytarabine). In another aspect, the malignant cells are deficient in nucleoside membrane transport via nucleoside or nucleobase transporter proteins, e.g., they lack or comprise mutant forms of known nucleoside transporters such as, for example, es, ei, cit, cib, cif, csg, and cs. In another aspect, the drug (compound) enters the cancer cell predominantly (e.g., at least about 50%) by passive diffusion.

While it is possible that, for use in therapy, a compound of the invention may be administered as the raw chemical it is preferable to present the active ingredient as a pharmaceutical formulation.

The invention thus further provides a pharmaceutical composition comprising a compound of formula (I) or a pharmaceutically acceptable salt thereof together with one or more pharmaceutically acceptable carriers therefor and, optionally, other therapeutic and/or prophylactic ingredients. The carrier(s) must be ‘acceptable’ in the sense of being compatible with the other ingredients of the formulation and not deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical formulations suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, emulsiying agents, non-aqueous vehicles (which may include edible oils), or preservatives.

The compounds according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

For topical administration to the epidermis the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or coloring agents.

Formulations suitable for topical administration in the mouth include lozenges comprising active ingredient in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical formulations suitable for rectal administration wherein the carrier is a solid are most preferably presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

Formulations suitable for vaginal administration may be presented as pessaries, tampons, creams, gels, pastes, foams or sprays containing in addition to the active ingredient such carriers as are known in the art to be appropriate.

For intra-nasal administration the compounds of the invention may be used as a liquid spray or dispersible powder or in the form of drops.

Drops may be formulated with an aqueous or non-aqueous base also comprising one more more dispersing agents, solubilising agents or suspending agents. Liquid sprays are conveniently delivered from presurrised packs.

For administration by inhalation the compounds according to the invention are conveniently delivered from an insufflator, nebuliser or a pressurised pack or other convenient means of delivering an aerosol spray. Pressurised packs may comprise a suitable propellant such as dichlorodifluoromethane, trichlorofluoromethane, dichlorotetrafluoroethane, carbon dioxide or other suitable gas. In the case of a presurrised aerosol the dosage unit may be determined by providing a valve to deliver a metered amount.

Alternatively, for administration by inhalation or insufflation, the compounds according to the invention may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

When desired the above described formulations adapted to give sustained release of the active ingredient may be employed.

The pharmaceutical compositions according to the invention may also contain other active ingredients such as antimicrobial agents, or preservatives.

The compounds of the invention may also be used in combination with each other and/or with other therapeutic agents. In particular the compounds of the invention may be employed together with known anticancer agents.

The invention thus provides, in a further aspect, a combination comprising a compound of formula (I) or a physiologically acceptable salt thereof together with another therapeutically active agent, in particular an anticancer agent.

The combinations referred to above may conveniently be presented for use in the form of a pharmaceutical formulation and thus pharmaceutical formulations comprising a combination as defined above together with a pharmaceutically acceptable carrier therefor comprise a further aspect of the invention. Suitable therapeutic agents for use in such combinations include:

1) Alkylating agents such as:

2-haloalkylamines (e.g. melphalan and chlorambucil),

2-haloalkylsulfides,

N-alkyl-N-nitrosoureas (e.g. carmustine, lomustine or

semustine),

aryltriazines (e.g. decarbazine),

mitomycins (e.g. mitomycin C),

methylhydrazines (e.g. procarbazine),

bifunctional alkylating agents (e.g. mechlorethamine),

carbinolamines (e.g. sibiromycin),

streptozotocins and chlorozotocins,

phosphoramide mustards (e.g. cyclophosphamide),

urethane and hydantoin mustards,

busulfan,

oncovin;

2) Antimetabolites such as:

mercaptopurines (e.g. 6-thioguanine and 6-[methylthio]purine),

nucleoside (e.g. β-L-dioxolane cytidine),

azapyrimidines and pyrimidines,

hydroxyureas,

5-fluorouracil,

folic acid antagonists (e.g. amethopterin),

cytarabines,

prednisones,

diglycoaldehydes,

methotrexate, and

cytosine rabinoside;

3) Intercalators such as:

bleomycins and related glycoproteins,

anthracylines (e.g. doxorubicin, daunorubicin, epirubicin, esorubicin, idarubicin, aclacinomycin A),

acridines (e.g. m-AMSA),

hycanthones,

ellipticines (e.g. 9-hydroxyellipticine),

actinomycins (e.g. actinocin),

anthraquinones (e.g. 1,4-bis[(aminoalkyl)-amino]-9,10-anthracenediones),

anthracene derivatives (e.g. pseudourea and bisanthrene),

phleomycins,

aureolic acids (e.g. mithramycin and olivomycin), and

Camptothecins (e.g. topotecan);

4) Mitotic inhibitors such as:

dimeric catharanthus alkaloids

vincristine, vinblastine and vindesine),

colchicine derivatives (e.g. trimethylcolchicinic acid)

epipodophyllotoxins and podophylotoxins

etoposide and teniposide),

maytansinoids (e.g. maytansine and colubrinol),

terpenes (e.g. helenalin, tripdiolide and taxol),

steroids (e.g. 4β-hyroxywithanolide E),

quassiniods (e.g. bruceantin),

pipobroman, and

methylglyoxals (e.g. methylglyoxalbis-(thiosemicarbazone);

5) Hormones(e.g. estrogens, androgens, tamoxifen, nafoxidine, progesterone, glucocorticoids, mitotane, prolactin);

6) Immunostimulants such as:

human interferons, cytokines, levamisole and tilorane;

7) Monoclonal and polyclonal antibodies;

8) Radiosensitizing and radioprotecting compounds such as:

metronidazole and misonidazole;

9) Other miscellaneous cytotoxic agents such as:

camptothecins,

quinolinequinones,

streptonigrin and isopropylidene azastreptonigrin),

cisplatin, cisrhodium and related platinum series complexes,

tricothecenes (e.g. trichodermol or vermicarin A), and

cephalotoxines (e.g. harringtonine);

10)Enzymes, such as

L-asparaginase;

11)Drug-resistance reversal compounds such as P-glycoprotein inhibitors, for example Verapamil, cyclosporin-c, and fujimycin;

12)Cytotoxic cells such as lymphokine activated killer -cells or T-cells;

13)Other Immunostimulants such as interleukin factors or antigens;

14)Polynucleotides of sence or antisensing nature;

15)Polynucleotides capable of forming triple helices with DNA or RNA;

16)Polyethers;

17)Distamycin and analogs;

18)Taxanes such as taxol and taxotere; and

19)Agents that are protective against drug induced toxicities such as granulocyte macrophage colony stimulating factor (GM-CSF) and granulocyte colony stimulating factor (G-CSF).

The above list of possible therapeutic agents is not intended to limit this invention in any way.

The individual components of such combinations may be administered either sequentially or simultaneously in separate or combined pharmaceutical formulations.

When a compound of formula (I), or a pharmaceutically acceptable salt thereof is used in combination with a second therapeutic agent the dose of each compound may be either the same as or differ from that when the compound is used alone. Appropriate doses will be readily appreciated by those skilled in the art.

The compounds of formula (I) and their pharmaceutically acceptable salts may be prepared by any method known in the art for the preparation of compounds of analogous structure, for example as described in international application No PCT/CA92/00211 published under No Wo 92/20669 which is herein incorporated by reference.

Certain intermediates useful in the synthesis of the compounds of the present invention can be synthesized as generally described in J. Med. Chem. 1994, 37, 1501-1507, Lyttle et al.

It will be appreciated by those skilled in the art that for certain of the methods the desired stereochemistry of the compounds of formula (I) may be obtained either by commencing with an optically pure starting material or by resolving the racemic mixture at any convenient stage in the synthesis. In the case of all the processes the optically pure desired product may be obtained by resolution of the end product of each reaction. It is also possible to resolve the final compound using chiral HPLC (high pressure liquid chromatography) as it is well known in the art.

### BRIEF DESCRIPTION OF THE DRAWINGS

Various other features and attendant advantages of the present invention will be more fully appreciated as the same becomes better understood when considered in conjunction with the accompanying figures, wherein:

FIG. 1 Comparative uptake of 30 μM [³H]-troxacitabine in CEM (Panel A) and CEM/ARAC8C (Panel B) cells. [³H]-Uridine uptake in either the presence or absence of the hENT1 inhibitor, NBMPR or 5 mM non-radioactive uridine was included for comparison as a control substrate. Each data point represents the mean (±standard deviation) of three determinations.

FIG. 2 Comparative uptake of 10 μM [³H]troxacitabine (0-240 min) (Panel B) and 10 μM [³H]D-uridine (0-6 min) (Panel A) in the presence (▴) or absence ( [symbol] ) of the hENT1 inhibitor, 100 nM NBMPR, in DU145 cells. Each data point represents the mean (±standard deviation) of three determinations.

FIG. 3 Comparative uptake of 10 AM [³H]troxacitabine and 10 μM [³H]D-uridine in HeLa cells. A. Uptake of [³H]troxacitabine ( [symbol] ) and [³H]D-uridine ( [symbol] ) in the presence of the hENT1 inhibitor, 100 nM NBMPR using a scale of 0-1500 pmol/10⁶ cells. B.Uptake of [³H]troxacitabine either in the absence ( [symbol] ) or presence of 100 nM NBMPR (▴), 100 μM dilazep (▾), 1 mM non-radioactive troxacitabine (♦) or 20 μM dipyridamole (●), using an expanded scale of 0-15 pmol/10⁶ cells. Each data point represents the mean (±standard deviation) of three determinations.

FIG. 4 Comparative uptake of 10 μM [³H]troxacitabine and 10 μM [³H]D-uridine in HeLa cells transiently transfected with recombinant pcDNA3 containing either the coding sequence for: (A) hCNT1 or (B) hCNT2. Transport assays were conducted in the presence of the equilibrative transport inhibitor, 100 μM dilazep and either in the presence ( [symbol] ) or absence (▴) of with the empty vector control plasmid (▾).sodium, and compared to HeLa cells transiently transfected with the empty vector control plasmic (▾).

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever. In the foregoing and in the following examples, all temperatures are set forth uncorrected in degrees Celsius; and, unless otherwise indicated, all parts and percentages are by weight.

The entire disclosures of all applications, patents and publications, cited above and below, including provisional applications Ser. Nos. 60,239,885 (filed Oct. 13, 2000) and No. 60/288,424 (filed May 4, 2001), are hereby incorporated by reference.

### EXAMPLE 1

Preparation of 2-(prolyloxymethyl)-4-cytosin-1″-yl-1,3-dioxolane Hydrochloride (1, 1a, and 1b)

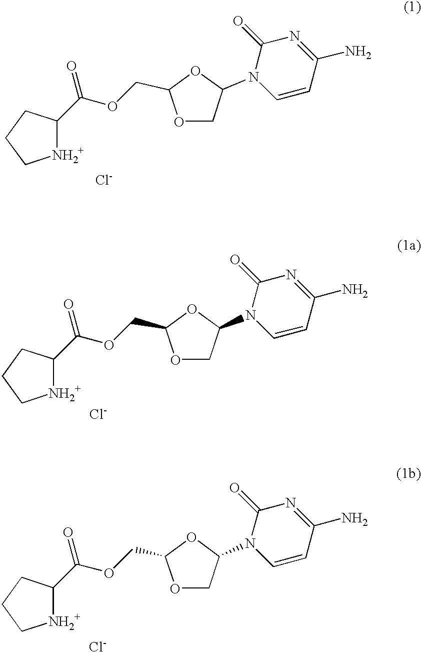

Step 1

Preparation of 4-Acetoxy-2-(O-Benzoyloxymethyl)-dioxolane

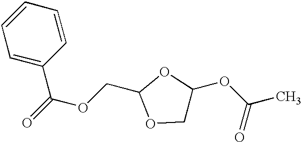

A mixture of Benzyl-1,2-Dihydroxy Butyrate (116 mg; 0.97 mmol), Benzoyloxybenzaldehyde (159mg; 0.97 mmol) and ρ-toluene sulfonic acid (9mg; 0.047 mnmol) in dry benzene (25 ml) under argon is heated at reflux for 4 h. Solvent is then removed under reduced pressure and the remaining solid is worked-up by washing with 5% sodium bicarbonate. A purification of the crude mnaterial by chromatography on silica gel gives the expected benzyl ester. The resulting compound is dissolved in ethanol (25 ml) and treated with Pd/C (excess) under hydrogen atmosphere overnight. Filtration of the catalyst and evaporation of the solvent affords the expected deprotected acid.

Lead acetate (146 mg; 0.34 mmol) and pyridine (0.03 ml, 0.33 mmol) are added to a solution of the crude solid (90 mg; 0.33 mmol) in dry tetrahydrofuran (THF) (25 ml) under argon atmosphere. The mixture is stirred for 4 h under argon and the solid is removed by filtration. The crude material is washed with ethyl acetate(EtOAc) and purified by chromatography on silica gel. This affords the pure dioxolane derivative.

Step 2

Preparation of 1-[2-benzoyloxy methyl-1,3-dioxolan-4-yl]cytosine.

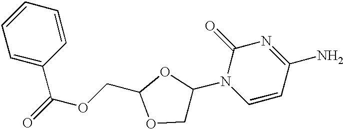

A mixture of N⁴-acetylcytosine (124 mg; 0.75 mmol), dry hexamethyl disilazane (20 ml) and ammonium sulfate (2-3 mg; catalyst) is refluxed for 5 h. under an argon atmosphere. The clear solution is cooled to room temperature and the solvent evaporated under reduced pressure. The resulting residue is dissolved in dry dichloromethane (15 ml). A solution of the dioxolane derivative obtained in step 1 (102 mg; 0.55 mmol) in dry dichloromethane (10 ml) and iodotrimethyl silane (0.076 ml; 0,54 mmol) is added to the silylated cytosine. The resulting mixture is stirred for 4 h. and worked-up by treating the solution with a 5% solution of sodium bicarbonate. The solvent of the resulting organic layer is evaporated under reduced pressure. The crude material is purified by chromatography on silica gel to give the expected nucleoside derivative.

Step 3

1-[2-hydroxymethyl-1,3-dioxolan-4-yl] N-[(dimethylamino)methylenel cytosine (268 mg; 1 mmol) is dissolved in dichloromethane (10 ml). To this solution is added dicyclohexylcarbodiimide (206 mg; 1 mmol); 4-(dimethylamnino)-pyridine (12 mg; 0.1 mmol); and Boc-praline (215 mg; 1 mmol) at 0° C. The reaction is stirred at this temperature overnight. Insoluble is filtered off and the solvent is evaporated to dryness. The solid is redissolved in dry ether (15 ml) and the solution is bubbled with HCl gas at 0° C. for ten minutes. The reaction is kept at room temperature for 2 h. The white precipitate is filtered and dried.

### EXAMPLE 2

Preparation of 2-(isoleucinyloxymethyl)-4-cytosin-1″-yl-1,3-dioxolane Hydrochloride Salt (2, 2a, and 2b)

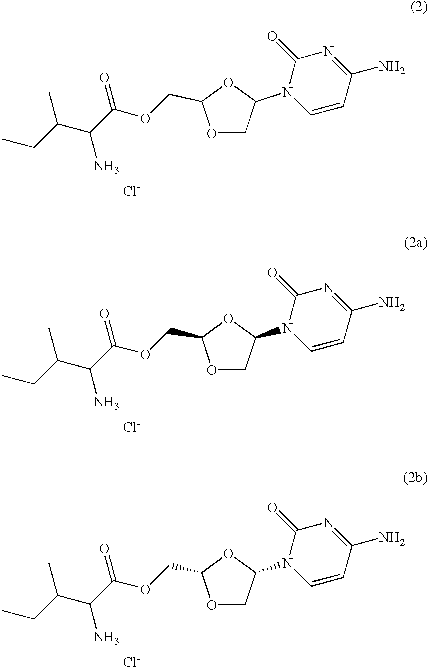

The above compound is synthesized according to the procedure described in example 1 except that proline is replaced by isoleucine.

### EXAMPLE 3

Preparation of 2-(leucinyloxymethyl)-4-cytosin-1″-yl-1,3-dioxolane Hydrochloride Salt (3, 3a, and 3b)

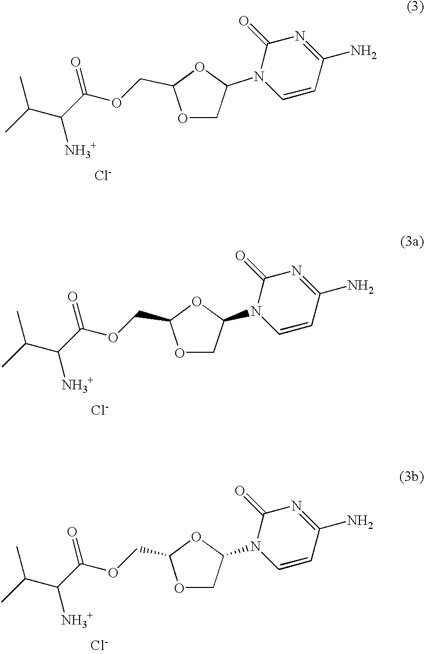

The above compound is synthesized according to the procedure described in example 1 except that proline is replaced by leucine.

### EXAMPLE 4

Preparation of 2-(cysteinyloxymethyl)-4-cytosin-1″-yl-1,3-dioxolane Hydrochloride Salt (4, 4a, and 4b)

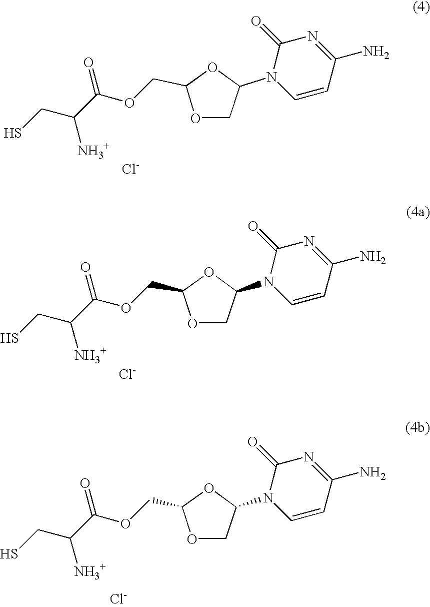

The above compound is synthesized according to the procedure described in example 1 except that proline is replaced by cysteine.

### EXAMPLE 5

Preparation of 2-(prolylglycinyloxymethyl)-4-cytosin-1″-yl-1,3-dioxolane Hydrochloride Salt (5, 5a, and 5b)

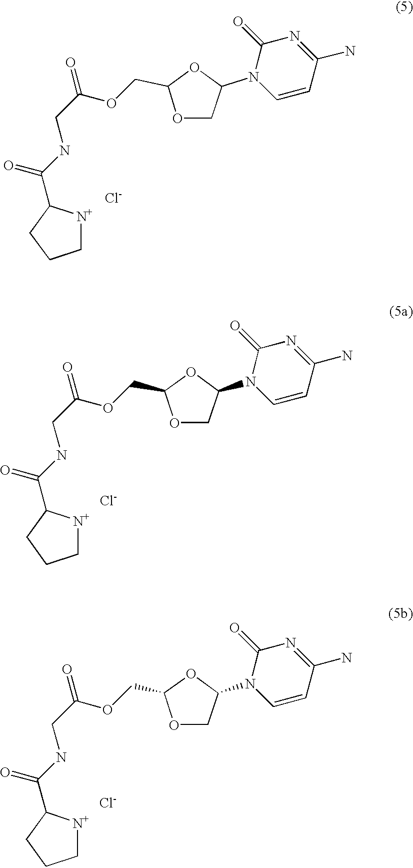

The compound is synthesized according to the procedure described in example 1 except that proline is replaced by prolylglycine.

### EXAMPLE 6

Preparation of 2-(prolylprolynyloxymethyl)-4-cytosin-1″-yl-1,3-dioxolane Hydrochloride Salt (6, 6a, and 6b)

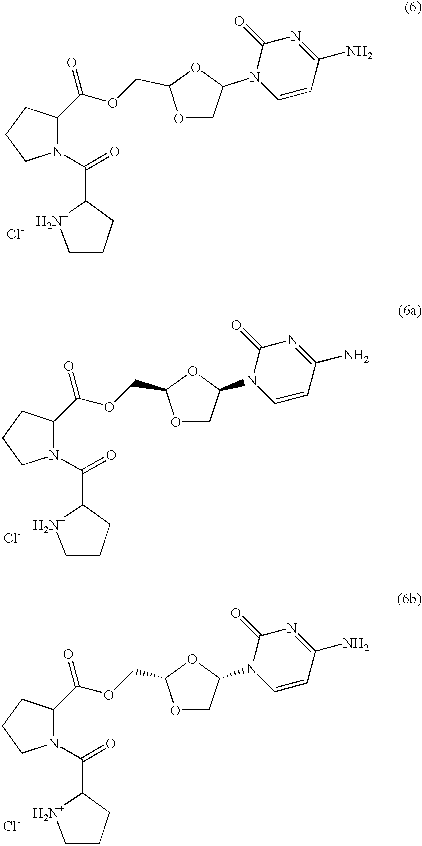

The above compound is synthesized according to the procedure described in example 1 except that proline is replaced by prolylproline.

### EXAMPLE 7

Preparation of 2-(prolylleucinyloxymethyl)-4-cytosin-1′-yl-1,3-dioxolane Hydrochloride Salt (7, 7a, and 7b)

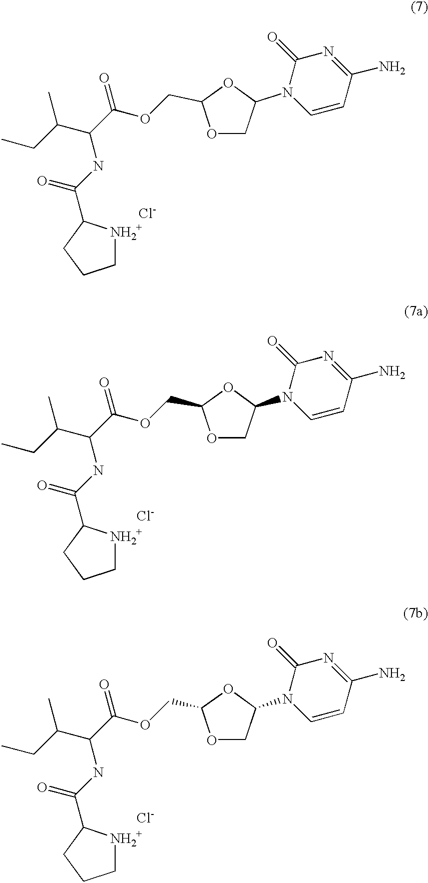

The above compound is synthesized according to the procedure described in example 1 except that proline is replaced by prolylleucine.

### EXAMPLE 8

Preparation of 2-(1′-methylthio-2′-O-methyl-3′glycerolphosphonate)-4-cytosin-1″-yl-1,3-dioxolane (8 8a, and 8b)

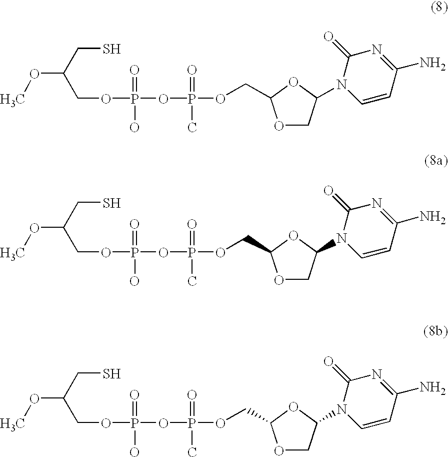

Step 1

Preparation of 1-methylthio-2-O-methyl-3 Glycerolphosphonate

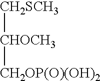

To an ice-cold mixture of Phosphorus oxychloride (445 mg; 2.9 mmol) and hexanes (5 ml) is added dropwise triethyl amine (295.35 mg; 2.9 mmol) in hexanes (5 ml). To this mixture is added dropwise a solution of dried 1-methylthio-2-O-methyl 3-glycerol (98 mg; 1.9 mmol) in toluene (100 ml) at 0-5° C. over a period of 1.5 h, and then the mixture is stirred at room temperature overnight. Water is added to the mixture and the organic layer is evaporated to give the desired product.

Step 2

Preparation of 2-(1′-methylthio-2′-O-methyl-3′glycerolphosphonate)-4-cytosin-1″-yl-1,3-dioxolane (8 8a, and 8b)

The phosphonate prepared in the first step (242 mg; 0.39 mmol) is dissolved in pyridine (10 ml). To this solution is added the dioxolane monophosphate morpholidate (198 mg; 0.31 mmol) and the mixture is stirred at room temperature for three days. Solvent is evaporated and the residue was purified by ion exchange column.

### EXAMPLE 9

Preparation of 4-cytosin-1″-yl-1,3-dioxolane-2-(tetrahydropyranylmethyl) ether (9, 9a, and 9b)

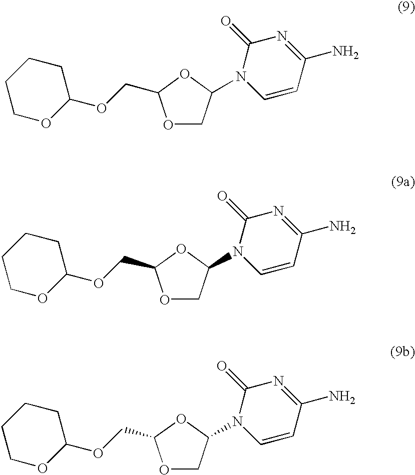

A mixture of cytosine nucleoside (684 mg; 1.9 mmol), 3,4-dihydro-2H-pyran (336 mg; 4 mmol), and p-toluene sulfonic acid (38 mg; 0.19 mmol) in dichioromethane (20 ml) is stirred for 3 h. Solvent is removed under reduced pressure and the residue is purified by chromatography.

### EXAMPLE 10

Preparation of 4-cytosin-1″-yl-1,3-dioxolane-2-(tetrahydrofuranylmethyl) ether (10, 10a, and 10b)

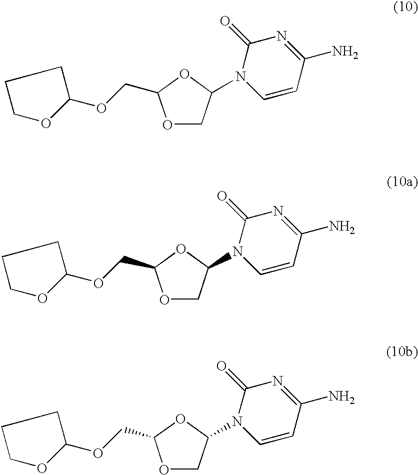

The above compound is synthesized according to the procedure described in example 9 except that 3,4-dihydro-2H-pyran is replaced by Ph₂CHCO₂-2-tetrahydrofuranyl.

### EXAMPLE 11

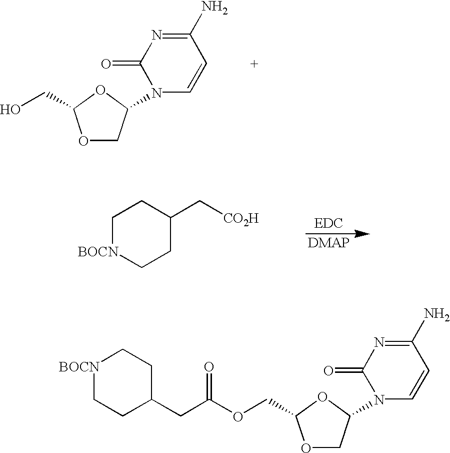

Procedure:

EDC (407 mg, 2.12 mmol, 1.0 eq) and DMAP (27 mg, 0.21 mmol, 0.1 eq) were added to a suspension of the nucleoside (451 mg, 2.12 mmol, 1.0 eq) and the acid (486 mg, 2.12 mmol, 1.0 eq) in DMF (10 mL) and the clear mixture stirred over night at room temperature. All solvent was evaporated to dryness and residue purified by chromatography (from 100% ethyl acetate to 15% methanol in ethyl acetate) 385 mg of ester was recovered.

### EXAMPLE 12

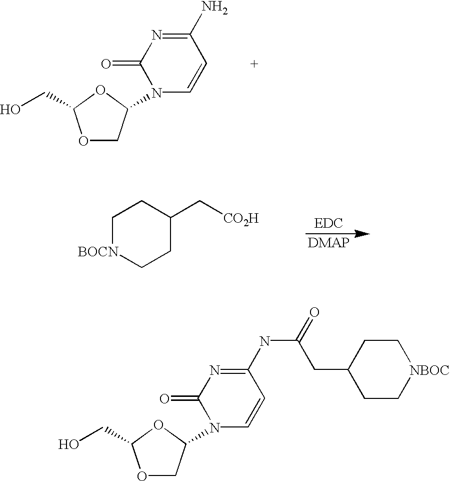

Procedure:

EDC (407 mg, 2.12 mmol, 1.0 eq) and DMAP (27 mg, 0.21 mmol, 0.1 eq) were added to a suspention of the nucleoside (451 mg, 2.12 mmol, 1.0 eq) and the acid (486 mg, 2.12 mmol, 1.0 eq) in DMF (10 mL) and the clear mixture stirred over night at room temperature. All solvent was evaporated to dryness and residue purified by chromatography (from 100% ethyl acetate to 15% methanol in ethyl acetate) 85 mg of amide was recovered.

### EXAMPLE 13

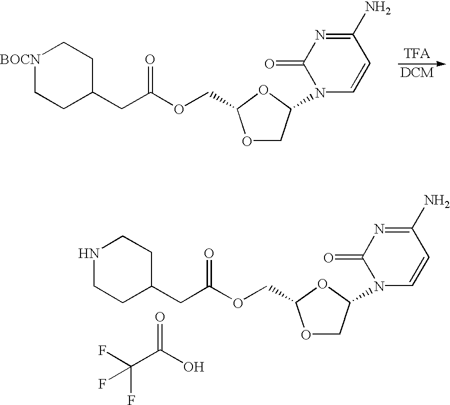

Procedure: TFA (3 mL) was added to a dichloromethane solution (7 mL) of BOC protected compound (124 mg, 0.28 mmol) and stirred for 2 hours. All solvent was evaporated to dryness. The crude was redissolved in minimal amount of methanol (0.5 mL) and slowly added to ether (10 mL) with strong agitation. The supernatant was removed and the solid dried under vacuum. 125 mg was isolated.

¹H NMR (400 MHz, DMSO-d6): 8.50 (br s, 1H), 8.25 (br s, 2H), 7.80 (d, J=7.5 Hz, 1H), 6.23 (d, J=4.0 Hz, 1H), 6.01 (d, J=8.0 Hz, 1H), 5.19 (t, J=3.0 Hz, 1H), 4.35-4.25 (m, 3H), 4.16 (m, 1H), 3.25 (d, J=13.5 Hz, 2H), 2.88 (q, J=11.0 Hz, 2H), 2.36 (d, J=7.0 Hz, 2H), 1.95 (m, 1H), 1.81 (d, J=13.0 Hz, 2H), 1.33 (q, J=10.0 Hz, 2H).

### EXAMPLE 14

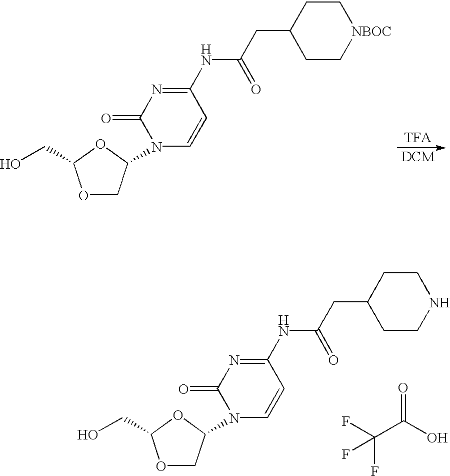

Procedure:

TFA (3 mL) was added to a dichloromethane solution (7 mL) of BOC protected compound (81 mg, 0.19 mmol) and stirred for 2 hours. All solvent was evaporated to dryness. The crude was redissolved in minimal amount of methanol (0.5 mL) and slowly added to ether (10 mL) with strong agitation. The supernatant was removed and the solid dried under vacuum. 54 mg was isolated.

¹H NMR (400 MHz, DMSO-d6): 10.92 (s, 1H), 8.50 (br s, 1H), 8.38 (d, J=7.5 Hz, 1H), 8.15 (br s, 1H), 7.22 (d, J=7.5 Hz, 1H), 6.15 (m, 1H), 5.00 (s, 1H), 4.17 (d, J=4.5 Hz, 2H), 3.71 (s, 2H), 3.24 (d, J=12.0 Hz, 2H), 2.89 (q, J=8.5 Hz, 2H), 2.39 (d, J=7.0 Hz, 2H), 2.00 (br s, 1H), 1.79 (d, J=14.0 Hz, 2H), 1.34 (q, 12.0 Hz, 2H).

### EXAMPLE 15

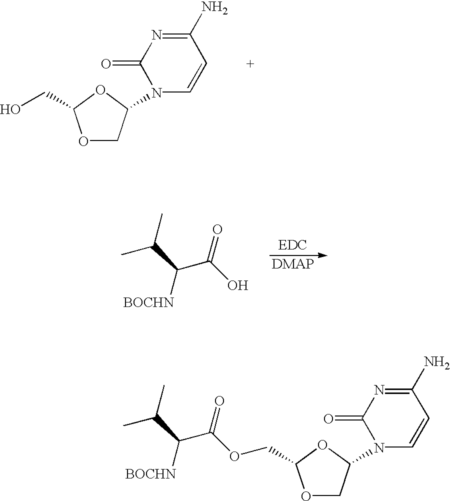

Procedure:

EDC (512 mg, 2.67 mmol, 1.0 eq) and DMAP (34 mg, 0.27 mmol, 0.1 eq) were added to a suspention of the nucleoside (568 mg, 2.67 mmol, 1.0 eq) and the acid (565 mg, 2.67 mmol, 1.0 eq) in DMF (10 mL) and the clear mixture stirred over night at room temperature. All solvent was evaporated to dryness and residue purified by chromatography (from 100% ethyl acetate to 15% methanol in ethyl acetate) 355 mg of ester was recovered.

### EXAMPLE 16

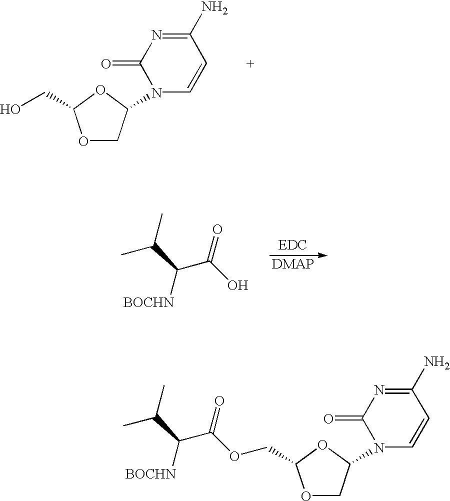

Procedure:

EDC (512 mg, 2.67 mmol, 1.0 eq) and DMAP (34 mg, 0.27 mmol, 0.1 eq) were added to a suspention of the nucleoside (568 mg, 2.67 mmol, 1.0 eq) and the acid (565 mg, 2.67 mmol, 1.0 eq) in DMF (10 mL) and the clear mixture stirred over night at room temperature. All solvent was evaporated to dryness and residue purified by chromatography (from 100% ethyl acetate to 15% methanol in ethyl acetate) 355 mg of ester was recovered.

### EXAMPLE 17

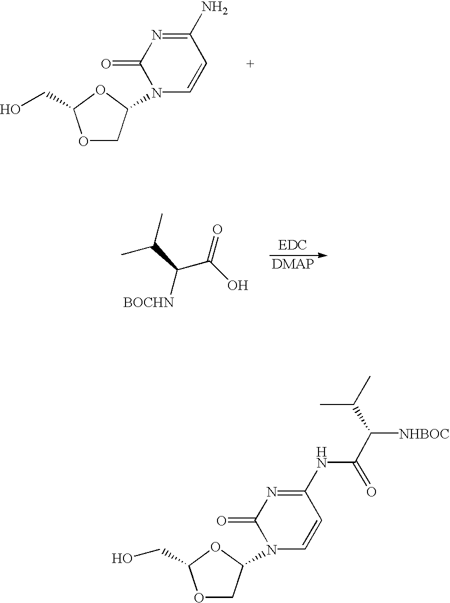

Procedure:

EDC (512 mg, 2.67 mmol, 1.0 eq) and DMAP (34 mg, 0.27 mmol, 0.1 eq) were added to a suspention of the nucleoside (568 mg, 2.67 mmol, 1.0 eq) and the acid (565 mg, 2.67 mmol, 1.0 eq) in DMF (10 mL) and the clear mixture stirred over night at room temperature. All solvent was evaporated to dryness and residue purified by chromatography (from 100% ethyl acetate to 15% methanol in ethyl acetate) 102 mg of amide was recovered.

### EXAMPLE 18

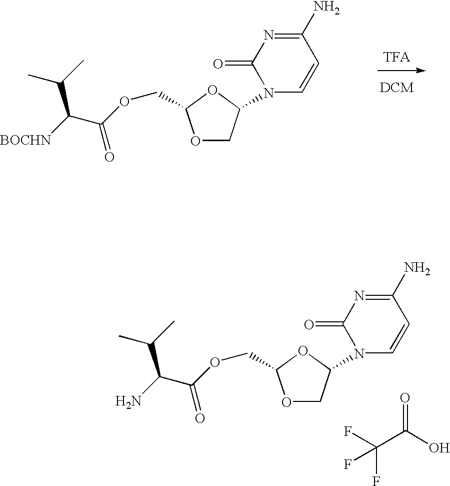

Procedure:

TFA (3 mL) was added to a dichloromethane solution (7 mL) of BOC protected compound (127 mg, 0.31 mmol) and stirred for 2 hours. All solvent was evaporated to dryness. The crude was redissolved in minimal amount of methanol (0.5 mL) and slowly added to ether (10 mL) with strong agitation. The supernatant was removed and the solid dried under vacuum. 111 mg was isolated. ¹H NMR (400 MHz, DMSO-d6): 8.40 (br s, 2H), 8.15 (br s, 1H), 7.75 (d, J=7.5 Hz, 1H), 6.27 (d, J=4.0 Hz, 1H), 6.00 (d, J=7.5 Hz, 1H), 5.23 (t, J=3.5 Hz, 1H), 4.49 (qd, J=12.0 Hz, J=3.0 Hz, 2H), 4.29 (d, J=10.0 Hz, 1H), 4.19 (m, 1H), 4.04 (s, 1H), 2.14 (m, 1H), 0.95 (D, J=7.0 Hz, 6H).

### EXAMPLE 19

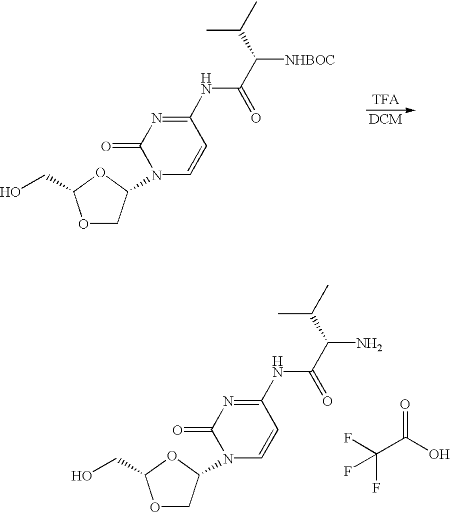

Procedure: TFA (3 mL) was added to a dichloromethane solution (7 mL) of BOC protected compound (100 mg, 0.24 mmol) and stirred for 2 hours. All solvent was evaporated to dryness. The crude was redissolved in minimal amount of methanol (0.5 mL) and slowly added to ether (10 mL) with strong agitation. The supernatant was removed and the solid dried under vacuum. 54 mg was isolated.

¹H NMR (400 MHz, DMSO-d6): 8.48 (d, J=7.5 Hz, 1H), 8.25 (br s, 3H), 7.17 (d, J=7.5 Hz, 1H), 6.16 (d, J=4.0 Hz, 1H), 5.29 (m, 1H), 5.03 (t, J=2.5 Hz, 1H), 4.25-4.15 (m, 2H), 3.90 (s, 1H), 3.72 (s, 2H), 2.18 (m, 1H), 0.95 (m, 6H).

### EXAMPLE 20

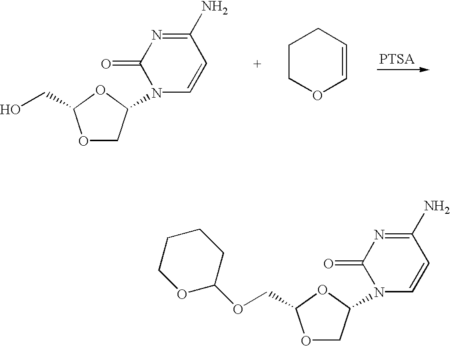

Procedure:

Paratoluene sulfonic acid (82 mg, 0.43 mmol, 1.0 eq.) was added to asolution of BCH-4556 (92mg, 0.43 mmol, 1.0 eq.) in DMF (1 mL) and 3,4-dihydropyran (3 mL). The reaction was stirred for 16 hours and potassium carbonate (119 mg, 0.86 mmol, 2.0 eq.) added and stirred for 1 hour. The solid was filtered off and the solvent evaporated to dryness. The crude was purified by flash using a gradient of 5 to 10% methanol in dichloromethane. 100 mg of desired compound was isolated.

¹H NMR (400 MHz, DMSO-d6): 7.79 (t, J=8.0 hz, 1H), 7.18 (br d, J=20.0 hz, 2H), 6.20 (m, 1H), 5.71 (d, J=7.0 hz, 1H), 5.09 (m, 1H), 4.68 (m, 1H), 4.09 (m, 2H), 3.86 (m, 1H), 3.80-3.65 (m, 2H), 3.48 (m, 1H), 1.80-1.60 (m, 2H), 1.60-1.45 (m, 4H).

### EXAMPLE 21

Preparation of Cis-L-2-[2″-cyanoethyl methoxy-L-phenylalaninylphosphoroamidyloxymethyl-4-(cytosin-1′-yl)]-1,3-dioxolane

Procedure:

Dry BCH 4556 (dimethylaminomethylene derivative, 0.1 g, 0.373 mmol) was dissolved in dry DMA (2 ml) under nitrogen and cooled in an ice bath. Diisopropylethylamine(0.2 ml) and 2,cyanoethyl-N,N-diisopropylchlorophosphoramidite (0.17 ml, 1.12 mmol) were added in respective order. After 1 hour ¹Tetrazole (0.1 g, 1.49 mmol) was added and after 10 minutes dry methanol (0.05 ml) was introduced. The reaction mixture was allowed to warm to room temperature over 2 hours. L-phenylalanine methyl ester (hydrochloride, 0.39 g, 2.18 mmol) and iodine (0.19 g, 0.746 mmol) were added in respective order. Combined mixture was allowed to stir for 2 hours and excess iodine was quenched with saturated sodium thiosulphate solution. It was evaporated to dryness and the residue was extracted with dichloromethane, washed with brine and dried over an hydrous MgSO₄. After evaporation the crude product was purified on a flash silica gel column which was eluted with a mixture of dichloromethane and methanol (ratio 10:1). Tare of the title compound was 0.072 g.

¹H-NMR (400 MHz, CDCl₃): δ:7.95(1H, d); 6.7(1H, dd); 6.2(1H, dd); 5.01(1H,s); 4.9-2.5 (m, 14H) ppm.

Appearance oil

Ref. Abraham, T. W.; Wagner, C. R. Nucleosides &

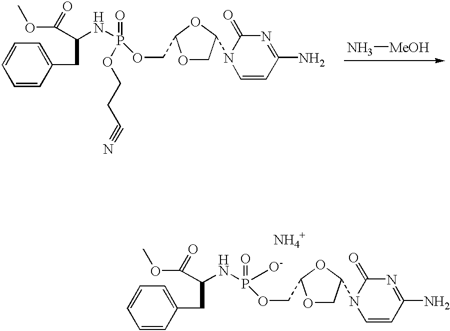

Nucleotides, 13(9), 1891-1903 (1994)

### EXAMPLE 22

Preparation of Cis-L-2-methoxy-L-phenylalaninylphosphoro-amidyloxymethyl-4-(cytosin-1′-yl)]-1,3-dioxolane Ammonium Salt

Ref Abraham, T. W.; Wagner, C. R. Nucleosides & Nucleotides, 13(9), 1891-1903 (1994)

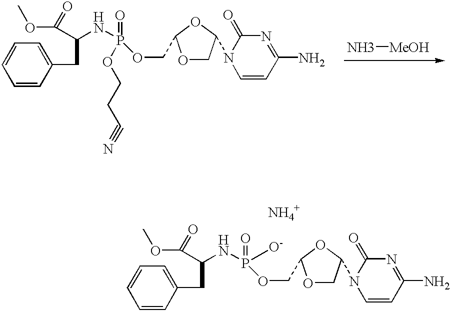

Appearance Foam

Procedure:

Dry Cis-L-2-[2″-cyanoethyl methoxy-L-phenylalaninylphosphoroamidyloxymethyl-4-(cytosin-1′-yl)]-1,3-dioxolane (0.072 g, 0.128 mmol) was dissolved in dry methanol (9.7 ml) and mixed with a saturated solution of ammonia in dry methanol (5.8 ml). Combined mixture was allowed to stir for 1 hour. Solvent was evaporated and the crude product was purified ona silica gel column which was eluted with a mixture of dichloromethane and methanol (ratio 2:1). Tare of the title compound was 0.031 g.

¹H NMR(400 MHz, CD₃OD) δ: 8.15(1H,d); 7.2(5H,m); 6.25(1H,t); 6.05(1H,d); 5.08(1H,s); 4.05(5H,m); 3.55(3H,s); 3.0(2H,qq) ppm.

UV: λₘₐₓ (MeOH) 272 nm.

MS: m/e 453.2

### EXAMPLE 23

Preparation of Cis-1-Cyclosaligenyl-2-oxymethyl-[(4-cytosin-1′-yl)-1,3-dioxolane]-phosphate Diastereomers

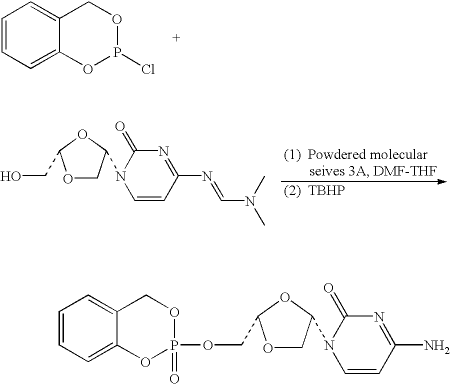

Procedure:

Dry BCH 4556(dimethylaminomethylene derivative, 0.05g, 0.1865 mmol) was dissolved in dry DMF (2 ml) and dry THF (1 ml). It was cooled to −40° C. in an argon atmosphere. Freshly activated powdered molecular sieves (0.05 g) were added. Cyclic saligenylchloroposphanes (0.071 g, 0.373 mmol) was dissolved in dry THF (0.5 ml) and introduced over 30 minutes. Combined mixture was stirred at −40° C. for another half an hour. Tert-Butylhydroproxide (3 M solution in 2,2,4-trimethylpentane, 0.125 ml) was added. After stirring for half an hour, the reaction mixture was allowed to wam to room temperature. The solvent was evaporated and the crude product was extracted with ethyl acetate. It was purified on a silica gel column using a mixture of ethyl acetate and methanol (ratio 5:2). Further purification and the separation of diastereomers was carried on reverse phase HPLC.

¹H NMR(400 MHZ, DMSO-D6) δ: 8.25(1H,d); 7.4(5H,m); 6.15(1H,t); 5.75(1H,d), 5.5(2H,m); 5.2(1H,s); 4.2(4H,m) ppm.

UV: λₘₐₓ (MeCN) 277 nm

MS: m/e 381

Ref Meier, C.; Knispel, T.; Appearance Foam Marquez, V. E.; Siddiqui, M. A.; De Clercq, E.; Balzarini, J. J. Med. Chem. 1999, 42, 1615-1624.

### EXAMPLE 24

Preparation of Cis-L-2-methoxy-L-tryptophanyllphosphoroamidyloxyethyl-4-(cytosin-1′-yl)]-1,3-dioxolane Ammonium Salt

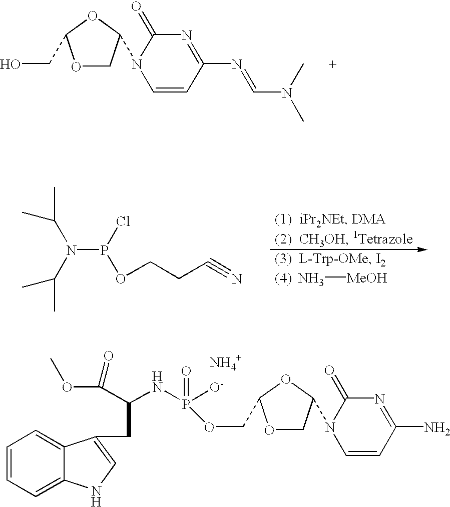

Procedure:

Dry BCH 4556 (dimethylaminomethylene derivative, 0.16 g, 0.597 mmol) was dissolved in dry DMA (3.2 ml) under nitrogen and cooled in an ice bath. Diisopropylethylamine (0.32 ml) and 2,cyanoethyl-N,N-diisopropylchlorophosphoramidite (0.27 ml, 1.79 mmol) were added in respective order. After 1 hour ¹Tetrazole (0.16 g, 2.38 mmmol) was added and after 10 minutes dry methanol (0.08 ml) was introduced. The reaction mixture was allowed to warm to room temperature over 2 hours. L-tryptophan methyl ester (hydrochloride, 0.74 g, 3.5 mmol) and iodine (0.32 g, 1.2 mmol) were added in respective order. Combined mixture was allowed to stir for 2 hours and excess iodine was quenched with saturated sodium thiosulphate solution. It was evaporated to dryness and the residue was extracted with dichloromethane, washed with brine and dried over an hydrous MgSO₄. After evaporation the crude product was purified on a flash silica gel column which was eluted with a mixture of dichloromethane and methanol (ratio 5:1).

The product was dissolved in dry methanol (15 ml) and mixed with a saturated solution of ammonia in dry methanol (9.3 ml). Combined mixture was allowed to stir for 1 hour. Solvent was evaporated and the crude product was purified on a silica gel column which was eluted with a mixture of dichloromethane and methanol (ratio 2:1). Tare of the title compound was 0.016 g.

¹H NMR(400 MHz, CD₃OD) δ: 8.1(1H,d); 7.2(5H,m); 6.2(1H,t); 5.95(1H,d); 5.05(1H,s); 4.1(5H,m); 3.35(SH,m) ppm.

### EXAMPLE 25

Preparation of (2S, 4S)-2-[bis (S-pivaloyl-2-thioethyl)phosphono]-4-cytosin-1′-yl-1.3-dioxolane

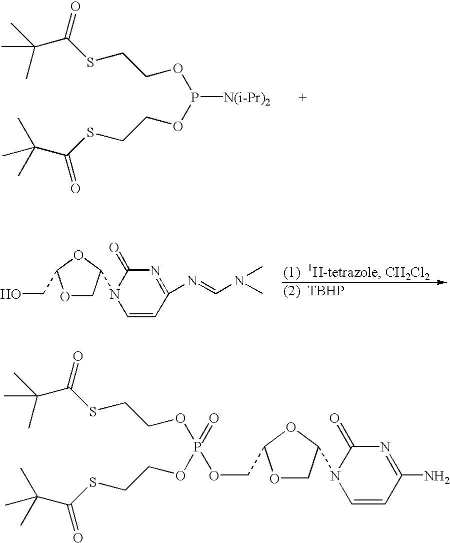

Procedure:

Dry BCH 4556 (dimethylaminomethylene derivative, 0.095 g, 0.354 mmol) was mixed with bis-(S-pivaloyl-2-thioethyl)-N,N-diisipropylphosphoramidite (0.18 g, 0.5 mmol, prepared following the procedure described in P.R.No.27-25) and dissolved in dry dichloromethane (15 ml). ¹H-tetrazole (0.075 g, 1.06 mmol) was added and the combined solution was stirred under nitrogen atmosphere at room temperature for 1 hour. It was cooled to −40° C. and treated with tert-butylhydroproxide (3 M solution in 2,2,4-trimethylpentane, 0.25 ml). Reaction mixture was allowed to warm up to room temperature during overnight. Solvent was evaporated and the residue was purified on a silica gel column using a mixture of ethyl acetate and methanol (ratio 40:1). Tare of the title product 0.055 g.

¹H NMR(400 MHz, CDCl₃) δ: 7.8(1H, d); 6.3(1H, t); 5.95(1H, d); 4.18(8H, m); 3.15(4H, m); 1.2(18H, s) ppm.

31P NMR(16 MHz, CDCl₃) δ: −0.13

UV: λₘₐₓ (MeCN) 271 nm

MS: m/e 582.4

### EXAMPLE 26

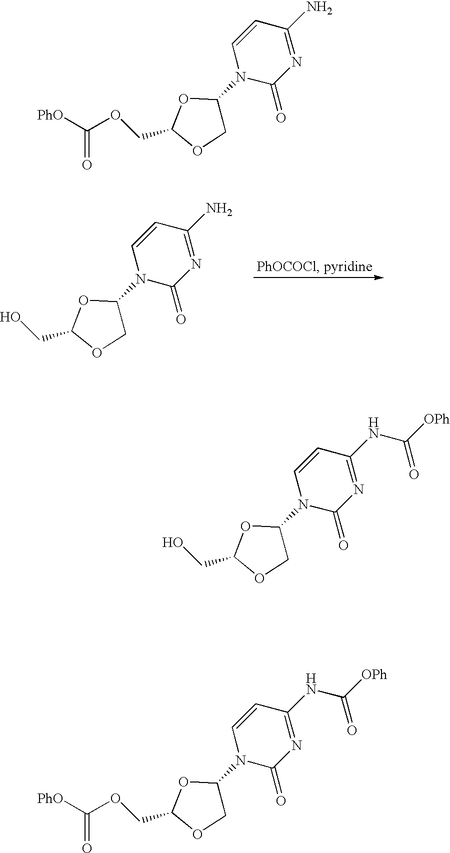

Typical Procedure for the Reaction with Alkyl(or Aryl) Chloroformate

BCH-4556 (1 mmole) and phenyl chloroformate (1 mmole) were stirred for 24 hours in 10 mL of pyridine. Pyridine was then evaporated, the residue was dissolved in 10 mL of water and extracted with dichloromethane. The organic phase is dried on sodium sulfate evaporated and the residue is chromnatographed on silica gel eliuuting firdt with 50/50 ethyl acetate/hexane, then ethyl acetate and finally with 10% MeOH/dichloromethane. The three compounds were isolated separately. The final products can be further purified using reverse phase preparative HPLC.

### EXAMPLE 27

The following are additional synthesis reaction schemes.

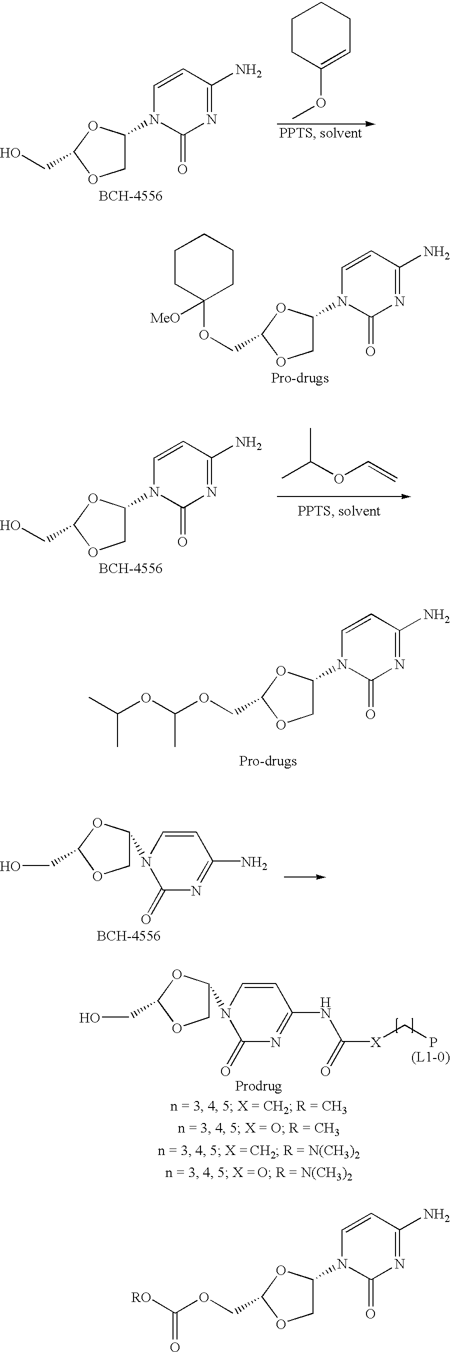

### EXAMPLE 28

Preparation of [1-(2-Hydroxymethyl-[1,3]dioxolan-4-yl)cysosyl]carbamic Acid Benzyl Ester

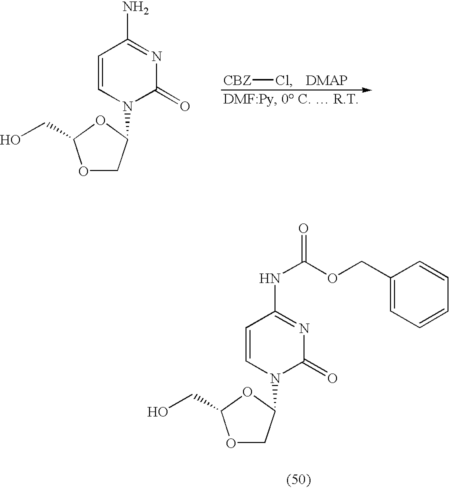

Procedure:

Benzylchloroformate (0.80 mL, 5.6 mmol) was added dropwise to a 0° C. solution of BCH-4556 (955 mg, 4.48 mmol) and DMAP (657 mg, 5.38 mmol) in dimethylformamide and pyridine and stirred at room temperature for 18 h. The reaction mixture was concentrated in vacuo. The oil obtained was partitioned between water (20 mL) and dichloromethane (30 mL). Aqueous layer was extracted with DCM. Organic layers were combined, dried over MgSO₄, filtered and concentrated to a yellow gum. The crude residue was purified by silica gel biotage (40S) (100% DCM to 10% MeOH: 90% DCM) to give 837 mg (54% yield) of [1-(2-Hydroxymethyl-[1,3]dioxolan-4-yl)cysosyl]carbamic acid benzyl ester as a white powder, M.F. C₁₆H₁₇N₃O₆, M.W. 347.33.

¹H NMR (400 MHz, CDCl₃), δ ppm: 8.44 (d, 1H, J=7.4 Hz), 7.39-7.37 (m, 5H), 7.25 (m, 1H), 6.18 (d, 1H, J=3.9 Hz), 5.21 (s, 2H), 5.13-5.12 (m, 1H), 4.34 (d, 1H, J=10.1 Hz), 4.25 (dd, 1H, J=5.2, 10.1 Hz), 4.01-3.97 (m, 2H). MS: ES⁺ 348.4 (M+1), ES⁻ 346.3 (M-1).

### EXAMPLE 29

Preparation of [1{2-(trans-4-pentylcyclohexylcarboxy) oxy-methyl-[1,3]dioxolan-4-yl}cysosyl]carbamic Acid Benzyl Ester

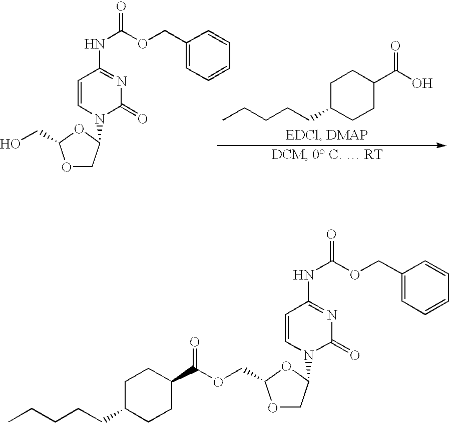

Procedure:

EDCI (1.66 g, 8.64 mmol) was added to a 0° C. solution of [1-(2-Hydroxymethyl-[1,3]dioxolan-4-yl)cysosyl]carbamic acid benzyl ester (2.5 g, 7.20 mmol), DMAP (1.05 g, 8.64 mmol) and trans-4-pentylcyclohexylcarboxylic acid (1.71 g, 8.64 mmol) in dichloromethane and stirred at room temperature for 18 h. The reaction was washed with HCl, saturated NaHCO₃ and brine. Organic layer was separated, dried over MgSO₄, filtered and concentrated in vacuo. The crude residue was purified by silica gel biotage (40M) (100% DCM to 3% MeOH: 97% DCM) to give 3.92 g (100 k yield) of [1{2-(trans-4-pentylcyclohexylcarboxy) oxymethyl-1,3]dioxolan-4-yl}cysosyl]carbamic acid benzyl ester as a white powder, M.F. C₂₈H₃₇N₃O₇, M.W. 527.62.

¹H NMR (400 MHz, CDCl₃), δ ppm: 8.15 (d, 1H, J=7.4 Hz), 7.39-7.31 (m, 5H), 7.30 (d, 1H, J=7.4 Hz), 6.19 (d, 1H, J=4.1 Hz), 5.24-5.22 (m, 3H), 4.55 (dd, 1H, J=3.3, 12.7 Hz), 4.32-4.22 (m, 3H), 2.31-2.23 (m, 1H), 1.99-1.91 (m, 2H), 1.85-1.80 (m, 2H), 1.49-1.37 (m, 1H), 1.31-1.16 (m, 10H), 0.98-0.86 (m, 5H).

### EXAMPLE 30

Preparation of trans-4-Pentylcyclohexylcarboxylic acid 4-cytosyl-[1,3]dioxolan-2-ylmethyl Ester

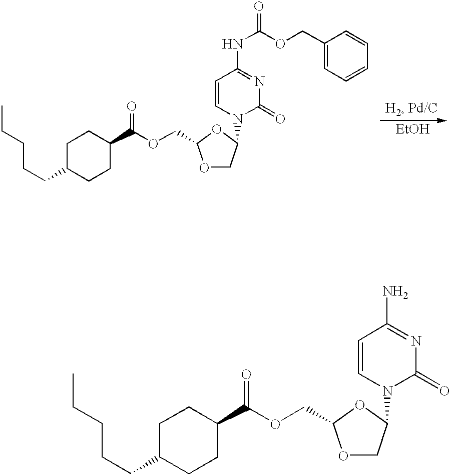

Procedure:

[1{2-(trans-4-pentylcyclohexylcarboxy)oxymethyl-[1,3]dioxolan-4-yl}cysosyl]carbamic acid benzyl ester (3.8 g, 7.20 mmol) and Pd/C₁₀% (600 mg) were suspended in ethanol and EtOAc. The reaction was treated three times with a vacuum-nitrogen sequence and left under nitrogen. It was then submitted to a vacuum-hydrogen sequence and the reaction stirred under hydrogen for 3 hrs. The reaction was filtered on a celite pad and washed with EtOH and the solution concentrated in vacuo. The crude solid was purified by silica gel biotage (40M) to give 2.44 g (86% yield) of trans-4-pentylcyclohexylcarboxylic acid 4-cytosyl-[1,3]dioxolan-2-ylmethyl ester as a white powder, M.F. C₂₀H₃₁N₃O₅, M.W. 393.49.

¹H NMR (400 MHz, CD₃OD), δ ppm: 7.85 (d, 1H, J=7.5 Hz), 6.23 (dd, 1H, J=1.9, 5.3 Hz), 5.90 (d, 1H, J=7.5 Hz), 5.21 (t, 1H, J=2.7 Hz), 4.43 (dd, 1H, J=2.7, 12.7 Hz), 4.29 (dd, 1H, J=2.6, 12.7 Hz), 4.25-4.17 (m, 2H), 2.29-2.22 (m, 1H), 1.95-1.89 (m, 2H), 1.83-1.80 (m, 2H), 1.44-1.19 (m, 11H), 0.99-0.88 (m, 5H).

### EXAMPLE 31

Preparation of trans-4-Pentylcyclohexylcarboxylic Acid 4-cytosyl-[1,3]dioxolan-2-ylmethyl Ester Hydrochloride Salt

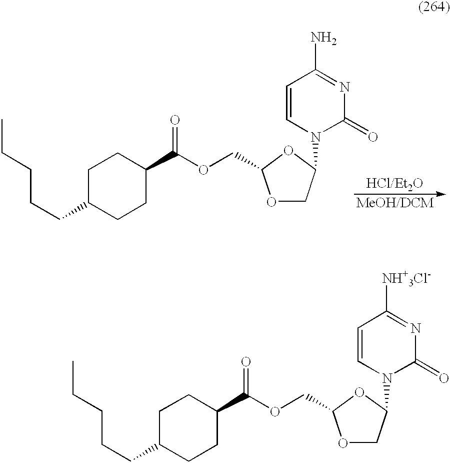

Procedure:

A 1M ether solution of HCl was added to a 0° C. solution of trans-4-pentylcyclohexylcarboxylic acid 4-cytosyl-1,3]dioxolan-2-ylmethyl ester in a 1:1 mixture of MeOH and DCM and the reaction strirred at room temperature for 1.5 h. Solvent was then removed in vacuo to give 99% yield of trans-4-pentylcyclohexylcarboxylic acid 4-cytosyl-1,3]dioxolan-2-ylmethyl ester hydrochloride salt as a white powder, M.F. C₂₀H₃₁N₃O₅HCl, M.W. 429.95.

¹H NMR (400 MHz, CD₃OD), δ ppm: 8.13 (d, 1H, J=7.8 Hz), 6.26 (dd, 1H, J=1.5, 5.5 Hz), 6.11 (d, 1H, J=7.8 Hz), 5.24 (t, 1H, J=2.8 Hz), 4.47 (dd, 1H, J=2.8, 12.6 Hz), 4.40 (dd, 1H, J=1.2, 10.3), 4.31 (dd, 1H, J=2.8, 12.6 Hz), 4.22 (dd, 1H, J=5.5, 10.3 Hz), 2.31-2.25 (s, 1H), 1.96-1.91 (m, 2H), 1.85-1.82 (m, 2H), 1.42-1.19 (m, 11H), 0.96-0.88 (m, 5H).

### EXAMPLE 32

Preparation of Octadecen-9-enoic[1-(2-hydroxymethyl-[1,3]dioxolan-4-yl)-2-oxo-1,2-dihydro-pyrimidin-4-yl]-amide

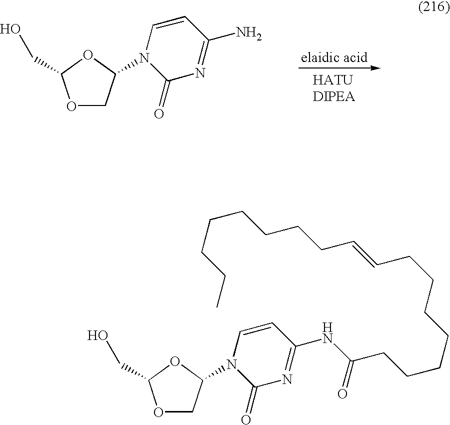

Procedure:

The starting material (BCH-4556, 86,3 mg, 0,405 mmole) is dissolved in DMF. Diisopropylethyl amine is then added (0,486 mmole, 1,2 eq) followed by the acid (0,521 mmole, 1,3 eq.). CH₂Cl₂ is then added to put everything in solution. HATU (168 mg, 0,446 mmole, 1,1 eq) is then added and the solution is stirred for 2 days. A saturated aqueous solution of NaHCO₃ is then added and extracted with CH₂Cl₂. The organic phase is evaporated and the residue is purified by Biotage with a Flash 12S column using 2% MeOH in CH₂Cl₂ followed by 4% MeOH in CH₂Cl₂. The desired fractions are recovered and evaporated to afford 39% of the desired compound.

¹H NMR (400 MHz, CDCl₃) δ 8,98 (s, 1H), 8,46 (d, 1H, J=7,6 Hz), 7,42 (d, 1H, J=7,6 Hz), 6,18 (dd, 1H, J=5,2 and 1,4 Hz), 5,36 (m, 2H), 5,11 (t, 1H, J=1,8 Hz), 4,31 (dd, 1H, J=10,2 and 1,3 Hz), 4,23 (m, 1H), 3,86 (s, 2H), 3,02 (s, 1H), 2,44 (t, 2H, J=7,6 Hz), 1,94 (m, 4H), 1,64 (m, 2H), 1,43 (m, 20H), 0,86 (t, 3H, J=6,9 Hz).

### EXAMPLE 33

Preparation of Carbonic acid 4-(2-oxo-4-phenoxycarbonylamino-2H-pyrimidin-1-yl)-[1,3]dioxolan-2-ylmethyl Ester Phenyl Ester

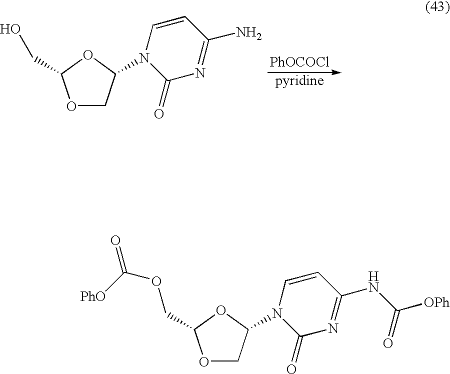

Procedure:

The starting material (BCH-4556, 105 mg, 0,493 mmole) is dissolved in 2 mL of pyridine and cooled to 0° C. Phenyl chloroformate (68 pL, 0,542 mmole, 1,1 eq.) is added and the reaction mixture is warmed to room temperature and stirred overnight. The solvent is then evaporated and water is added. The aqueous phase is extracted with methylene chloride. The organic extracts are dried over Na₂SO₄ and evaporated. The residue is purified by Biotage with 50/50 AcOEt/Hexane then AcOEt followed by 10% MeOH/CH₂Cl₂. The fractions contaning the fastest eluting spots are evaporated and repurified with preparative HPLC (C18 Deltapak 30×300 mm, 15% to 70% CH₃CN in water).

¹H nmr (400 MHz, CDCl₃) δ 8,31 (d, 1H, J=7,6 Hz), 7,39 (m, 4H), 7,26 (m, 3H), 7,16 (m, 4H), 6,31 (d, 1H, J=4,4 Hz), 5,32 (t, 1H, J=2,3 Hz), 4,69 (dd, 1H, J=12,6 and 2,6 Hz), 4,52 (dd, IH, J=12,6 and 2,0 Hz), 4,38 (d, 1H, J=10,2 Hz), 4,30 (m, 1H).

### EXAMPLE 34

3,5-Di-tert.-butyl-benzoic acid 4-(4-amino-2-oxo-2H-pyrimidin-1-yl)-[1,3]dioxolan-2-ylmethyl Ester

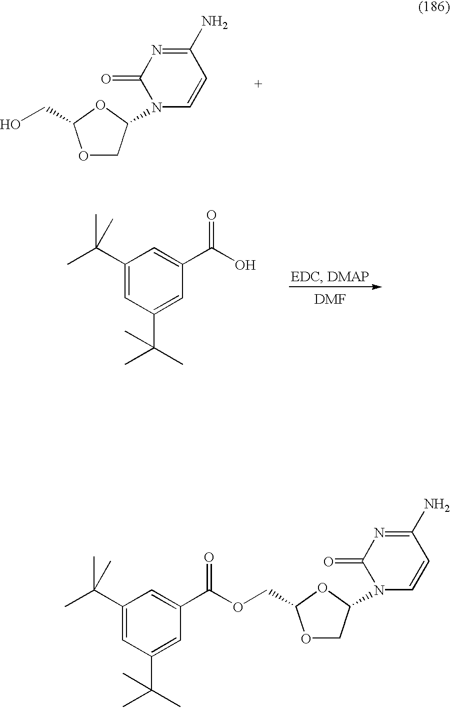

Procedure:

The nucleoside (495 mg, 2.32 mmol, 1.0 eq), 3,5-di-tButylbenzoic acid (545 mg, 2.32 mmol, 1.0 eq), DMAP (30 mg, 0.23 mmol, 0.1 eq) and EDC (445 mg, 2.32 mmol, 1.0 eq) were mixed in DMF and stirred at room temperature. The solvent was mostly evaporated and the crude diluted in dichloromethane. The organic layer was washed twice with water, brine, dried over magnesium sulfate, filtered and evaporated to dryness. The desired compound was isolated by flash chromatography using a gradient of 3%-10% methanol in dichloromethane. 281 mg was obtained.

¹H NMR (400 MHz, DMSO-d6): 7.76 (s, 2H), 7.70 (s, 1H), 7.49 (d, J=7.5 Hz, 1H), 7.18 (br d, J=24.2 Hz, 2H), 6.23 (m, 1H), 5.46 (d, J=7.5 Hz, 1H), 5.26 (t, J=3.3 Hz, 1H), 4.55 (m, 2H), 4.15-4.05 (m, 2H), 1.28 (m, 18H).

### EXAMPLE 35

Preparation of 2-Benzyl-benzoic acid 4-(4-amino-2-oxo-2H-pyrimidin-1-yl)-[1,3]dioxolan-2-ylmethyl ester

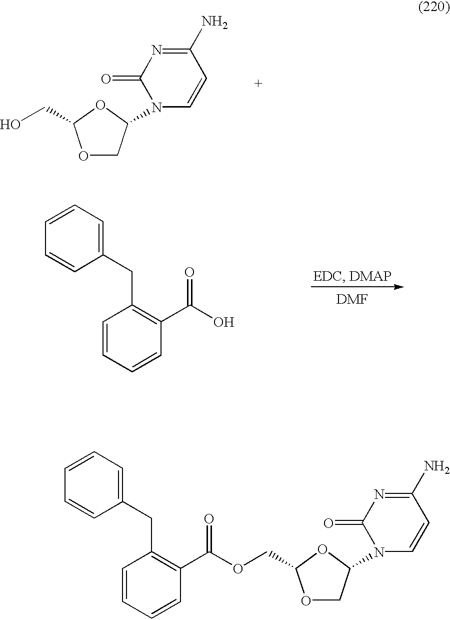

Procedure:

The nucleoside (444 mg, 2.10 mmol, 1.0 eq), alphaphenyl-o-toluic acid (445 mg, 2.10 mmol, 1.0 eq), DMAP (27 mg, 0.21 mmol, 0.1 eq) and EDC (400 mg, 2.10 mmol, 1.0 eq) were mixed in DMF and stirred at room temperature. The solvent was mostly evaporated and the crude diluted in dichloromethane. The organic layer was washed twice with water, brine, dried over magnesium sulfate, filtered and evaporated to dryness. The desired compound was isolated by flash chromatography using a gradient of 3%-10% methanol in dichloromethane.

¹H NMR (400 MHz, DMSO-d6): 7.77 (m, 1H), 7.56-7.48 (m, 2H), 7.38-7.31 (m, 2H), 7.24-7.08 (m, 7H), 6.23 (m, 1H), 5.44 (d, J=7.5 Hz, 1H), 5.19 (t, J=3.0 Hz, 1H), 4.47 (m, 2H), 4.27 (m, 2H), 4.11 (m, 2H).

### EXAMPLE 36

Preparation of 4-Hexyl-Benzoic Acid 4-(4-Methylamino-2-Oxo-2H-Pyrimidin-1-Yl)-[1,3]Dioxolan-2-Ylmethyl Ester

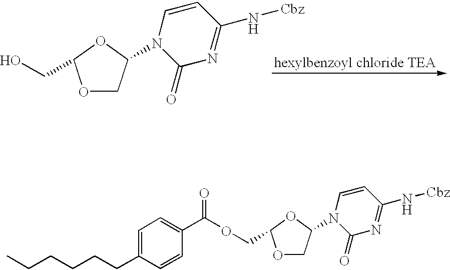

Procedure:

Acid chloride (64 [symbol] L, 0.29 mmol, leq.) was added to the mixture of the Cbz-protected BCH-4556 (101 mg, 0.29 mmol) in CH₂Cl₂ with TEA (0.12 mL, 0.87 mmol, 3 eq.). Reaction mixture was stirred at room temperature for 2 days. Solvent was evaporated. Purification was done by flash chromatography using MeOH/CH₂Cl₂ 5% to give the desired compound plus some impurities.

¹H NMR (400 MHz; CDC₃): 8.12 (d, 1H, J=7.6 Hz); 7.96-7.93 (m, 2H); 7.39-7.34 (m, 5H); 7.30-7.25 (m, 3H); 6.22 (dd, 1H; J=4.8 and 1.8 Hz); 5.34 (t, 1H, J=3 Hz); 5.21 (s, 2H); 4.77 (dd, 1H, J=3 and 12.7 Hz); 4.58 (dd, 1H, J=3 and 12.7 Hz); 4.32-4.24 (m, 2H); 2.69-2.65 (m, 2H); 1.66-1.60 (m, 2H); 1.35-1.27 (m, 6H); 0.88-0.85(m, 3H) ppm

### EXAMPLE 37

Preparation of 4-Hexyl-Benzoic Acid 4-(4-Amino-2-Oxo-2H-Pyrimidin-1-Yl)-[1,3]Dioxolan-2-Ylmethyl Ester

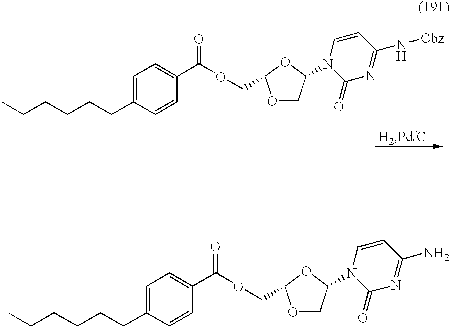

Procedure:

The protected compound (194 mg, 0.29 mmol) was dissolved in ethanol at 50° C., then purged with nitrogen. Pd/C was added, then the solution was put under H₂ atmosphere and stirred at 50° C. The solution was filtered and concentrated to give a foamy white solid. Purification by flash chromatography using MeOH/CH₂Cl₂ 3%.

¹H NMR (400 MHz; DMSO): 7.87 (d, 1H, J=8.2 Hz); 7.60 (d, 1H, J=7.4 Hz); 7.37 (d, 1H, J=8.2 Hz); 6.27 (t, 1H, J=3.7 Hz); 5.64 (d, 1H, J=7.5 Hz); 4.68-4.53 (m, 2H); 4.15 (d, 2H, J=3.9 Hz); 2.67 (t, 2H, J=7.5 Hz); 1.61-1.58 (m, 2H); 1.28 (m,6H) and 0.87-0.84 (m, 3H).ppm.

### EXAMPLE 38

Preparation of 7-Isopropyl-2,4A-Dimethyl-1,2,3,4,4A,4B,5,6,10,10A-Decahydro-Phenanthrene-2-Carboxylic Acid [1-(2-Hydroxymethyl-[1,3]Dioxolan-4-Yl)-2-Oxo-1,2-Dihydro-Pyrimidin-4-Yl]-Amide or Ester

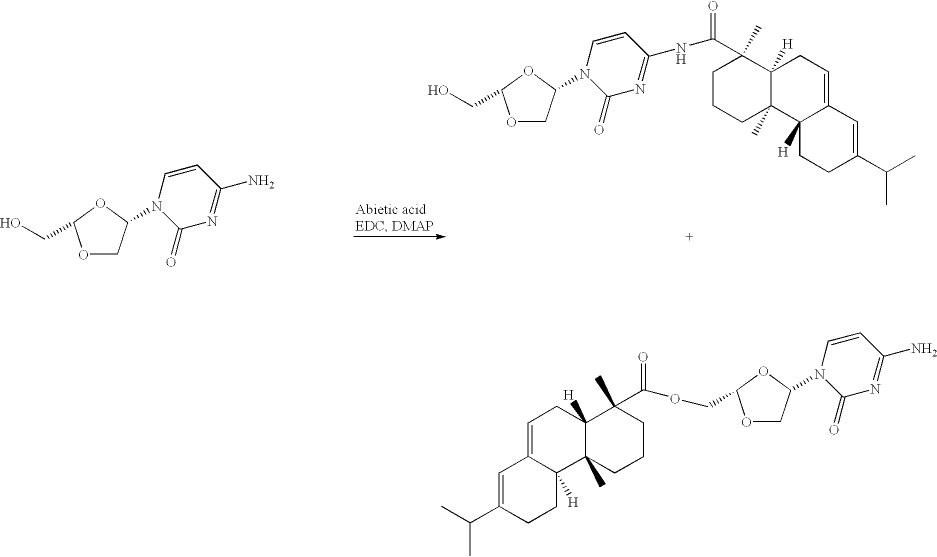

Procedure:

EDC (90 mg, 0.47 mmol) was added to a solution of the acid (143 mg, 0.47 mmol) and the alcohol (101 mg, 0.47 mmol) in DMF followed by the addition of DMAP(6 mg, 0.047 mmol, 0.1 eq.). Reaction mixture was stirred at room temperature overnight. Reaction mixture was poured into brine, extracted with EtOAc, combined extracts were washed with NaHCO₃ sat. solution, dried and concentrated to give a yellow oil.

Purification by flash chromatography using MeOH/EtOAc 100to give two compounds.

Compound 1: Amide (207)

¹H NMR (400 MHz; CDCl₃): 8.42 (d, 1H, J=7.4 Hz); 8.20 (bs,NH); 7.42 (d, 1H, J=7.6HZ); 6.18 (dd, 1H, J=5.2 and 1.2 Hz); 5.74 (s, 1H); 5.30 (bt, 1H); 5.12 (t, 1H, J=1.8 Hz); 4.36-4.24 (m, 2H); 3.98(s, 2H); 2.63-0.85 (multiplets abietic part; similar to abietic acid) ppm

Compound 2: Ester (281)

H NMR (400 MHz; CDCl₃): 7.67 (d, 1H, J=7.5 Hz); 6.19 (dd, 1H, J=2.8 and 4.5 Hz); 5.71 (t, 1H, J=7.5 Hz); 5.36 (d, 1H, J=3.lHz); 5.18 (dd, 1H, J=2.1 and 4.7 Hz); 4.48-4.09 (2m, 3H) and 2.24-0.83 (multiplets abietic part; similar to abietic acid) ppm

### EXAMPLE 39

Preparation of 4-Pentyl-Bicyclo[2.2.2]Octane-1-Carboxylic Acid [1-(2-Hydroxymethyl-[1,3]Dioxolan-4-Yl)-2-Oxo-1,2-Dihydro-Pyrimidin-4-Yl]-Amide or Ester

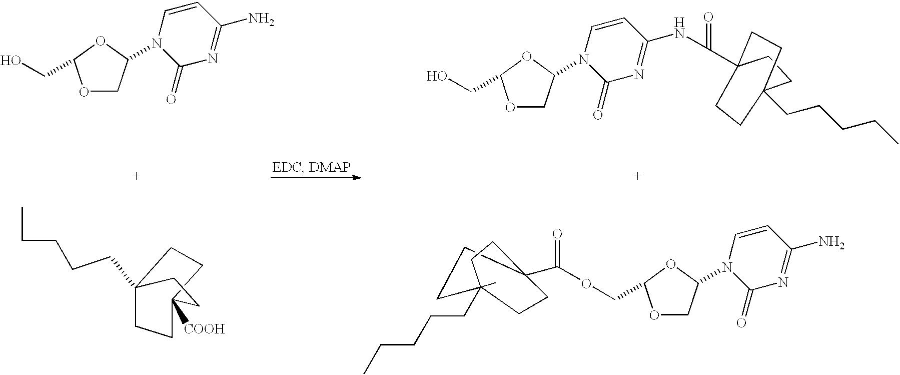

Procedure:

EDC (95 mg, 0.50 mmol) was added to a solution of the acid (112 mg, 0.50 mmol) and the alcohol (106 mg, 0.50 mmol) in DMF (0.5 mL) followed by the addition of DMAP (6 mg, 0.050 mmol, 0.1 eq.). Reaction mixture was stirred at room temperature overnight. Reaction mixture was poured into brine, extracted with EtOAc, combined extracts were washed with NaHCO₃ sat. solution, dried and concentrated to give a yellow oil.

Purification by flash chromatography using MeOH/EtOAc 10% to give two compounds.

Compound 1: Amide (210)

¹H NMR (400MHZ; CDCl₃): 8.34 (d, 1H, J=7.6 Hz); 7.36 (d, 1H, J=7.6 Hz); 6.11 (dd, 1H, J=5.1 and 1.3 Hz); 5.06 (t, 1H, J=1.8 Hz); 4.28-4.16 (m, 2H); 3.91 (d, 1H, J=1.6 Hz); 1.74-1.70 (m, 6H); 1.38-1.25 (m, 6H); 1.21 0.98(m, 8H); 0.81 (t, 3H, J=7.0 Hz)ppm

Compound 2: Ester (211)

H NMR (400 MHz; CDCl₃): 7.64 (d, 1H, J=7.4 Hz); 6.22 (dd, 1H, J=2.8 and 4.3 Hz); 5.77 (d, 1H, J=7.5 Hz); 5.15 (t, 1H, J=3.5 Hz); 4.41 (dd, 2H, J=3.7 and 12.2 Hz); 4.23-4.17 (m, 1H); 1.78-1.74 (m, 6H); 1.39-1.25 (m, 6H); 1.21 1.05(m, 8H); 0.86 (t, 3H, J=7.3 Hz)ppm

### EXAMPLE 40

Hexahydro-2,5-Methano-Pentalene-3A-Carboxylic Acid [1-(2-Hydroxymethyl-[1,3]Dioxolan-4-Yl)-2-Oxo-1,2-Dihydro-Pyrimidin-4-Yl]-Amide or Ester

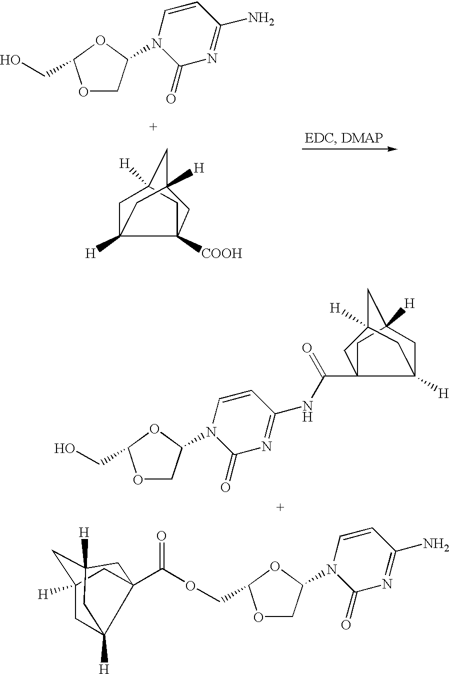

Procedure:

EDC (128 mg, 0.67 mmol) was added to a solution of the acid (lllmg, 0.67 mmol) and the alcohol (142 mg, 0.67 mmol) in DMF followed by the addition of DMAP (8 mg, 0.067 mmol, 0.1 eq.). Reaction mixture was stirred at room temperature overnight. Reaction mixture was poured into brine, extracted with EtOAc, combined extracts were washed with NaHCO₃ sat. solution, dried and concentrated to give a yellow oil.

Purification by flash chromatography using MeOH/EtOAc 5% to give two compounds.

Compound 1: Amide (231)

¹H NMR (400 MHz; CDCl₃): 8.46 (d, 1H, J=7.5 Hz); 7.98 (bs, 1H); 7.40 (d, 1H, J=7.5 Hz); 6.19 (d, 1H, J=4.9 Hz); 5.12 (s, 1H); 4.33-4.21 (m, 2H); 3.98 (s, 2H); 3.28 (bs, 1H); 2.74 (t, 1H, J=6.7 Hz); 2.37 (s, 1H); 2.16 (s, 2H); 2.04-2.01 (m, 2H); 1.86-1.82 (m, 4H) and 1.70-1.62 (m, 4H)ppm

Compound 2: Ester (232)

H NMR (400 MHz; CDCl₃): 7.74 (d, 1H, J=7.4 Hz); 6.25 (t, 1H, J=3.8 Hz); 5.72 (d, 1H, J=7.4 Hz); 5.23 (t, 1H, J=3.6 Hz); 4.55-4.29 (m, 2H); 4.24 (d, 2H, J=3.7 Hz); 2.72-2.71 (m, 1H); 2.33 (m, 2H); 2.11-2.08 (m, 2H); 1.85-1.82 (m, 4H) and 1.68-1.61 (m, 4H)ppm

### EXAMPLE 41

Preparation of 8-Phenyl-octanoic Acid 4-[2-oxo-4-(8-phenyl-octanoylamino)-2H-pyrimidin-1-yl]-[1,3]dioxolan-2-ylmethyl Ester

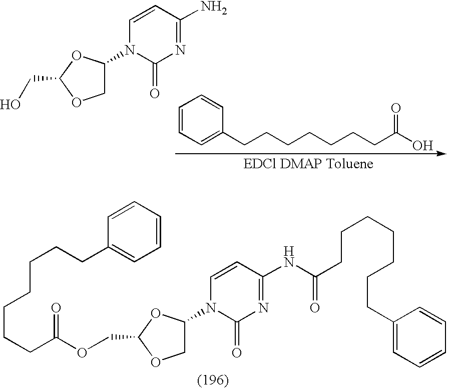

Procedure:

4-Amino-1-(2-hydroxymethyl-[1,3]dioxolan-4-yl)-1H-pyrimidin-2-one (0.23 mmol) was treated with 8-phenyl-octanoic acid (0.23 mmol), EDCI (0.35 mmol) and DMAP (catalytic amount) in DMF for 14 hours. The solution was neutralized with NaHCO₃ sat. and extracted with AcOEt. The combined organic layers were dried over sodium sulfate, filtered and concentrated in vacuum. The residue was purified by bond elute (2MeOH/CH₂Cl₂ to 10% MeOH/CH₂Cl₂) to afford 8-Phenyl-octanoic acid 4-[2-oxo-4-(8-phenyl-octanoylamino)-2H-pyrimidin-1-yl]-[1,3]dioxolan-2-ylmethyl ester.

HNMR (CDCl₃) 8.70 (s, 1H), 8.15 (d, J=7.5 Hz, 1H), 7.50 (d, J=7.4 Hz, 1H), 7.30-7.17 (m, 10H), 6.22 (d, J=4.7 Hz, 1H), 5.24 (t, J=2.6 Hz, 1H), 4.58 (dd, J=12.6, 2.8 Hz, 1H), 4.32-4.25 (m, 3H), 2.63-2.59 (m, 4H), 2.48-2.36 (m, 4H), 1.80-1.60 (m, 8H), 1.45-1.25 (m, 12H).

### EXAMPLE 42

8-Phenyl-octanoic Acid [1-(2-hydroxymethyl-[1,3]dioxolan-4-yl)-2-oxo-1,2-dihydro-pyrimidin-4-yl]-amide

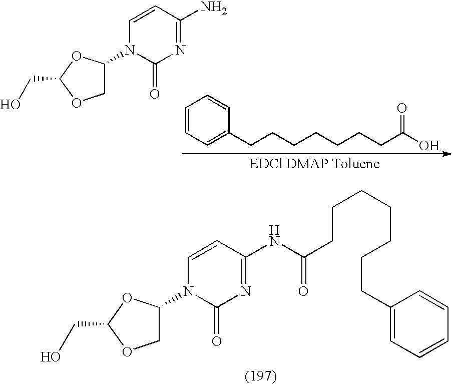

Procedure:

4-Amino-1-(2-hydroxymethyl-[1,3]dioxolan-4-yl)-1H-pyrimidin-2-one (0.23 mmol) was treated with 8-Phenyl-octanoic acid (0.23 mmol), EDCI (0.35 mmol) and DMAP (catalytic amount) in DMF for 14 hours. The solution was neutralized with NaHCO₃ sat. and extracted with AcOEt. The combined organic layers were dried over sodium sulfate, filtered and concentrated in vacuum. The residue was purified by bond elute (2% MeOH/CH₂Cl₂ to 10% MeOH/CH₂Cl₂) to produce 8-Phenyl-octanoic acid [1-(2-hydroxymethyl-[1,3]dioxolan-4-yl)-2-oxo-1,2-dihydro-pyrimidin-4-yl]-amide.

HNMR (CDCl₃) 8.62 (s, 1H), 8.49 (d, J=7.5 Hz, 1H), 7.45 (d, J=7.5 Hz, 1H), 7.30-7.27 (m, 2H), 7.20-7.17 (m, 3H), 6.20 (d, J=4.5 Hz, 1H), 5.14 (s, 1H), 4.33-4.26 (m, 2H), 3.98 (s, 2H), 2.60 (t, J=7.6 Hz, 2H), 2.45 (t, J=7.5 Hz, 2H), 1.68-1.60 (m, 4H), 1.40-1.30 (m, 6H).

### EXAMPLE 43

8-Phenyl-octanoic Acid 4-(4-amino-2-oxo-2H-pyrimidin-1-yl)-[1,3]dioxolan-2-ylmethyl Ester

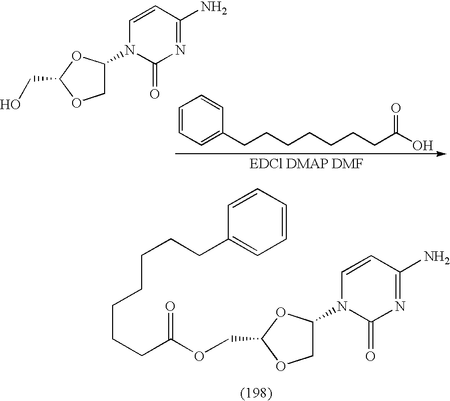

Procedure:

4-Amino-1-(2-hydroxymethyl-[1,3]dioxolan-4-yl)-1H-pyrimidin-2-one (0.23 mmol) was treated with 8-phenyl-octanoic acid (0.23 mmol), EDCI (0.35 mmol) and DMAP (catalytic amount) in DMF for 14 hours. The solution was neutralized with NaHCO₃ sat. (20 mL) and extracted with AcOEt. The combined organic layers were dried over sodium sulfate, filtered and concentrated in vacuum. The residue was purified by bond elute (2% MeOH/CH₂Cl₂ to 100 MeOH/CH₂Cl₂) to afford 0.015 g (16%) of 8-phenyl-octanoic acid 4-(4-amino-2-oxo-2H-pyrimidin-1-yl)-[1,3]dioxolan-2-ylmethyl ester.

HNMR (CDCl₃) 9.4 (s, 1H), 7.71 (d, J=7.5 Hz, 1H), 7.51-7.06 (m, 5H), 6.26 (dd, J=5, 2 Hz, 1H), 5.78 (d, J=7.5 Hz, 1H), 5.19 (t, J=3.2 Hz, 1H), 4.48 (dd, J=12.3, 3.3 Hz, 1H), 4.39-4.07 (m, 3H), 2.61 (t, J=7.2 Hz, 2H), 2.36 (t, J=7.4 Hz, 2H), 1.77-1.50 (m, 4H), 1.49-1.06 (m, 6H)

### EXAMPLE 44

(6-Iodo-hexyl)-benzene

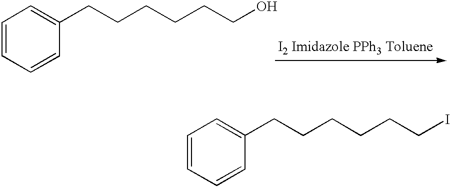

Procedure:

In a solution of 6-phenyl-hexan-1-ol (5.54 mmol) in toluene (0.2 M) was added in order PPh₃ (12.1 mmol), imidazole (24.9 mmol) and I₂ (11.6 mmol). The solution was mixed to reflux for 1.5 h and was cooled to room temperature. The solution was dissolved in Et₂O and washed with H₂O and brine. The organic layer was dried over sodium sulfate, filtered and concentrated in vacuum. The residue was purified by biotage (100% pentane to 5% Et₂O/pentane) to produce (6-iodo-hexyl)-benzene.

HNMR (CDCl₃) 7.68-7.14 (m, 5H), 3.18 (t, J=7 Hz, 2H), 2.61 (t, J=7.6 Hz, 2H), 1.86-1.79 (m, 2H), 1.67-1.60 (m, 2H), 1.46-1.33 (m, 4H)

### EXAMPLE 45

2,2-Dimethyl-8-phenyl-octanoic Acid Methyl Ester

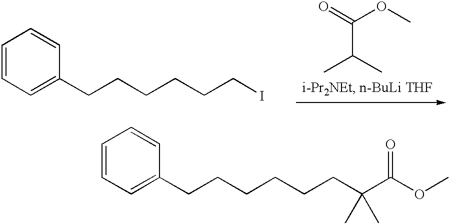

Procedure:

To a solution of 1-Pr₂Net (2.12 mmol) in THF (0.2 M) was added a solution of 1.4 M n-BuLi in hexane (2.12 mmol) at 0° C. The mixture was stirred at 0° C. for 30 minutes and cooled to −78° C. for addition of isobutyric acid methyl ester (2.12 mmol). Then, the solution was stirred at −78° C. for 1 hour and (6-Iodo-hexyl)-benzene (1.92 mmol) dissolved in THF was added slowly. This mixture was stirred 1 hour at 78° C. and 3 hours at room temperature. The solution was dissolved in Et₂(and washed with NH₄01 sat. and brine. The organic layer was dried over sodium sulfate, filtered and concentrated in vacuum. The residue was puri fied by bond elute (31.4 Et2O/pentane) to afford 0.45 g (90%) of 2,2-dimethyl-8-phenyl-octanoic acid methyl ester.

HNMR (CDCl₃) 7.29-7.25 (m, 2H), 7.18-7.15 (m, 3H), 3.64 (s, 3H), 3.48 (q, J=7 Hz, 2H), 2.58 (t, J=7.6 Hz, 2H), 1.59-1.47 (m, 2H), 1.32-1.25 (m, 2H), 1.20-1.14 (m, 10H).

### EXAMPLE 46

2,2-Dimethyl-8-phenyl-octanoic Acid

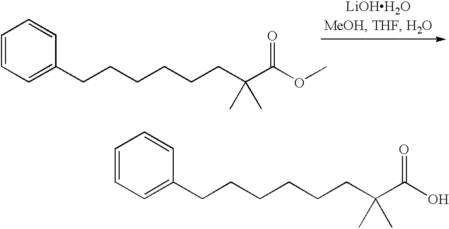

Procedure:

2,2-Dimethyl-8-phenyl-octanoic acid methyl ester (1.7 mmol) was dissolved in a MeOH, THF, H₂O solution (10:5:2). LiOH monohydrate was added and the solution was stirred and refluxed for 7 hours. The mixture was diluted with AcOEt and extracted with a solution of saturated NaHCO₃. The aqueous layers was combined, acidified with HCl 1 N and extracted with AcOEt. The organic layer was dried over sodium sulfate, filtered and concentrated in vacuum to afford 2,2-dimethyl-8-phenyl-octanoic acid.

HNMR (CDCl₃) 7.23-7.18 (m, 2H), 7.12-7.08 (m, 3H), 2.52 (t, J=7.9 Hz, 2H), 1.55-1.43 (m, 4H), 1.26-1.18 (m, 6H), 1.11 (s, 6H).

### EXAMPLE 47

2,2-Dimethyl-8-phenyl-octanoic Acid 4-(4-benzyloxycarbonylamino-2-oxo-2H-pyrimidin-1-yl)-[1,3]dioxolan-2-ylmethyl Ester

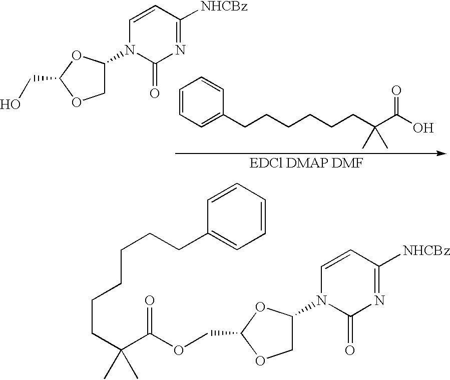

Procedure:

[1-(2-Hydroxymethyl-[1,3]dioxolan-4-yl)-2-oxo-1,2-dihydro-pyrimidin-4-yl]-carbamic acid benzyl ester (0.058 mmol) was treated with 2,2-dimethyl-8-phenyl-octanoic acid (0.058 mmol), EDCI (0.087 mmol) and DMAP (catalytic amount) in DMF. The solution was diluted in AcOEt and washed with NaHCO₃ sat. and brine. The organic layer was dried over sodium sulfate, filtered and concentrated in vacuum. The residue was purified by bond elute (5% MeOH/CH₂Cl₂) to afford 2,2-Dimethyl-8-phenyl-octanoic acid 4-(4-benzyloxycarbonylamino-2-oxo-2H-pyrimidin-1-yl)-[1,3]dioxolan-2-ylmethyl ester.

HNMR (MeOD) 8.20 (d, J=7.5 Hz, 1H), 7.44-7.34 (m, 5H), 7.27-7.10 (m, 7H), 6.19 (t, J=3.6 Hz, 1H), 5.27 (t, J=3.2 Hz, 1H), 5.23 (s, 2H), 4.70-4.47 (m, 2H), 4.31-4.23 (m, 2H), 2.62-2.54 (m, 2H), 1.63-1.49 (m, 4H), 1.39-1.15 (m, 12H).

### EXAMPLE 48

2,2-Dimethyl-8-phenyl-octanoic Acid 4-(4-amino-2-oxo-2H-pyrimidin-1-yl)-[1,3]dioxolan-2-ylmethyl Ester

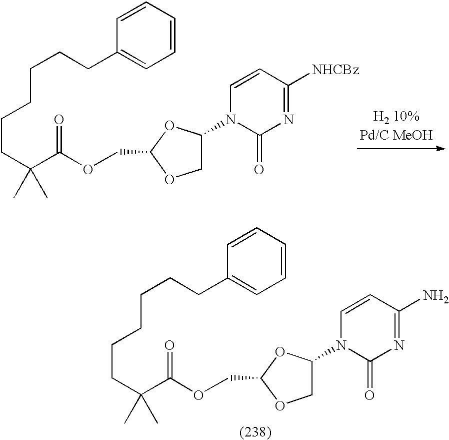

Procedure:

2,2-Dimethyl-8-phenyl-octanoic acid 4-(4-benzyloxycarbonylamino-2-oxo-2H-pyrimidin-1-yl)-[1,3]dioxolan-2-ylmethyl ester (0.048 mmol) was dissolved in MeOH. 10% Pd/C (30% w/w) was added and the solution was mixed under H₂. The solution was filtered on celite and concentrated in vacuum. The residue was purified by bond elute (5% MeOH/CH₂Cl₂) to afford of 2,2-dimethyl-8-phenyl-octanoic acid 4-(4-amino-2-oxo-2H-pyrimidin-1-yl)-[1,3]dioxolan-2-ylmethyl ester.

HNMR (MeOD) 7.76 (d, J=7.5 Hz, 1H), 7.24-7.20 (m, 2H), 7.14-7.11 (m, 3H), 6.20 (dd, J=4.5, 2.9 Hz, 1H), 5.91 (d, J=7.5 Hz, 1H), 5.18 (t, J=3.4 Hz, 1H), 4.46 (dd, J=12.4, 3.5 Hz, 1H), 4.24 (dd, J=12.4, 3.2 Hz, 1H), 4.14 (t, J=2.5 Hz, 2H), 2.56 (t, J=7.6 Hz, 2H), 1.56-1.48 (m, 4H), 1.28-1.22 (m, 6H), 1.17 (s, 3H), 1.16 (s, 3H).

### EXAMPLE 49

{1-[2-(tert-Butyl-dimethyl-silanyloxymethyl)-[1,3]dioxolan-4-yl]-2-oxo-1,2-dihydro-pyrimidin-4-yl}-carbamic Acid 2-benzenesulfonyl-ethyl Ester

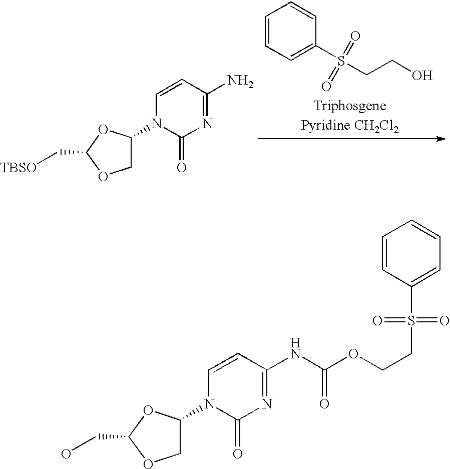

Procedure:

To a solution of triphosgene and 2-benzenesulfonyl-ethanol in CH₂Cl₂ was added pyridine at 0° C. This solution was mixed at 0° C. added to a solution of 4-amino-1-[2-(tert-butyl-dimethyl-silanyloxymethyl)-[1,3]dioxolan-4-yl]-1H-pyrimidin-2-one and pyridine in CH₂Cl₂. The resulting solution was mixed and diluted in CH₂Cl₂. The mixture was washed with water and the organic layer was dried over sodium sulfate, filtered and concentrated in vacuo. The residue was purified by bond elute (3% MeOH/CH₂Cl₂) to afford {1-[2-(tert-butyl-dimethyl-silanyloxymethyl)-[1,3]dioxolan-4-yl]-2-oxo-1,2-dihydro-pyrimidin-4-yl}-carbamic acid 2-benzenesulfonyl-ethyl ester.

HNMR (CDCl₃) 8.36 (d, J=7.2 Hz, 1H), 7.84-7.80 (m, 2H), 7.62-7.45 (m, 4H), 6.98 (s, 1H), 6.10 (dd, J=4.7, 1.9 Hz, 1H), 4.94 (t, J=1.9 Hz, 1H), 4.43 (t, J=5.4 Hz, 2H), 4.16-4.08 (m, 2H), 3.93-3.84 (m, 2H), 3.46-3.42 (m, 2H), 0.82 (s, 9H), 0.02 (s, 3H), 0.00 (s, 3H).

### EXAMPLE 50

[1-(2-Hydroxymethyl-[1,3]dioxolan-4-yl)-2-oxo-1,2-dihydro-pyrimidin-4-yl]-carbamic acid 2-benzenesulfonyl-ethyl Ester

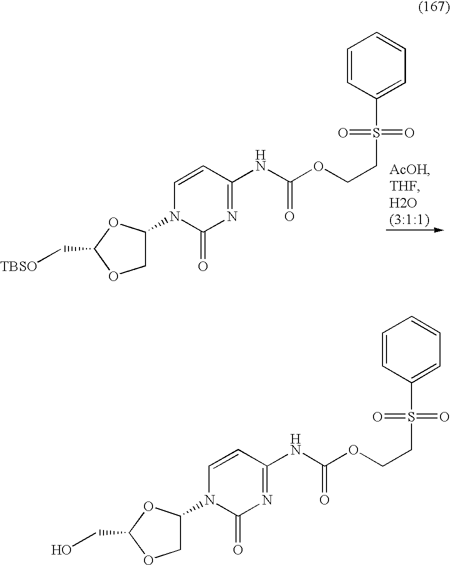

Procedure:

{1-[2-(tert-Butyl-dimethyl-silanyloxymethyl)-[1,3]dioxolan-4-yl]-2-oxo-1,2-dihydro-pyrimidin-4-yl}-carbamic acid 2-benzenesulfonyl-ethyl ester (0.087 mmol) was dissolved in a solution of AcOH, THF, H₂O (3:1:1) and was mixed. The mixture was dissolved in AcOEt and washed with H₂O, brine. The organic layer was dried over sodium sulfate, filtered and concentrated in vacuo. The residue was purified by bond elute (5% MeOH/CH₂Cl₂) to afford [1-(2-Hydroxymethyl-[1,3]dioxolan-4-yl)-2-oxo-1,2-dihydro-pyrimidin-4-yl]-carbamic acid 2-benzenesulfonyl-ethyl ester.

HNMR (CDCl₃) 8.45 (d, J=7.5 Hz, 1H), 7.93-7.90 (m, 2H), 7.70-7.65 (m, 2H), 7.59-7.55 (m, 2H), 7.08 (s, 1H), 6.17 (dd, J=5.1, 1.2 Hz, 1H), 5.12 (t, J=1.6 Hz, 1H), 4.53 (d, J=5.9 Hz, 2H), 4.33 (dd, J=10.6, 1.3 Hz, 1H), 4.23 (dd, J=10.2, 5.1 Hz, 1H), 3.97 (s, 2H), 3.54-3.51 (m, 2H), 2.6 (s, 1H).

### EXAMPLE 51

5-(Benzyl-tert-butoxycarbonyl-amino)-2,2-dimethyl-5-oxo-pentanoic Acid

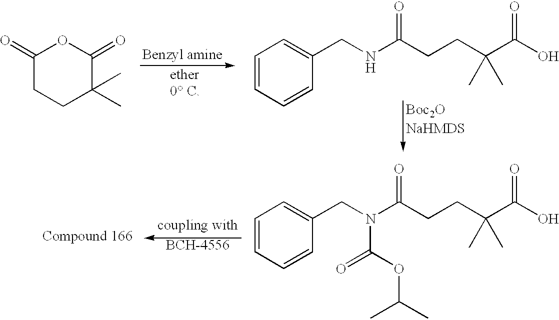

A) 4-Benzylcarbamoyl-2,2-dimethyl-butyric Acid

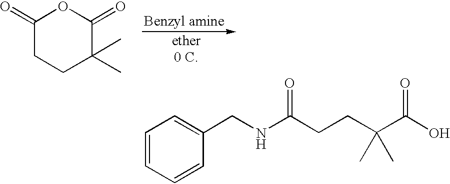

Procedure:

To a solution of 3, 3-dimethyl-dihydro-pyran-2, 6-diane (1.76 mmole) in diethyl ether at 0° C. was added benzyl amine (1.76 mmole) dropwise. As soon as addition was made, solid started to separate. The mixture was stirred at 0° C. for 15 minutes. It was diluted with ether. The solution was washed with 0.1 N HCl, and with saturated sodium chloride solution and dried over sodium sulfate. The crude product obtained after removing the solvent was passed through a bondelute (eluents: CH₂Cl₂, 2 and 4% MeOH in CH₂Cl₂) yielding 4-benzylcarbamoyl-2,2-dimethyl-butyric acid (57%).

HNMR (δ, CD₃OD): 7.23-7.32 (5H, m), 4.34 (2H, s), 2.21-2.26 (2H, m), 1.83-1.87 (2H, m), 1.18 (6H, s).

B) 5-(Benzyl-tert-butoxycarbonyl-amino)-2,2-dimethyl-5-oxo-pentanoic Acid

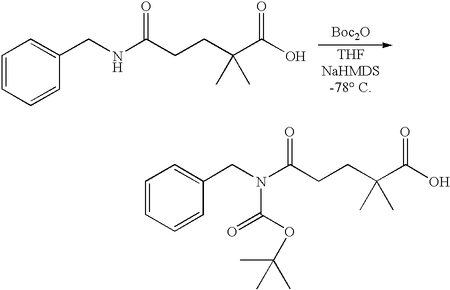

Procedure:

To a solution of 4-benzylcarbamoyl-2,2-dimethyl-butyric acid (0.09 mmole) in THF at −78° C. was added NaHMDS in THF (1M) dropwise. It was stirred at −78° C. for 15 minutes. Di-tert-butyl dicarbonate (0.1 mmole) in THF was added. It was stirred at this temperature for 15 minutes. Saturated NH₄Cl solution was added and the mixture was allowed to come to room temperature. It was acidified with dil. HCi and extracted with ethyl acetate. The extract was washed with saturated sodium chloride solution and dried over sodium sulfate. The solvent was removed and the residue was passed through a bond-elute (eluents: CH₂Cl₂ and 5% MeOH in CH₂Cl₂) yielding 5-(benzyl-tert-butoxycarbonyl-amino)-2,2-dimethyl-5-oxo-pentanoic acid (39%).

HNMR (δ, CDCl₃): 7.22-7.31 (5H, m), 4.87 (2H, s), 2.91-2.95 (2H, m), 1.93-1.97 (2H, m), 1.40 (9H, s), 1.24 (6H, s).

### EXAMPLE 52

5-(Benzyl-tert-butoxycarbonyl-amino)-2,2-dimethyl-5-oxo-pentanoic Acid 4-[4-(dimethylamino-methyleneamino)-2-oxo-2H-pyrimidin-1-yl]-[1,3]dioxolan-2-ylmethyl Ester

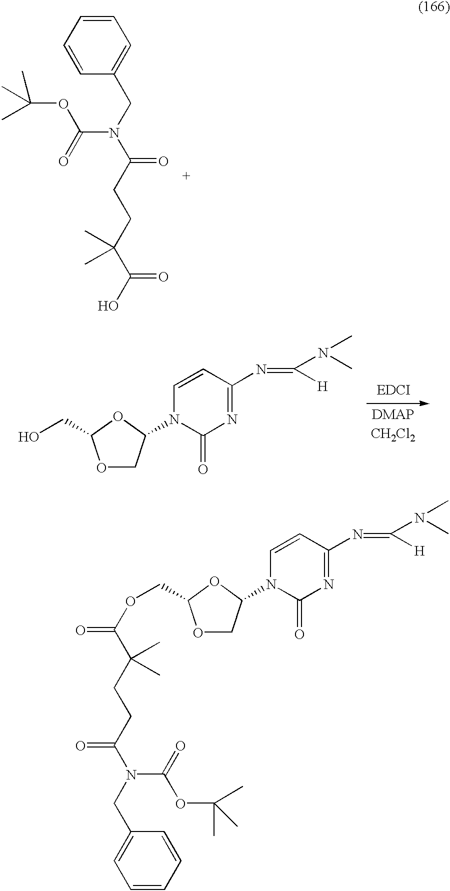

Procedure:

To a solution of N′-[1-(2-hydroxymethyl-[1,3]dioxolan-4-yl)-2-oxo-1,2-dihydro-pyrimidin-4-yl]-N,N-dimethyl-formamidine (0.034 mmole), 5-(benzyl-tert-butoxycarbonyl-amino)-2,2-dimethyl-5-oxo-pentanoic acid (0.034 mmole) and DMAP in CH₂Cl₂ at 0° C. was added EDCI (0.078 mmole) in CH₂Cl₂ dropwise. The mixture was stirred at 0° C. for 0.5 hr and then at room temperature for 18 hrs. It was diluted with CH₂Cl₂, washed with water and saturated sodium chloride solution. The solution was dried over sodium sulfate and the solvent was evaporated. The pure ester was obtained after flash chromatography over bond-elute (eluents: CH₂Cl₂, 2 and 4% MeOH in CH₂Cl₂) in 44% yield.

HNMR (δ, CD₃OD): 8.67 (1H, s), 7.97 (1H, d, J=7.2 Hz) 7.16-7.30 (5H, m), 6.20 (1H, d, J=7.2 Hz), 6.17 (1H, t, J=3.7 Hz), 5.25 (1H, dd, J=2.9, 3.4 Hz), 4.83 (2H, fine split signal), 4.57 (1H, dd, J=3.5, 12.6 Hz), 4.27 (1H, dd, J=2.9, 12.5 Hz), 4.21 (2H, d, J=3.7 Hz), 3.21, 3.13 (3H each, fine split singlets), 2.86-2.92 (2H, m), 1.89-1.93 (2H, m), 1.36 (9H, s), 1.24, 1.22 (3H each, s).

### EXAMPLE 53

6-(Benzyl-tert-butoxycarbonyl-amino)-2,2-dimethyl-hexanoic Acid and 6-(benzyl-tert-butoxycarbonyl-amino)-2-methyl-hexanoic Acid

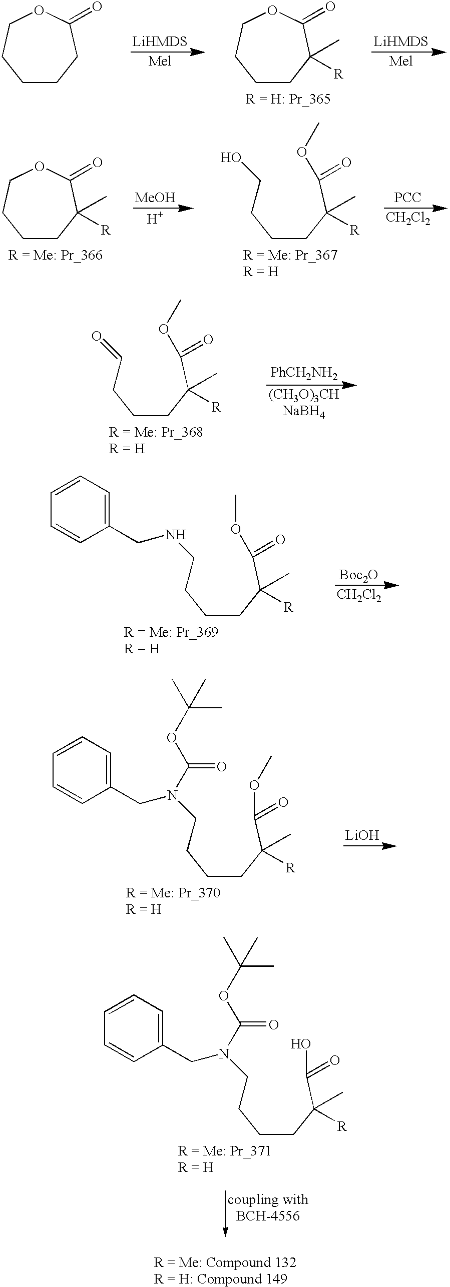

A) 3-Methyl-oxepan-2-one

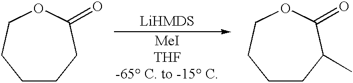

Procedure:

A solution of oxepan-2-one (4.54 mmole) in THF cooled to −65° C. was treated with LIHMDS (1M). The mixture was stirred at −65° C. Methyl iodide (8.03 mmole) was added. The temperature was raised slowly to −15° C. Saturated NH₄Cl solution was added. The mixture was extracted with diethyl ether. The solution was dried over sodium sulfate and the solvent was evaporated. The crude was passed through a bond-elute (eluent: pentane-ether mixture—1:1) yielding 3-methyl-oxepan-2-one contaminated with small amount of 3,3-dimethyl-oxepan-2-one (about 13% from NMR) (around 52 %).

HNMR (δ, CDCl₃): 4.20-4.34 (2H, m), 2.71-2.76 (1H, m), 1.93-2.01 (2H, m), 1.52-1.76 (4H, m), 1.23 (3H, d, J=6.7 Hz)

B) 3,3-Dimethyl-oxepan-2-one

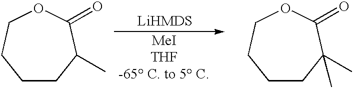

Procedure:

A solution of 3-methyl-oxepan-2-one (containing 13% of 3,3-dimethyl-oxepan-2-one) in THF at −65° C. was treated with LiHMDS (1M) dropwise. The mixture was stirred at −65° C. and methyl iodide (28.6 mmole) was added. The temperature was slowly raised to 5° C. It was stirred at 5° C. and saturated NH₄Cl solution was added. The mixture was extracted with diethyl ether. The extracts were dried over sodium sulfate and the solvent was removed. The crude on passing through a bond-elute (eluent: pentane-ether-1:1) gave pure 3,3-dimethyl-oxepan-2-one (approx. 26%).

HNMR (δ, CDCd₃) 4.24-4.27 (2H, m), 1.71-1.79 (4H, m), 1.55-1.58 (2H, m), 1.25 (6H, s).

C) 6-Hydroxy-2,2-dimethyl-hexanoic Acid Methyl Ester

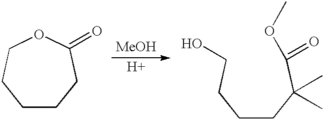

Procedure:

Methanolic HCl was prepared by adding acetyl chloride to dry MeOH slowly. 3,3-Dimethyl-oxepan-2-one (0.7 mmole) was treated with this solution. The mixture was stirred at room temperature. The solvent was removed. The residue was dissolved in diethyl ether. The solution was washed with NaHCO₃ solution and saturated sodium chloride solution and dried over sodium sulfate. The solvent was removed. The crude product was pure enough for the next step.

D) 2,2-Dimethyl-6-oxo-hexanoic acid methyl ester

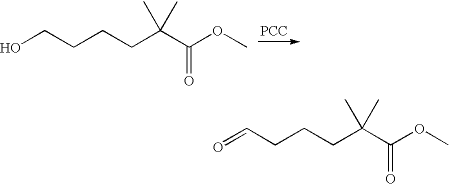

Procedure:

A mixture of 6-hydroxy-2,2-dimethyl-hexanoic acid methyl ester, molecular sieves 4A° and PCC in CH₂Cl₂ was stirred at 0° C. for 1 hr. It was diluted with diethyl ether and filtered through a bed of silica gel. The solvent was removed from the filtrate. The crude aldehyde thus obtained was pure enough for the next step.

E) 6-Benzylamino-2,2-dimethyl-hexanoic Acid Methyl Ester

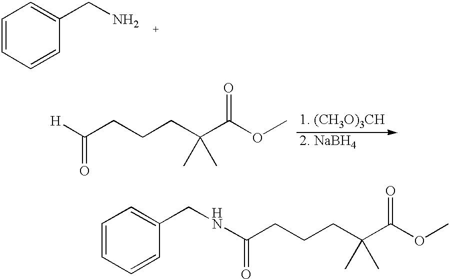

Procedure:

A mixture of benzyl amine (0.38 mmole) and methyl orthoformate (7.3 mmole) was stirred at room temperature for 5 minutes. This solution was added to crude 2,2-dimethyl-6-oxo-hexanoic acid methyl ester (0.33 mmole) It was stirred for 6 hrs. and evaporated to dryness. The residue was dissolved in MeOH and the solution was cooled to 0° C. Sodium borohydride was added in portions and the mixture was stirred. MeOH was removed and the residue was taken up in ethyl acetate. The solution was washed with saturated sodium chloride solution, dried and evaporated. The crude was passed through a bond-elute (eluents: CH₂Cl₂, and 1 and 2% MeOH in CH₂Cl₂) yielding pure 6-benzylamino-2,2-dimethyl-hexanoic acid methyl ester (13% in three steps)

HNMR (δ, CDCl₃): 7.24-7.33 (5H, m), 3.78 (2H, s), 3.64 (3H, s), 2.61 (2H, t, J=7.2 Hz), 1.45-1.53 (4H, m), 1.21-1.26 (2H, m), 1.15 (6H, s).

F) 6-(Benzyl-tert-butoxycarbonyl-amino)-2,2-dimethyl-hexanoic Acid Methyl Ester

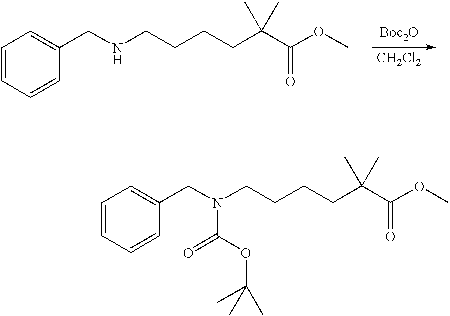

Procedure:

To a solution of 6-benzylamino-2,2-dimethyl-hexanoic acid methyl ester (0.09 mmole)in CH₂Cl₂ (3 ml) at 0° C. was added di-tert-butyl dicarbonate (0.14 mmole) in CH₂Cl₂. The mixture was stirred at room temperature for 2 hrs. It was evaporated to dryness and passed through a bond-elute yielding pure 6-(benzyl-tert-butoxycarbonyl-amino)-2,2-dimethyl-hexanoic acid methyl ester (85%).

HNMR (δ, CDCl₃) 7.21-7.33 (5H, m), 4.39-4.42 (2H, two broad signals), 3.63 (3H, s), 3.10-3.19 (2H, broad signal), 1.43-1.48 (13H, two broad signals), 1.13 (8H, broad singlet).

G) 6-(Benzyl-tert-butoxycarbonyl-amino)-2,2-dimethyl-hexanoic Acid

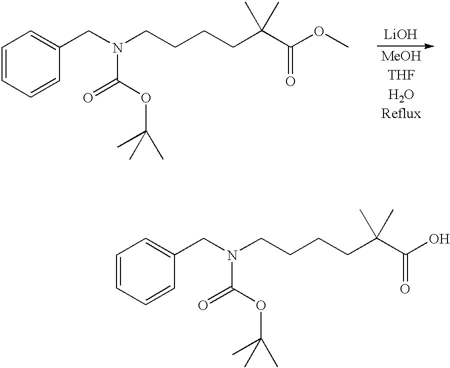

Procedure:

To a solution of 6-(benzyl-tert-butoxycarbonyl-amino)-2,2-dimethyl-hexanoic acid methyl ester (0.06 mmole) in THF and MeOH (2:1) was added LiOH.H₂O (0.26 mmole) in H₂O. The mixture was refluxed for 7 hrs and stirred at room temperature for 16 hrs. It was evaporated to dryness. The residue was taken up in water and acidified with 0.1 N HCl. It was extracted with ethyl acetate. The extract was washed with saturated sodium chloride solution, dried over sodium sulfate and evaporated. The crude was passed through a bond-elute (eluents: CH₂Cl₂ and 5% acetone in CH₂Cl₂) yielding pure 6-(benzyl-tert-butoxycarbonyl-amino)-hexanoic acid (12 mg; 57%).

HNMR (δ, CDCl₃): 7.22-7.33 (5H, m), 4.40-4.43 (2H, broad signal), 3.12-3.20 (2H, broad signal), 1.43-1.48 (13H, two broad signals), 1.21-1.25 (2H, m), 1.16 (6H, s).

### EXAMPLE 54

6-(Benzyl-tert-butoxycarbonyl-amino)-2,2-dimethyl-hexanoic acid 4-[4-(dimethylamino-methyleneamino)-2-oxo-2H-pyrimidin-1-yl]-[1,3]dioxolan-2-ylmethyl Ester

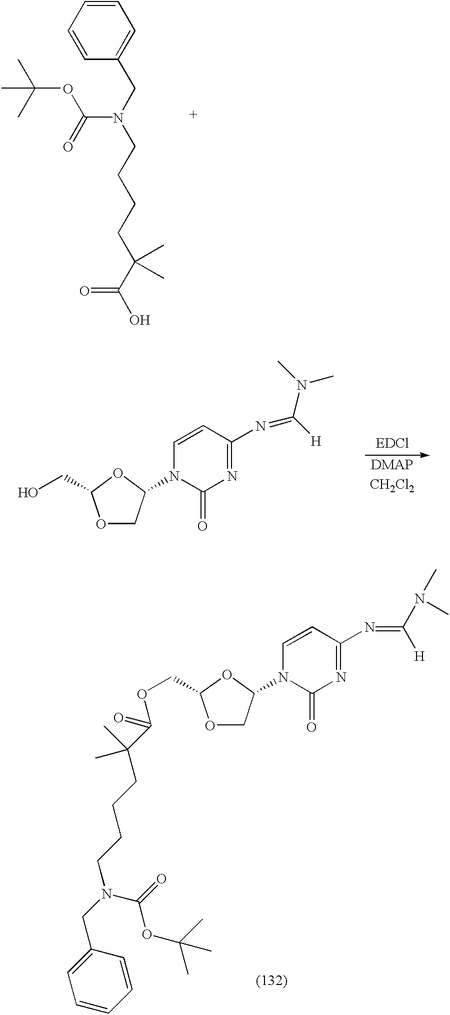

Procedure:

To a mixture of N′-[1-(2-hydroxymethyl-[1,3]dioxolan-4-yl)-2-oxo-1,2-dihydro-pyrimidin-4-yl]-N,N-dimethyl-formamidine (0.03 mmole), 6-(benzyl-tert-butoxycarbonyl-amino)-2,2-dimethyl-hexanoic acid (0.03 mmole) and DMAP (0.3 mg) in dichloromethane (0.3 ml) at 0° C. was added EDCI (0.063 mmole) in dichloromethane dropwise. It was stirred for 30 minutes at this temperature and at room temperature for 18 hrs. The mixture was diluted with dichloromethane, washed with water and saturated sodium chloride solution. The solution was dried over sodium sulfate and evaporated. The crude product was passed through a bond-elute (eluents: dichloromethane, 1 and 2% MeOH in dichloromethane) yielding the ester (28% yield)

HNMR (δ, CD₃OD): 8.69 (1H, s), 7.96 (1H, d, J=7.3 Hz), 7.19-7.32 (5H, m), 6.19-6.23 (2H, m), 5.23 (1H, t, J=3.2 Hz), 4.49 (1H, dd, J 3.4, 12.5 Hz), 4.39 (2H, s), 4.22-4.28 (3H, m), 3.22, 3.14 (3H each, s), 1.29-1.47 (15H, three broad signals), 1.17, 1.16 (3H each, s).

### EXAMPLE 55

6-(Benzyl-tert-butoxycarbonyl-amino)-2-methyl-hexanoic Acid

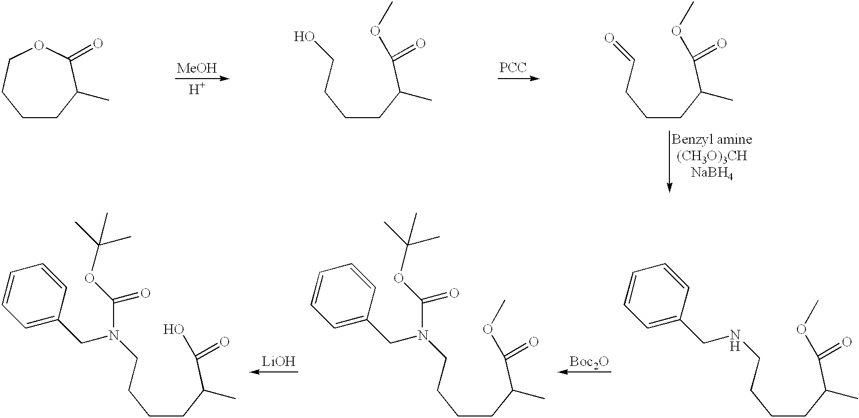

Procedure:

The procedure to obtain this compound is similar to procedures described in previous examples.

### EXAMPLE 56

6-(Benzyl-tert-butoxycarbonyl-amino)-2-methyl-hexanoic Acid 4-[4-(dimethylamino-methyleneamino)-2-oxo-2H-pyrimidin-1-yl]-[1,3]dioxolan-2-ylmethyl Ester

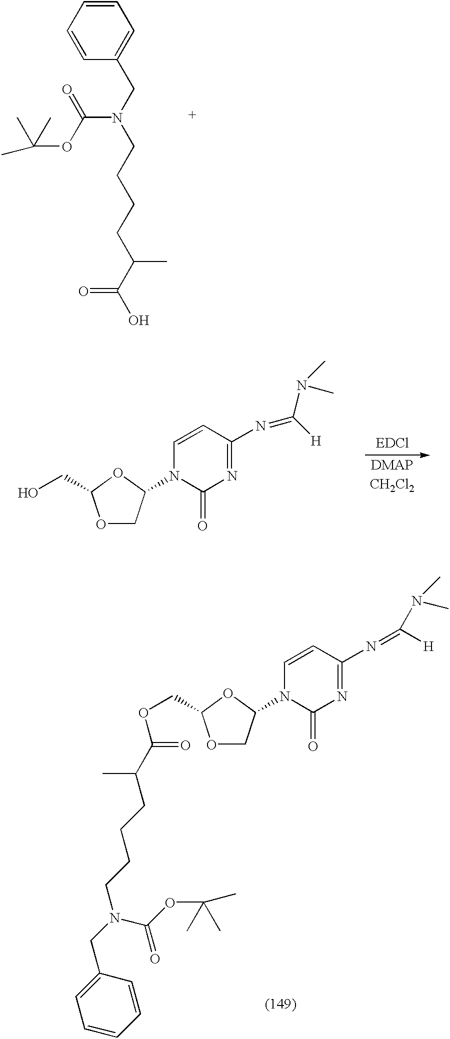

Procedure:

To a solution of N′-[1-(2-hydroxymethyl-[1,2]dioxolan-4-yl)-2-oxo-1,2-dihydro-pyrimidin-4-yl]-N,N-dimethyl-formamidine (0.036 mmole), 6-(benzyl-tert-butoxycarbonyl-amino)-2-methyl-hexanoic acid (0.036 mmole) and DMAP (0.4 mg) in dichioromethane at 0° C. was added EDCI (0.078 mmole) in dichioromethane dropwise. The mixture was stirred at 0° C. for 30 minutes and then at room temperature for 2.5 hrs. It was diluted with dichloromethane (50 ml), washed with water and saturated sodium chloride solution. The solution was dried over sodium sulfate and evaporated. The crude was passed through a bond-elute (eluents: CH₂Cl₂, 1 and 2% MeOH in CH₂Cl₂) and the pure ester was obtained in 62% yield.

HNMR (δ, CD₃OD): 8.68 (1H, s), 8.02 (H₁, two doublets, J=7.3 Hz), 7.20-7.32 (5H, multiplets), 6.17-6.25 (2H, m), 5.23-5.25 (1H, broad signal), 4.52 (1H, two dd, J=2.4, 12.1 Hz), 4.39-4.40 (total 2H, broad signals), 4.20-4.31 (3H, m), 3.21, 3.12 (3H each, s), 2.46 (1H, q, J=7.0 Hz), 1.20-1.67 (15H, multiplets), 1.12, 1.11 (total 3H, two doublets, J=7.0 Hz).

### EXAMPLE 57

6-(Benzyl-tert-butoxycarbonyl-amino)-hexanoic Acid

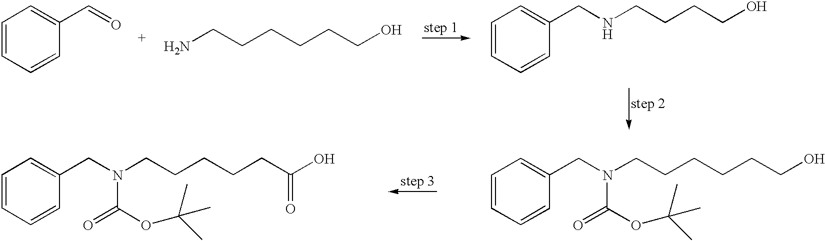

Procedure

Steps 1 and 2 were carried out as described in N. Mourier, M. Camplo, G. S. Della Bruna, F. Pellacini, D. Ungheri, J. -C. Chermann and J. -L. Kraus, Nucleosides, Nucleotides & Nucleic Acids, 19 (7), 1057-91 (2000), step 3 was substituted by a Jones oxidation as described in R. N. Rej, J. N. Glushka, W. Chew and A. S. Perlin, Carbohydrate Research, 189 (1989), 135-148.

### EXAMPLE 58

6-(Benzyl-tert-butoxycarbonyl-amino)-hexanoic Acid 4-(4-amino-2-oxo-2H-pyrimidin-1-yl)-[1,3]dioxolan-2-ylmethyl Ester

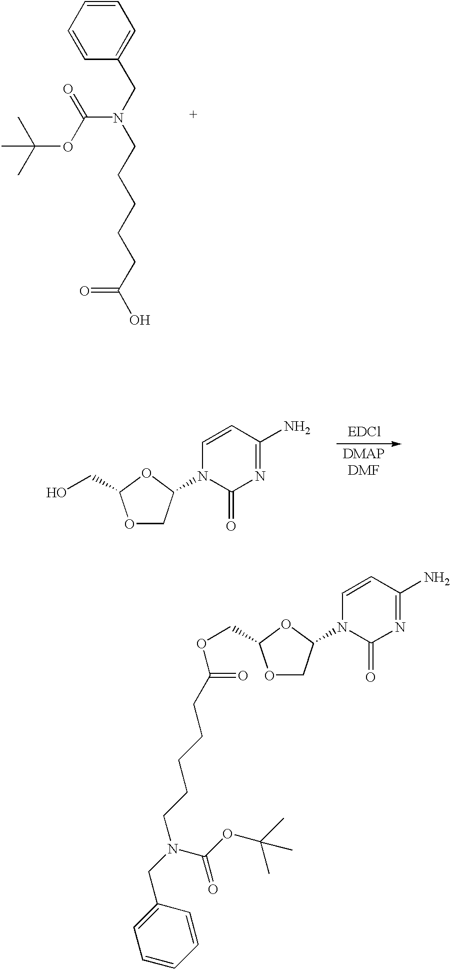

Procedure:

A mixture of 4-amino-1-(2-hydroxymethyl-[1,3]dioxolan-4-yl)-1H-pyrimidin-2-one (0.11 mmole), 6-(benzyl-tert-butoxycarbonyl-amino)-hexanoic acid (0.11 mmole), EDCI (0,156 mmole) and DMAP (3 mg) in DMF was stirred at room temperature for 16 hrs. DMF was removed in vacuum. The residue was taken up in ethyl acetate, washed with water and saturated sodium chloride solution. The solution was dried over sodium sulphate and evaporated. The pure ester was obtained by chromatography over bond-elute (eluents: CH₂Cl₂, 2 and 4% MeOH in CH₂Cl₂) (17 mg, 31% yield)

HNMR (δ, CDCl₃): 7.78 (1H, broad signal), 7.23-7.34 (5H, m), 6.28-6.29 (2H, broad signal), 5.70-5.87 (1H, broad signal), 5.21 (1H, broad signal), 4.21-4.48 (6H, two multiplets), 3.20 (2H, broad signal), 2.35 (2H, t, J=7.7 Hz), 1.45-1.65 (13H, m), 1.26-1.38 (2H, m).

### EXAMPLE 59

5-(Benzyl-tert-butoxycarbonyl-amino)-pentanoic Acid 4-(4-amino-2-oxo-2H-pyrimidin-1-yl)-[1,3]dioxolan-2-ylmethyl Ester

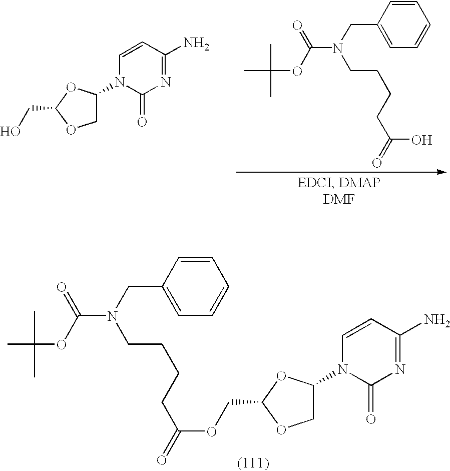

Procedure:

4-Amino-1-2-hydroxymethyl-[1,3]dioxolan-4-yl)-1H-pyrimidin-2-one (0.06 mmol) was treated 5-(Benzyl-tert-butoxycarbonyl-amino)-pentanoic acid (0.07 mmol) (Nucleosides, nucleotides & nucleic acids, 2000, 19 (7), 1057-1091), EDCI (0.09 mmol) and DMAP (catalytic amount) in DMF for 14 hours. The solution was neutralized with NaHCO₃ sat. and extracted with AcOEt. The combined organics layers was dried over sodium sulfate, filtered and concentrated in vacuo. The residue was purified by bond elute (2% MeOH/CH₂Cl₂ to 10% MeOH/CH₂Cl₂) to afford 36% of 5-(Benzyl-tert-butoxycarbonyl-amino)-pentanoic acid 4-(4-amino-2-oxo-2H-pyrimidin-1-yl)-[1,3]dioxolan-2-ylmethyl ester.

HNMR (CDCl₃) 7.86 (d, J=6.4 Hz, 1H), 7.34-7.19 (m, 5H), 6.28 (broad s, 2H), 6.00 (d, J=6.9 Hz, 1H), 5.07 (s, 2H), 4.50-4.31 (m, 3H), 4.28-4.15 (m, 3H), 3.18-3.08 (m, 2H), 2.17-2.16 (m, 2H), 1.60-1.40 (m, 13H).

### EXAMPLE 60

2,2-Dimethylpropionic Acid 4-(1-{2-[4-(2,2-dimethylpropionyloxy)benzyloxy Carbonyloxymethyl]-[1,3]dioxolan-4-yl}-2-oxo-1,2-dihydropyrimidin-4-ylcarbamoyloxymethyl)-phenyl Ester (212)

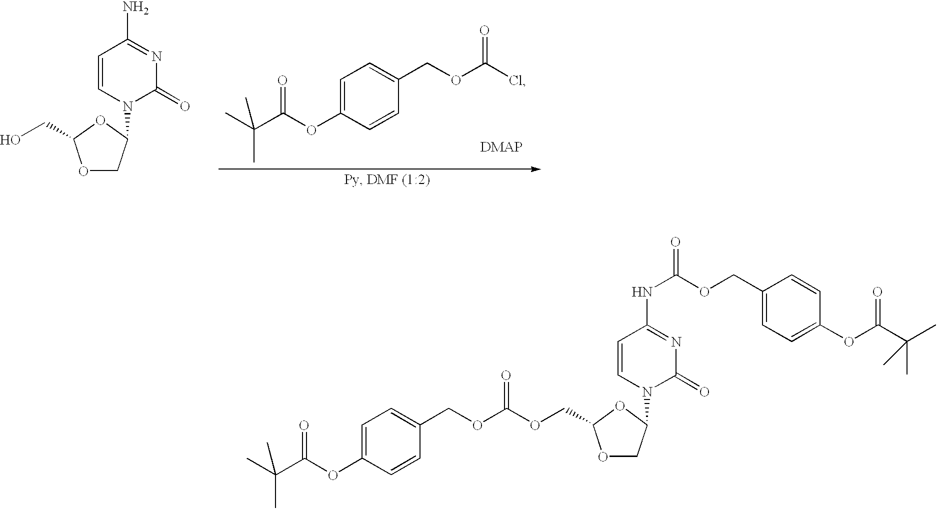

Procedure:

2,2-Dimethylproprionyloxybenzylchloroformate (1.56 mmol) was added dropwise to a 0° C. solution of BCH-4556 (1.30 mmol) and DMAP (1.56 mmol) in dimethylformamide and pyridine and stirred at room temperature for 18 h. The reaction mixture was concentrated in vacuo. The oil obtained was partitioned between NH₄Clₛₐₜ/water and dichloromethane. Aqueous layer was extracted with DCM. Organic layers were combined, dried over MgSO₄, filtered and concentrated to a yellow gum. The crude residue was purified by silica gel biotage (40S) (40% EtOAc: 60% hexanes to 80% EtOAc: 20% hexanes) to give 1% yield of 2,2-Dimethylpropionic acid 4(1-{2-[4-(2,2-dimethylpropionyloxy)benzyloxycarbonyloxymethyl]-[1,3]dioxolan-4-yl}-2-oxo-1,2-dihydropyrimidin-4-ylcarbamoyloxymethyl)-phenyl ester (212) as a white powder.

¹H NMR (400 MHz, CDCl₃), δ ppm: 8.16 (d, 1H, J=7.5 Hz), 7.42-7.38 (m, 4H), 7.23 (d, 1H, J=7.5 Hz), 7.09-7.06 (m, 4H), 6.22-6.21 (m, 1H), 5.24-5.22 (m, 1H), 5.21 (s, 2H), 5.18 (s, 2H), 4.60 (dd, 1H, J=2.6, 12.6 Hz), 4.41 (dd, 1H, J=2.4, 12.6 Hz), 4.30-4.21 (m, 2H), 1.36 (s, 9H), 1.34 (s, 9H).

### EXAMPLE 61

Acetic acid 4-(1-{2-[4-(Acetyloxy)benzyloxycarbonyl oxymethyl]-[1,3]dioxolan-4-yl}2-oxo-1,2-dihydropyrimidin-4-ylcarbamoyloxymethyl)-phenyl Ester (202)

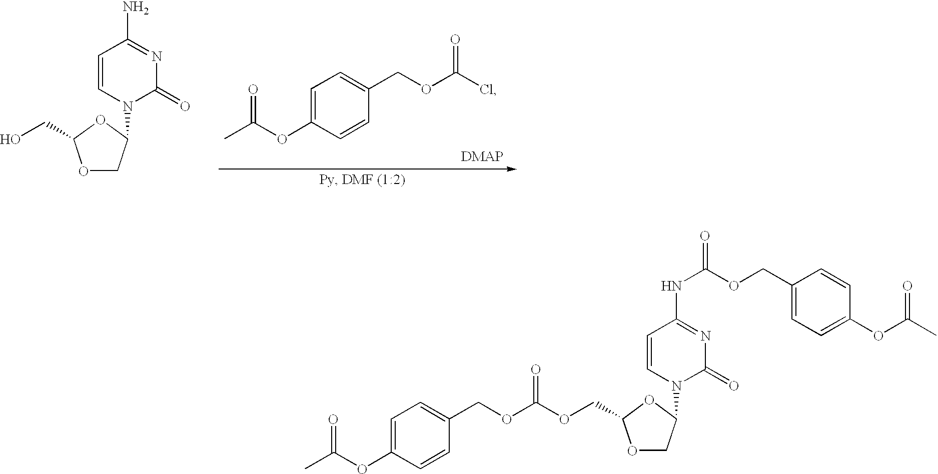

Procedure:

Acetyloxybenzylchloroformate (1.14 mmole, 1,2 eq.) was added dropwise to a 0° C. solution of ECH-4556 (0,952 mmole, 1 eq.) and DMAP (1,14 mmole, 1,2 eq.) in dimethylformamide and pyridine and stirred at room temperature for 18 h. The reaction mixture was concentrated in vacuo. The oil obtained was partitioned between saturated NH₄Cl and dichloromethane. Aqueous layer was extracted with dichloromethane. Organic layers were combined, dried over MgSO₄, filtered and concentrated to a yellow gum. The crude residue was purified by silica gel biotage (40S) (50% EtOAc: 50% hexanes to 100% EtOAc) to give 20,2 mg (4% yield) of the desired product.

¹H NMR (400 MHz, CDCl₃), δ ppm: 8,14 (dd 1H, J=7,5 and 5,2 Hz), 7,64 (s 1H), 7,40 (m 4H), 7,24 (m 1H), 7,10 (m 4H), 6,20 (t 1H, J=5,0 Hz), 5,19 (m 5H), 4,58 (m 2H) 2,30 (s 3H), 2,28 (s 3H).

### EXAMPLE 62

### Cell Proliferation Assays/NT Inhibitor Studies

The chemosensitivity of suspension cells lines (e.g., CEM or CEM-derivatives) is assessed using the CellTiter 96 [symbol] proliferation assay. Cells are seeded in 96-well plates (8 replicates) in three separate experiments and exposed to graded concentrations (e.g., 0.001-100 μM) of a nucleoside of interest (e.g., cytarabine, gemcitabine or troxacitabine), for 48 h. Chemosensitivity is expressed as 50% (EC₅₀) of the dose response curve determined, e.g., using GraphPad Prism 2.01 (GraphPad Software, San Diego, Calif.). Adherent cell lines (e.g., DU145 or DU145^{R}) are seeded (^{˜}10⁵ cells) in triplicate dishes, 24 h before drug exposure. Growth inhibition is determined by trypsinization and counting cells electronically.

In this example, troxacitabine is shown to enter cells by a mechanism other than via the NT, es (defective in CEM/ARA89C), or via the four other NTs which are not present in CEM cells, ei, cit, cif, and cib (See, e.g., Ullman (1989). Advances in Experimental Medicine & Biology 253B: 415-20). This is consistent with entry into the cells by passive diffusion. The ability of troxacitabine to inhibit cell proliferation of CEM and CEM-derivative cell lines was directly compared to other cytosine-containing nucleoside analogs, gemcitabine and cytarabine, in a cell proliferation assay (See Table 1). The growth of CEM cells was inhibited by all three nucleoside analogs, and troxacitabine was 16 and 8-fold less toxic than cytarabine and gemcitabine, respectively. The presence of the es transport inhibitor, NBMPR, signif icantly increased resistance of CEM cells to gemcitabine and cytarabine but not to troxacitabine. CEM cells are reported to exhibit primarily es. Therefore, this example suggests that that the uptake of troxacitabine is less dependent on the presence of a functional hENT1 transporter (es) in CEM cells than cytarabine or gemcitabine. In addition, there was a much lower level of resistance observed for the nucleoside-transport deficient CEM/ARAC8C cells exposed to troxacitabine (8-fold) compared to cytarabine (1150-fold) or gemcitabine (431-fold), further implying lack of transport of troxacitabine (by es NT). Taken together, the data suggested that troxacitabine has a different uptake mechanism than cytarabine and gemcitabine. This again is consistent with entry into the cells by passive diffusion.

Table 1. Comparative Chemosensitivities of CEM and CEM-derivative Cell Lines to Troxacitabine, Gemcitabine and Cytarabine.

Cultures were exposed to graded concentrations (0.001-100 μM) of cytarabine, gemcitabine or troxacitabine for 48 h. Chemosensitivity was measured using the Promega CellTiter 96 cell proliferation assay and expressed as 50% of the dose response curve (EC₅₀).

The effect of the es transport inhibitor, NBMPR (100 nM) on the EC₅₀ values of CEM cells exposed to cytarabine, gemcitabine or troxacitabine was also determined. Each value represents the average (+standard deviation) of three separate experiments (each experiment had 8 replicates).

| Cell line | Cytarabine | Gemcitabine | Troxacitabine |
|---|---|---|---|
| CEM | 0.01 | ± 0.02 | ± 0.16 ± 0.012 |
| | 0.002 | .0004 | |
| CEM + NBMPR | 0.05 | ± 0.07 | ± 0.21 ± 0.019 |
| | 0.006 | 0.018 | |
| CEM/ARAC8C | 11.50 | ± 8.63 | ± 1.18 ± 0.315 |
| | 2.654 | 0.881 | |
| CEM/dCK | >50 | >50 | >100 |

### EXAMPLE 63

### Cellular Uptake Assays

Measurements of nucleoside uptake are performed by conventional methods, as described, e.g., in Rabbani et al. (1998) Cancer Res. 58: 3461; Weitman et al. (2000). Clinical Cancer Res., 6:1574-1578; or Grove et al. (1996). Cancer Res., 56: 4187-4191. Briefly, for adherent cells, uptake assays are conducted at room temperature under zero-trans conditions in either sodium-containing transport buffer (20 mM Tris/HCl, 3 mM K₂HPO₄, 1 mM MgCl₂.6H₂O, 2 mM CaCl₂, 5 mM glucose and 130 mM NaCl, pH 7.4, 300±15 mOsm) or sodium-free transport buffer with NaCl replaced by N-methyl-D-glucamine. Cells are washed twice with the appropriate transport buffer and then either processed immediately, or in some experiments, incubated with transport inhibitors, NBMPR (100 mM), dipyridamole (20 μM) or dilazep (100 μM) during the second wash at room temperature for 15 min before the uptake assay. Precisely timed intervals are initiated by adding transport buffer containing [³H]troxacitabine or [³Hluridine and terminated by immersion in ice-cold transport buffer. After the plates are drained, the cells are lysed with 5% Triton X-100 and mixed with Ecolite scintillation fluid to measure the cell-associated radioactivity (Beckman LS 6500 scintillation counter; Beckman-Coulter Canada, Mississauga, ON). Uptake at the zero time-point is determined by treating cells for 10 min at 4° C. with transport buffer containing 100 μM dilazep, then adding the radioactive nucleoside for 2 s before reaction termination as described above. Uptake assays for suspension cells are conducted in microfuge tubes and permeant fluxes are terminated using the ‘‘inhibitor-oil [symbol] stop method; dilazep is used at a final concentration of 200 μM. Uptake at the zero time-point is determined by adding cells to cold transport buffer containing radiolabeled permeant and dilazep, and immediate centrifugation. Cell pellets are lysed and cell-associated radioactivity measured.

### EXAMPLE 64

### NT Inhibitor Studies/Competition with an Excess of the Nucleoside of Interest, Itself, in Non-radioactive Form

CEM Cells:

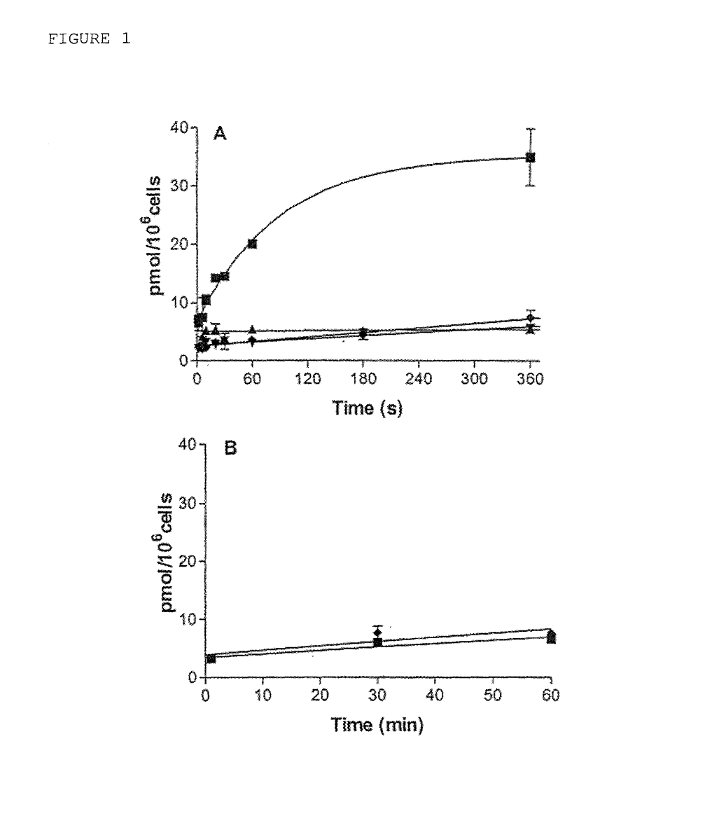

CEM cells contain primarily one type of nucleoside transport activity (es), and the functionality of this transporter (hENT1) was first demonstrated by the uptake of the physiological substrate, uridine (FIG. 1A), using methods as described in Example 29. The transport of [³H]uridine was inhibited in the presence either of the hENT1 inhibitor, NBMPR, or excess non-radioactive uridine. [³H]troxacitabine was taken up to a lesser degree over the 6-min time course in CEM and in CEM/ARAC8C cells (FIG. 1B). Lack of [³H]uridine uptake in the latter cell line demonstrated the absence of functional hENT1 transporters. The data suggest that troxacitabine uptake in CEM cells is not mediated by es activity and is consistent with it being taken up by passive diffusion.

DU145 Cells:

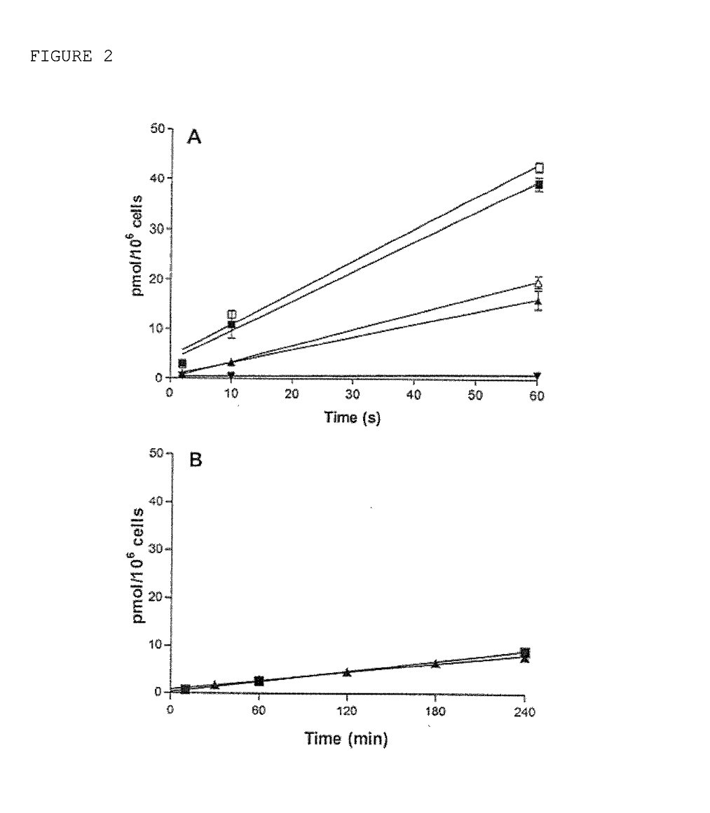

The presence of functional es-mediated transport (hENT1) in DU145 cells was first demonstrated in a cellular uptake assay with 10 μM [³H]uridine, as a control substrate in the presence and absence of the hENT1 inhibitor, NBMPR. In the presence of NBMPR, total [³H]uridine uptake over a 6-min time course was inhibited by ^{˜}75% (FIG. 2A). In contrast, low levels of [³H]troxacitabine were taken up and uptake was not affected by the presence of NBMPR (FIG. 2B). The results are consistent with the uptake of troxacitabine observed in CEM cells and provide further evidence that troxacitabine is a very poor substrate for hENT1, and probably enters the cell by passive diffusion.

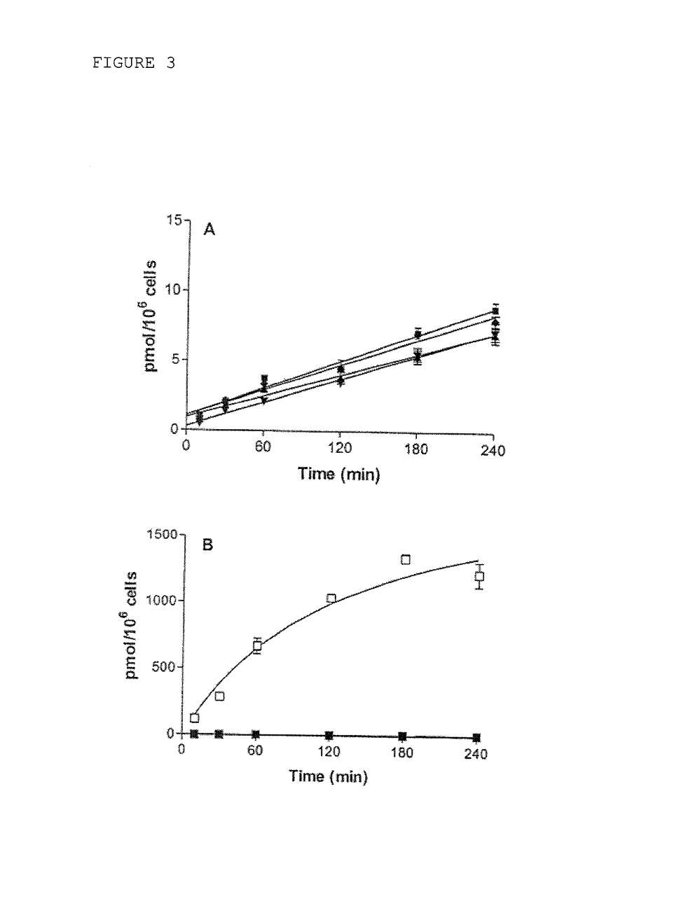

HeLa Cells:

[³H]Troxacitabine and [³H]uridine cellular uptake by hENT2 (ei NT) in HeLa cells. In the presence of the hENT1 inhibitor, NBMPR, the functionality of hENT2 was first demonstrated in a cellular uptake assay with 10 μM [³H]uridine (FIG. 3A). A high total uptake of uridine was observed over a long time course of 240 min of about 1200 pmol/10⁶ cells. In an expanded scale over the same time period, low levels of [3H]troxacitabine were taken up with a total uptake of about 10 pmol/10⁶ cells, 120-fold lower than uridine (FIG. 3B). In the presence of nucleoside transport inhibitors, NBMPR, dilazep, and dipyridamole or excess non-radioactive troxacitabine, no substantial inhibition of troxacitabine uptake was observed. Taken together, the results demonstrate that compared to uridine, troxacitabine is a very poor substrate for hENT2. Furthermore, the fact that an excess of unlabeled troxacitabine failed to inhibit the uptake of the labeled troxacitabine indicates that troxacitabine is not mediated by a nucleoside transporter, i.e., that it enters the cells by passive diffusion.

DU145 Cells:

This experiment is designed to show whether [³H]L-troxacitabine (10 μM) is taken up by DU145 cells and if the rate of uptake is affected by the addition of high concentrations (1 mM) of non-radioactive troxacitabine. The results show that the uptake of [³H]L-troxacitabine is very slow during both short (0-30s) and prolonged exposures (0-4 h). The addition of non-radioactive troxacitabine has no significant effect on the uptake of [³H]L-troxacitabine, an indication that uptake in these cells is not mediated by a NT, but instead is taken up by passive diffusion.

### EXAMPLE 65

### Uptake by hCNT1, hCNT2 and hCNT3

[1H]Troxacitabine and [³H]uridine Uptake by Recombinant hCNT1 and hCNT2 in Transient-transfection Assays in HeLa Cells:

Expression plasmids encoding recombinant hCNT1 and hCNT2 are prepared using conventional methods. Genes encoding the hCNT1 and hCNT2 transporter proteins are subcloned from the plasmids pMHK2 (Ritzel et al. (1997). Am. J. Physiology 272: C707-C₇₁₄) and pMH15 (Ritzel et al. (1998). Mol Membr Biol. 15: 203-11) into the mammalian expression vector, pcDNA3, to produce pcDNA3-hCNT1 (Graham et al. (2000). Nucleosides Nucleotides Nucleic Acids 19: 415-434) and pcDNA3-hCNT2. The expression vectors are separately introduced into actively proliferating HeLa cells, following conventional methods. See, e.g., Fang et al (1996). Biochemical Journal 317: 457-65.

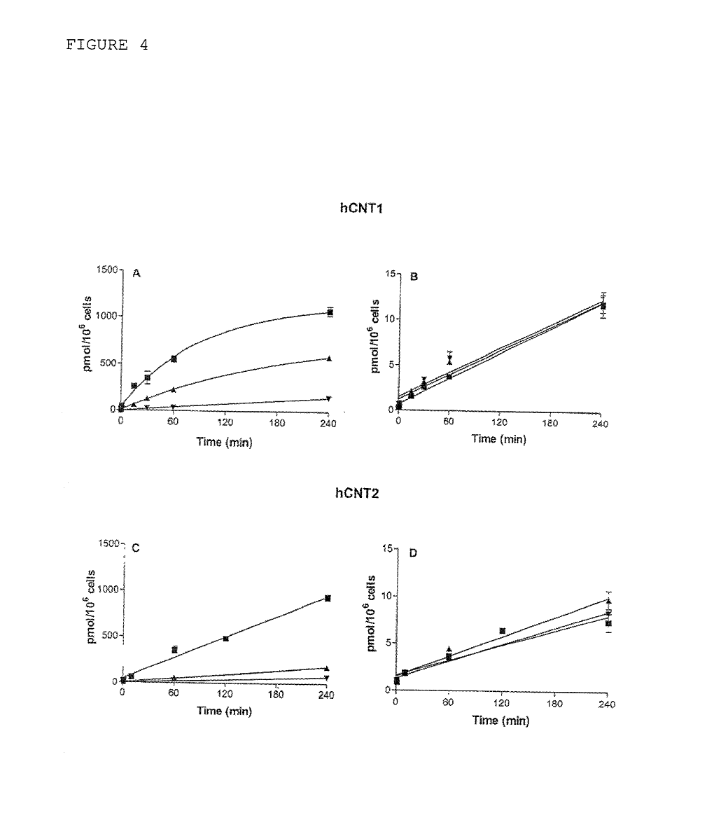

Recombinant hCNT1 and hCNT2 were separately introduced into HeLa cells by transient transfection of pcDNA3 plasmids containing the coding sequences of the relevant nucleoside transporter protein. After transfection, functionality of each transporter was demonstrated by comparing the uptake of 10 μM [³H]uridine in the presence of the equilibrative transporter (hENT1, hENT2) inhibitor, 100 μM dilazep, to cells transfected with the empty vector pcDNA3 control plasmid (FIG. 4). Uptake of 10 μM [³H]troxacitabine was not mediated either by hCNT1 or by hCNT2. Troxacitabine uptake by cib-activity (hCNT3) in differentiated HL-60 cells:

The ability of a high concentration (100-fold) of non-radioactive troxacitabine to inhibit the uptake of ³H]uridine by hCNT3 was examined in a differentiated HL-60 model system [Ritzel et al. (2000), supra]. Under these conditions, troxacitabine had no effect on uridine uptake and suggested that troxacitabine was not substrate of hCNT3.

The examination of troxacitabine uptake in several cell lines has shown that uptake is not mediated by any of the characterized equilibrative (hENT1, hENT2) or sodium-dependent (hCNT1, hCNT2, hCNT3) nucleoside transporters. The low uptake observed for troxacitabine is consistent with a diffusion model.

Table of IC50 Values (μM) for Controls

Exposition of 24 hr to drug, wash, incubated for another 48 hr (total of 72 hr assay)

(3H-Thymidine Incorporation Assay)

IC50 in μM (3H-TdR incorporation at 72 hr)

| | H-460 | MCF-7 | SF-268 | CCRF-CEM | CEM/dCK- | |
|---|---|---|---|---|---|---|
| Compound | 24 h | 24 h | 24 h | 24 h | 24 h | Factor* |
| Gemcitabine | 0.0084 | 0.0090 | 0,0030 | 0.0035 | 51 | 14571 |
| | 0.0140 | 0.0048 | 0,0110 | 0.0064 | 51 | 7969 |
| | 0.0420 | ND | 0,0094 | 0.0034 | 30 | 8824 |
| | 0.0083 | 0.0019 | 0,0077 | 0.0086 | 41 | 4767 |
| | 0,0066 | 0.0083 | 0,0073 | 0.0092 | 30 | 3260 |
| | 0.0100 | 0.0024 | 0,0110 | 0.0048 | 77 | 16041 |
| | 0.0110 | 0.0049 | 0,0100 | 0.0094 | 85 | 9043 |
| | 0,0160 | 0,0093 | 0,0130 | 0,0100 | 86 | 8600 |
| | 0,0094 | 0,0100 | 0,0140 | 0,0086 | 80 | 9302 |
| | 0,0097 | 0,0086 | 0,0100 | 0,0092 | >100 | 10870 |
| | 0,0110 | 0,0056 | 0,0091 | 0,0100 | 91 | 9100 |
| | 0,0110 | 0,0060 | 0,0094 | 0,0092 | 93 | 10109 |
| | 0,0110 | 0,0087 | 0,0090 | 0,0084 | 92 | 10952 |
| | 0,0130 | 0,0120 | 0,0081 | 0,0120 | >100 | >8333 |
| | 0,0041 | 0,0087 | 0,0045 | 0,0028 | 41 | 14643 |
| | 0,0079 | 0,0059 | 0,0075 | 0,0079 | 87 | 11013 |
| | 0,0055 | 0,0031 | 0,0045 | 0,0200 | 61 | 3050 |
| | 0,0110 | 0,0100 | 0,0083 | ND | 88 | ND |
| | 0,0100 | 0,0094 | 0,0100 | 0,0061 | 66 | 10820 |
| | 0,0091 | 0,0029 | 0,0037 | 0,0051 | 34 | 6667 |
| | 0,0074 | 0,0051 | 0,0089 | 0,0090 | 40 | 4444 |
| | 0,0091 | 0,0068 | 0,0078 | 0,0096 | 48 | 5000 |
| | 0,0100 | 0,0089 | 0,0086 | 0,0100 | 72 | 7200 |
| | 0,0110 | 0,0034 | 0,0100 | 0,0099 | 36 | 3636 |
| | 0,0083 | 0,0041 | 0,0029 | 0,0073 | >100 | >13700 |
| Average | 0,011 ± 0,007 | 0,0068 ± 0,0028 | 0,0086 ± 0,0027 | 0,0084 ± 0,0035 | 66 ± 24 | 8618 ± 3614 |
| Cytosine | 0.0140 | 0.0088 | 0.140 | 0.0024 | 21 | 8750 |
| Arabinoside | 0.0190 | 0.0220 | 0.450 | 0.0034 | 24 | 7059 |
| | 0.0500 | ND | 0.470 | 0.0030 | 23 | 7667 |
| | 0.0100 | 0.0098 | 0.077 | 0.0028 | 18 | 6428 |
| | 0.0130 | 0.0100 | 0.320 | 0.0037 | 19 | 5135 |
| | 0.0130 | 0.0140 | 0.033 | 0.0032 | 29 | 8906 |
| | 0.0160 | 0.0160 | 0.300 | 0.0049 | 27 | 5510 |
| | 0,0360 | 0,0170 | 0,300 | 0,0068 | 32 | 4706 |
| | 0,0078 | 0,0200 | ND | 0,0280 | >100 | 6250 |
| | 0,0990 | 0,1000 | 2,100 | 0,0370 | >100 | 2700 |
| | 0,1500 | 0,1500 | 1,900 | 0,0350 | >100 | 2857 |
| | 0,1200 | 0,1700 | 0,890 | 0,0410 | >100 | 2439 |
| | 0,0990 | 0,1000 | 3,600 | 0,0250 | >100 | 4000 |
| | 0,1400 | 0,1500 | 1,200 | 0,0470 | >100 | >2128 |
| | 0,0350 | 0,0960 | 0,120 | 0,0089 | >100 | >11236 |
| | 0,0160 | 0,1100 | 1,600 | 0,0590 | >100 | 1695 |
| | 0,0540 | 0,0340 | 0,930 | 0,0084 | >100 | >11905 |
| | 0,1100 | 0,1000 | 2,600 | ND | >100 | ND |
| | 0,0750 | 0,0810 | 1,100 | 0,0100 | 41 | 4100 |
| | 0,0160 | 0,0095 | 0,770 | 0,0056 | 41 | 7321 |
| | 0,0200 | 0,0210 | 0,660 | 0,0094 | 40 | 4255 |
| | 0,0160 | 0,0270 | 0,920 | 0,0092 | 78 | 8478 |
| | 0,0780 | 0,0520 | 0,720 | 0,0100 | 59 | 5900 |
| | 0,0370 | 0,0120 | 0,490 | 0,0071 | 40 | 5634 |
| | 0,0250 | 0,0310 | 0,110 | 0,0053 | 75 | 14150 |
| Average | 0,052 ± 0,045 | 0,061 ± 0,0520 | 0,94 ± 0,89 | 0,016 ± 0,017 | 62 ± 35 | 5872 ± 2783 |
| BCH-4556 | 0,040 | 0,066 | 0,096 | 0,076 | >100 | >1315 |
| | (72 h) | (72 h) | (72 h) | (24 h) | (24 h) | |
| | 0.130 | 0.005 | 0.27 | 0.045 | 56 | 1244 |
| | 0.140 | 0.140 | 0.33 | 0.040 | >100 | 2500 |
| | 0.049 | ND | 0.43 | 0.091 | >100 | 1099 |
| | 0.110 | 0.140 | 0.17 | 0.073 | >100 | 1370 |
| | 0.086 | 0.180 | 0.24 | 0.065 | >100 | 1538 |
| | 0.150 | 0.190 | 0.68 | 0.120 | >100 | 833 |
| | 0.110 | 0.200 | 0.33 | 0.099 | >100 | 1010 |
| | 0,170 | 0,160 | 0,41 | 0,080 | >100 | 1250 |
| | 0,100 | 0,420 | ND | 0,028 | >100 | 3571 |
| | 0,140 | 0,160 | 0,40 | 0,100 | >100 | 1000 |
| | 0,180 | 0,340 | 0,74 | 0,096 | >100 | 1041 |
| | 0,140 | 0,015 | 0,15 | 0,100 | >100 | 1000 |
| | 0,110 | 0,310 | 0,71 | 0,083 | >100 | 1200 |
| | 0,160 | 0,280 | 0,49 | 0,130 | >100 | >769 |
| | 0,100 | 0,150 | 0,19 | 0,013 | >100 | >7692 |
| | 0,140 | 0,210 | 0,63 | 0,063 | >100 | >1587 |
| | 0,078 | 0,097 | 0,51 | 0,021 | >100 | >4762 |
| | 0,150 | 0,220 | 0,66 | ND | >100 | ND |
| | 0,160 | 0,140 | 0,59 | 0,072 | >100 | >1389 |
| | 0,110 | 0,150 | 0,47 | 0,086 | >100 | >1163 |
| | 0,130 | 0,220 | 0,66 | 0,059 | >100 | >1695 |
| | 0,110 | 0,170 | 0,38 | 0,100 | >100 | >1000 |
| | 0,130 | 0,220 | 0,53 | 0,074 | >100 | >1351 |
| | 0,100 | 0,043 | 0,36 | 0,087 | >100 | >1150 |
| | 0,180 | 0,031 | 0,11 | 0,0053 | >100 | >1136 |
| | 0,12 ± 0,03 | 0,18 ± 0,10 | 0,44 ± 0,18 | 0,078 ± 0,028 | >100 | 1792 ± 1584 |
| 27 | 0,0053 | 0,0073 | 0,023 | nd | nd | nd |
| | (72 h) | (72 h) | (72 h) | | | |
| 275 | 0,0012 | 0,0044 | 0,013 | 0.0056 | 51.6 | 9,214 |
| | (72 h) | (72 h) | (72 h) | | | |
| 276 | 0.025 | 0.0017 | 0,018 | 0.028 | 26.8 | 957 |
| | (72 h) | (72 h) | (72 h) | | | |
| 277 | 0.20 | 0.013 | 0.21 | 0.049 | >100 | 2040 |
| | 0.29 | 0.016 | 0.19 | 0.100 | >100 | >1000 |
| 278 | 0.0024 | 0.023 | 0,013 | 0,028 | 71,2 | 2543 |
| | (72 h) | (72 h) | (72 h) | | | |
| | 0,079 | 0,038 | 0,093 | 0,028 | 91 | 3250 |
| 279 | 0,073 | 0,021 | 0,044 | 0,026 | 48,2 | 1854 |
| | (72 h) | (72 h) | (72 h) | | | |
| | 0,58 | 0,24 | 0,39 | 0,083 | >100 | >1205 |
| 280 | 1.9 | 3.1 | 18 | 1.9 | >100 | >53 |
| 38 | 0.34 | 1 | 0.90 | 0.11 | >100 | 909 |
| 39 | 0.16 | 0.38 | 0.32 | 0.047 | >100 | 2128 |
| | 0.12 | 0.12 | 0.39 | 0.062 | >100 | 1667 |
| 40 | 0.32 | 0.070 | 0.90 | 0.089 | >100 | 1,123 |
| 41 | 40 | 91 | >100 | 21 | >100 | 5 |
| 42 | 0.010 | 0.014 | 0.022 | 0.0022 | 82 | 37272 |
| | 0.007 | 0.005 | 0.026 | 0.0023 | >100 | 43378 |
| 43 | 0.010 | 0.0041 | 0.029 | <0,0001 | >100 | 1,000,000 |
| 44 | 0.37 | 0.97 | 0.89 | 0.077 | >100 | 1,300 |
| 45 | 3.2 | 2.7 | 9 | 1.6 | >100 | 63 |
| 46 | 0.086 | 0.16 | 0.56 | 0.060 | >100 | 1,667 |
| 47 | 1.8 | 2.4 | 38 | 2.9 | >100 | 34 |
| 48 | 0,34 | 1,2 | 0,56 | 0,17 | >100 | 588 |
| | 0,59 | 4,7 | 23 | 3,5 | >100 | >29 |
| 49 | 4.5 | 8.8 | 7.1 | 0.57 | >100 | 175 |
| 50 | 1.2 | 0.82 | 1.3 | 0.17 | >100 | 588 |
| 51 | 0.83 | 0.57 | 0.86 | 0.024 | 47 | 1,958 |
| 52 | 0.0068 | 0.088 | 0.032 | 0.0012 | 0.48 | 400 |
| 53 | 8.9 | 10 | 10 | 2 | 37 | 19 |
| 54 | 0.17 | 0.50 | 0.70 | 0.12 | 65 | 542 |
| 55 | 0.029 | 0.0078 | 0.047 | 0.012 | 64 | 5,333 |
| 56 | 7 | 2 | 25 | 1.6 | >100 | 63 |
| 57 | 0.0061 | 0.019 | 0.047 | 0.0048 | 32 | 6,667 |
| 58 | 0.012 | 0.016 | 0.13 | 0.014 | 38 | 2,714 |
| 59 | 1.4 | 0.19 | 0.69 | 0.54 | >100 | 185 |
| 60 | 2,0 | 0,86 | 0,86 | 0,29 | 2,9 | 10 |
| | 3,1 | 0,95 | 4,7 | 0,31 | 1,8 | 6 |
| 61 | 0.13 | 0.0770 | 0.054 | 0.040 | >100 | >2500 |
| | 0.20 | 0.0088 | 0.013 | 0.013 | >100 | >7692 |
| | 0.076 | 0.015 | 0.064 | 0.0074 | >100 | >13513 |
| 62 | 0.89 | 1.7 | 4.3 | 0.35 | >100 | 288 |
| 63 | 0.11 | 0.37 | 0.076 | 0.036 | >100 | 2,778 |
| 64 | 0.0017 | 0.0044 | 0.0071 | 0.0018 | 3.6 | 2,000 |
| 65 | 0.011 | 0.012 | 0.033 | 0.0039 | 26 | 6,667 |
| 66 | <0,00010 | <0,0001 | <0,0001 | <0,00010 | 3 | >28000 |
| | 0.00025 | 0.000074 | 0.0011 | 0.000009 | >0.1 | 11627 |
| 67 | 0.082 | ND | 0.40 | 0.18 | >100 | 556 |
| 68 | 0.019 | 0.076 | 0.21 | 0.030 | >100 | 3,333 |
| 69 | 0.045 | 0.028 | 0.050 | 0.0069 | 43 | 6,231 |
| 70 | 0.036 | 0.047 | 0.27 | 0.0088 | 30 | 3,409 |
| 71 | 0.31 | 0.13 | 0.81 | 0.18 | >100 | 556 |
| 72 | 0.018 | 0.015 | 0.130 | 0.0160 | 23 | 1450 |
| | 0.027 | 0.017 | 0.075 | 0.0062 | 23 | 3710 |
| 73 | 0.27 | 0.26 | 0.030 | 0.10 | 99 | 990 |
| 74 | 5.2 | 1.4 | 4.4 | 0.33 | 1.3 | 4 |
| 75 | >100 | 64.00 | >100 | >100 | >100 | 1 |
| 76 | >100 | >100 | >100 | >100 | >100 | 1 |
| 77 | 0.059 | 0.030 | 0.38 | 0.054 | 74 | 1,370 |
| 78 | 0.042 | 0.045 | 0.095 | 0.037 | 13 | 351 |
| 79 | 0.12 | 0.17 | 0.16 | 0.014 | 63 | 4,500 |
| 80 | 1.8 | 0.67 | 3.5 | 0.46 | >100 | 217 |
| 81 | 3.1 | 2.2 | 7.9 | 1.2 | >100 | 83 |
| 82 | 0.17 | 0.12 | 0.30 | 0.053 | >100 | 1,887 |
| 83 | 0.054 | 0.083 | 0.26 | 0.022 | >100 | 4,545 |
| 84 | 0.014 | 0.0094 | 0.36 | 0.012 | 60 | 5,000 |
| 85 | 0.69 | 6.8 | 16 | 2.6 | >100 | 38 |
| 86 | 0.0020 | 0.0019 | 0.013 | 0.0011 | 4 | 3,636 |
| 87 | 0,41 | 0,6 | 0,65 | 0,10 | >100 | >1000 |
| | 1,2 | 1,9 | 5,2 | 0,42 | >100 | >238 |
| | 0,48 | 1,2 | 1,9 | 0,39 | >100 | >256 |
| 88 | 0.14 | 0.19 | 0.61 | 0.088 | 82 | 931 |
| 89 | 3.8 | 0.22 | 11 | 2.5 | >100 | 40 |
| 90 | 95 | 61 | >100 | 65 | >100 | 1.5 |
| 91 | 0.63 | 1.8 | 5.5 | 2.8 | >100 | 36 |
| 92 | 2.1 | 1.6 | 4.2 | 1.3 | >100 | 77 |
| 93 | 0.04 | >100 | >100 | 19 | >100 | >5 |
| | 74 | 13.6 | >100 | 4.2 | >100 | >24 |
| 94 | 0.025 | 24 | 38 | 17 | 51 | 3 |
| | 14 | 13 | 92 | 6 | 85 | 16 |
| 95 | <0.0001 | 0.15 | 0.61 | 0.240 | 30 | 123 |
| | nd | 0.10 | 0.25 | 0.057 | 86 | 1503 |
| 96 | 0.0061 | 0.19 | 1.4 | 1.8 | >100 | >56 |
| | 1.5 | 0.21 | 9.6 | 1.9 | >100 | >52 |
| 97 | N.D | 5,0 | 56 | 9.2 | >100 | >11 |
| | 22 | 4,0 | 25 | 5.9 | >100 | >19 |
| 98 | nd | 0.13 | >100 | 35 | >100 | >3 |
| | 36 | 0.15 | 2.2 | 22 | >100 | >4 |
| | 11 | 0.22 | 2.3 | 61 | >100 | >3 |
| 99 | N.D. | 6.3 | 33.0 | 5 | >100 | >20 |
| 100 | nd | 2.70 | 4.80 | 2.70 | 19 | 7 |
| | 0.030 | 1.40 | 0.09 | 0.52 | 55 | 105 |
| | 0,044 | 0,96 | 5,80 | 2,50 | 45 | 18 |
| | nd | 0,25 | 1,00 | 0,64 | 15 | 23 |
| 101 | 0.33 | 0.41 | 2.1 | 0.36 | 16 | 44 |
| 102 | 0.19 | 1.7 | 1.0 | 0.41 | 11 | 27 |
| 103 | 0.052 | 0.018 | 0.063 | 0.011 | 50 | 4,545 |
| 104 | 0.27 | 0.47 | 0.47 | 0.21 | >100 | >476 |
| 105 | 0.080 | 0.068 | 0.071 | 0.033 | 79 | 2393 |
| 106 | 0.014 | 0.037 | 0.095 | 0.010 | 46 | 4,600 |
| 107 | 0.0280 | 0.012 | 0.220 | 0.0120 | 37 | 3100 |
| | 0.0094 | 0.019 | 0.078 | 0.0056 | 30 | 5428 |
| | 0.0340 | 0.030 | 0.034 | 0.0088 | 83 | 9432 |
| | 0,0200 | 0,013 | 0,068 | 0,0200 | 82 | 4100 |
| | 0,0037 | 0,023 | 0,071 | 0,0140 | 59 | 4214 |
| | 0,0084 | 0,035 | 0,260 | 0,0210 | 20 | 952 |
| 108 | 1.8 | 27 | 3.8 | 3.4 | >100 | >29 |
| 109 | 2.6 | 31 | 4.8 | 1.0 | >100 | >100 |
| 110 | 0.0010 | 0.010 | 0.0049 | 0.0013 | 4.3 | 3307 |
| 111 | 0.00013 | 0.00026 | 0.0021 | 0.00020 | 2.6 | 13000 |
| 112 | 0.011 | 0.016 | 0.0067 | 0.0058 | 0.057 | 10 |
| 113 | 0.24 | 0.48 | 1.1 | 0.060 | >100 | >1667 |
| 114 | 0.066 | 0.017 | 0.041 | 0.016 | 8 | 500 |
| 115 | 0.38 | 0.15 | 0.62 | 0.20 | >100 | >500 |
| 116 | 1.4 | 0.11 | 2.5 | 0.38 | >100 | >263 |
| 117 | 0.46 | 0.46 | 0.68 | 0.18 | 89 | 494 |
| 118 | 0.022 | 0.077 | 0.16 | 0.028 | >100 | >3571 |
| 119 | 17 | 27 | 94 | 56 | 96 | ˜2 |
| 120 | >100 | 64 | >100 | >100 | >100 | 1 |
| 121 | 28 | 37 | >100 | 17 | >100 | >6 |
| 122 | 1.9 | 0.21 | 0.57 | 0.71 | 61 | 86 |
| 123 | 1.0 | 1.4 | 2.0 | 0.87 | 15 | 17 |
| 124 | 13 | 14 | 49 | 14 | 27 | ˜2 |
| 125 | 0.24 | 0.016 | 0.60 | 0.072 | 7 | 97 |
| 126 | 0.0041 | 0.0020 | 0.0085 | 0.0016 | 13 | 8,125 |
| 127 | 35.0 | 16 | 23 | 15 | >100 | >7 |
| | 4,9 | 15 | >100 | 22 | >100 | >4,5 |
| 128 | 0.14 | 0.090 | 0.17 | 0.22 | >100 | >454 |
| 129 | 0.15 | 0.020 | 0.20 | 0.072 | 15 | 208 |
| 130 | 0.058 | 0.050 | 0.11 | 0.057 | 75 | 1,316 |
| 131 | 0.11 | 0.10 | 0.012 | 0.021 | 83 | 3,952 |
| 132 | 0.0021 | 0.0011 | <0.0001 | <0.00010 | 8 | >80000 |
| | 0.0190 | 0.0200 | 0.0180 | 0.00091 | >1 | >1100 |
| | 0,0130 | 0,0130 | 0,0130 | 0,00370 | 11 | 2973 |
| | 0,0016 | 0,0010 | 0,0045 | <0.00010 | 10 | >100000 |
| 133 | 0.021 | 0.10 | 0.016 | 0.027 | 31 | 1,148 |
| 134 | 12 | 11 | 3 | 7 | 20 | 3 |
| 135 | 0,15 | 0,23 | 0,25 | 0,097 | 59 | 608 |
| | 9,00 | 11,0 | ND | 4,1 | 19 | 5 |
| 136 | 9 | 12 | 3 | 4 | >100 | >25 |
| 137 | 6.00 | 17.0 | 18,4 | 5.0 | 84 | 17 |
| | 0,35 | 5,1 | 16.0 | 6,5 | 53 | 8 |
| 138 | 0.92 | 1.5 | 2.1 | 0.53 | 58 | 109 |
| 139 | 0.81 | 1.4 | 1.3 | 0.40 | >100 | >250 |
| | 0.51 | 1.7 | 1.7 | 0.42 | >100 | >250 |
| 140 | 10 | 20 | 3 | 11 | >100 | >9 |
| 141 | 0.034 | 0.066 | 0.040 | 0.019 | 69 | 3,632 |
| 142 | 0.038 | 0.029 | 0.13 | 0.0072 | 46 | 6,389 |
| 143 | 0.012 | 0.0037 | 0.14 | 0.0039 | 32.0 | 8,205 |
| 144 | 3 | 5.2 | 1.9 | 0.71 | 78 | 110 |
| 145 | 0.24 | 0.77 | 0.12 | 0.084 | 69 | 821 |
| 146 | 0.78 | 1.2 | 0.028 | 0.13 | 50 | 385 |
| 147 | 0.060 | 0.11 | 0.017 | 0.025 | >100 | >4000 |
| 148 | 36 | 6.30 | 9.90 | 6.3 | 24 | 4 |
| 149 | <0.0001 | 0.00150 | <0.0001 | <0.00010 | 2 | >19000 |
| | 0.0028 | 0.00039 | 0.0070 | 0.00012 | >1,8 | >15000 |
| 150 | 0.96 | 1.6 | 1.3 | 0.13 | 90 | 692 |
| 151 | 9.7 | 8.3 | 4.4 | 0.59 | >100 | >169 |
| 152 | 3.5 | 3.0 | 31.00 | 0.79 | >100 | >127 |
| 153 | 46 | 39 | 59 | 0.21 | >100 | >476 |
| 154 | 0.76 | 1.6 | 4.4 | 0.14 | >100 | >714 |
| 155 | 1,6 | 3,7 | 5,9 | 0,10 | >100 | >1000 |
| | 0,093 | 0,060 | 0,97 | 0,15 | >100 | >667 |
| | 0,43 | 0,76 | 1,7 | 0,54 | >100 | >185 |
| 156 | 0.12 | 0.068 | 0.93 | 0.0070 | 81 | 11,571 |
| 157 | 0.024 | 0.55 | 2.2 | 0.012 | >100 | >8333 |
| 158 | 0.63 | 0.040 | 3.7 | 0.094 | 58 | 617 |
| 159 | 0.87 | 0.72 | 1.6 | 0.38 | >100 | >263 |
| 160 | 0.92 | 0.36 | 1.2 | 0.36 | >100 | >278 |
| 162 | 8.4 | 9.4 | 1.1 | 2.2 | >100 | >44 |
| | 6.4 | 3.9 | 7.0 | 2.8 | >100 | >36 |
| | 9,2 | 5,7 | 12 | 3,3 | >100 | >30 |
| | 2,9 | 3,6 | 17 | 4,1 | >100 | >24 |
| 163 | 0.0092 | 0.033 | 0.025 | 0.0033 | 27 | 8,182 |
| 164 | 0.13 | 0.14 | 0.28 | 0.060 | >100 | 1667 |
| 165 | 3.4 | 10 | 16 | 1.8 | >100 | >56 |
| 166 | 0.0073 | 0.0012 | 0.0046 | 0.0001 | 10 | >90000 |
| | 0.0044 | 0.0014 | 0.0092 | 0.0077 | >1 | >130 |
| | 0,0180 | 0,0090 | 0,0580 | 0,0047 | 10 | 2128 |
| | 0,0170 | 0,0110 | 0,0640 | 0,0024 | >100 | >41667 |
| 167 | 0,160 | 0,20 | 0,64 | 0,073 | 10 | 137 |
| | 0,062 | 0,12 | 0,12 | 0,031 | >100 | 3225 |
| | 0,230 | 0,30 | 0,54 | 0,110 | 12 | 109 |
| 168 | 96 | 16 | 98 | 31 | >100 | >3 |
| | 25 | 2,4 | 31 | 22 | >100 | >4 |
| | 45 | 44 | 59 | 20 | >100 | >5 |
| 169 | 8.2 | 5.1 | 7.1 | 2.0 | >100 | >50 |
| 170 | 0.63 | 0.49 | 1.0 | 0.21 | >100 | >476 |
| 171 | 45 | 41 | 82 | 38 | >100 | >2.6 |
| 172 | 0,014 | 0,019 | 0,0037 | 0,0074 | 2 | 270 |
| | 0,015 | 0,036 | 0,0210 | 0,0085 | 5 | 588 |
| 173 | 6.1 | 17 | 2.0 | 2.6 | >100 | >38 |
| 174 | 11 | 21 | 38 | 9.0 | >100 | >11 |
| 175 | 6.3 | 3.1 | 32 | 3.5 | >100 | >29 |
| 176 | 0,040 | 0,094 | 0,057 | 0,014 | 38 | 2714 |
| | 0,043 | 0,032 | 0,032 | 0,011 | 68 | 6182 |
| 177 | 0.19 | 0.22 | 0.92 | 0.095 | >100 | >1052 |
| 178 | 88 | 5.8 | 41 | 25 | >100 | >4 |
| 179 | 1.7 | 2.8 | 0.56 | 2.4 | >100 | >42 |
| 180 | >100 | 65 | 49 | >100 | >100 | >1 |
| 181 | 0.14 | 0.49 | 0.17 | 0.037 | >100 | >2700 |
| 182 | 0.13 | 0.22 | 0.21 | 0.047 | >100 | >2100 |
| 183 | 0.037 | 0.038 | 0.12 | 0.018 | 45 | 2,500 |
| 184 | 0.94 | 0.92 | 1.1 | 0.81 | 40 | 49 |
| 185 | 0.059 | 0.064 | 0.054 | 0.066 | 17 | 258 |
| 186 | <0.0001 | 0,0300 | 0,0270 | 0,0087 | >100 | >11494 |
| | <0.0001 | 0,0210 | 0,0017 | 0,0220 | >100 | >4545 |
| | 0,0039 | 0,0062 | 0,0770 | 0,0049 | >100 | >20408 |
| 187 | 0,0014 | 0,0042 | 0,0200 | 0,0017 | 4,1 | 2412 |
| | 0,0011 | 0,0051 | 0,0080 | 0,0016 | 0,66 | 413 |
| 188 | 0,097 | 3,0 | 0,46 | 0,79 | >100 | >127 |
| | 0,068 | 3,8 | 2,40 | 1,50 | >100 | >67 |
| | 0,120 | 4,9 | 2,40 | 1,10 | >100 | >91 |
| 189 | 0,00120 | 0,0033 | 0,0092 | 0,0021 | 2,8 | 1333 |
| | 0,00068 | 0,0037 | 0,0016 | 0,0010 | 1,3 | 1300 |
| 190 | 0,0061 | 0,027 | 0,0400 | 0,0084 | 22 | 2619 |
| | 0,0039 | 0,016 | 0,0056 | 0,0036 | 9,8 | 2722 |
| 191 | <1E-04 | <1E-04 | <1E-04 | <1E-04 | 0,54 | >5400 |
| | <1E-11 | <1E-11 | <1E-11 | <1E-11 | >1E-04 | >1E07 |
| | ND | ND | ND | 1,6E-11 | 11 | 7,0E11 |
| 192 | 0.29 | 0.0016 | 0.40 | 0.0084 | 48 | 5,714 |
| 193 | 0.64 | 0.16 | 2.0 | 0.059 | >100 | >1695 |
| 194 | 0.011 | 0.0040 | 0.041 | 0.0024 | 10 | 4167 |
| 195 | 1.1 | 1.9 | 1.5 | 0.064 | >100 | >1563 |
| 196 | <1E-04 | <1E-04 | <1E-04 | <1E-04 | 2,5 | >25000 |
| | 1.1E-08 | <1E-11 | 2.5E-07 | <1E-11 | >1E-04 | >1E07 |
| | ND | ND | ND | 1,2E-06 | 26 | 2,2E07 |
| 197 | <1E-04 | <1E-04 | <1E-04 | <1E-04 | 0,94 | >9400 |
| | <1E-11 | <1E-11 | <1E-11 | <1E-11 | >1E-04 | >1E07 |
| | ND | ND | ND | ND | 11 | ND |
| 198 | <1E-04 | <1E-04 | <1E-04 | <1E-04 | 2,1 | >21000 |
| | 1.4E-08 | 1.2E-05 | 1.0E-07 | 1.1E-08 | >1E-04 | >10000 |
| | ND | ND | ND | ND | 17 | ND |
| 199 | 0.033 | 0.21 | 0.0078 | 0.0094 | >100 | >10638 |
| 200 | 0.30 | 1.1 | 0.12 | 0.31 | 72 | 232 |
| 201 | 17 | 18 | 7.3 | 14 | >100 | >7 |
| 202 | <1E-04 | <1E-04 | <1E-04 | <1E-04 | 0,1 | >1000 |
| | 2,1E-05 | ND | 1,2E-05 | ND | 1,1 | ND |
| 203 | <1E-04 | <1E-04 | <1E-04 | <1E-04 | 1,3 | >13000 |
| | ND | ND | ND | 3,3E-04 | 8,6 | 26060 |
| 204 | 0.015 | 0.0086 | 0.025 | 0.012 | 19 | 1600 |
| 205 | 0.28 | 0.90 | 0.10 | 0.26 | >100 | >385 |
| 206 | 0.012 | 0.056 | 0.043 | 0.0090 | 80 | 8,889 |
| 207 | 0.0061 | 0.0044 | 0.0023 | 0.0027 | 15 | 5,556 |
| 208 | <1E-04 | <1E-04 | <1E-04 | <1E-04 | 1,42 | >14000 |
| | 0,0027 | 0,00063 | 0,0062 | 0,000052 | 11 | 211538 |
| 209 | 0.31 | 1.3 | 0.59 | ND | >100 | ND |
| 210 | 0.0026 | 0.0050 | 0.26 | ND | >100 | ND |
| 211 | ≦0,0001 | ≦0,0001 | ≦0,0001 | ND | 0,71 | ND |
| | 0,0000086 | 0,000015 | 0,00016 | 0,000027 | >1 | >3704 |
| | 0,0000400 | 0,000030 | 0,00087 | 0,000053 | >0,1 | >1887 |
| 212 | 0.00011 | 0.00059 | 0.018 | ND | 3.5 | ND |
| 213 | ≦0,0001 | 0.00027 | 0.012 | ND | 1.1 | ND |
| 214 | 9.4 | 9.4 | 89 | ND | >100 | ND |
| 215 | 3.9 | 33 | 96 | ND | >100 | ND |
| 216 | 0.00088 | ≦0,0001 | 0.018 | ND | 14 | ND |
| 217 | ≦0,0001 | ≦0,0001 | 0.00013 | ND | 1.2 | ND |
| 218 | 0.0091 | 0.052 | 0.081 | ND | 60 | ND |
| 219 | ≦0,0001 | ≦0,0001 | 0.00012 | ND | 2.1 | ND |
| 220 | 0.0034 | 0.029 | 0.042 | 0.0035 | >100 | >28571 |
| 221 | 0.43 | 0.39 | 1.6 | 0.13 | >100 | >769 |
| 222 | 0.21 | 0.19 | 0.85 | 0.11 | >100 | >909 |
| 223 | 0.035 | 0.15 | 0.25 | 0.062 | >100 | >1613 |
| 224 | 5.3 | 6.9 | 21 | 0.10 | >100 | >1000 |
| 225 | 11 | 11 | 43 | 0.88 | >100 | >113 |
| 226 | 0,00063 | 0,0017 | 0,035 | 0,00076 | 28 | 36842 |
| | 0,02600 | 0,0330 | 0,016 | 0,02100 | >0,1 | >5 |
| 227 | 0.84 | 0.012 | 3.0 | 0.043 | 22 | 512 |
| 228 | 0.68 | 1.5 | 5.3 | 0.44 | >100 | >227 |
| 229 | 13 | 15 | 11 | 11 | >100 | >9 |
| | 14 | 18 | 57 | ND | >100 | ND |
| 230 | 1.5 | 3.8 | 9.5 | 1.0 | >100 | >100 |
| 231 | 0.015 | 0.15 | 1.1 | 0.076 | >100 | >1315 |
| 232 | 0,00053 | 0,0096 | 0,0190 | 0,0037 | 5,8 | 1568 |
| | 0,00038 | 0,0017 | 0,0041 | 0,0019 | 4,5 | 2368 |
| 233 | 1,5 | 13 | 12 | 11 | 18 | 1,7 |
| | 5,4 | 9,6 | 17 | ND | 18 | ND |
| | 4,4 | 11 | 15 | 9,7 | 22 | 2 |
| 234 | 1.5 | 0.10 | 0.10 | 0.95 | >100 | >105 |
| 235 | 1.6 | 1.1 | 0.38 | 1.2 | 61 | 51 |
| 236 | 3.7 | 8.6 | 0.12 | 5.1 | >100 | >20 |
| 237 | 0.0026 | ≦0.0001 | 0.088 | 0.0016 | 18 | 11,250 |
| 238 | 0.00045 | ≦0.0001 | 0.025 | 0.0025 | 59 | 23,600 |
| 239 | 0.0065 | 0.00033 | 0.19 | 0.0030 | 20 | 6667 |
| 240 | ≦0.0001 | ≦0.0001 | ≦0.0001 | ≦0.0001 | 2.5 | ≧25000 |
| 241 | 0.047 | 0.17 | 14 | 1.4 | ≧100 | ≧74 |
| 242 | 0.25 | 0.0010 | 1.1 | 0.23 | 93 | 404 |
| 243 | 0.0011 | 0.00050 | 0.32 | 0.027 | 72 | 2,667 |
| 244 | 1.9 | 0.019 | 26 | 11 | ≧100 | ≧9 |
| 245 | <1E-4 | <1E-4 | <1E-4 | <1E-4 | 0.68 | >6800 |
| 246 | 47 | 1.4 | 28 | 25 | >100 | >4 |
| 247 | 0.13 | 0.00078 | 0.13 | 0.10 | 15 | 150 |
| 249 | 8.6 | 0.78 | 8.4 | 3.9 | >100 | >25 |
| 250 | 0.17 | 0.16 | 0.17 | 0.063 | 31 | 492 |
| 254 | 0.17 | 0.18 | 0.29 | 0.098 | 31 | 316 |
| 256 | 4.6 | 5.1 | 14 | 5.3 | 20 | 4 |
| 257 | 9.7 | 5 | 1.6 | 4.2 | >100 | >24 |

*Resistance Factor = Ratio of dCK- on Wild-type CCRF-CEM
ND: Not Determined
NIH lines:
MCF-7: Human Breast Carcinoma
H-460: Human Lung Carcinoma
SF-268: Human Central Nervous System Tumor
CCRF-CEM: T-cell Leukemia
Dck-: CCRF-CEM deoxycytidine kinase-deficient

Table 2 of IC50 Values (uM) for Pro-drugs of BCH-4556

Exposition of 24hr to drug, washed, and incubated for another 48hr (total of 72hr assay)

IC50 μM (MTT at 72 hr) IC50 μM (MMT
or WST-1 at 72 hr)

| | | | | | CEM/d | |
|---|---|---|---|---|---|---|
| | H-460 | MCF-7 | SF-268 | CCRF-CEM | CK- | Resistance |
| BCH | 24 h | 24 h | 24 h | 24 h | 24 h | Factor* |
| Gemcitabine | 0.012 | 0.0060 | 0.015 | ND | >100 | ND |
| | 0.017 | 0.0092 | 0.064 | 0.0740 | >100 | >1351 |
| | 0.086 | 0.2800 | 0.180 | ND | >100 | ND |
| | 0.420 | 0.2600 | 0.220 | 0.0240 | 6.7 | 279 |
| | 0.046 | 0.0770 | 0.056 | 0.0250 | 19 | 760 |
| | 0.012 | 0.1100 | 0.048 | 0.0100 | 49 | 4900 |
| | 0.086 | 0.0070 | 0.270 | 0.0071 | 34 | 4789 |
| | 0.013 | 0.0150 | 0.082 | 0.0067 | 11 | 1642 |
| | 0.014 | 0.0078 | 0.017 | 0.0088 | 56 | 6364 |
| | 0.012 | 0.0120 | 0.840 | 0.0083 | 98 | 11807 |
| | 0.070 | 0.1200 | 0.130 | 0.0051 | 65 | 12745 |
| | 0.055 | 0.0270 | 0.023 | 0.0038 | >10 | >2631 |
| Average | 0.072 ± 0.1 | 0.078 ± 0. | 0.18 ± 0.25 | 0.020 ± 0.023 | 57 ± 39 | 3987 ± 3871 |
| | 26 | 107 | | | | |
| Cytosine | 0.150 | 0.110 | 4.1 | ND | >100 | ND |
| Arabinoside | 0.088 | 0.058 | 26 | 0.0820 | >100 | >1220 |
| | 0.250 | 0.510 | 7.2 | ND | >100 | ND |
| | 0.780 | 0.920 | 73 | 0.0370 | >100 | >2700 |
| | 0.130 | 0.210 | 39 | 0.0380 | 69 | 1816 |
| | 0.063 | 0.830 | 16 | 0.0130 | 83 | 6385 |
| | 0.180 | 0.054 | 42 | 0.0085 | 15 | 1765 |
| | 0.081 | 0.056 | 15 | 0.0079 | 11 | 1392 |
| | 0.066 | 0.050 | 1.9 | 0.0100 | 29 | 2900 |
| | 0.073 | 0.061 | ND | 0.0100 | 69 | 6900 |
| | 0.350 | 0.860 | 7.8 | 0.0094 | 91 | 9680 |
| | 0.095 | 0.160 | 5.9 | 0.0078 | >10 | >1282 |
| Average | 0.19 ± 0.22 | 0.29 ± 0.3 | 25 ± 23 | 0.026 ± 0.026 | 68 ± 36 | 3135 ± 2246 |
| | | 4 | | | | |
| BCH-4556 | 0.35 | 0.12 | 16 | ND | >100 | ND |
| | 0.78 | 0.63 | 17 | 0.44 | >100 | >227 |
| | 3.50 | 3.20 | 9.8 | ND | >100 | ND |
| | 5.10 | 7.70 | 45 | 0.72 | >100 | >139 |
| | 1.70 | 1.30 | 15 | 0.79 | >100 | >126 |
| | 0.51 | 3.30 | 32 | 0.14 | >100 | >714 |
| | 1.30 | 0.53 | 28 | 0.21 | >100 | >476 |
| | 0.76 | 0.51 | 19 | 0.21 | 10 | 48 |
| | ND | ND | ND | ND | ND | ND |
| | 0.54 | 0.72 | 83 | 0.14 | >100 | >714 |
| | 2.30 | 1.60 | 16 | 0.16 | >100 | >625 |
| | 0.78 | 1.50 | 7.1 | 0.14 | >10 | >71 |
| Average | 1.6 ± 1.6 | 2.0 ± 2.4 | 29 ± 23 | 0.38 ± 0.28 | >100 | 349 ± 283 |
| 277 | 2.0 | 0.32 | 7.3 | 0.48 | >100 | >208 |
| 107 | 0.27 | 0.25 | 3.4 | 0.024 | 49 | 2.042 |
| 110 | 0.01300 | 0.018 | 1.10 | 0.0034 | 1.3 | 382 |
| (HCl salt: | 0.00049 | 0.120 | 0.14 | 0.0025 | 7.1 | 2840 |
| 251) | 0.00060 | 0.240 | 7.50 | 0.0040 | 9.4 | 2350 |
| 172 | 0.21 | 0.17 | 0.76 | 0.09 | 1.3 | 14 |
| | 2.70 | 1.30 | 9.70 | 0.28 | 32 | 114 |
| | 3.30 | 0.97 | 54 | 0.20 | 80 | 400 |
| 185 | 0.86 | 1.4 | 4.9 | 0.18 | 12 | 67 |
| | 1.70 | 1.4 | 5.9 | 0.18 | 12 | 67 |
| | 1.80 | 2.3 | 17 | 0.45 | 30 | 67 |
| 186 | 0.0057 | 0.047 | 1.7 | 0.0086 | 26 | 3023 |
| | 0.0270 | 3.4 | >10 | 0.0790 | 14 | 177 |
| 191 | ≦0.0001 | ≦0.0001 | 0.010 | ND | 1.1 | ND |
| | 0.0078 | 0.0041 | >0.1 | 0.0029 | >0.1 | >34 |
| | 0.0017 | 0.0054 | 0.065 | 0.0710 | 12 | 169 |
| 196 | 0.010 | 0.0010 | 0.045 | ND | 7.7 | ND |
| | 0.098 | 0.0064 | 0.650 | 0.010 | >1 | >100 |
| | | | | | | 43 |
| 197 | ≦0.0001 | ≦0.0001 | 0.01 | ND | 7.4 | ND |
| | 0.0097 | 0.00250 | >0.1 | 0.0018 | >0.1 | >56 |
| | 0.0038 | 0.00014 | 0.22 | 0.0530 | >100 | >1886 |
| 198 | ≦0.0001 | 0.0001 | 0.0054 | ND | 10 | ND |
| (HCl salt: | 0.0062 | 0.0028 | >0.1 | 0.0083 | >0.1 | >12 |
| 261) | 0.0068 | 0.0046 | 0.73 | 0.1400 | 23 | 164 |
| 202 | ≦0.0001 | 0.0001 | 0.043 | ND | 0.05 | ND |
| | 0.021 | 0.0850 | >0.1 | 0.014 | >0.1 | >7 |
| 203 | 0.120 | 0.010 | 0.72 | ND | 1.2 | ND |
| | 0.250 | 0.089 | >1 | 0.010 | >1 | >100 |
| | 0.050 | 0.120 | 7.4 | 0.460 | 20 | 43 |
| 207 | 0.53 | 0.13 | >1 | 0.074 | >1 | >14 |
| | 0.65 | 0.49 | >1 | 0.190 | >1 | >5 |
| 208 | 0.11 | 0.031 | 0.47 | 0.0590 | 25 | 424 |
| | 0.20 | 0.066 | 2.20 | 0.0093 | >1 | >108 |
| 210 | 0.37 | 0.130 | ≧100 | 0.24 | 51 | 204 |
| | 1.70 | 0.065 | >100 | 0.46 | >100 | >217 |
| | 0.11 | 0.270 | 51 | 0.13 | >100 | >770 |
| | 0.22 | 0.110 | >100 | 0.50 | 47 | 94 |
| 211 | 0.0053 | 0.00100 | 0.038 | 0.0028000 | >1 | >357 |
| (HCl salt: | 0.0030 | 0.00015 | 0.050 | 0.0350000 | 13 | 371 |
| 248) | 0.0140 | 0.00770 | 0.034 | 0.0003300 | >0.1 | >303 |
| | ND | 0.00013 | 0.012 | ND | 8.70 | ND |
| | <1e−6 | <1e−6 | 0.029 | <1e−6 | 1.50 | >1500000 |
| | 0.0087 | 0.00130 | 0.034 | 0.0000023 | 0.44 | >191300 |
| 216 | 0.064 | 0.0094 | 0.40 | 0.34 | 31 | 91 |
| 217 | 0.011 | 0.0039 | 0.12 | 0.36 | 27 | 75 |
| 219 | 0.014 | 0.0037 | 0.18 | 0.018 | 51 | 2833 |
| | 0.058 | 0.0220 | 1.60 | 0.010 | >1 | >100 |
| 223 | 1.70 | 1.7 | 15 | 0.12 | >100 | >833 |
| | 0.78 | 2.1 | 47 | 0.13 | >100 | >769 |
| | 4.00 | 1.4 | 45 | 0.45 | >100 | >222 |
| 226 | 0.850 | 0.40 | >1 | 0.0600 | >1 | >17 |
| | 0.250 | 0.26 | 1.8 | 0.0410 | >10 | >244 |
| | 0.065 | 0.22 | 3.9 | 0.0011 | 15 | 13636 |
| | 0.420 | 0.14 | 17 | 0.0260 | 35 | 1346 |
| 232 | 0.0069 | 0.020 | 0.16 | 0.010 | 2.1 | 210 |
| 237 | 0.042 | 0.0011 | 3.3 | 0.0014 | 2.7 | 1928 |
| | 5.200 | 0.0220 | 1.8 | 0.0100 | 22 | 2200 |
| | 0.170 | 0.1700 | 2.7 | 0.0040 | 15 | 3750 |
| 238 | 0.064 | 0.00460 | 5.7 | 0.0170 | 23 | 1353 |
| (HCl salt: | 0.046 | 0.00130 | 1.9 | 0.0050 | 10 | 2000 |
| 269) | 0.017 | 0.00020 | 5.6 | 0.0048 | 5.2 | 1080 |
| | 0.062 | 0.01000 | 2.7 | 0.0014 | 28 | 20000 |
| 239 | 0.49 | 0.0021 | 9.0 | 0.0045 | 20 | 4444 |
| | 0.20 | 0.0031 | 4.9 | 0.0022 | 28 | 12727 |
| | 0.20 | 0.6400 | 25 | 0.0110 | 17 | 1545 |
| 240 | <1e−6 | <1e−6 | 0.053 | <1e−6 | 1.70 | >1700000 |
| (HCl salt: | 0.0091 | 0.00045 | 0.016 | 0.000011 | 0.11 | 10000 |
| 264) | 0.0014 | 0.00068 | 0.031 | 0.000029 | 0.84 | 28965 |
| | 0.0069 | 0.00190 | 0.028 | 0.000002 | 1.40 | 700000 |
| 243 | 0.140 | 0.00640 | 14 | 0.0480 | 30 | 625 |
| (HCl salt: | 0.038 | 0.00079 | 7.7 | 0.0081 | 21 | 2593 |
| 260) | 0.024 | 0.12000 | 68 | 0.0400 | 51 | 1275 |
| 245 | 0.00021 | <1E−5 | 0.0440 | <1E−5 | 2.2 | >220000 |
| (HCl salt: | 0.00290 | 0.00300 | 0.0950 | 0.000021 | 3.4 | 161904 |
| 268) | 0.00110 | 0.00013 | 0.0047 | >1E−6 | 6.0 | >6E6 |
| 247 | 0.39 | 0.00089 | 6.1 | 0.024 | 61 | 2542 |
| | 0.54 | 0.30000 | >10 | 0.140 | 49 | 350 |
| | 0.46 | 0.01600 | 14 | 0.170 | 61 | 359 |
| 257 | 89 | 36 | >100 | 4.1 | >100 | >24 |
| | 42 | 21 | >100 | 5.4 | >100 | >19 |
| 262 | 0.90 | 16 | >100 | 0.88 | >100 | >114 |
| 263 | 66 | 73 | >100 | 19 | >100 | >5 |
| | >100 | 12 | >100 | 14 | >100 | >7 |
| 265 | >100 | 77 | >100 | 30 | >100 | >3 |
| 266 | 0.00690 | 0.0120 | 1.00 | 0.00190 | 21 | 11050 |
| | 0.00053 | 0.0013 | 0.42 | 0.00067 | 26 | 37143 |
| 267 | 93 | 34 | >10 | 2.9 | >10 | >3 |

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method of treating a patient having a cancer comprising administering to said patient a compound having the following formula: 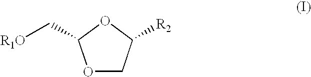
wherein:
R₁ is H; C₁₋₂₄ alkyl; C₂₋₂₄ alkenyl; C₆-24 aryl; C₅₋₂₀ heteroaromatic ring; C₃₋₂₀ non-aromatic ring optionally containing 1-3 heteroatoms selected from the group comprising O, N, or S; —C(O)R₆; —(O)OR₆; —C(O)NHR₆; or an amino acid radical or a dipeptide or tripeptide chain or mimetic thereof, wherein the amino acid radicals are selected from the group comprising Glu, Gly, Ala, Val, Leu, Ile, Pro, Phe, Tyr, Trp, Ser, Thr, Cys, Met, Asn and Gln, and which in each case is optionally terminated by —R₇;
R₁ can also be a P(O) (OR′)₂ group wherein R′ is in each case independently H, C₁₋₂₄ alkyl, C₂₋₂₄ alkenyl, C₆₋₂₄ aryl, C₇₋₁₈ arylmethyl, C₂₋₁₈ acyloxymethyl, C₃₋₈ alkoxycarbonyloxymethyl, C₃₋₈ S-acyl-2-thioethyl; saleginyl, t-butyl, phosphate or diphosphate;
R₁ can also be monophosphate, diphosphate, triphosphate or mimetics thereof;
R₂ is 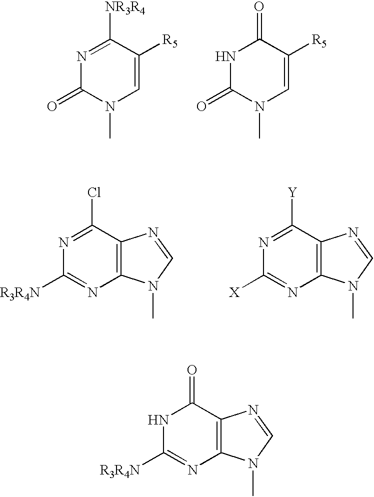
R₃ and R₄ are in each case independently H; C₁₋₂₄ alkyl; C₂₋₂₄ alkenyl; C₆₋₂₄ aryl; C₅₋₁₈ heteroaromatic ring; C₃₋₂₀ non-aromatic ring optionally containing 1-3 heteroatoms selected from the group comprising O, N, or S; —C(O)R₆; —C(O)OR₆; —C(O)NHR₆; or an amino acid radical or a dipeptide or tripeptide chain or mimetic thereof wherein the amino acids radicals are selected from the group comprising Glu, Gly, Ala, Val, Leu, Ile, Pro, Phe, Tyr, Trp, Ser, Thr, Cys, Met, Asn and Gln, and which in each case is optionally terminated by —R₇;
R₆ is, in each case, H, C₁₋₂₀ alkyl, C₂₋₂₀ alkenyl, C₀₋₂₀ alkyl-C₆₋₂₄ aryl, C₀₋₂₀ alkyl-C₅₋₂₀ heteroaromatic ring, C₃₋₂₀ non-aromatic ring optionally containing 1-3
heteroatoms selected from the group comprising O, N or S; and
R₇ is, in each case, C₁₋₂₀ alkyl, C₂₋₂₀ alkenyl, C₆₋₁₀ aryl, C₀₋₂₀ heteroaromatic ring, C₃₋₂₀ non-aromatic ring optionally containing 1-3 heteroatoms selected from the group comprising O, N or S, —C(O)R₆, —C(O)OR₆; and
X and Y are each independently Br, Cl, I, F, OH, OR₃ or NR₃R₄ and at least one of X and Y is NR₃R₄; or a pharmaceutically acceptable salt thereof.

2. A method according to claim 1, wherein at that least one of R₁, R₃ and R₄ is other than H, and if R₃ and R₄ are both H and R₁ is —C(O)R₆, —C(O)OR₆ or —C(O)NHR₆, then R₆ is other than H.

3. A method according to claim 1, wherein R₂ is of the formula:

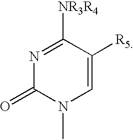

4. A method of treating a patient with cancer, wherein the cancer cells are def icient in nucleoside or nucleobase transporter proteins, comprising administering to said patient a compound according to the following formula: 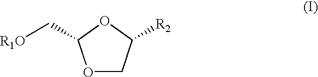
wherein:
R₁ is H; Cl₂₄ alkyl; C₂₋₂₄ alkenyl; C₆₋₂₄ aryl; C₅₋₂₀ heteroaromatic ring; C₃₋₂₀ non-aromatic ring optionally containing 1-3 heteroatoms selected from the group comprising O, N, or S; —C(O)R₆; —C(O)OR₆; —C(O)NHR₆; or an amino acid radical or a dipeptide or tripeptide chain or mimetic thereof wherein the amino acid radicals are selected from the group comprising Glu, Gly, Ala, Val, Leu, Ile, Pro, Phe, Tyr, Trp, Ser, Thr, Cys, Met, Asn and Gln, and which in each case is optionally terminated by —R₇;
R₁ can also be a P(O) (OR′)₂ group wherein R′ is in each case independently H, C₁₋₂₄ alkyl, C₂₋₂₄ alkenyl, C₆₋₂₄ aryl, C₇₋₁₈ arylmethyl, C₂₋₁₈ acyloxymethyl, C₃₋₈ alkoxycarbonyloxymethyl, or C₃₋₈ S-acyl-2-thioethyl, saleginyl, t-butyl, phosphate or diphosphate;
R₁ can also be monophosphate, diphosphate or triphosphate or mimetics thereof;
R₂ is 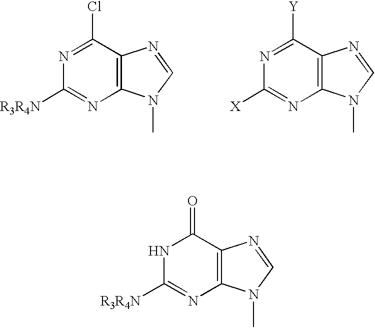
R₃ and R₄ are in each case independently H; C₁₋₂₄ alkyl; C₂₋₂₄ alkenyl; C₆₋₂₄ aryl; C₅₋₁₈ heteroaromatic ring; C₃₋₂₀ non-aromatic ring optionally containing 1-3 heteroatoms selected from the group comprising O, N, or S; —C(O)R₆; —C(O)OR₆; —C(O)NHR₆; or an amino acid radical or a dipeptide or tripeptide chain or mimetic thereof wherein the amino acid radicals are selected from the group comprising Glu, Gly, Ala, Val, Leu, Ile, Pro, Phe, Tyr, Trp, Ser, Thr, Cys, Met, Asn and Gln, and which in each case is optionally terminated by —R₇;
R₆ is, in each case, H, C₁₋₂₄ alkyl, C₂₋₂₄ alkenyl, C₀₋₂₀ alkyl-C₆₋₂₄ aryl, C₀₋₂₀ alkyl-C₅₋₈ heteroaromatic ring, C₃₋₂₀ non-aromatic ring optionally containing 1-3 heteroatoms selected from the group comprising O, N or S;
R₇ is, in each case, C₁₋₂₀ alkyl, C₂₋₂₀ alkenyl, C₆₋₁₀ aryl, C₅₋₁₀ heteroaromatic ring, C₃₋₂₀ non-aromatic ring optionally containing 1-3 heteroatoms selected from the group comprising O, N or S, —C(O)R₆, —C(O)OR₆; and
X and Y are each independently Br, Cl, I, F, OH, OR₃ or NR₃R₄ and at least one of X and Y is NR₃R₄; or a pharmaceutically acceptable salt thereof.

5. A method according to claim 4, wherein at least one of R₁, R₃ and R₄ is other than H, and if R₃ and R₄ are both H and R₁ is —C(O) R₆, —C(O)OR₆₁ or —C(O)NHR₆ then R₆ is other than H.

6. A method according to claim 4, wherein said cancer cells are deficient in one or more nucleoside or nucleobase transporter proteins that provide sodium-independent, bidirectional equilibrative transport.

7. A method according to claim 4, wherein said cancer cells are deficient in nucleoside or nucleobase transporter proteins that provide sodium-dependent, inwardly directed concentrative processes.

8. A method according to claim 7, wherein said cancer cells are deficient in nucleoside or nucleobase transporter proteins that provide sodium-dependent, inwardly directed concentrative processes.

9. A method according to claim 4, wherein said cancer cells are deficient in es transporter proteins, ei transporter proteins or both.

10. A method according to claim 4, wherein said cancer cells are deficient in cit transporter proteins, cib transporter proteins, cif transporter proteins, csg transporter proteins, Cs transporter proteins, or combinations thereof.

11. A method according to claim 4, wherein R₂ is of the formula:

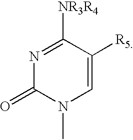

12. A method of treating patients with cancer comprising administering to said patient a compound of the following formula: 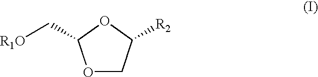
wherein:
R₁ is H; C₁₋₂₄ alkyl; C₂₋₂₄ alkenyl; C₆₋₂₄ aryl; C₅₋₂₀ heteroaromatic ring; C₃₋₂₀ non-aromatic ring optionally containing 1-3 heteroatoms selected from the group comprising O, N, or S; —C(O)R₆; —C(O)OR₆; —C(O)NHR₆; or an amino acid radical or a dipeptide or tripeptide chain or mimetic thereof wherein the amino acids radicals are selected from the group comprising Glu, Gly, Ala, Val, Leu, Ile, Pro, Phe, Tyr, Trp, Ser, Thr, Cys, Met, Asn and Gly, and which in each case is optionally terminated by —R₇;
R₁ can also be a P(O) (OR′)₂ group wherein R′ is in each case independently H, C₁₋₂₄ alkyl, C₂₋₂₄ alkenyl, C₆₋₂₄ aryl, C₇₋₁₈ arylmethyl, C₂₋₁₈ acyloxymethyl, C₃₋₈ alkoxycarbonyloxymethyl, C₃₋₈ S-acyl-2-thioethyl, saleginyl, t-butyl, phosphate or diphosphate;
R₁ can also be monophosphate, diphosphate, triophosphate or mimetics thereof;
R₂ is 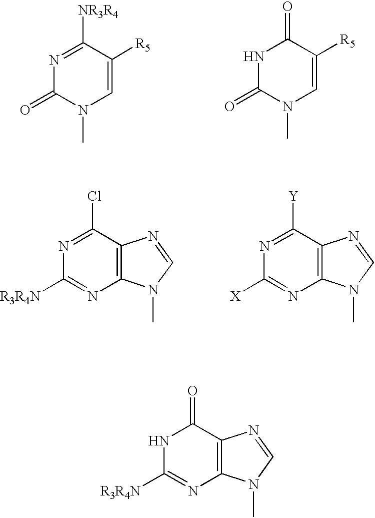
R₃ and R₄ are in each case independently H; C₁₋₂₀ alkyl; C₂₋₂₀ alkenyl; C₆₋₁₀ aryl; C₅₋₁₀ heteroaromatic ring; C₃₋₂₀ non-aromatic ring optionally containing 1-3 heteroatoms selected from the group comprising O, N, or S; —C(O)R₆; —C(O)OR₆; —C(O)NHR₆; or an amino acid radical or dipeptide or tripeptide chain or mimetic thereof wherein the amino acids radicals are selected from the group comprising Glu, Gly, Ala, Val, Leu, Ile, Pro, Phe, Tyr, Trp, Ser, Thr, Cys, Met, Asn and Gln, and at least one amino acid is not Gly, and which in each case is optionally terminated by —R₇;
R₆ is, in each case, H, C₁₋₂₀ alkyl, C₂₋₂₀ alkenyl, C₀₋₂₀ alkyl-C₆₋₁₀ aryl, C₀₋₂₀ alkyl-CO₅₋₁₀ heteroaromatic ring, C₃₋₂₀ non-aromatic ring optionally containing 1-3 heteroatoms selected from the group comprising O, N or S;
R₇ is, in each case, C₁₋₂₀ alkyl, C₂₋₂₀ alkenyl, C₆₋₁₀ aryl, C₅₋₁₀ heteroaromatic ring, C₃₋₂₀ non-aromatic ring optionally containing 1-3 heteroatoms selected from the group comprising O, N or S, —C(O)R₆, —C(O)OR₆; and
X and Y are each independently Br, Cl, I, F, OH, OR₃ or NR₃R₄ and at least one of X and Y is NR₃R₄;
with the proviso that least one of R₁, R₃ and R₄ is other than H, and if R₃ and R₄ are both H and R, is —C(O)R₆, —C(O)OR₆, or —C(O)NHR₆ then R₆ is other than H; or
a pharmaceutically acceptable salt thereof;
wherein said compound is administered at least daily for a period of 2 to 10 days.

13. A method according to claim 12, wherein R₂ is of the formula:

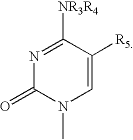

14. A method of treating a patient with cancer wherein the cancer is resistant to cytarabine, said method comprising administering to said patient a compound according to the following formula:
R₁ is H; C₁₋₂₄ alkyl; C₂₋₂₄ alkenyl; C₆₋₂₄ aryl; C₅₋₂₀ heteroaromatic ring; C₃₋₂₀ non-aromatic ring optionally containing 1-3 heteroatoms selected from the group comprising O, N, or S; —C(O)R₆; —C(O)OR₆; —C(Q)NRH₆; or an amino acid radical or a dipeptide or tripeptide chain or mimetic thereof wherein the amino acids radicals are selected from the group comprising Glu, Gly, Ala, Val, Leu, Ile, Pro, Phe, Tyr, Trp, Ser, Thr, Cys, Met, Asn and Gln, and which in each case is optionally terminated by —R₇;
R₁ can also be a P(O) (OR′) ₂ group wherein R′ is in each case independently H, C₁₋₂₄ alkyl, C₂₋₂₄ alkenyl, C₆₋₂₄ aryl, C₇₋₁₈ arylmethyl, C₂₋₁₈ acyloxymethyl, C₃₋₈ alkoxycarbonyloxymethyl, C₃₋₈ S-acyl-2-thioethyl, saleginyl, t-butyl, phosphate or diphosphate;
R₁ can also be monophosphate, diphosphate, triphosphate or mimetics thereof;
R₂ is 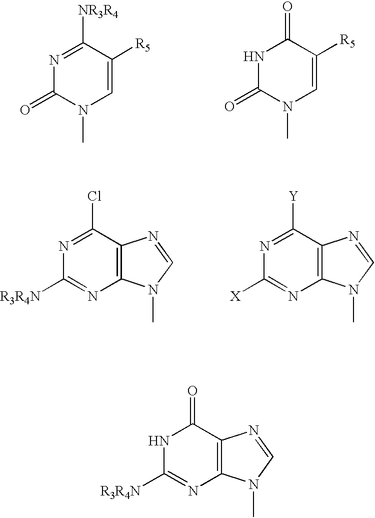
R₃ and R₄ are in each case independently H; C₁₋₂₄ alkyl; C₂₋₂₄ alkenyl; C₆₋₂₄ aryl; C₅₋₁₈ heteroaromatic ring; C₃₋₂₀ non-aromatic ring optionally containing 1-3 heteroatoms selected from the group comprising O, N, or S; —C(O)R₆; —C(O)OR₆; —C(O)NHR₆; or an amino acid radical or a dipeptide or a tripeptide chain or mimetic thereof wherein the amino acids are selected from the group comprising Glu, Gly, Ala, Val, Leu, Ile, Pro, Phe, Tyr, Trp, Ser, Thr, Cys, Met, Asn and Gln, and which in each case is optionally terminated by —R₇;
R₆ is, in each case, H, C₁₋₂₀ alkyl, C₂₋₂₀ alkenyl, C₀₋₂₀ alkyl-C₆₋₂₄ aryl, C₀₋₂₀ alkyl-C₅₋₂₄ heteroaromatic ring, C₃₋₂₄ non-aromatic ring optionally containing 1-3 heteroatoms selected from the group comprising O, N or S;
R₇ is, in each case, C₁₋₂₄ alkyl, C₂₋₂₄ alkenyl, C₆₋₂₄ aryl, C₅₋₂₄ heteroaromatic ring, C₃₋₂₀ non-aromatic ring optionally containing 1-3 heteroatoms selected from the group comprising O, N or S, —C(O)R₆, C(O)OR₆; and
X and Y are each independently Br, Cl, I, F, OH, OR₃ or NR₃R₄ and at least one of X and Y is NR₃R₄; or a pharmaceutically acceptable salt thereof.

15. A method according to claim 14, wherein at least one of R₁, R₃ and R₄ is other than H, and if R₃ and R₄ are both H and R₁ is —C(O)R₆; —C(O)OR₆, or —C(O)NHR₆ then R₆ is other than H.

16. A method according to claim 14, wherein R₂ is of the formula:

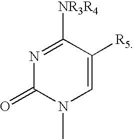

17. A method of treating a patient with cancer comprising:
determining that a compound enters cancer cells predominately by passive diffusion; and administering said compound to said patient; wherein said compound is a compound according to the formula: 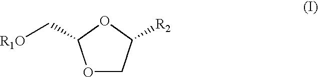
wherein:
R₁ is H; C₁₋₂₄ alkyl; C₂₋₂₄ alkenyl; C₆₋₂₄ aryl; C₅₋₂₄ heteroaromatic ring; C₃₋₂₄ non-aromatic ring optionally containing 1-3 heteroatoms selected from the group comprising O, N, or S; —C(O)R,; —C(O)OR₆; —C(O)NHRS; or an amino acid radical or dipeptide or tripeptide chain or mimetic thereof wherein the amino acid radicals are selected from the group comprising Glu, Gly, Ala, Val, Leu, Ile, Pro, Phe, Tyr, Trp, Ser, Thr, Cys, Met, Asn and Gln, and which in each case is optionally terminated by —R₇; R, can also be a P(O) (OR′)₂ group wherein R′ is in each case independently H, C₁₋₂₄ alkyl, C₂₋₂₄ alkenyl, C₆₋₂₄ aryl, C₇₋₂₄ arylmethyl, C₂₋₁₈ acyloxymethyl, C₃₋₈ alkoxycarbonyloxymethyl, C₃₋₈ S-acyl-2-thioethyl, saleginyl, t-butyl, phosphate or diphosphate;
R₁ can also be monophosphate, diphosphate, triphosphate or mimetics thereof;
R₂ is 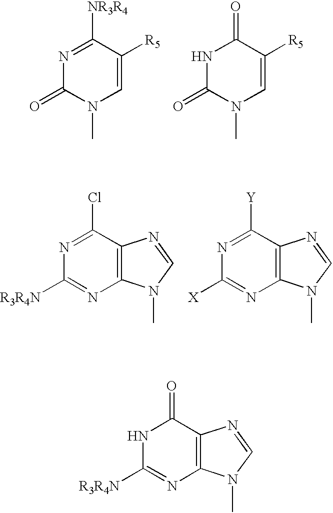
R₃ and R₄ are in each case independently H; C₂₋₂₄ alkyl; C₁₋₂₄ alkenyl; C₆₋₂₄ aryl; C₅₋₂₄ heteroaromatic ring; C₃₋₂₄ non-aromatic ring optionally containing 1-3 heteroatoms selected from the group comprising O, N, or S; —C(O)R₆; —C(O)OR₆; —C(O)NHR₆; or an amino acid radical or dipeptide or tripeptide chain or mimetic thereof wherein the amino acid radicals are selected from the group comprising Glu, Gly, Ala, Val, Leu, Ile, Pro, Phe, Tyr, Trp, Ser, Thr, Cys, Met, Asn and Gln, and which in each case is optionally terminated by —R₇;
R₆ is, in each case, H, C₁₋₂₄ alkyl, C₂₋₂₄ alkenyl, C⁰⁻²⁰ alkyl-C₆₋₂₄ aryl, C₀₋₂₀ alkyl-C₀₋₂₄ heteroaromatic ring, C₃₋₂₀ non-aromatic ring optionally containing 1-3 heteroatoms selected from the group comprising O, N or S;
R₇ is, in each case, C₁₋₂₄ alkyl, C₂₋₂₄ alkenyl, C₆₋₂₄ aryl, C₉₋₂₄ heteroaromatic ring, C₃₋₂₀ nonaromatic ring optionally containing 1-3 heteroatoms selected from the group comprising O, N or S, —C(O)R₆, —C(O)OR₆; and
X and Y are each independently Br, Cl, I, F, OH, OR₃ or NR₃R₄ and at least one of X and Y is NR₃R₄; or
a pharmaceutically acceptable salt thereof.

18. A method according to claim 17, wherein at least one of R₁, R₃ and R₄ is other than H, and if R₃ and R₄ are both H and R, is —C(O)R₆ or —C(O)OR₆, then R₆ is other than H.

19. A method according to claim 17, wherein R₂ is of the formula:

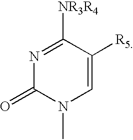

20. A method of treating a patient with cancer comprising:
administering to said patient a compound which has been determined to enter the cancer cells predominately by passive diffusion, wherein said compound is a compound according to the formula: 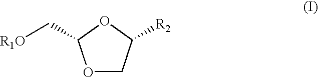
wherein:
R₁ is H; C₁₋₂₄ alkyl; C₂₋₂₄ alkenyl; C₆₋₂₄ aryl; C₅₋₂₄ heteroaromatic ring; C₃₋₂₄ non-aromatic ring optionally containing 1-3 heteroatoms selected from the group comprising O, N, or S; —C(O)R; —C(O)OR₆; —C(O)NHR₆; or an amino acid radical or dipeptide or tripeptide chain or mimetic thereof wherein the amino acid radicals are selected from the group comprising Glu, Gly, Ala, Val, Leu, Ile, Pro, Phe, Tyr, Trp, Ser, Thr, Cys, Met, Asn and G1n, and which in each case is optionally terminated by —R₇;
R₁ can also be a P(O)(OR′)₂ group wherein R′ is in each case independently H, C₁₋₂₄ alkyl, C₂₋₂₄ alkenyl, C₆₋₂₄ aryl, C₇₋₁₈ arylmethyl, C₂₋₁₈ acyloxymethyl, C₃₋₈ alkoxycarbonyloxymethyl, C₃₋₈ S-acyl-2-thioethyl, saleginyl, t-butyl, phosphate or diphosphate;
R₁ can also be monophosphate, diphosphate, triphosphate or mimetics thereof;
R₂ is 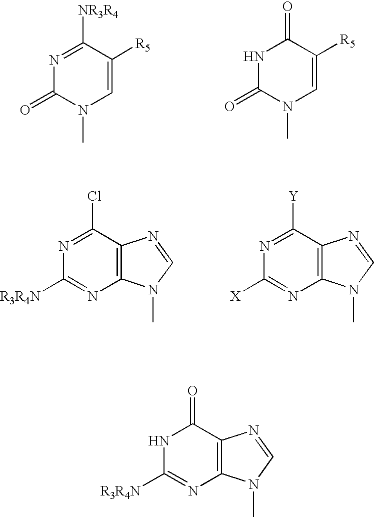
R₃ and R₄ are in each case independently H; C₁₋₂₄ alkyl; C₂₋₂₄ alkenyl; C₆₋₂₄ aryl; C₅₋₂₄ heteroaromatic ring; C₃₋₂₀ non-aromatic ring optionally containing 1-3 heteroatoms selected from the group comprising O, N, or S; —C(O)R₆; —C(O)OR₆; —C(O)NHR₆; or an amino acid radical or dipeptide or tripeptide chain or mimetic thereof wherein the amino acid radicals are selected from the group comprising Glu, Gly, Ala, Val, Leu, Ile, Pro, Phe, Tyr, Trp, Ser, Thr, Cys, Met, Asn and Gln, and which in each case is optionally terminated by —R₇;
R₆ is, in each case, H, C₁₋₂₄ alkyl, C₂₋₂₄ alkenyl, C₀₋₂₀ alkyl-C₆₋₂₄ aryl, C₀₋₂₀ alkyl-C₅₋₂₀ heteroaromatic ring, C₃₋₂₀ non-aromatic ring optionally containing 1-3 heteroatoms selected from the group comprising O, N or S;
R₇ is, in each case, C₁₋₂₄ alkyl, C₂₋₂₄ alkenyl, C₆₋₂₄ aryl, C₅₋₂₀ heteroaromatic ring, C₃₋₂₀ non-aromatic ring optionally containing 1-3 heteroatoms selected from the group comprising O, N or S, —C(O)R₆, —C(O)OR₆; and
X and Y are each independently Br, Cl, I, F, OH, OR₃ or NR₃R₄ and at least one of X and Y is NR₃R₄; or a
pharmaceutically acceptable salt thereof.

21. A method according to claim 20, wherein at least one of R₁, R₃ and R₄ is other than H, and if R₃ and R₄ are both H and R₁ is —C(O)R₆; —C(O)OR₆ or —C(O)NHR₆ then R₆ is other than H.

22. A method according to claim 20, wherein R₂ is of the formula:

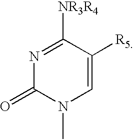

23. A method of treating a patient with cancer resistant to troxacitabine, comprising administering to said patient a troxacitabine derivative having a greater lipophilicity than troxacitabine.

24. A method according to claim 23, wherein said derivative is a compound of the following formula: 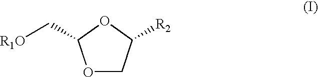
wherein:
R₁ is H; C₁₋₂₄ alkyl; C₂₋₂₄ alkenyl; C₆₋₂₄ aryl; C₅₋₂₄ heteroaromatic ring; C₃₋₂₀ non-aromatic ring optionally containing 1-3 heteroatoms selected from the group comprising O, N, or S; —C(O)R₆; —C(O)OR₆; —C(O)NHR₆; or an amino acid radical or dipeptide or tripeptide chain or mimetic thereof wherein the amino acid radicals are selected from the group comprising Glu, Gly, Ala, Val, Leu, Ile, Pro, Phe, Tyr, Trp, Ser, Thr, Cys, Met, Asn and Gln and the amino acid chain contains at least one amino acid other than Gly, and which in each case is optionally terminated by —R₇;
R₁ can also be a P(O) (OR′)₂ group wherein R′ is in each case independently H, C₁₋₂₄ alkyl, C₂₋₂₄ alkenyl, C₆₋₂₄ aryl, C₇₋₂₄ arylmethyl, C₂₋₁₇ acyloxymethyl, C₃₋₈ alkoxycarbonyloxymethyl, C₃₋₈ S-acyl-2-thioethyl, saleginyl, t-butyl, phosphate or diphosphate;
R₁ can also be monophosphate, diphosphate, triphosphate or mimetics thereof;
R₂ is 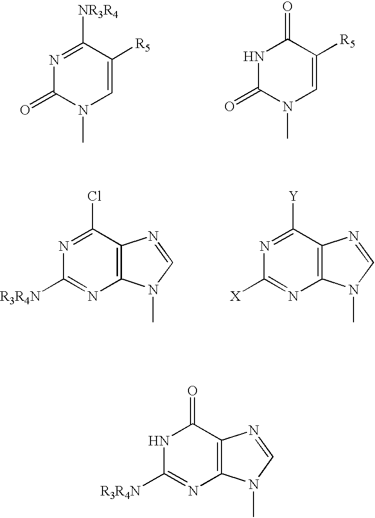
R₃ and R₄ are in each case independently H; C₁₋₂₀ alkyl; C₂₋₂₀ alkenyl; C₆₋₁₀ aryl; C₅₋₁₀ heteroaromatic ring; C₃₋₂₀ non-aromatic ring optionally containing 1-3 heteroatoms selected from the group comprising O, N, or S; —C(O)R₆; —C(O)OR₆; —C(O)NHR₆; or an amino acid radical or dipeptide or tripeptide chain or mimetic thereof wherein the amino acid radicals are selected from the group comprising Glu, Gly, Ala, Val, Leu, Ile, Pro, Phe, Tyr, Trp, Ser, Thr, Cys, Met, Asn and Gln and the amino acid chain contains at least one amino acid other than Gly, and which in each case is optionally terminated by —R₇;
R₆ is, in each case, H, C₁₋₂₀ alkyl, C₂₋₂₀ alkenyl, C₀₋₂₀ alkyl-C₆₋₁₀ aryl, C₀₋₂₀ alkyl-C₅₋₁₀ heteroaromatic ring, C₃₋₂₀ non-aromatic ring optionally containing 1-3 heteroatoms selected from the group comprising O, N or S;
R₇ is, in each case, C₁₋₂₀ alkyl, C₂₋₂₀ alkenyl, C₆₋₁₀ aryl, C₅₋₁₀ heteroaromatic ring, C₃₋₂₀ non-aromatic ring optionally containing 1-3 heteroatoms selected from the group comprising O, N or S, —C(O)R₆, —C(O)OR₆; and
X and Y are each independently Br, Cl, I, F, OH, OR₃ or NR₃R₄ and at least one of X and Y is NR₃R₄;
with the proviso that least one of R₁, R₃ and R₄ is other than H, and if R₃ and R₄ are both H and R, is —C(O)R₆, —C(O)OR₆ or —C(O)NHR₆, then R₆ is other than H; or
a pharmaceutically acceptable salt thereof.

25. A method according to claim 24, wherein R₂ is of the formula:

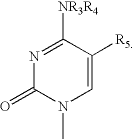

26. A method of treating a patient with cancer comprising:
determining that a compound does not enter cancer cells predominately by nucleoside or nucleobase transporter proteins; and administering said compound to said patient; wherein said compound is a compound according to the formula: 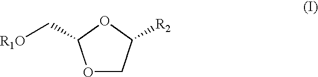
wherein:
R₁ is H; C₁₋₂₄ alkyl; C₂₋₂₄ alkenyl; C₆₋₂₄ aryl; C₅₋₂₀ heteroaromatic ring; C₃₋₂₀ non-aromatic ring optionally containing 1-3 heteroatoms selected from the group comprising O, N, or S; —C(O)R₆; —C(O)OR₆; —C(O)NHR₆; or an amino acid radical or dipeptide or tripeptide chain or mimetic thereof wherein the amino acid radicals are selected from the group comprising Glu, Gly, Ala, Val, Leu, Ile, Pro, Phe, Tyr, Trp, Ser, Thr, Cys, Met, Asn and Gln, and which in each case is optionally terminated by —R₇;
R₁ can also be a P(O) (OR′)₂ group wherein R′ is in each case independently H, C₁₋₂₄ alkyl, C₂₋₂₄ alkenyl, C₆₋₂₄ aryl, C₇₋₂₄ arylmethyl, C₂₋₁₇ acyloxymethyl, C₃₋₈ alkoxycarbonyloxymethyl, C₃₋₈ S-acyl-2-thioethyl, saleginyl, t-butyl, phosphate or diphosphate;
R₁ can also be monophosphate, diphosphate, triphosphate or mimetics thereof;
R₂ is 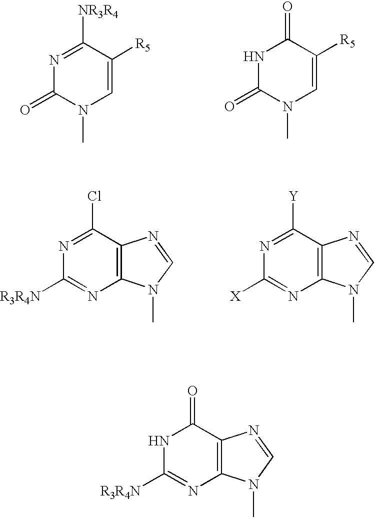
R₃ and R₄ are in each case independently H; C₁₋₂₄ alkyl; C₂₋₂₄ alkenyl; C₆₋₂₄ aryl; C₅₋₂₄ heteroaromatic ring; C₃₋₂₀ non-aromatic ring optionally containing 1-3 heteroatoms selected from the group comprising O, N, or S; —C(O)R₆; —C(O)OR₆; —C(O)NHR₆; or an amino acid radical or dipeptide or tripeptide chain or mimetic thereof wherein the amino acid radicals are selected from the group comprising Glu, Gly, Ala, Val, Leu, Ile, Pro, Phe, Tyr, Trp, Ser, Thr, Cys, Met, Asn and Gln, and which in each case is optionally terminated by —R₇;
R₆ is, in each case, H, C₁₋₂₄ alkyl, C₂₋₂₄ alkenyl, C₀₋₂₀ alkyl-C₆₋₂₄ aryl, C₀₋₂₀ alkyl-C₅₋₂₀ heteroaromatic ring, C₃₋₂₀ non-aromatic ring optionally containing 1-3 heteroatoms selected from the group comprising O, N or S;
R₇ is, in each case, C₁₋₂₄ alkyl, C₂₋₂₄ alkenyl, C₆₋₂₄ aryl, C₅₋₂₀ heteroaromatic ring, C₃₋₂₀ non-aromatic ring optionally containing 1-3 heteroatoms selected from the group comprising O, N or S, —C(O)R₆, —C(O)OR₆; and
X and Y are each independently Br, Cl, I, F, OH, OR₃ or NR₃R₄ and at least one of X and Y is NR₃R₄; or a
pharmaceutically acceptable salt thereof.

27. A method according to claim 26, wherein at least one of R₁, R₃ and R₄ is other than H, and if R₃ and R₄ are both H and R₁ is —C(O)R₆, —C(O)OR₆ or —C(O)NHR₆ then R₆ is other than H.

28. A method according to claim 27, wherein R₂ is of the formula:

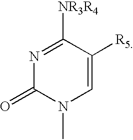

29. A method according to any one of claims 1-28, wherein said cancer is prostate cancer, colon cancer, lung cancer, melanoma, ovarian cancer, renal cancer, breast cancer, lymphoma, pancreatic cancer or bladder cancer.

30. A method according to any one of claims 3-28, wherein said cancer is leukemia.

31. A method according to any one of claims 1-28, wherein at least one of R₁, R₃, or R₄ is piperazinyl, piperidinyl, morpholinyl, pyrrolidinyl, adamantyl or quinuclidinyl.

32. A method according to any one of claims 1-28, wherein at least one of R₁, R₃ or R₄ is acetyl, propionyl, butyryl, valeryl, caprioic, caprylic, capric, lauric, myristic, palmitic, stearic, oleic, linoleic, or linolenic.

33. A method according to any one of claims 1-28, wherein at least one of R₁, R₃ or R₄ is cyclopropyl, cyclobutyl, cyclopentyl, cyclohexyl, phenyl, napthyl or biphenyl.

34. A method according to any one of claims 1-28, wherein at least one of R₁, R₃ or R₄ contains a heterocyclic group selected from the following group:
furyl, thiophenyl, pyrrolyl, imidazolyl, pyrazoyl, oxazolyl, isoxazolyl, thiazolyl, isothiazolyl, pyridyl, pyrimidinyl, triazolyl, tetrazolyl, oxadrazolyl, thiadiazolyl, thiopyranyl, pyrazinyl, benzofuryl, benzothiophenyl, indolyl, benzimidazolyl, benzopyrazolyl, benzoxazolyl, benzisoxazolyl, benzothiozolyl, benzisothiazolyl, benzoxadiazolyl, quinolinyl, isoquinolinyl, carbazolyl, acridinyl, cinnolinyl and quinazolinyl.

35. A method according to any one of claims 1-28, wherein said compound is administered at least daily for a period of 2 to 10 days every 2 to 5 weeks.

36. A method according to any one of claims 1-28, wherein said compound is administered at least daily for a period of 2 to 10 days every 3 to 4 weeks.

37. A method according to any one of claims 1-28, wherein said compound is administered at least daily for 3 to 7 days every 2 to 5 weeks.

38. A method according to any one of claims 1-28, wherein said compound is administered at least daily 4 to 6 days every 2 to 5 weeks.

39. A compound having the following formula: 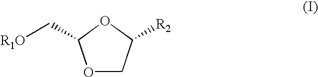
wherein:
R₁ is H; C₁₋₂₀ alkyl; C₂₋₂₀ alkenyl; C₆₋₁₀ aryl; C₅₋₁₀ heteroaromatic ring; C₃₋₂₀ non-aromatic ring optionally containing 1-3 heteroatoms selected from the group comprising O, N, or S; —C(O)R₆;
—C(O)OR₆; —C(O)NRH₆; or an amino acid radical or dipeptide or tripeptide chain wherein the amino acid radicals are selected from the group comprising Glu, Gly, Ala, Val, Leu, Ile, Pro, Phe, Tyr, Trp, Ser, Thr, Met, Cys, Asn and Gln, and which in each case is optionally terminated by —R₇;
R₁ can also be a P(O) (OR′)₂ group wherein R′ is in each case independently H, C₁₋₂₀ alkyl, C₂₋₂₀ alkenyl, C₆₋₁₀ aryl, C₇₋₁₁ arylmethyl, C₂₋₇ acyloxymethyl, C₃₋₈ alkoxycarbonyloxymethyl, C₃₋₆ S-acyl-2-thioethyl, saleginyl, t-butyl, phosphate or diphosphate;
R₁ can also be monophosphate, diphosphate, triphosphate or mimetics thereof;
R₂ is 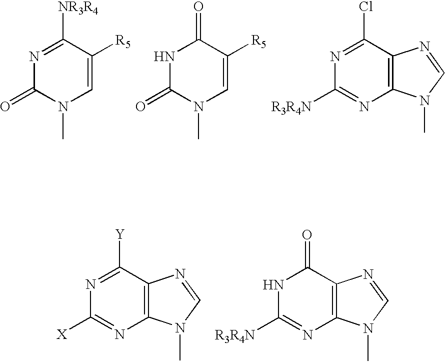
R₃ and R₄ are in each case Independently H; C₁₋₂₀ alkyl; C₂₋₂₀ alkenyl; C₆₋₁₀ aryl; C₅₋₁₀ heteroaromatic ring; C₃₋₂₀ non-aromatic ring optionally containing 1-3 heteroatoms selected from the group comprising O, N, or S; —C(O)R₆; —C(O)OR₆; —C(O)NRH₆; or an amino acid radical or dipeptide or tripeptide chain or mimetic thereof wherein the amino acid radicals are selected from the group comprising Glu, Gly, Ala, Val, Leu, Ile, Pro, Phe, Tyr, Trp, Ser, Thr, Cys, Met, Asn and Gln, and which in each case is optionally terminated by —R₇;
R₆ is, in each case, H, C₁₋₂₀ alkyl, C₂₋₂₀ alkenyl, C₀₋₂₀ alkyl-C₆₋₁₀ aryl, C₀₋₂₀ alkyl-C₉₋₁₀ heteroaromatic ring, C₃₋₂₀ non-aromatic ring optionally containing 1-3 heteroatoms selected from the group comprising O, N or S;
R₇ is, in each case, C₁₋₂₀ alkyl, C₂₋₂₀ alkenyl, C₆₋₁₀ aryl, C₅₋₁₀ heteroaromatic ring, C₃₋₂₀ nonaromatic ring optionally containing 1-3 heteroatoms selected from the group comprising O, N or S, —C(O)R₆, —C(O)OR₆; and
X and Y are each independently Br, Cl, I, F, OH, OR₃ or NR₃R₄ and at least one of X and Y is NR₃R₄; or a pharmaceutically acceptable salt thereof;
with the proviso that at least one of R₁, R₃ and R, is
C₇₋₂₀ alkyl;
C₇₋₂₀ alkenyl;
C₆₋₁₀ aryl;
C₅₋₁₀ heteroaromatic ring;
C₄₋₂₀ non-aromatic ring optionally containing 1-3 heteroatoms selected from the group comprising O, N, or S;
C(O)R₆ in which R₆ is, C₇₋₂₀ alkyl, C₇₋₂₀ alkenyl, C₀₋₂₀ alkyl-C₆₋₁₀ aryl, C₀₋₂₀ alkyl-C₅₋₁₀ heteroaromatic ring, C₄₋₂₀ non-aromatic ring optionally containing 1-3 heteroatoms selected from the group comprising O, N or S;
—C(O)OR₆ in which R₆ is C₇₋₂₀ alkyl, C₇₋₂₀ alkenyl, C₀₋₂₀ alkyl-C₆₋₁₀ aryl, C₀₋₂₀ alkyl-C₅₋₁₀ heteroaromatic ring, C₄₋₂₀ non-aromatic ring optionally containing 1-3 heteroatoms selected from the group comprising O, N or S; or
a dipeptide or tripeptide or mimetic thereof where the amino acid radicals are selected from the group comprising Glu, Gly, Ala, Val, Leu, Ile, Pro, Phe, Tyr, Trp, Ser, Thr, Cys, Met, Asn and Gln, and which is optionally terminated by —R₇.

40. A method of treating a patient with cancer comprising administering to said patient a prodrug form of troxacitabine, having a lipophilic structure to enhance entry of the prodrug into the cancer cells by passive diffusion, wherein said lipophilic structure is cleavable by cellular enzymes, thereby increasing the amount of troxacitabine within the cancer cells to a level greater than that allowable by administration of troxacitabine in nonprodrug form.

41. A method of treating a patient having cancer which is resistant to gemcitabine, cytarabine or both, comprising administering to said patient a troxacitabine derivative having a lipophilic structure which enhances the entry of the derivative into the cancer cell by the passive diffusion.

42. A method of treating a patient having cancer wherein the cancer cells are deficient in nucleoside or nucleobase transporter proteins, comprising administering to said patient a troxacitabine derivative having a lipophilic structure which enhances entry of the derivative into the cancer cells by passive diffusion.

43. A method according to claim 4, wherein said cancer cells are deficient in one or more nucleobase transporter proteins.

44. A method according to any one of claims 1-28, wherein the compound is of the formulas

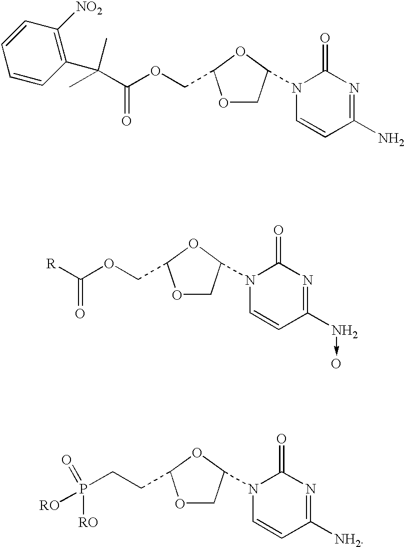

45. A method according to any one of claims 1 to 28 wherein the compound is of the formula

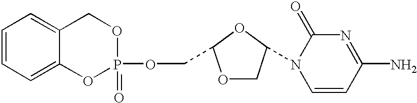

46. A method according to any one of claims 1 to 28, wherein the compound is of the formula

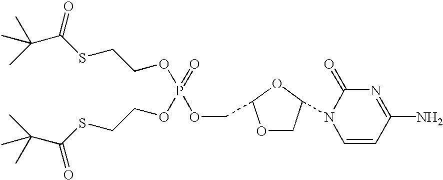

46. A method according to any one of claims 1 to 28, wherein the compound is selected from
4-HEXYL-BENZOIC ACID 4-(4-AMINO-2-OXO-2H-PYRIMIDIN-1-YL)-[1,3]DIOXOLAN-2-YLMETHYL ESTER (No. 191)
8-PHENYL-OCTANOIC ACID [1-(2-HYDROXYMETHYL-[1,3]DIOXOLAN-4-YL)-2-OXO-1,2-DIHYDRO-PYRIMIDIN-4-YL]-AMIDE (No. 197);
8-PHENYL-OCTANOIC ACID 4-(4-AMINO-2-OXO-2H-PYRIMIDIN-1-YL)-[1,3]DIOXOLAN-2-YLMETHYL ESTER (No. 198);
4-PENTYL-BICYCLO[2.2.2]OCTANE-1-CARBOXYLIC ACID 4-(4-AMINO-2-OXO-2H-PYRIMIDIN-1-YL)-[1,3]DIOXOLAN-2-YLMETHYL ESTER (No. 211);
4-PENTYL-CYCLOHEXANECARBOXYLIC ACID 4-(4-AMINO-2-OXO-2H-PYRIMIDIN-1-YL)-[1,3]DIOXOLAN-2-YLMETHYL ESTER (No. 240) or mixtures thereof.
